(19) United States
(12) Patent Application Publication
(10) Pub. No.: US 2025/0306045 A1
(43) Pub. Date: Oct. 2, 2025

(54) Fully Automatic Instrument System for Biochemical Assays

(71) Applicant: Alamar Biosciences, Inc., Fremont, CA (US)

(72) Inventors: Yuling Luo, San Ramon, CA (US); Shiping Chen, Fremont, CA (US); Cheng Peng, Dublin, CA (US); Kaiyuan Zhang, Fremont, CA (US); Taber Smith, Saratoga, CA (US); Kenneth Miller, Aptos, CA (US); Faisal Maniar, San Jose, CA (US); Christina Kozlovsky, Sunnyvale, CA (US); Bruce denDulk, Ripon, CA (US); Wayne Hopp, Walnut Creek, CA (US)

(21) Appl. No.: 19/183,502

(22) Filed: Apr. 18, 2025

Related U.S. Application Data
(63) Continuation of application No. 18/661,031, filed on May 10, 2024.
(60) Provisional application No. 63/501,355, filed on May 10, 2023.

(51) Int. Cl. G01N 35/00; G01N 35/04

(52) U.S. Cl. CPC G01N 35/00584; G01N 35/00722; G01N 35/00732; G01N 35/0099; G01N 35/04; G01N 2035/00564; G01N 2035/00752; G01N 2035/0091; G01N 2035/0436

(57) ABSTRACT

Disclosed herein is an instrument and associated methods for a fully automated bench-top NULISA platform, comprising an X-Y-Z-gantry, a microtiter plate stage, an incubator, a quantitative PCR module, a decontamination cleaner for microtiter plates, a microtiter plate sealer, a magnetic probe and comb assembly for sample mixing and magnetic bead extraction, a storage unit for reagents and supplies, and a controller.

### CROSS-REFERENCE TO RELATED APPLICATIONS

This application is a continuation of U.S. application Ser. No. 18/661,031, filed May 10, 2024, which further claims the benefit of priority from U.S. Provisional Application No. 63/501,355, filed May 10, 2023. The foregoing related applications, in their entirety, are incorporated by reference herein.

In addition, all publications and patent applications mentioned in this specification are also herein incorporated by reference.

### 1. BACKGROUND

A biochemical assay deploys a set of reagents and follows a defined procedure to detect one or multiple target analytes in a biological sample. It is highly desirable to execute an assay run automatically using an instrument system. Most such instruments on the market are designed to run a specific assay. Alternatively, a user may have to identify and install multiple off-the-shelf instruments, each of which performs a particular process of the assay, such as liquid transfer, plate washing etc., and use a robotic arm to integrate them into a “work cell”. Such systems are usually custom designed and integrated by an end user and are typically bulky, unreliable and require intense effort in up-keeping.

To improve operating efficiency, it is common to run an assay to multiple samples in parallel on a carrier plate with multiple vessels. The standard microtiter plates with 24, 96, 384 etc. well format are the most widely used assay plates. For a complex assay with many steps, a set of assay plates are usually deployed in an assay run.

This invention discloses a compact instrument system designed to execute assays based on NULISA technology (WO2021113290). Because it includes functional modules to execute critical procedures of most biochemical assays, it can be reconfigured to run a wide range of assays on microtiter plates in a fully automated fashion.

An immunoassay is a bioanalytical test that follows a defined procedure to detect one or multiple target analytes in a biological sample. For example, enzyme-linked immunosorbent assay (ELISA) is a technique used to detect and quantify the presence of specific analytes such as proteins, peptides, antibodies, and antigens. However, the modest performance of current immunoassays for detection of biological analytes, such as proteins, is still a technical bottleneck for many applications because of limits in sensitivity.

In a conventional ELISA, plates or wells coated with capture antibodies are used to immobilize target antigens from the sample. In magnetic bead-based ELISA, the capture antibodies may be attached to magnetic beads.

Using magnetic beads may enhance sensitivity because magnetic beads may allow for a higher binding capacity. Magnetic beads may also save the end user time because magnetic bead-based ELISA may often require shorter incubation times. Magnetic beads are also automation-friendly, so assays using them may be well-suited for high-throughput applications.

Another method to achieve higher sensitivity in immunoassays is the use of a sandwich pair of antibodies. But other immunoassays such as immuno-PCR, proximity ligation assay (PLA), proximity extension assay (PEA), single molecular array (SIMOA), and single molecule counting (SMC), remain inadequate to analyze the low abundance portion of a proteome because they have limited sensitivity.

Nucleic acid-linked immune-sandwich assay (NULISA) improved on prior immunoassays by utilizing multiple mechanisms of background suppression, such as a sequential capture/release mechanism with paramagnetic beads, as described in U.S. application Ser. No. 17/330,331, the entire content of which is incorporated by reference in its entirety.

Further, immunoassays typically involve many manual steps that are time-consuming and have the potential for costly errors, such as contamination or human error. Automation of immunoassay procedures by instruments may improve operating efficiency and may minimize contamination, impurities, and errors. To keep up with increasing testing demands, there is a push to semi-automate or fully automate immunoassay procedures.

Semi-automation of a multi-step immunoassay involves a combination of manual operations and automated instruments that may use a large quantity of consumables (e.g., plates, combs, pipette tips, reagents) to minimize impurities and may achieve high sensitivity of low concentration analytes. For example, ThermoFisher's KingFisher Flex system is an automated extraction instrument that may be used for extraction and purification of nucleic acids and proteins after manual preparation of samples and plates. While semi-automation may eliminate some time-consuming manual steps, there remain key manual steps (e.g., manual transfer of plates or manual initiation of the next automated instrument) that ultimately may limit efficiency of a procedure.

Alternatively, automated workstations may further improve operating efficiency and sensitivity by limiting manual operations almost entirely. In some cases, automated workstations for immunoassays have been attempted using multiple dedicated instruments, each of which may perform a particular process, such as liquid transfer, plate washing, etc., and may use a robotic arm to integrate them into a workstation. For example, Beckman Coulter Biomek workstations are single pod workstations that allow for interchangeable tools. These automated workstations are commonly custom configured and integrated by the end user, and are typically bulky and require intense up-keep. Operation and implementation of a specific procedure on these custom designed workstations is another significant drawback for end users.

The large footprints of these automated workstations also pose a concern for lab space setup. For example, the SIMOA HD-1 Analyzer is a floor-standing instrument that takes up a sizeable workspace with dimensions of 55.7×35.3×63.5 inches. Meanwhile, bench-top workstations are compact, portable, and designed to be placed on a lab bench, and therefore may be suitable for both smaller and larger laboratory settings.

This invention discloses a compact instrument system designed to execute a multi-step, highly sensitive immunoassay involving sequential capture/release mechanism based on NULISA technology (U.S. application Ser. No. 17/330,331).

### 2. BRIEF SUMMARY OF INVENTION

Embodiment 1. A compact, fully-automated instrument for executing a dual-capture and release multiplexed immunoassay, comprising:
A) a bench-top housing, comprising:
   1) a controller;
   2) a robotic gantry capable of moving in three degrees of freedom;
   3) a hotel capable of holding multi-vessel carrier plates that are accessible by a user and the robotic gantry;
   4) a controlled stand that provides for a plurality of multi-vessel carrier plate positions, wherein the stand is movable along the Y-axis (from front-to-back within the instrument housing);
   5) a mixer that operates as a magnetic bead processor and mixer comprising multiple multi-vessel carrier plate platforms vertically positioned on top of each other;
   6) a washer for washing multi-vessel carrier plates; and
7) a reader.

Embodiment 2. A compact instrument designed to execute a multi-step, highly sensitive immunoassay involving a sequential capture/release process, comprising:
A) a bench-top housing with a touchscreen display, comprising:
   1) a controller;
   2) a robotic gantry capable of moving in three degrees of freedom;
   3) a hotel capable of holding multi-vessel carrier plates that are accessible by a user and the robotic gantry;
   4) a controlled stand that provides for a plurality of multi-vessel carrier plate positions, wherein the stand is movable along the Y-axis (from front-to-back within the instrument housing);
   5) a mixer that operates as a magnetic bead processor and mixer comprising multiple multi-vessel carrier plate platforms vertically positioned on top of each other;
   6) a washer for washing multi-vessel carrier plates;
   7) an incusealer for incubating and sealing at least one multi-vessel carrier plate; and
   8) a reader.

Embodiment 3. A fully automated, high-throughput precision proteomics instrument for ultra-high sensitivity analysis across a range of multiplex levels to support broad biomarker profiling and translation of validated biomarkers, comprising:
A) a bench-top housing with a touchscreen display, a user-accessible compartment for bulk reagents, and a compartment for user-accessible set of bays, the bench-top housing comprising:
   1) a controller;
   2) a controlled robotic gantry equipped with an end-effector, comprising pipettors, a multi-vessel carrier plates gripper, a laser position sensor, and a barcode scanner, the robotic gantry capable of moving the end-effector in three degrees of freedom in X, Y and Z axis;
   3) a hotel comprising a set of multi-vessel carrier plate bays for receiving and holding at least one universal reagent cartridge, and a consumables carrier;
   4) a controlled stand that provides for a plurality of multi-vessel carrier plate positions, wherein the stand is movable along the Y-axis (from front-to-back within the instrument housing);
   5) a controlled mixer that operates as a magnetic bead processor and mixer comprising multiple multi-vessel carrier plate platforms vertically positioned on top of each other;
   6) a controlled washer for washing multi-vessel carrier plates;
   7) an incusealer for incubating and sealing at least one multi-vessel carrier plate;
   8) a bulk fluid station comprising a plurality of containers; and
   9) a controlled reader.

Embodiment 4. A bench-top instrument for automatically conducting in parallel a plurality of multiplexing oligonucleotide-conjugated antibody proximity ligation assays on a plurality of biological samples, comprising:
A) a bench-top housing with a touchscreen display, a user-accessible compartment for bulk reagents, and a compartment for user-accessible set of bays, the bench-top housing comprising:
   1) a controller;
   2) a robotic gantry equipped with an end-effector, comprising pipettors, a multi-vessel carrier plates gripper, a laser position sensor, and a barcode scanner, the robotic gantry capable of moving the end-effector in three degrees of freedom in X, Y and Z axis;
   3) a hotel comprising a set of multi-vessel carrier plate bays for receiving and holding at least one universal reagent cartridge, and a consumables carrier;
   4) a stand that provides for a plurality of multi-vessel carrier plate positions, wherein the stand is movable along the Y-axis (from front-to-back within the instrument housing);
   5) a mixer that operates as a magnetic bead processor and mixer comprising multiple multi-vessel carrier plate platforms vertically positioned on top of each other;
   6) a washer for washing multi-vessel carrier plates;
   7) a reader.

Embodiment 5. In some embodiments, for example, including the embodiments of 1-4 and any of the other embodiments herein, wherein at least one of the multi-vessel carrier plates comprises a set of paired-binding moieties that are pre-selected to bind specific analytes, wherein the pair-binding moieties comprise:
(i) a first moiety comprising a first antibody or a first antibody fragment that is pre-selected to bind a specific analyte, a first nucleic acid target label comprising a first identity that is analyte-specific to the specific analyte, and a first nucleic tag; and
(ii) a second moiety comprising a second antibody or a second antibody fragment that is pre-selected to bind the same specific analyte as the first antibody or antibody fragment of the first moiety of the paired-binding moieties, a second nucleic acid target label comprising a second identity that is analyte-specific to the specific analyte, and a second nucleic acid tag.

Embodiment 6. A bench-top automated instrument for conducting a biochemical assay on a biological sample comprising a plurality of analytes, comprising:
a) a controller;
b) a controlled gantry;
c) a controlled stand;
d) a set of 5 multi-vessel carrier plates;
e) a controlled quantitative PCR unit;
f) a controlled plate washer;
g) a controlled plate sealer;
h) a controlled plate incubator;
i) a controlled mixer comprising a paramagnetic bead extractor comprising a magnetic pole and comb sub-assembly;
j) a gantry accessible hotel comprising:
   1) a plurality of first moieties comprising first antibodies or capture antibody fragments conjugated with a first nucleic acid tag that is hybridized with a first nucleic acid target label and is not covalently attached to the first antibody or first antibody fragment;
   2) a plurality of second moieties comprising second antibodies or second antibody fragments conjugated with a second nucleic acid target label that is hybridized with a second nucleic acid tag and is not covalently attached to the second antibody or second antibody fragment;
   3) a first set of first substrate;
   4) a second set of second substrate;
   5) a set of buffers;
   6) a plurality of double-stranded nucleic acid sample labels that comprise a 5′ overhang and 3′ overhang;
   7) a frame comprising a sealing film; and
k) the controller comprising a processor and a non-transitory machine-readable storage medium comprising instructions executable by the processor to provide controlled operations of the unit operations within the instrument, the operations controlled by the controller comprise:
   1) forming a first solution in a first multi-vessel carrier plate by mixing:
      i) a portion of at least one sample,
      ii) a portion of the plurality of first moieties, and
      iii) a portion of the plurality of second moieties;
   2) incubating the first solution to form immunocomplexes comprising one of the first moieties from the portion of the plurality of first moieties, one of the second moieties from the portion of the plurality of second moieties and with the analyte in the at least one sample;
   3) performing a sequential capture and release mechanism of the immunocomplexes to remove contaminants and improve sensitivity by introducing:
      i) a portion of the first set of first substrate to capture at least a portion of the immunocomplexes from the 1st solution in the 1st multi-vessel carrier plate wherein the 1st solution in the 1st multi-vessel carrier plate is incubated, and the first set of paramagnetic beads are transferred by the mixer to a 2nd multi-vessel carrier plate to form a 2nd solution to release at least a portion of the immunocomplexes, and the first set of first substrate are removed by the mixer,
      ii) a portion of the second set of second substrate to capture at least a portion of the immunocomplexes from the 2nd solution in the 2nd multi-vessel carrier plate wherein the 2nd solution is incubated, and the second set of second substrate are removed by the mixer to form a 3rd solution in a 3rd multi-vessel plate,
   4) introducing at least a portion of the plurality of double-stranded nucleic acid sample labels into the 3rd solution to form at least one multiplexed analyte-specific reporter comprising the first nucleic acid target label associated with one of the first antibodies in at least one of the immunocomplexes and at least one of the second nucleic acid target labels conjugated to one of the second antibodies in the at least one of the immunocomplexes, and a sample-specific identity barcode, by hybridizing one of the overhangs with the first nucleic acid target label associated with one of the first antibodies of the at least a portion of the immunocomplexes and hybridizing the other overhang with the second nucleic acid target label conjugated to one of the second antibodies of the at least a portion of the immunocomplexes to complete proximity-based ligation between the first nucleic acid target label and the nucleic acid sample label and the second nucleic acid target label and the nucleic acid sample label to generate the at least one multiplexed analyte-specific reporter that is conjugated to the second antibodies or second antibody fragments and is not covalently attached to the first antibodies or first antibody fragments;
   5) collecting the at least one multiplexed analyte-specific reporters in a 4th multi-vessel carrier plate and then pooling the multiplexed analyte-specific reporter to form a pooled sample for detection of the analyte by NGS at attomolar level of sensitivity.

Embodiment 7. A bench-top automated instrument for conducting a biochemical assay on a biological sample comprising a plurality of analytes, comprising:
a) a controller;
b) a controlled gantry;
c) a controlled stand;
d) a set of 5 multi-vessel carrier plates;
e) a controlled quantitative PCR unit;
f) a controlled plate washer;
g) a controlled plate sealer;
h) a controlled plate incubator;
i) a controlled mixer comprising a paramagnetic bead extractor comprising a magnetic pole and comb sub-assembly;
j) a gantry accessible hotel comprising:
   1) a plurality of first moieties comprising first antibodies or capture antibody fragments conjugated with a first nucleic acid tag that is hybridized with a first nucleic acid target label and is not covalently attached to the first antibody or first antibody fragment;
   2) a plurality of second moieties comprising second antibodies or second antibody fragments conjugated with a second nucleic acid target label that is hybridized with a second nucleic acid tag and is not covalently attached to the second antibody or second antibody fragment;
   3) a first set of first substrate;
   4) a second set of second substrate;
   5) a set of buffers;
   6) a plurality of double-stranded nucleic acid sample labels that comprise a 5′ overhang and 3′ overhang;
   7) a frame comprising a sealing film; and
k) the controller for controlling the unit operations of the instrument, the controller comprising a non-transitory computer-readable storage medium having an executable file therein, the executable file executable by the instrument controller to cause the unit operations of the instrument to perform operations, the instrument operations comprising:
   1) forming a first solution in a first multi-vessel carrier plate by mixing:
      i) a portion of at least one sample,
      ii) a portion of the plurality of first moieties, and
      iii) a portion of the plurality of second moieties;
   2) incubating the first solution to form immunocomplexes comprising one of the first moieties from the portion of the plurality of first moieties, one of the second moieties from the portion of the plurality of second moieties and with the analyte in the at least one sample;
   3) performing a sequential capture and release mechanism of the immunocomplexes to remove contaminants and improve sensitivity by introducing:
      i) a portion of the first set of first substrate to capture at least a portion of the immunocomplexes from the 1st solution in the 1st multi-vessel carrier plate wherein the 1st solution in the 1st multi-vessel carrier plate is incubated, and the first set of paramagnetic beads are transferred by the mixer to a 2nd multi-vessel carrier plate to form a 2nd solution to release at least a portion of the immunocomplexes, and the first set of first substrate are removed by the mixer,
      ii) a portion of the second set of second substrate to capture at least a portion of the immunocomplexes from the 2nd solution in the 2nd multi-vessel carrier plate wherein the 2nd solution is incubated, and the second set of second substrate are removed by the mixer to form a 3rd solution in a 3rd multi-vessel carrier plate,
   4) introducing at least a portion of the plurality of double-stranded nucleic acid sample labels into the 3rd solution to form at least one multiplexed analyte-specific reporter comprising the first nucleic acid target label associated with one of the first antibodies in at least one of the immunocomplexes and at least one of the second nucleic acid target labels conjugated to one of the second antibodies in the at least one of the immunocomplexes, and a sample-specific identity barcode, by hybridizing one of the overhangs with the first nucleic acid target label associated with one of the first antibodies of the at least a portion of the immunocomplexes and hybridizing the other overhang with the second nucleic acid target label conjugated to one of the second antibodies of the at least a portion of the immunocomplexes to complete proximity-based ligation between the first nucleic acid target label and the nucleic acid sample label and the second nucleic acid target label and the nucleic acid sample label to generate the at least one multiplexed analyte-specific reporter that is conjugated to the second antibodies or second antibody fragments and is not covalently attached to the first antibodies or first antibody fragments;
   5) collecting the at least one multiplexed analyte-specific reporters in a 4th multi-vessel carrier plate and then pooling the multiplexed analyte-specific reporter to form a pooled sample for detection of the analyte by NGS at attomolar level of sensitivity.

Embodiment 8. A bench-top automated instrument for conducting a biochemical assay to detect an analyte in at least a first biological sample and a second biological sample, comprising:
a) a gantry;
b) a stand;
c) a plurality of multi-vessel carrier plates, comprising a first plate, a second plate, a third plate, and a fourth plate;
d) a quantitative PCR unit;
e) a plate washer;
f) a plate sealer;
g) a plate incubator;
h) a mixer comprising a paramagnetic bead extractor comprising a magnetic pole and comb sub-assembly;
i) a hotel comprising:
   1) a plurality of first moieties comprising first antibodies or capture antibody fragments conjugated with a first nucleic acid tag that is hybridized with a first nucleic acid target label and is not covalently attached to the first antibody or first antibody fragment;
   2) a plurality of second moieties comprising second antibodies or second antibody fragments conjugated with a second nucleic acid target label that is hybridized with a second nucleic acid tag and is not covalently attached to the second antibody or second antibody fragment;
   3) a first set of first substrate;
   4) a second set of second substrate;
   5) a set of buffers;
   6) a plurality of double-stranded nucleic acid sample labels that comprise a 5′ overhang and 3′ overhang;
   7) a frame comprising a sealing film; and
j) a controller comprising a processor and a non-transitory machine-readable storage medium comprising instructions executable by the processor to provide controlled operations of the unit operations within the instrument, the operations controlled by the controller comprise:
   1) forming a first solution in the first multi-vessel carrier plate for each of a first assay reaction comprising the first biological sample and a second assay reaction comprising the second biological sample, by mixing:
      i) a portion of at least one sample,
      ii) a portion of the plurality of first moieties, and
      iii) a portion of the plurality of second moieties;
   2) incubating the first solution of the first assay reaction and the second assay reaction to form immunocomplexes with the analyte;
   3) performing a dual capture and release mechanism of the immunocomplexes in each of the first reaction and the second assay reaction to remove contaminants and improve sensitivity by introducing:
      i) a portion of the first set of first substrate to each of the first assay reaction and the second assay reaction, to capture at least a portion of the immunocomplexes from the 1st solution in the 1st multi-vessel carrier plate wherein the 1st solution in the 1st multi-vessel carrier plate is incubated, and the first set of paramagnetic beads are transferred by the mixer to a 2nd multi-vessel carrier plate to form a 2nd solution to release at least a portion of the immunocomplexes, and the first set of first substrate are removed by the mixer,
      ii) a portion of the second set of second substrate to each of the first assay reaction and the second assay reaction to capture at least a portion of the immunocomplexes from the 2nd solution in the 2nd multi-vessel carrier plate wherein the 2nd solution is incubated, and the second set of second substrate are removed by the mixer to form a 3rd solution in a 3rd multi-vessel carrier plate,
   4) introducing at least a portion of the plurality of double-stranded nucleic acid sample labels into the 3rd solution to each of the first assay reaction and the second assay reaction, to form at least one multiplexed analyte-specific reporter comprising the first nucleic acid target label associated with one of the first antibodies in at least one of the immunocomplexes and at least one of the second nucleic acid target labels conjugated to one of the second antibodies in the at least one of the immunocomplexes, and a sample-specific identity barcode, by hybridizing one of the overhangs with the first nucleic acid target label associated with one of the first antibodies of the at least a portion of the immunocomplexes and hybridizing the other overhang with the second nucleic acid target label conjugated to one of the second antibodies of the at least a portion of the immunocomplexes to complete proximity-based ligation between the first nucleic acid target label and the nucleic acid sample label and the second nucleic acid target label and the nucleic acid sample label to generate the at least one multiplexed analyte-specific reporter that is conjugated to the second antibodies or second antibody fragments and is not covalently attached to the first antibodies or first antibody fragments;
   5) collecting the at least one multiplexed analyte-specific reporters in a 4th multi-vessel carrier plate and then pooling the multiplexed analyte-specific reporters from the first assay reaction with the multiplexed analyte-specific reports from the second assay reaction to form a pooled sample for detection of the analyte by NGS at attomolar level of sensitivity.

Embodiment 9. A method of automatically conducting in parallel a plurality of multiplexing oligonucleotide-conjugated antibody proximity ligation assays on a plurality of biological samples, comprising:
A) introducing in parallel in a first plate a portion of a plurality of multiplexed paired-binding moieties into a plurality of biological samples to form a plurality of immunocomplex forming solutions, wherein the multiplexed paired-binding moieties of the plurality of multiplexed paired-binding moieties, comprises different paired-binding moieties that are pre-selected to bind different specific analytes, the paired-binding moieties comprising:
   i) a first moiety of the paired-binding moieties comprising a first antibody or a first antibody fragment that is pre-selected to bind a specific analyte, a first nucleic acid target label comprising a first identity that is analyte-specific to the specific analyte, and a first nucleic tag; and
   ii) a second moiety of the paired-binding moieties comprising a second antibody or a second antibody fragment that is pre-selected to bind the same specific analyte as the first antibody or antibody fragment of the first moiety of the paired-binding moieties, a second nucleic acid target label comprising a second identity that is analyte-specific to the specific analyte, and a second nucleic acid tag; wherein the first nucleic acid tag of the first moiety of the paired-binding moieties is pre selected to bind to a first substrate that is the same for all the different paired-binding moieties in the multiplex and the second nucleic acid tag of the second moiety of the paired-binding moieties is pre-selected to bind a second substrate that is the same for all the different paired-binding moieties in the multiplex;
B) incubating in parallel the plurality of immunocomplex forming solutions to form a plurality of multiplexed immunocomplexes;
C) a first combining a portion of a first substrate solution comprising a plurality of the first substrates with the plurality of immunocomplex forming solutions;
D) a first enabling the first nucleic acid tag of the first moiety of the paired-binding moieties to bind to a portion of the first substrates;
E) a first extracting in parallel, via the portion of the plurality of first substrates, the plurality of multiplexed immunocomplexes from the plurality of immunocomplex forming solutions to form in a second plate a plurality of first immunocomplex purification solutions;
F) eluting in parallel the plurality of first substrates from the plurality of multiplexed immunocomplexes in the plurality of first immunocomplex purification solutions;
G) removing in parallel the plurality of first substrates from the plurality of first immunocomplex purification solutions;
H) a second combining in parallel a portion of a second substrate solution comprising a plurality of the second substrates with the plurality of first immunocomplex purification solutions;
I) a second enabling the second nucleic acid tag of the second moiety of the paired binding moieties to bind to a portion of the second substrates,
J) a second extracting in parallel, via the portion of the plurality of second substrates, the plurality of multiplexed immunocomplexes from the plurality of first immunocomplex purification solutions to form in a third plate a plurality of second immunocomplex purification solutions;
K) ligating in parallel a plurality of the first nucleic acid target label from the first moiety of the paired binding moieties (directly or indirectly) with the second nucleic acid target label from the second moiety of the paired binding moieties to form a plurality of multiplexed analyte-specific reporters;
L) a third extracting in parallel, via the portion of the plurality of second substrates, the plurality of multiplexed immunocomplexes (or the plurality of multiplexed analyte-specific reporters) from the plurality of second immunocomplex purification solutions to form in the second plate a plurality of third immunocomplex purification solutions;
M) a further eluting in parallel the plurality of second substrates from the plurality of multiplexed analyte specific reporter in the plurality of third immunocomplex purification solutions;
N) replicating the plurality of multiplexed analyte-specific reporters; and
O) detecting the replicated plurality of multiplexed analyte-specific reporters to identify (and quantify) the specific analytes in the plurality of biological samples.

Embodiment 10. A method of automatically conducting in parallel a plurality of multiplexing oligonucleotide-conjugated antibody proximity ligation assays on a plurality of biological samples, comprising:
A) introducing in parallel a portion of a plurality of multiplexed paired-binding moieties into a plurality of biological samples in a first multi-vessel carrier plate to form a plurality of immunocomplex forming solutions;
wherein the multiplexed paired-binding moieties of the plurality of multiplexed paired-binding moieties, comprises different paired-binding moieties that are pre-selected to bind different specific analytes, the paired-binding moieties comprising:
   i) a first moiety of the paired-binding moieties comprising a first antibody or a first antibody fragment that is pre-selected to bind a specific analyte, a first nucleic acid target label comprising a first identity that is analyte-specific to the specific analyte, and a first nucleic tag; and
   ii) a second moiety of the paired-binding moieties comprising a second antibody or a second antibody fragment that is pre-selected to bind the same specific analyte as the first antibody or antibody fragment of the first moiety of the paired-binding moieties, a second nucleic acid target label comprising a second identity that is analyte-specific to the specific analyte, and a second nucleic acid tag;
wherein the first nucleic acid tag of the first moiety of the paired-binding moieties is pre-selected to bind to a first substrate that is the same for all the different paired-binding moieties in the multiplex and the second nucleic acid tag of the second moiety of the paired-binding moieties is pre-selected to bind a second substrate that is the same for all the different paired-binding moieties in the multiplex;
B) incubating in parallel the plurality of immunocomplex forming solutions to form a plurality of multiplexed immunocomplexes in a first multi-vessel carrier plate;
C) a first combining a portion of a first substrate solution comprising a plurality of the first substrates with the plurality of immunocomplex forming solutions in a first multi-vessel carrier plate;
D) a first enabling the first nucleic acid tag of the first moiety of the paired-binding moieties to bind to a portion of the first substrates in a first multi-vessel carrier plate;
E) a first extracting in parallel, via the portion of the plurality of first substrates, the plurality of multiplexed immunocomplexes from in a first multi-vessel carrier plate and depositing them into a second multi-vessel carrier plate;
F) Washing the plurality of multiplexed immunocomplexes in the second multi-vessel carrier plate;
G) a second extracting the plurality of multiplexed immunocomplexes from the second multi-vessel carrier plate and depositing them in a third multi-vessel carrier plate;
H) eluting in parallel the plurality of first substrates from the plurality of multiplexed immunocomplexes in the third multi-vessel carrier plate;
I) removing in parallel the plurality of first substrates from the third multi-vessel carrier plate;
J) a second combining in parallel a portion of a second substrate solution comprising a plurality of the second substrates with the plurality of the plurality of multiplexed immunocomplexes in the third multi-vessel carrier plate;
K) a second enabling the second nucleic acid tag of the second moiety of the paired binding moieties to bind to a portion of the second substrates the third multi-vessel carrier plate,
L) a third extracting in parallel, via the portion of the plurality of second substrates, the plurality of multiplexed immunocomplexes from the third multi-vessel carrier plate and depositing them into the second multi-vessel carrier plate;
M) ligating in parallel a plurality of the first nucleic acid target label from the first moiety of the paired binding moieties (directly or indirectly) with the second nucleic acid target label from the second moiety of the paired binding moieties on the plurality of multiplexed immunocomplexes to form a plurality of multiplexed analyte-specific reporters in the second multi-vessel carrier plate;
N) a fourth extracting in parallel, via the portion of the plurality of second substrates, the plurality of multiplexed immunocomplexes with analyte-specific reporters from the second multi-vessel carrier plate and depositing them into the a third multi-vessel carrier plate;
O) eluting in parallel the plurality of second substrates from the plurality of multiplexed immunocomplexes with analyte-specific reporters in the third multi-vessel carrier plate;
P) replicating the plurality of multiplexed analyte-specific reporters; and
Q) detecting the replicated plurality of multiplexed analyte-specific reporters to identify (and quantify) the presence of the specific analytes in the plurality of biological samples.

Embodiment 11. A method of automatically conducting in parallel a plurality of multiplexing oligonucleotide-conjugated antibody proximity ligation assays on a plurality of biological samples, comprising:
A) introducing in parallel a portion of a plurality of multiplexed paired-binding moieties into a plurality of biological samples in a first multi-vessel carrier plate to form a plurality of immunocomplex forming solutions;
wherein the multiplexed paired-binding moieties of the plurality of multiplexed paired-binding moieties, comprises different paired-binding moieties that are pre-selected to bind different specific analytes, the paired-binding moieties comprising:
   i) a first moiety of the paired-binding moieties comprising a first antibody or a first antibody fragment that is pre-selected to bind a specific analyte, a first nucleic acid target label comprising a first identity that is analyte-specific to the specific analyte, and a first nucleic tag; and
   ii) a second moiety of the paired-binding moieties comprising a second antibody or a second antibody fragment that is pre-selected to bind the same specific analyte as the first antibody or antibody fragment of the first moiety of the paired-binding moieties, a second nucleic acid target label comprising a second identity that is analyte-specific to the specific analyte, and a second nucleic acid tag;
wherein the first nucleic acid tag of the first moiety of the paired-binding moieties is pre-selected to bind to a first substrate that is the same for all the different paired-binding moieties in the multiplex and the second nucleic acid tag of the second moiety of the paired-binding moieties is pre-selected to bind a second substrate that is the same for all the different paired-binding moieties in the multiplex;
B) incubating in parallel the plurality of immunocomplex forming solutions to form a plurality of multiplexed immunocomplexes in a first multi-vessel carrier plate;
C) a first combining a portion of a first substrate solution comprising a plurality of the first substrates with the plurality of immunocomplex forming solutions in a first multi-vessel carrier plate;
D) a first enabling the first nucleic acid tag of the first moiety of the paired-binding moieties to bind to a portion of the first substrates in a first multi-vessel carrier plate;
E) a first extracting in parallel, via the portion of the plurality of first substrates, the plurality of multiplexed immunocomplexes from in a first multi-vessel carrier plate and depositing them into a second multi-vessel carrier plate;
F) Washing the plurality of multiplexed immunocomplexes in the second multi-vessel carrier plate;
G) a second extracting the plurality of multiplexed immunocomplexes from the second multi-vessel carrier plate and depositing them in a third multi-vessel carrier plate;
H) eluting in parallel the plurality of first substrates from the plurality of multiplexed immunocomplexes in the third multi-vessel carrier plate;
I) removing in parallel the plurality of first substrates from the third multi-vessel carrier plate;
J) a second combining in parallel a portion of a second substrate solution comprising a plurality of the second substrates with the plurality of the plurality of multiplexed immunocomplexes in the third multi-vessel carrier plate;
K) a second enabling the second nucleic acid tag of the second moiety of the paired binding moieties to bind to a portion of the second substrates the third multi-vessel carrier plate,
L) a third extracting in parallel, via the portion of the plurality of second substrates, the plurality of multiplexed immunocomplexes from the third multi-vessel carrier plate and depositing them into the a fourth multi-vessel carrier plate;
M) ligating in parallel a plurality of the first nucleic acid target label from the first moiety of the paired binding moieties (directly or indirectly) with the second nucleic acid target label from the second moiety of the paired binding moieties on the plurality of multiplexed immunocomplexes to form a plurality of multiplexed analyte-specific reporters in the fourth multi-vessel carrier plate;
N) a fourth extracting in parallel, via the portion of the plurality of second substrates, the plurality of multiplexed immunocomplexes with analyte-specific reporters from the fourth multi-vessel carrier plate and depositing them into the a third multi-vessel carrier plate;
O) eluting in parallel the plurality of second substrates from the plurality of multiplexed immunocomplexes with analyte-specific reporters in the third multi-vessel carrier plate;
P) replicating the plurality of multiplexed analyte-specific reporters; and
Q) detecting the replicated plurality of multiplexed analyte-specific reporters to identify (and quantify) the presence of the specific analytes in the plurality of biological samples.

Embodiment 12. A method for conducting a biochemical assay on a biological sample on a bench-top automated system to detect at least one analyte comprising:
1) transferring at least one sample from the sample multi-vessel carrier plate to the first multi-vessel carrier plate;
2) diluting the samples in the first multi-vessel carrier plate;
3) forming a first solution in the second multi-vessel carrier plate by mixing:
   i) a portion of at least one sample,
   ii) a portion of the plurality of first moieties comprising a first antibody or a first antibody fragment that is pre-selected to bind a specific analyte, a first nucleic acid target label comprising a first identity that is analyte-specific to the specific analyte, and a first nucleic tag, and
   iii) a portion of the plurality of second moieties comprising a second antibody or a second antibody fragment that is pre-selected to bind the same specific analyte as the first antibody or antibody fragment of the first moiety of the paired-binding moieties, a second nucleic acid target label comprising a second identity that is analyte-specific to the specific analyte, and a second nucleic acid tag;
4) incubating the first solution to form immunocomplexes comprising one of the first moieties from the portion of the plurality of first moieties, one of the second moieties from the portion of the plurality of second moieties and with the analyte in the at least one sample;
5) performing a dual capture and release mechanism of the immunocomplexes to remove contaminants and improve sensitivity by:
   i) adding a portion of a first substrate to capture at least a portion of the immunocomplexes from the first solution in the second multi-vessel carrier plate;
   ii) incubating the first solution in the second multi-vessel carrier plate to immobilize the immunocomplex on the surface of the first substrate;
   iii) transferring the first substrate by a mixer to a third multi-vessel carrier plate to form a second solution with an elution buffer;
   iv) washing the first substrate to remove unbound impurities;
   v) incubating the second solution in the third multi-vessel carrier plate to release at least a portion of the immunocomplexes;
   vi) removing the first substrate by the mixer;
   vii) adding a portion of a second substrate to the second solution in the third multi-vessel carrier plate;
   viii) incubating the second solution in the second multi-vessel carrier plate to immobilize the immunocomplex on the surface of the second substrate;
   ix) transferring the second substrate by the mixer to a third multi-vessel carrier plate to form a third solution;
   x) washing the second substrate to remove unbound impurities;
6) adding a ligation reagent into the third solution in the fourth multi-vessel carrier plate;
7) introducing at least a portion of the plurality of double-stranded nucleic acid sample labels into the third solution in the fourth multi-vessel carrier plate to form at least one multiplexed analyte-specific reporter comprising the first nucleic acid target label associated with one of the first moieties in at least one of the immunocomplexes and at least one of the second nucleic acid target labels conjugated to one of the second moieties in the at least one of the immunocomplexes, and a sample-specific identity barcode, by hybridizing one of the overhangs with the first nucleic acid target label associated with one of the first moieties of the at least a portion of the immunocomplexes and hybridizing the other overhang with the second nucleic acid target label conjugated to one of the second moieties of the at least a portion of the immunocomplexes to complete proximity-based ligation between the first nucleic acid target label and the nucleic acid sample label and the second nucleic acid target label and the nucleic acid sample label to generate the at least one multiplexed analyte-specific reporter that is conjugated to the second antibodies or second antibody fragments and is not covalently attached to the first antibodies or first antibody fragments;
8) collecting the at least one multiplexed analyte-specific reporter in the first multi-vessel carrier plate and pooling solutions of the multiplexed analyte-specific reporters in the first multi-vessel carrier plate; and
9) transferring the pooled solutions of the multiplexed analyte-specific reporter from the first plate to a qPCR plate for detection of the analyte by NGS at attomolar level of sensitivity.

Embodiment 13. A fast, compact instrument for automatically conducting a plurality of multiplexing oligonucleotide-conjugated antibody proximity ligation assays on a plurality of biological samples, comprising:
a) a target kit comprising:
a plurality of paired-binding moieties that are pre-selected to bind specific analytes, the paired-binding moieties comprising:
   i) a first moiety comprising a first antibody or a first antibody fragment that is pre-selected to bind a specific analyte, a first nucleic acid target label comprising a first identity that is analyte-specific to the specific analyte, and a first nucleic tag;
   ii) a second moiety comprising a second antibody or a second antibody fragment that is pre-selected to bind the same specific analyte as the first antibody or antibody fragment of the first moiety of the paired-binding moieties, a second nucleic acid target label comprising a second identity that is analyte-specific to the specific analyte, and a second nucleic acid tag; and
b) a gantry equipped with an end-effector having pipettors and a plate gripper, an incubator and a substrate extractor/mixer for carrying out a dual capture and release immunocomplex purification using a portion of the plurality of paired-binding moieties.

Embodiment 14. A fast, compact instrument for automatically conducting a plurality of multiplexing oligonucleotide-conjugated antibody proximity ligation assays on a plurality of biological samples, comprising:
a) a target kit comprising:
a plurality of paired-binding moieties that are pre-selected to bind specific analytes, the paired-binding moieties comprising:
i) a first moiety comprising a first antibody or a first antibody fragment that is pre-selected to bind a specific analyte, a first nucleic acid target label comprising a first identity that is analyte-specific to the specific analyte, and a first nucleic tag;
ii) a second moiety comprising a second antibody or a second antibody fragment that is pre-selected to bind the same specific analyte as the first antibody or antibody fragment of the first moiety of the paired-binding moieties, a second nucleic acid target label comprising a second identity that is analyte-specific to the specific analyte, and a second nucleic acid tag; and
b) a gantry equipped with an end-effector having pipettors and a plate gripper, an incubator and a substrate extractor/mixer for carrying out a dual capture and release immunocomplex purification using a portion of the plurality of paired-binding moieties.

Embodiment 15. A fast, compact instrument for automatically conducting a plurality of multiplexing oligonucleotide-conjugated antibody proximity ligation assays on a plurality of biological samples, comprising:
a) a target kit comprising:
a plurality of paired-binding moieties that are pre-selected to bind specific analytes, the paired-binding moieties comprising:
   i) a first moiety comprising a first antibody or a first antibody fragment that is pre-selected to bind a specific analyte, a first nucleic acid target label comprising a first identity that is analyte-specific to the specific analyte, and a first nucleic tag;
   ii) a second moiety comprising a second antibody or a second antibody fragment that is pre-selected to bind the same specific analyte as the first antibody or antibody fragment of the first moiety of the paired-binding moieties, a second nucleic acid target label comprising a second identity that is analyte-specific to the specific analyte, and a second nucleic acid tag;
b) a detection kit comprising:
   i) a first substrate solution comprising a plurality of first substrates and
   ii) a second substrate solution comprising a plurality of second substrates and
   iii) a ligation reagent and optionally a nucleic acid sample specific label
c) a microtiter samples plate comprising at least 96 wells;
d) a gantry equipped with an end-effector having pipettors and a plate gripper, an incubator and a substrate extractor/mixer for carrying out a dual capture and release immunocomplex purification using a portion of the plurality of paired-binding moieties, and first and second substrate solutions; and additionally using the ligation reagent to cause a portion of the first nucleic acid target label of the plurality of paired-binding moieties to ligate (directly or indirectly) with a portion of the second nucleic acid target label of the plurality of paired-binding moieties to a form a plurality of analyte-specific reporters.

Embodiment 16. A bench-top instrument for automatically conducting in parallel a plurality of multiplexing oligonucleotide-conjugated antibody proximity ligation assays on a plurality of biological samples, comprising:
1) a multi-vessel carrier plate comprising a plurality of biological samples;
2) a hotel comprising:
   a) a target kit comprising:
   a plurality of multiplexed paired-binding moieties, the multiplexed paired-binding moieties comprising different paired-binding moieties that are pre-selected to bind different specific analytes, the paired-binding moieties comprising:
      i) a first moiety of the paired-binding moieties comprising a first antibody or a first antibody fragment that is pre-selected to bind a specific analyte, a first nucleic acid target label comprising a first identity that is analyte-specific to the specific analyte, and a first nucleic acid tag; and
      ii) a second moiety of the paired-binding moieties comprising a second antibody or a second antibody fragment that is pre-selected to bind the same specific analyte as the first antibody or antibody fragment of the first moiety of the paired-binding moieties, a second nucleic acid target label comprising a second identity that is analyte-specific to the specific analyte, and a second nucleic acid tag;
wherein the first nucleic acid tag of the first moiety of the paired-binding moieties is pre-selected to bind to a first substrate that is the same for all the different paired-binding moieties in the multiplex and the second nucleic acid tag of the second moiety of the paired binding moieties is pre-selected to bind a second substrate that is the same for all the different paired binding moieties in the multiplex;
   b) a detection kit comprising:
      i) a first substrate solution comprising a plurality of first substrates; and
      ii) a second substrate solution comprising a plurality of second substrates;
3) a gantry equipped with an end-effector having pipettors, and a plate gripper;
4) an incubator;
5) a substrate extractor/mixer;
6) a qPCR;
7) a first plate comprising a plurality of first wells;
8) a second plate comprising a plurality of second wells;
9) a third plate comprising a plurality of third wells; and
10) a controller, wherein
   a) the controller controls the gantry to introduce in parallel in a portion of the plurality of first wells in the first plate a portion of the plurality of multiplexed paired-binding moieties from the target kit with a portion of the plurality of biological samples to form a plurality of immunocomplex forming solutions in the portion of the plurality of first wells in the first plate;
   b) the controller controls the gantry and the incubator to incubate in parallel the plurality of immunocomplex forming solutions in the portion of the plurality of first wells in the first plate to form a plurality of multiplexed immunocomplexes;
   c) the controller controls the gantry to combine a portion of the first substrate solution comprising the plurality of the first substrates from the detection kit with the plurality of immunocomplex forming solutions in the portion of the plurality of first wells in the first plate;
   d) the controller controls the gantry and the incubator to enable the first nucleic acid tag of the first moiety of the multiplexed paired-binding moieties to bind to a portion of the first substrates in the portion of the plurality of first wells in the first plate,
   e) the controller controls the substrate extractor to extract in parallel, via the portion of the plurality of first substrates, the plurality of multiplexed immunocomplexes from the plurality of immunocomplex forming solutions to form in at least a portion of the plurality of second wells in the second plate a plurality of first immunocomplex purification solutions, wherein the portion of the plurality of first substrates are eluted from the plurality of multiplexed immunocomplexes in the plurality of first immunocomplex purification solutions;
   f) the controller controls the substrate extractor to extract in parallel the portion of the plurality of first substrates from the plurality of first immunocomplex purification solutions;
   g) the controller controls the gantry to combine a portion of the second substrate solution comprising the plurality of second substrates from the detection kit with the plurality of first immunocomplex purification solutions in the portion of the plurality of second wells in the second plate;
   h) the controller controls the gantry and the incubator to enable a portion of the second nucleic acid tag of the second moiety of the paired-binding moieties to bind to a portion of the second substrates in the portion of the plurality of first immunocomplex purification solutions in the portion of the plurality of second wells in the second plate;
   i) the controller controls the substrate extractor to extract in parallel, via the portion of the plurality of second substrates, the portion of the second substrates in the portion of the plurality of first immunocomplex purification solutions in the portion of the plurality of second wells in the second plate to form in a portion of the plurality of third wells in the third plate a plurality of multiplexed analyte-specific reporters by ligating in parallel a plurality of the first nucleic acid target label from the first moiety of the paired binding moieties (directly or indirectly) with a plurality of the second nucleic acid target label from the second moiety of the paired binding moieties;
   j) the controller controls the substrate extractor to extract in parallel, via the portion of the plurality of second substrates, the plurality of multiplexed analyte-specific reporters from the portion of the plurality of third wells in the third plate back to the portion of the plurality of second wells in the second plate to elute in parallel the plurality of second substrates from the plurality of multiplexed analyte-specific reporters in the portion of the plurality of second wells in the second plate;
   k) the controller controls the gantry and the thermocycler to replicate the plurality of multiplexed analyte-specific reporters; and
   l) the controller controls the qPCR to detect the replicated plurality of multiplexed analyte-specific reporters to identify (and quantify) the specific analytes in the plurality of biological samples.

Embodiment 17. A method of automatically carrying out a dual-capture and release multiplexed immunoassay on a plurality of biological samples, comprising:
operating an instrument comprising:
1) a controller;
2) a robotic gantry capable of moving in three degrees of freedom;
3) a hotel capable of holding multi-vessel carrier plates that are accessible by a user and the robotic gantry;
4) a mixer that operates as a magnetic bead processor and mixer comprising multiple multi-vessel carrier plate platforms;
5) a reader; and
6) positioned within the hotel,
   a) a plurality of multi-vessel carrier plates, comprising a first plate comprising a plurality of first wells, a second plate comprising a plurality of second wells, a third plate comprising a plurality of third wells, a fourth plate comprising a plurality of fourth wells and a multi-vessel carrier assay plate comprising the plurality of biological samples; and
   b) a multi-vessel carrier target kit comprising:
a plurality of multiplexed paired-binding moieties, the multiplexed paired-binding moieties comprising different paired-binding moieties that are pre-selected to bind different specific analytes, the paired-binding moieties comprising:
   i) a first moiety of the paired-binding moieties comprising a first antibody or a first antibody fragment that is pre-selected to bind a specific analyte, a first nucleic acid target label comprising a first identity that is analyte-specific to the specific analyte, and a first nucleic acid tag; and
   ii) a second moiety of the paired-binding moieties comprising a second antibody or a second antibody fragment that is pre-selected to bind the same specific analyte as the first antibody or antibody fragment of the first moiety of the paired-binding moieties, a second nucleic acid target label comprising a second identity that is analyte-specific to the specific analyte, and a second nucleic acid tag;
wherein the first nucleic acid tag of the first moiety of the paired-binding moieties is pre-selected to bind to a first substrate that is the same for all the different paired-binding moieties in the multiplex and the second nucleic acid tag of the second moiety of the paired binding moieties is pre-selected to bind a second substrate that is the same for all the different paired binding moieties in the multiplex;
   c) a multi-vessel carrier detection kit comprising:
      i) a first substrate solution comprising a plurality of first substrates; and
      ii) a second substrate solution comprising a plurality of second substrates;
   the instrument operates to control:
   a) the robotic gantry to introduce in parallel in a portion of the plurality of first wells in the first plate a portion of the plurality of multiplexed paired-binding moieties from the target kit with a portion of the plurality of biological samples from the assay plate to form a plurality of immunocomplex forming solutions in the portion of the plurality of first wells in the first plate;
   b) the gantry and the substrate mixer to incubate in parallel the plurality of immunocomplex forming solutions in the portion of the plurality of first wells in the first plate to form a plurality of multiplexed immunocomplexes;
   c) the gantry to combine a portion of the first substrate solution comprising the plurality of the first substrates from the detection kit with the plurality of multiplexed immunocomplexes in the portion of the plurality of first wells in the first plate;
   d) the gantry and the mixer to enable a portion of the portion of the first nucleic acid tag of the first moiety of the multiplexed paired-binding moieties to bind to a portion of the first substrates in the portion of the plurality of first wells in the first plate,
   e) the mixer to extract in parallel, via the portion of the plurality of first substrates, the plurality of multiplexed immunocomplexes bound to the plurality of first substrates from the portion of the plurality of first wells in the first plate and deposit into the portion of the plurality of second wells in the second plate, and elute the portion of the plurality of first substrates from the plurality of multiplexed immunocomplexes in the portion of the plurality of second wells in the second plate;
   f) the mixer to extract in parallel the portion of the plurality of first substrates from the portion of the plurality of multiplexed immunocomplexes in at least a portion of the plurality of second wells in the second plate;
   g) the gantry to combine a portion of the second substrate solution comprising the plurality of second substrates with the plurality of multiplexed immunocomplexes in the portion of the plurality of second wells in the second plate;
   h) the gantry and the mixer to enable a portion of the portion of the second nucleic acid tag of the second moiety of the paired-binding moieties to bind to the portion of the second substrates in the portion of the plurality of second wells in the second plate;
   i) the mixer to extract in parallel, via the portion of the plurality of second substrates, the portion of the plurality of multiplexed immunocomplexes bound to the portion of the second substrates in the portion of the plurality of second wells in the second plate and deposit into a portion of the plurality of third wells in the third plate and form a plurality of multiplexed analyte-specific reporters by ligating in parallel a plurality of the first nucleic acid target label from the first moiety of the paired binding moieties (directly or indirectly) with a plurality of the second nucleic acid target label from the second moiety of the paired binding moieties;
   j) the mixer to extract in parallel, via the portion of the plurality of second substrates, the plurality of multiplexed analyte-specific reporters bound to the portion of the second substrates from the portion of the plurality of third wells in the third plate and deposit back to the portion of the plurality of second wells in the second plate and elute in parallel the plurality of second substrates from the plurality of multiplexed analyte-specific reporters in the portion of the plurality of second wells in the second plate;
   k) the gantry to transfer at least a portion of the plurality of multiplexed analyte-specific reporters from the portion of the plurality of second wells in the second plate to a portion of the plurality of fourth wells in the fourth plate, and
   l) the gantry to move the fourth plate to the reader.

Embodiment 18. A compact, fully-automated instrument for executing a dual-capture and release multiplexed immunoassay, comprising:
A) a bench-top housing, comprising:
1) a robotic gantry capable of moving in three degrees of freedom;
2) a hotel capable of holding multi-vessel carrier plates that are accessible by a user and the robotic gantry;
3) a controlled stand that provides for a plurality of multi-vessel carrier plate positions, wherein the stand is movable along the Y-axis (from front-to-back within the instrument housing); and
4) a mixer that operates as a magnetic bead processor and mixer comprising multiple multi-vessel carrier plate platforms vertically positioned on top of each other.

Embodiment 19. The instrument of any embodiments 1-5 and 18, carrying out the process of any of the methods of embodiments 9-12 and 17.

In some embodiments, for example, including the embodiments of 1-19 and any of the other embodiments herein, wherein one of the first or second nucleic acid tags comprise a nucleotide rich sequence. The first or second nucleic acid tags comprise a nucleic acid rich sequence of one or more of the following: a poly-A, poly-T, poly-C, or poly-G sequence. The first nucleic acid tag comprises a poly-A or a poly-T sequence.

In some embodiments, for example, including the embodiments of 1-19 and any of the other embodiments herein, wherein one of the first or second nucleic acid tag comprise an immobilization reagent. The first or second nucleic acid tags comprise an immobilization reagent of one or more of the following: biotin, streptavidin, EDC, DCC, NHS esters, imidoesters, maleimides, haloacetyls, pyridyl disulfides, hydrazides, alkoxyamines, aryl azides, dizirines, or a chemoselective ligation group. The second nucleic acid tag is conjugated with biotin.

In some embodiments, for example, including the embodiments of 1-19 and any of the other embodiments herein, wherein the first nucleic acid tag is not covalently attached to the first antibody or antibody fragment.

In some embodiments, for example, including the embodiments of 1-19 and any of the other embodiments herein, the first nucleic acid target label is not covalently attached to the first antibody or the first antibody fragment.

In some embodiments, for example, including the embodiments of 1-19 and any of the other embodiments herein, wherein second nucleic acid tag is not covalently attached to the second antibody or the second antibody fragment.

In some embodiments, for example, including the embodiments of 1-19 and any of the other embodiments herein, wherein the first antibody or the first antibody fragment and the second antibody or the second antibody fragment are paired to bind to non-overlapping epitopes of the analyte and form an immunocomplex.

In some embodiments, for example, including the embodiments of 1-19 and any of the other embodiments herein, wherein the first antibody or the first antibody fragment has a binding affinity to the analyte of at least 10⁻⁴ M, at least 10⁻⁶ M, at least 10⁻⁹ M or higher, and the second antibody or second antibody fragment has a binding affinity to the analyte of at least 10⁻⁴ M, at least 10⁻⁶ M, at least 10⁻⁹ M or higher.

In some embodiments, for example, including the embodiments of 1-19 and any of the other embodiments herein, wherein the first nucleic acid target label comprises a first identity barcode.

In some embodiments, for example, including the embodiments of 1-19 and any of the other embodiments herein, wherein the second nucleic acid target label comprises a second identity barcode.

In some embodiments, for example, including the embodiments of 1-19 and any of the other embodiments herein, wherein the instrument further comprises a target kit. The target kit comprises a plurality of wells of the multi-vessel plate, (i) wherein a first well of at least one of the plurality of wells in the target kit comprises the first moiety; and (ii) a second well of at least one of the plurality of wells in the target kit comprises the second moiety. The first well and the second well are the same well of the multi-vessel plate. Further, the first well and the second well may be different wells of the multi-vessel plate.

In some embodiments, for example, including the embodiments of 1-19 and any of the other embodiments herein, wherein the target kit further comprises a plurality of wells of the multi-vessel plate, (i) wherein a third well of at least one of the plurality of wells of the target kit, comprises a first nonfunctional binder comprising a third antibody or a third antibody fragment that specifically binds the analyte; and (ii) wherein a fourth well of at least one of the plurality of wells of the target kit, comprises a second nonfunctional binder comprising a fourth antibody or a fourth antibody fragment that specifically binds the analyte. The first nonfunctional binder binds the same epitope of the analyte in the same manner as the first moiety. The third antibody or the third antibody fragment are identical to the first antibody or the first antibody fragment of the first moiety. The fourth antibody or the fourth antibody fragment are identical to the second antibody or the second antibody fragment of the second moiety. The third well and the fourth well may be the same well of the multi-vessel plate. The third well and the fourth well may be different wells of the multi-vessel plate. Further, the first, second, third, and fourth wells may be the same well of the multi-vessel plate. The first, second, third, and fourth wells may be different wells of the multi-vessel plate.

In some embodiments, for example, including the embodiments of 1-19 and any of the other embodiments herein, wherein the first nonfunctional binder is mixed with the first moiety in a predetermined proportion.

In some embodiments, for example, including the embodiments of 1-19 and any of the other embodiments herein, wherein the second nonfunctional binder is mixed with the second moiety in a predetermined proportion.

In some embodiments, for example, including the embodiments of 1-19 and any of the other embodiments herein, wherein the instrument further comprises a detection kit. The detection comprises a plurality of wells of a multi-vessel plate,
(i) wherein a first well of at least one of the plurality of wells comprises a first substrate solution comprising a plurality of first substrates;
(ii) wherein a second well of at least one of the plurality of wells comprises a second substrate solution comprising a plurality of second substrates; and
(iii) wherein a third well of at least one of the plurality of wells comprises a ligation reagent and optionally a nucleic acid sample specific label.

In some embodiments, for example, including the embodiments of 1-19 and any of the other embodiments herein, wherein the first substrates are a first paramagnetic beads. The first paramagnetic beads are coupled with a first binding sequence that is capable of binding with the first nucleic acid tag on the first antibody or first antibody fragment. The first paramagnetic beads may be coated with a first binding sequence that is capable of binding with the first nucleic acid tag on the first antibody or first antibody fragment.

In some embodiments, for example, including the embodiments of 1-19 and any of the other embodiments herein, wherein the second substrates are a second paramagnetic beads. The second paramagnetic beads are coupled with a second binding group that is capable of binding with the second nucleic acid tag associated with the second antibody or second antibody fragment. The second paramagnetic beads may be coated with a second binding group that is capable of binding with the second nucleic acid tag associated with the second antibody or second antibody fragment.

In some embodiments, for example, including the embodiments of 1-19 and any of the other embodiments herein, wherein the first binding sequence is pre-selected for hybridizing with the first nucleic acid tag on the first antibody or first antibody fragment.

In some embodiments, for example, including the embodiments of 1-19 and any of the other embodiments herein, wherein the first binding sequence coupled to the first paramagnetic beads is a poly-A or poly-T sequence. The poly-A sequence from the first nucleic acid tag hybridizes with the poly-T sequence from the first paramagnetic beads. Alternatively, the poly-T sequence from the first nucleic acid tag hybridizes with the poly-A sequence from the first paramagnetic beads.

In some embodiments, for example, including the embodiments of 1-19 and any of the other embodiments herein, wherein the second binding group coupled to the second paramagnetic beads is streptavidin or avidin.

In some embodiments, for example, including the embodiments of 1-19 and any of the other embodiments herein, wherein the biotin from the second nucleic acid tag binds to streptavidin from the second paramagnetic beads. Alternatively, the biotin from the second nucleic acid tag binds to avidin from the second paramagnetic beads.

In some embodiments, for example, including the embodiments of 1-19 and any of the other embodiments herein, wherein the bond between the first nucleic acid tag and the binding sequence is an orthogonal bond.

In some embodiments, for example, including the embodiments of 1-19 and any of the other embodiments herein, wherein the bond between the second nucleic acid tag and the binding group is an orthogonal bond.

In some embodiments, for example, including the embodiments of 1-19 and any of the other embodiments herein, wherein the first, second, and third wells of the multi-vessel plate of the detection kit, are in the multi-vessel plate of the target kit. Alternatively, the first and second wells of the target kit are in the detection kit.

In some embodiments, for example, including the embodiments of 1-19 and any of the other embodiments herein, wherein the instrument further comprises an incusealer for incubating and/or sealing at least one multi-vessel carrier plate.

In some embodiments, for example, including the embodiments of 1-19 and any of the other embodiments herein, wherein the instrument further comprises a bulk fluid station comprising a plurality of containers.

In some embodiments, for example, including the embodiments of 1-19 and any of the other embodiments herein, wherein the robotic gantry further comprises an end-effector, comprising pipettors, a multi-vessel carrier plates gripper, a laser position sensor, and a barcode scanner, the robotic gantry capable of moving the end-effector in three degrees of freedom in X, Y and Z axis.

In some embodiments, for example, including the embodiments of 1-19 and any of the other embodiments herein, wherein the reader comprises a qPCR unit for qPCR readout.

In some embodiments, for example, including the embodiments of 1-19 and any of the other embodiments herein, wherein the reader comprises a qPCR for preparing a pool library ready for next-gen sequencing (NGS).

In some embodiments, for example, including the embodiments of 1-19 and any of the other embodiments herein, wherein the reader is capable of identifying and/or quantifying nucleic acid reporters.

In some embodiments, for example, including the embodiments of 1-19 and any of the other embodiments herein, wherein the housing further comprises a touchscreen display.

In some embodiments, for example, including the embodiments of 1-19 and any of the other embodiments herein, wherein the hotel comprises a set of multi-vessel carrier bays for receiving and holding at least one universal reagent cartridge, and a consumable carrier.

In some embodiments, for example, including the embodiments of 1-19 and any of the other embodiments herein, wherein the hotel further comprises:
a) the target kit;
b) the detection kit; and
c) the at least one consumable carrier comprising:
   i) a PCR multi-vessel carrier plate,
   ii) a framed sealing film,
   iii) a plurality of pipette tips, and
   iv) a plurality of multi-vessel carrier plates.

In some embodiments, for example, including the embodiments of 1-19 and any of the other embodiments herein, wherein the framed sealing film has one or more perforated lines that are configured to allow easy and complete separation of the film from the frame by tearing. The framed sealing film seals the wells when heat welded to the edges of the multi-vessel carrier plate. The framed sealing film comprises a metallic layer. The framed sealing film is pierceable by a pipette tip.

In some embodiments, for example, including the embodiments of 1-19 and any of the other embodiments herein, wherein the controller comprises a processor and a non-transitory machine-readable storage medium comprising instructions executable by the processor to provide controlled operations of the components within the instrument. Alternatively, the controller comprises a processor and a non-transitory machine-readable storage medium comprising preprogrammed instructions executable by the processor to execute the dual-capture and release multiplexed immunoassay.

In some embodiments, for example, including the embodiments of 1-19 and any of the other embodiments herein, the controller comprises a processor and a non-transitory machine-readable storage medium comprising instructions executable by the processor to provide controlled operations of the unit operations with the system, the operations controlled by the controller comprise:
a) forming a first solution in a first multi-vessel carrier plate by mixing:
i) a portion of at least one of the biological samples,
ii) a portion of the plurality of the first moiety, and
iii) a portion of the plurality of the second moiety;
b) incubating the first solution in the incubator to form immunocomplexes comprising one of the first moieties from the portion of the plurality of first moieties, one of the second moieties from the portion of the plurality of second moieties and the analyte, in the at least one biological sample;
c) performing the dual capture and release mechanism of the immunocomplexes to remove unbound contaminants and improve sensitivity by introducing:
   i) a portion of the first paramagnetic beads to capture at least a portion of the immunocomplexes from the first solution in the first multi-vessel carrier plate wherein the first solution in the first multi-vessel carrier plate is incubated, and the first paramagnetic beads are transferred by the extractor/mixer to a second multi-vessel carrier plate to form a second solution to release at least a portion of the immunocomplexes, and the first paramagnetic beads are removed by the extractor/mixer,
   ii) a portion of the second paramagnetic beads to capture at least a portion of the immunocomplexes from the second solution wherein the second solution is incubated, and the second set of paramagnetic beads are removed by the extractor/mixer to form a third solution in a third multi-vessel carrier plate.

In some embodiments, for example, including the embodiments of 1-19 and any of the other embodiments herein, wherein the contaminants may include unbound first moieties or unbound second moieties. The contaminants may include nontarget analytes. The contaminants may include unbound target analytes.

In some embodiments, for example, including the embodiments of 1-19 and any of the other embodiments herein, wherein the operations controlled by the controller further comprise:
a) washing the first solid surface of the first substrate to remove the unbound contaminants; and
b) washing the second solid surface of the second substrate to remove the unbound contaminants.

In some embodiments, for example, including the embodiments of 1-19 and any of the other embodiments herein, wherein the operations controlled by the system controller further comprise:
a) introducing at least a portion of a plurality of nucleic acid sample labels into the third solution to form at least one nucleic acid reporter comprising the first nucleic acid target label associated with one of the first moieties in at least one of the immunocomplexes and at least one of the second nucleic acid target labels conjugated to one of the second moieties in the at least one of the immunocomplexes, and a sample-specific identity barcode, by hybridizing one of the overhangs with the first nucleic acid target label associated with one of the first moieties of the at least a portion of the immunocomplexes and hybridizing the other overhang with the second nucleic acid target label conjugated to one of the second moieties of the at least a portion of the immunocomplexes to complete proximity-based ligation between the first nucleic acid target label and the nucleic acid sample label and the second nucleic acid target label and the nucleic acid sample label to generate the at least one nucleic acid reporter that is conjugated to the first moiety and is not covalently attached to the second moiety; and
b) collecting the at least one nucleic acid reporters in a fourth multi-vessel carrier plate and then pooling the nucleic acid reporter to form a pooled sample for detection of the analyte by NGS at attomolar level of sensitivity with a broad dynamic measurement range.

In some embodiments, for example, including the embodiments of 1-19 and any of the other embodiments herein, the operations controlled by the controller further comprise the multi-vessel carrier plate washer washing the multi-vessel carrier plate previously used to perform the capture and release mechanism and re-introducing the immunocomplexes into the washed multi-vessel carrier plate.

In some embodiments, for example, including the embodiments of 1-19 and any of the other embodiments herein, wherein the instrument is pre-configured to execute the dual-capture and release multiplexed immunoassay.

In some embodiments, for example, including the embodiments of 1-19 and any of the other embodiments herein, wherein the instrument is an all-in-one instrument with integrated qPCR instrument processes the sample assays from sample preparation to data or to a pooled NGS library.

In some embodiments, for example, including the embodiments of 1-19 and any of the other embodiments herein, wherein the instrument is capable of attomolar sensitivity detection. The attomolar sensitivity detection may be at least 0.1, at least 1, at least 5, at least 10, at least 15, at least 20, at least 25, at least 30, or higher.

In some embodiments, for example, including the embodiments of 1-19 and any of the other embodiments herein, wherein the instrument is capable of attomolar sensitivity detection has a broad dynamic measurement range up to 12 logs, up to 14 logs, up to 16 logs, up to 18 logs, or up to 20 logs, comprising the upper limit of detection of one member of the target panel and the lowest limit of detection of the one member of the target panel.

In some embodiments, for example, including the embodiments of 1-19 and any of the other embodiments herein, wherein the instrument is capable of executing the dual-capture and release multiplexed immunoassay for over 10 samples, over 25 samples, over 35 samples, over 50 samples, over 75 samples, or over 100 samples in parallel.

In some embodiments, for example, including the embodiments of 1-19 and any of the other embodiments herein, wherein the instrument can complete, without human intervention, the dual-capture and release multiplexed immunoassay for a plurality of samples from assay samples.

In some embodiments, for example, including the embodiments of 1-19 and any of the other embodiments herein, wherein the instrument can complete, without human intervention, the dual-capture and release multiplexed immunoassay for a plurality of samples from assay samples in less than 1 hour, less than 2 hours, less than 3 hours, or less than 4 hours.

In some embodiments, for example, including the embodiments of 1-19 and any of the other embodiments herein, wherein the instrument can complete, without human intervention, the dual-capture and release multiplexed immunoassay for a plurality of samples from assay samples to qPCR readout in less than 3 hours, less than 4 hours, less than 5 hours, or less than 6 hours.

In some embodiments, for example, including the embodiments of 1-19 and any of the other embodiments herein, wherein the instrument completes the multiplexed oligonucleotide-conjugated antibody proximity ligation assays on the plurality of biological samples from sample preparation through the dual capture and release immunocomplex purification to formation of the plurality of analyte-specific reporters for a multiplex of analytes in the plurality of biological samples within 4 hours, within 5 hours, within 6 hours, within 7 hours, or within 8 hours automatically and with a sensitivity as low as 10 attomolars, as low as 20 attomolars, as low as 30 attomolars, as low as 40 attomolars, as low as 50 attomolars, as low as 100 attomolars, or as low as 150 attomolars.

In some embodiments, for example, including the embodiments of 1-19 and any of the other embodiments herein, wherein the instrument completes the dual capture and release mechanism in less than 0.5 hours, less than 1 hour, less than 2 hours, or less than 3 hours.

In some embodiments, for example, including the embodiments of 1-19 and any of the other embodiments herein, wherein the instrument contains sufficient amounts of reagents and other consumables for running at least two cycles, at least three cycles of assays, at least four cycles of assays, at least five cycles of assays, or at least six cycles of assays, without reloading of reagents and other consumables by users.

In some embodiments, for example, including the embodiments of 1-19 and any of the other embodiments herein, wherein the instrument is a bench-top instrument.

In some embodiments, for example, including the embodiments of 1-19 and any of the other embodiments herein, wherein the instrument has a height (not including the stand) of less than 50 inches, less than 48 inches, less than 46 inches, less than 44 inches, less than 42 inches, less than 40 inches, less than 38 inches, less than 36 inches, less than 34 inches, less than 32 inches, or less than 30 inches.

In some embodiments, for example, including the embodiments of 1-19 and any of the other embodiments herein, the base of the instrument has a depth of less than 36 inches, less than 32 inches, less than 30 inches, less than 28 inches, less than 26 inches, less than 24 inches, less than 22 inches, or less than 20 inches and a width of less than 50 inches, less than 48 inches, less than 46 inches, less than 44 inches, less than 42 inches, less than 40 inches, less than 38 inches, less than 36 inches, less than 34 inches, less than 32 inches, or less than 30 inches.

In some embodiments, for example, including the embodiments of 1-19 and any of the other embodiments herein, wherein the instrument is capable of high-throughput.

In some embodiments, for example, including the embodiments of 1-19 and any of the other embodiments herein, wherein the sample is a serum sample, a plasma sample, or a cerebrospinal fluid sample. The sample may be derived from a cell sample or a tissue sample.

In some embodiments, for example, including the embodiments of 1-19 and any of the other embodiments herein, wherein the sample has a plurality of analytes greater than 10, greater than 50, greater than 100, greater than 150, greater than 200, greater than 250, greater than 300, greater than 350, greater than 400, greater than 450 or greater than 500.

In some embodiments, for example, including the embodiments of 1-19 and any of the other embodiments herein, wherein the sample amount is less than 50 microliters, less than 20 microliters, less than 15 microliters, less than 10 microliters, less than 5 microliters, or less than 1 microliter.

In some embodiments, for example, including the embodiments of 1-19 and any of the other embodiments herein, wherein the analyte is a protein or polypeptide.

In some embodiments, for example, including the embodiments of 1-19 and any of the other embodiments herein, wherein the multiplex detects at least 50 analytes, at least 100 analytes, at least 150 analytes, or at least 200 analytes.

In some embodiments, for example, including the embodiments of 1-19 and any of the other embodiments herein, the automated instrument for executing a dual-capture and release immunoassay procedure, wherein the instrument consumes no more than three, no more than four, no more than five, no more than six, or no more than seven multi-vessel carrier plates for an assay run.

In some embodiments, for example, including the embodiments of 1-19 and any of the other embodiments herein, the automated instrument for executing a dual-capture and release immunoassay procedure, wherein the instrument consumes no more than one box, no more than two boxes, no more than three boxes, no more than four boxes of pipette tips for an assay run.

In some embodiments, for example, including the embodiments of 1-19 and any of the other embodiments herein, the instrument uses at least one plate for carrying a solution comprising the immunocomplex for capturing the immunocomplex on a substrate and re-uses the at least one plate for carrying a solution comprising the immunocomplex for eluting the immunocomplex from the substrate.

In some embodiments, for example, including the embodiments of 1-19 and any of the other embodiments herein, the instrument uses at least one plate for carrying a solution comprising the immunocomplex for capturing the immunocomplex on a substrate, washes the at least one plate and re-uses the at least one plate for carrying a solution comprising the immunocomplex for eluting the immunocomplex from the substrate.

In some embodiments, for example, including the embodiments of 1-19 and any of the other embodiments herein, the instrument uses at least one plate for carrying a solution comprising the immunocomplex bound on the first substrate and re-uses the at least one plate for carrying a solution comprising the immunocomplex bound on the second substrate.

In some embodiments, for example, including the embodiments of 1-19 and any of the other embodiments herein, the instrument uses at least one plate for carrying a solution comprising the immunocomplex bound on the first substrate for eluting the first substrate from the immunocomplex and uses the at least one plate for carrying a solution comprising the immunocomplex bound on the second substrate for eluting the second substrate from the immunocomplex.

In some embodiments, for example, including the embodiments of 1-19 and any of the other embodiments herein, the instrument uses at least one plate for carrying a solution comprising the immunocomplex for elusion of the first substrate from the immunocomplex and re-uses the at least one plate for carrying a solution comprising the immunocomplex for elusion of the second substrate from the immunocomplex.

In some embodiments, for example, including the embodiments of 1-19 and any of the other embodiments herein, the instrument uses at least one plate for carrying a solution comprising the immunocomplex for elusion of the first substrate from the immunocomplex, washes the at least one plate and re-uses the at least one plate for carrying a solution comprising the immunocomplex for elusion of the second substrate from the immunocomplex.

In some embodiments, for example, including the embodiments of 1-19 and any of the other embodiments herein, the instrument uses at least one plate for carrying a solution comprising the immunocomplex prior to capturing the immunocomplex on a second substrate and re-uses the at least one plate for carrying a solution comprising the immunocomplex for elusion of the second substrate from the immunocomplex.

In some embodiments, for example, including the embodiments of 1-19 and any of the other embodiments herein, the instrument uses at least one plate for carrying a solution comprising the immunocomplex prior to capturing the immunocomplex on a second substrate and re-uses the at least one plate for carrying a solution comprising the immunocomplex after capturing the immunocomplex on the second substrate.

In some embodiments, for example, including the embodiments of 1-19 and any of the other embodiments herein, the instrument uses at least one plate for carrying a solution comprising the immunocomplex bound to the first substrate and uses the at least one plate for carrying a solution comprising the immunocomplex for capturing the immunocomplex on the second substrate.

In some embodiments, for example, including the embodiments of 1-19 and any of the other embodiments herein, the instrument uses at least one plate for carrying a solution comprising the immunocomplex bound to the first substrate and re-uses the at least one plate for carrying a solution comprising the immunocomplex for eluting the immunocomplex from the second substrate.

In some embodiments, for example, including the embodiments of 1-19 and any of the other embodiments herein, the instrument uses at least one plate for carrying a solution comprising the immunocomplex bound to the first substrate and uses the at least one plate for carrying a solution comprising the immunocomplex for capturing the immunocomplex on the second substrate and re-uses the at least one plate for carrying the solution comprising the immunocomplex for eluting the immunocomplex from the second substrate.

In some embodiments, for example, including the embodiments of 1-19 and any of the other embodiments herein, the instrument uses at least one plate for carrying a solution comprising the immunocomplex bound to the first substrate during the elution of the first substrate and uses the at least one plate for carrying a solution comprising the immunocomplex for capturing the immunocomplex on the second substrate and re-uses the at least one plate for carrying the solution comprising the immunocomplex for eluting the immunocomplex from the second substrate.

In some embodiments, for example, including the embodiments of 1-19 and any of the other embodiments herein, the instrument uses at least one plate for carrying a solution comprising the immunocomplex prior to capturing the immunocomplex on a second substrate, washes the at least one plate and re-uses the at least one plate for carrying a solution comprising the immunocomplex after capturing the immunocomplex on the second substrate.

In some embodiments, for example, including the embodiments of 1-19 and any of the other embodiments herein, the instrument uses at least one plate for carrying a solution comprising the immunocomplex bound to the first substrate, washes the at least one plate and re-uses the at least one plate for carrying a solution comprising the immunocomplex for capturing the immunocomplex on the second substrate.

In some embodiments, for example, including the embodiments of 1-19 and any of the other embodiments herein, the instrument uses at least one plate for carrying a solution comprising the immunocomplex bound to the first substrate, washes the at least one plate and re-uses the at least one plate for carrying a solution comprising the immunocomplex for eluting the immunocomplex from the second substrate.

In some embodiments, for example, including the embodiments of 1-19 and any of the other embodiments herein, the instrument uses at least one plate for carrying a solution comprising the immunocomplex bound to the first substrate, washes the at least one plate and re-uses the at least one plate for carrying a solution comprising the immunocomplex for capturing the immunocomplex on the second substrate and re-uses the at least one plate for carrying the solution comprising the immunocomplex for eluting the immunocomplex from the second substrate.

In some embodiments, for example, including the embodiments of 1-19 and any of the other embodiments herein, the instrument uses at least one plate for carrying a solution comprising the immunocomplex bound to the first substrate during the elution of the first substrate, washes the at least one plate and re-uses the at least one plate for carrying a solution comprising the immunocomplex for capturing the immunocomplex on the second substrate and re-uses the at least one plate for carrying the solution comprising the immunocomplex for eluting the immunocomplex from the second substrate.

In some embodiments, for example, including the embodiments of 1-19 and any of the other embodiments herein, the instrument uses at least one plate for carrying the immunocomplex solution for capturing the immunocomplex on a substrate, then washes the at least one plate and re-uses the at least one plate for eluting the immunocomplex solution from the substrate.

In some embodiments, for example, including the embodiments of 1-19 and any of the other embodiments herein, the instrument uses at least one plate for holding the immunocomplex solution during washing and re-uses the at least one plate for holding the immunocomplex solution during the ligation for forming the reporters.

In some embodiments, for example, including the embodiments of 1-19 and any of the other embodiments herein, the instrument uses at least one plate for holding a solution comprising the immunocomplex bound to the first substrate and re-uses the at least one plate for holding a solution comprising the immunocomplex bound to the second substrate.

In some embodiments, for example, including the embodiments of 1-19 and any of the other embodiments herein, the instrument uses at least one plate for holding a solution comprising the immunocomplex bound to the first substrate during washing and re-uses the at least one plate for holding a solution comprising the immunocomplex bound to the second substrate during ligation.

In some embodiments, for example, including the embodiments of 1-19 and any of the other embodiments herein, the instrument uses at least one plate for holding a solution comprising the immunocomplex bound to the first substrate prior to elution of the first substrate and re-uses the at least one plate for holding a solution comprising the immunocomplex bound to the second substrate prior to elution of the second substrate.

In some embodiments, for example, including the embodiments of 1-19 and any of the other embodiments herein, the instrument uses at least one plate for holding the immunocomplex formation solution for forming the immunocomplex and uses the at least one plate for capturing the immunocomplex on a substrate.

In some embodiments, for example, including the embodiments of 1-19 and any of the other embodiments herein, the instrument uses at least one plate for holding the immunocomplex formation solution for forming the immunocomplex and uses the at least one plate for capturing the immunocomplex on a first substrate.

In some embodiments, for example, including the embodiments of 1-19 and any of the other embodiments herein, the instrument uses at least one plate for holding the immunocomplex formation solution for forming the immunocomplex and uses the at least one plate for capturing the immunocomplex on a first substrate and re-uses the at least one plate for holding a solution comprising the immunocomplex solution bound to the first substrate.

In some embodiments, for example, including the embodiments of 1-19 and any of the other embodiments herein, the instrument uses at least one plate for holding the immunocomplex formation solution for forming the immunocomplex and uses the at least one plate for capturing the immunocomplex on a first substrate, washes the at least one plate and re-uses the at least one plate for holding a solution comprising the immunocomplex solution bound to the first substrate.

In some embodiments, for example, including the embodiments of 1-19 and any of the other embodiments herein, the instrument uses at least one plate for holding the immunocomplex formation solution for forming the immunocomplex and uses the at least one plate for capturing the immunocomplex on a first substrate and re-uses the at least one plate for holding a solution comprising the immunocomplex solution bound to the first substrate for washing.

In some embodiments, for example, including the embodiments of 1-19 and any of the other embodiments herein, the instrument uses at least one plate for holding the immunocomplex formation solution for forming the immunocomplex and uses the at least one plate for capturing the immunocomplex on a first substrate, washes the at least one plate and re-uses the at least one plate for holding a solution comprising the immunocomplex solution bound to the first substrate for washing.

In some embodiments, for example, including the embodiments of 1-19 and any of the other embodiments herein, the instrument uses at least the same plate for holding the immunocomplex solution for capturing the immunocomplex on a substrate and uses at least the same plate for eluting the immunocomplex from the substate.

In some embodiments, for example, including the embodiments of 1-19 and any of the other embodiments herein, the instrument uses at least the same plate for holding the immunocomplex formation solution for forming the immunocomplex and uses at least the same plate for capturing the immunocomplex on the first substrate.

In some embodiments, for example, including the embodiments of 1-19 and any of the other embodiments herein, the instrument uses at least the same plate for holding the immunocomplex for eluting the first substrate and uses at least the same plate for capturing the immunocomplex on the first substrate.

In some embodiments, for example, including the embodiments of 1-19 and any of the other embodiments herein, the instrument uses at least the same plate for holding the immunocomplex solution for eluting the first substrate and uses at least the same plate for capturing the immunocomplex on the second substrate.

In some embodiments, for example, including the embodiments of 1-19 and any of the other embodiments herein, the instrument uses at least the same plate for holding the first substrate solution and the second substrate solution.

In some embodiments, for example, including the embodiments of 1-19 and any of the other embodiments herein, the instrument uses at least the same plate for holding the first substrate solution, washes at least the same plate, and re-uses the same plate for holding the second substrate solution.

In some embodiments, for example, including the embodiments of 1-19 and any of the other embodiments herein, the instrument re-uses at least one plate for carrying the solution comprising the target analyte from one step in the process in a later step in the process.

In some embodiments, for example, including the embodiments of 1-19 and any of the other embodiments herein, the instrument re-uses at least one plate for carrying the solution comprising the immunocomplex from one step in the process in a later step in the process.

In some embodiments, for example, including the embodiments of 1-19 and any of the other embodiments herein, the instrument uses at least one plate for holding a solution comprising at least one immunocomplex that is a pre-cursor to a target analyte and the re-uses the at least one plate for carrying a solution comprising the immunocomplex from one step in the process in a later step in the process.

In some embodiments, for example, including the embodiments of 1-19 and any of the other embodiments herein, the instrument transfers the samples, meaning solutions comprising the target analytes or the analyte captured by the immunocomplexes, from the formation of the immunocomplexes through various process steps to the formation of the corresponding nucleic acid analyte-reporters without having any pipettes tips contact the samples.

In some embodiments, for example, including the embodiments of 1-19 and any of the other embodiments herein, the instrument transfers the samples, meaning solutions comprising the target analytes or the analyte captured by the immunocomplexes, from the formation of the immunocomplexes through the process steps to the formation of the corresponding nucleic acid analyte-reporters exclusive of needing to have any pipettes tips contact the samples.

In some embodiments, an apparatus for performing an immunoassay is provided comprising a microtiter plate handler configured to be operatively coupled to a microtiter plate having a plurality of reaction wells (e.g., between 20-500 wells, such as 96-wells or 384-wells wherein the wells have a working volume per well of at least 5 μL, for example from 10⁻¹⁰⁰ μL, or 20-90 μl and/or a total volume per well of greater than 150 μL, for example from 190-250 μL)) for receiving, inter alia, assay sample solutions, a microtiter plate sealer to apply a sealing component to the surface of the microtiter plate to seal the solutions within the respective well of the microtiter plate and mitigate any contamination and/or lose of the solution within the respective wells, a magnetic-bead extraction system for mixing the solutions (e.g., assay samples) and extracting magnetic beads from solution and additives within the wells of the microtiter plate, an incubator for promoting the binding of antibodies to analytes within the assay samples, a washing system for cleaning samples and/or contaminates in the wells (inclusive of a decontamination wash system for decontaminating the microtiter plates), a polymerase chain reaction (PCR) system, and optionally an imaging system configured to acquire an image of at least a portion of the solutions (assay samples or the results of the assay sample after undergoing all, or at least a portion, of a NULISA procedure) in the respective wells; an x-y-z-gantry equipped with an end-effector having the microtiter plate gripper (plate handler), a pipette subsystem and at least one position sensor; and a computer implemented control system configured to automatically operate, manage and coordinate all, or at least a portion of, the systems within the apparatus (including at least the plate handler, the plate sealer, magnetic-bead extractor, the incubator, the PCR system, and/or the gantry) and/or receive information from the imaging system related to the image.

In some embodiments, an apparatus for sealing the microtiter plates is equipped with a frame comprising a film, wherein the film may be self-adhesive, thermoformable, transparent, and/or suitably air-tight, and wherein the frame comprising the film is operatively coupled to a microtiter plate. The frame is constructed and positioned to apply a sealing component to the surface of the microtiter plate form a seal over all or at least a portion of the wells within the microtiter plate, wherein the contents (e.g., solutions or assay samples) of each well substantially isolated from the contents of each of the other plurality of sealed assay sites; and a controller configured to automatically operate the sealer to apply the sealing component to the plurality of assay sites.

In some embodiments, an apparatus for inserting beads into assay sites on an assay consumable is provided comprising an assay consumable handler configured to be operatively coupled to an assay consumable having a surface comprising a plurality of assay sites; a bead loader configured to insert individual beads into individual assay sites, such that each assay site containing a bead will contain no more than one bead; and a controller configured to automatically operate the bead loader to insert individual beads into individual assay sites.

In some embodiments, an apparatus for performing an assay is provided comprising an assay consumable handler configured to be operatively coupled to an assay consumable having a surface comprising a plurality of assay sites; a sample loader configured to load an assay sample containing analyte molecules or particles having an unknown concentration to be measured into at least a portion of the plurality of assay sites, such that a plurality of assay sites into which assay sample is loaded contain either zero or a single analyte molecule or particle; a detector configured to interrogate at least a portion of the assay sites containing assay sample and determine a fraction of the plurality of assay sites interrogated that contain an analyte molecule or particle; and a computer implemented system configured receive information from the detector and from the information determine a measure of the unknown concentration of the analyte molecules or particles in the assay sample.

In some embodiments, an apparatus for inserting beads into assay sites on an assay consumable is provided comprising an assay consumable handler configured to be operatively coupled to the assay consumable, wherein the assay consumable comprises a surface comprising a plurality of the assay sites; a bead applicator configured to apply a plurality of magnetic beads to the surface of the assay consumable or place a plurality of magnetic beads in close proximity to the surface; a bead loader comprising a magnetic field generator positioned adjacent to the assay consumable and configured to create relative motion between the magnetic beads and the assay sites; and a controller configured to automatically operate the bead loader to create relative motion between the magnetic beads and the assay sites and insert beads into assay sites.

In some embodiments, an apparatus for removing excess beads from an assay consumable having a surface comprising a plurality of assay sites is provided comprising a assay consumable handler operatively coupled to the assay consumable, wherein the assay consumable comprises a plurality of beads, wherein a first portion of the beads are contained in the assay sites and a second portion of the beads are positioned on the surface of the assay consumable, but not contained within an assay site; a wiper configured to remove substantially all of the second portion of beads from the surface; and a controller configured to automatically operate the wiper to remove the second portion of the beads.

In some embodiments, an assay consumable is provided comprising a surface comprising a plurality of assay sites, wherein each of the assay sites has a volume between about 10 attoliters and about 50 picoliters; and at least one channel formed in the surface at least partially surrounding the plurality of assay sites that is positioned and configured to collect excess assay sample liquid applied to the surface that overflows the assay sites.

In some embodiments, an automated method for forming a plurality of sealed assay sites for performing an assay is provided comprising operatively associating an assay consumable having a surface comprising a plurality of assay sites with a sealer apparatus comprising a sealer and a controller; and applying a sealing component to the plurality of assay sites with the sealer apparatus such that a plurality of sealed assay sites are formed, wherein the contents of each sealed assay site is substantially isolated from the contents of each of the other plurality of sealed assay sites.

In some embodiments, a method for inserting beads into reaction vessels on an assay consumable is provided comprising generating a magnetic field in proximity to a surface of the assay consumable comprising a plurality of the reaction vessels such that a magnetic field vector of the magnetic field is directed from the surface towards a bottom of the reaction vessels and/or towards the perimeter of the surface; delivering a plurality of magnetic beads in proximity to the surface; and creating relative motion between the magnetic beads and the reaction vessels.

In some embodiments, a method for forming a plurality of sealed reaction vessels for performing an assay is provided comprising associating an assay consumable having a surface comprising a plurality of assay sites with a sealing component by applying the sealing component to the surface, wherein the contents of each assay site are substantially isolated from the contents of each of the other plurality of assay sites following association of the sealing component without maintaining any pressure applied to the sealing component, and wherein each of the assay sites has a volume between about 10 attoliters and about 50 picoliters.

In some embodiments, a method for forming a plurality of sealed reaction vessels for performing an assay is provided comprising associating an assay consumable having a surface comprising a plurality of assay sites with a sealing component by applying the sealing component to the surface of the assay consumable and applying pressure to the sealing component, wherein the contents of each assay site are substantially isolated from the contents of each of the other plurality of assay sites following association of the sealing component with the assay consumable; wherein the sealing component comprises a pressure-sensitive adhesive such that the pressure-sensitive adhesive is activated upon application of the pressure to the sealing component and the adhesive forms an adhesive bond between the sealing component and the surface of the assay consumable; and wherein each of the assay sites has a volume between about 10 attoliters and about 50 picoliters.

In some embodiments, a method for forming a plurality of sealed assay sites for performing an assay is provided comprising providing an assay consumable having a surface comprising a plurality of assay sites wherein each of the assay sites has a volume between about 10 attoliters and about 50 picoliters; and applying a liquid that is substantially immiscible with liquid contained in the plurality of assay sites to the plurality of assay sites such that a plurality of sealed assay sites are formed, wherein the contents of each sealed assay site is substantially isolated from the contents of each of the other plurality of sealed assay sites.

In some embodiments, an apparatus for removing beads from a surface of an assay consumable is provided comprising a first magnet, wherein the first magnet is located adjacent to a surface of the assay consumable and is positioned opposite the surface comprising the plurality of assay sites, a second magnet, a third magnet, and a metal object, wherein the second magnet and third magnet are located adjacent the surface comprising the plurality of assay sites and such that the opposite poles of the second magnet and the third magnet are directed towards each other; and wherein the metal object is positioned between the second magnet and the third magnet.

In some embodiments, the instrument in-part achieves its compact design by judicious use and re-use of the sample plates (for example, 96-well microtiter plates) to carry out the multiple complex steps in the assay. This presents numerous challenges as the instrument achieves an exceptionally high-level of immunocomplex solution purity without compromise of contaminants that is typically associated with plate re-use all the while still maintaining minimal use of precious resources, such as wash solutions and other reagents, waste materials and process time. For example, in the course of carrying out the process, the instrument in some embodiments may re-use sample plates (for example, 96-well microtiter plates) in the main stream of the process that requires taking the sample solution and forming an immunocomplex formation solution wherein the immunocomplexes are formed. The immunocomplex solution is then mixed with a first substrate to form a first immunocomplex-capture solution wherein the immunocomplex is captured on the first substrate. The first substrate with bound immunocomplexes is separated from the first immunocomplex-capture solution and washed to remove contaminants (for example: impurities, uncaptured immunocomplexes, incomplete immunocomplexes, antibodies, unbound analytes, blocked analytes, non-targeted analytes, process reagents and fragments thereof). The first substrate with bound immunocomplex is then mixed with an elusion solution to elute the first substrate from the immunocomplex to form a first purified immunocomplex solution. The first purified immunocomplex solution is then mixed with a second substrate to form a second immunocomplex-capture solution wherein the immunocomplex is captured on the second substrate. The second substrate with bound immunocomplexes is separated from the second immunocomplex-capture solution and washed to remove contaminants. The second substrate with bound immunocomplex is then mixed with ligation solution to form an immunocomplex-ligation solution wherein the immunocomplex reporters are formed. The second substrate with bound immunocomplex (and immunocomplex reporters) is then separated from the immunocomplex-ligation solution and washed to remove contaminants. The second substrate with bound immunocomplex is then mixed with an elusion solution to elute the second substrate from the immunocomplex to form a second purified immunocomplex solution comprising immunocomplex reporters.

In some embodiments, for example, in the course of carrying out the process, the instrument in some embodiments may wash and re-use sample plates (for example, 96-well microtiter plates) that are used in the main course of carrying out the analyte assay process. Specifically, in the course of processing the analyte via the immunocomplex and proceeding to purify the immunocomplex through the dual capture and release steps to minimize the presence of contaminants that might contribute to noise in the final detection and analysis, the re-use of plates, instead of additional sterile plates, is unique and, in-part, enables the process to be conducted in a smaller space. This contributes to the compact design of the instrument. For example, in the course of carrying out the process, the instrument in some embodiments may re-use plates in the main stream of the process that requires taking the sample solution and forming an immunocomplex formation solution in a 1st-plate wherein the immunocomplexes are formed. The immunocomplex solution while in the 1st-plate is then mixed with a first substrate to form a first immunocomplex-capture solution, also in the 1st-plate, wherein the immunocomplex is captured on the first substrate. The first substrate with bound immunocomplexes is removed from the 1st-plate and washed in a 2nd-plate to remove contaminants (for example: impurities, uncaptured immunocomplexes, incomplete immunocomplexes, antibodies, unbound analytes, blocked analytes, non-targeted analytes, process reagents and fragments thereof). Optionally, the first substrate with bound immunocomplexes is removed from the 2nd-plate and moved to the 1 st-plate for additional washing. The first substrate with bound immunocomplex is then moved to a 3rd-plate and mixed with an elusion solution to elute the first substrate from the immunocomplex to form a first purified immunocomplex solution in the 3rd-plate. The first purified immunocomplex solution is then mixed with a second substrate, in the 3rd-plate, to form a second immunocomplex-capture solution wherein the immunocomplex is captured on the second substrate, in the 3rd-plate. The second substrate with bound immunocomplexes is removed from the 3rd-plate and washed in a 4th-plate to remove contaminants. The second substrate with bound immunocomplex is then mixed with ligation solution in the (washed) 2nd-plate to form an immunocomplex-ligation solution in the 2nd-plate wherein the immunocomplex reporters are formed. The second substrate with bound immunocomplex (and immunocomplex reporters) is then separated from the 2nd-plate and washed in the (washed) 4th-plate to remove contaminants. The second substrate with bound immunocomplex is then removed from the 4th-plate mixed with an elusion solution in the (washed) 3rd-plate to elute the second substrate from the immunocomplex to form in the 3rd-plate a second purified immunocomplex solution comprising immunocomplex reporters.

In some embodiments, the instrument achieves its compact design, in part, by being equipped with a magnetic bead processor (“mixer”) equipped with a multi-tiered, multi-vessel carrier plate-platforms, a plate washer, and a controller programmed to efficiently control the components to carry out the NULISA multi-plex analysis, for example, in reference to FIGS. 85-90. Specifically, the compact instrument is capable of conducting the NULISA multi-plex analysis on large numbers of samples, for example more than 48 samples in parallel, in a fast processing time, for example less than 8 hours, 7 hours, or 6 hours, and for the dual-capture and release process portion in processing times as low as, less than 3 hours, 2 hours, 1.5 hours, 1 hour or 0.5 hours, and still achieve the high purity required to have high sensitivity, for example lower than 150 attomolar, 100 attomolar, or 50 attomolar, by utilizing the mixer and plate washer in combination with paramagnetic beads to make efficient use (and re-use) of consumables, such as pipette tips and multi-vessel carrier plates. The instrument utilizing, in part, the mixer to transfer the samples (meaning solutions comprising the specific analytes or target analytes or immunocomplexes captured analyte are present) minimizes steps in the process requiring pipette tips to contact the samples and thereby consuming less pipette tips. Similarly, the instrument's use of the plate washer to wash, in process, the multi-vessel carrier plates allows for plates to be re-used, and requires fewer plates in the process.

In some embodiments, the compact instrument carries out the efficient process, in reference to FIG. 85-86, without requiring any pipette tips to contact the sample from the point in the process wherein the samples are deposited into the multi-vessel carrier plate (1st plate) to form the immunocomplex solutions to form the immunocomplexes until after the second purified capture-immunocomplexes with ligated nucleic acid reporters are mixed with an elution solution in the multi-vessel carrier plate (3rd plate). In reference, for example, to the process set forth in FIG. 85, the compact instrument processes the samples, in the 1st plate (where the immunocomplexes are formed and captured onto a first set of paramagnetic beads), the samples are transferred, via the first set of paramagnetic beads, to the 2nd plate to be washed, and the samples are transferred back to the re-used 1st plate (that has been washed in the plate washer) for an optional second wash. The samples are then transferred, via the 1st set of paramagnetic beads, to the 3rd plate (where the immunocomplexes are eluted from the 1st set of paramagnetic beads, the 1st paramagnetic beads are removed from the 3rd plate while the sample solution comprising the immunocomplexes remain in the 3rd plate to be captured on a 2nd set of paramagnetic beads). The samples are transferred, via the 2nd paramagnetic beads, to the re-used 2nd plate (that has been washed in the plate washer) and washed. The samples are then transferred, via the 2nd paramagnetic beads, to a 4th plate (for ligation to form the nucleic acid reporters). The samples are transferred back into the re-used 2nd plate (that has been washed again in the plate washer) and washed. The samples are transferred, via the 2nd paramagnetic beads, to the re-used 3rd plate (that has been washed in the plate washer) to elute the 2nd paramagnetic beads. Finally, the samples using pipette tips are transferred to a per plate and placed into a PCR unit within the instrument.

In some embodiments, in reference, for example, to the process set forth in FIG. 86, the compact instrument processes the samples, in the 1st plate (where the immunocomplexes are formed and captured onto a first set of paramagnetic beads), the samples are transferred, via the first set of paramagnetic beads, to the 2nd plate to be washed, and the samples are transferred back to the re-used 1st plate (that has been washed in the plate washer) for an optional second wash. The samples are then transferred, via the 1st set of paramagnetic beads, to the 3rd plate (where the immunocomplexes are eluted from the 1st set of paramagnetic beads, the 1st paramagnetic beads are removed from the 3rd plate while the sample solution comprising the immunocomplexes remain in the 3rd plate to be captured on a 2nd set of paramagnetic beads). The samples are transferred, via the 2nd paramagnetic beads, to a 4th plate and washed. The samples are then transferred, via the 2nd paramagnetic beads, to the re-used 2nd plate (that has been washed in the plate washer) for ligation to form the nucleic acid reporters. The samples are transferred, via the 2nd paramagnetic beads, to the re-used 3rd plate (that has been washed in the plate washer) to elute the 2nd paramagnetic beads. Finally, the samples using pipette tips are transferred to a PCR plate and placed into a PCR unit within the instrument.

### 3. BRIEF DESCRIPTION OF FIGURES

FIG. 1 illustrates a “Cloud-centric” assay eco-system comprising a Cloud based software program (the “App”) providing a user interface on any computing device, a set of assay specific reagents and consumables and an automated assay instrument.

FIG. 2 illustrates the system-level hardware architecture of the disclosed instrument.

FIGS. 3A-C show an exemplary design embodiment of the disclosed system.

FIGS. 4A-B show a particular design embodiment of the Hotel, which comprises three almost identically constructed “bays”, each providing shelfing spaces to stage a set of reagents and consumables to execute an assay run.

FIG. 5 shows a particular embodiment of the gantry design, which moves the end-effector in three degrees of freedom in X, Y and Z axis.

FIG. 6 shows a particular embodiment of the end-effector, on which 2 air displacement pipettors (ADP), a plate gripper, a laser distance sensor and a barcode scanner are installed.

FIG. 7A-B depict a specific implementation where the relative position between a distance sensor and a specifically designed target are determined in all 3 dimensions. More specifically, FIGS. 7A-B shows gantry position calibration using an optical distance sensor fixed on its end effector. FIG. 7A shows a distance sensor approaching a target block with laser beam crossing the top edge of the target block. FIG. 7B shows change of measured distance (y-axis) when the sensor is scanned across the top edge of the block.

FIGS. 8A-B shows a particular embodiment of the stand, providing six plate positions. More specifically, FIG. 8A shows a perspective view; and FIG. 8B shows a top view.

FIGS. 9A-9B shows a particular implementation of the corner pusher mechanism where each pusher is spring loaded to the “close” position. More specifically, FIGS. 9A-B shows an exemplary embodiment of a pusher mechanism that aligns the plate to the edges of the pocket on the stand. In FIG. 9A, the cam 9004 under the Stand forces the spring loaded corner pusher 9002 to open, allowing plates to be placed into or removed from the pocket at one end of travel. When moving away from the end of travel, the cam 9004 releases the pusher 9002 to push the plate to align against the corner of the pocket.

FIGS. 10A-C shows a particular implementation of a mechanism to restrain a plate placed in a pocket from moving up. More specifically, FIG. 10A shows the pusher in an open position, when plate can be placed into or removed out of pocket with overhang taps at edges. FIG. 10B shows the pusher pushes the plate toward a corner of the pocket. The flange of the plate is constrained under the overhang of the tabs and cannot move up, as detailed in FIG. 10C.

FIGS. 11A-C depicts the concept and operating principle of a bead processing device with multiple plate platforms stacked vertically. More specifically, FIG. 11A depicts a front view of the structure, showing magnetic head with poles 11002, plastic comb with sleeves 11004, and assay plates staged on vertically positioned platforms 11006. FIG. 11B illustrates the bead processing device when accessing a plate on the upper platform, where magnetic poles are inserted in sleeves 11008. FIG. 11C shows the bead processing device when accessing a plate on a lower floor.

FIGS. 12A-C shows a design concept where the upper assay plate is placed on a “splitting” platform to further reduce the footprint of the module. More specifically, FIGS. 12A-C depicts the concept of splitable platforms. FIG. 12A shows the splitable platform when accessing plate on the upper platform, and FIGS. 12B-C show the splitable platform when accessing a plate on the lower platform.

FIGS. 13A-B shows another concept of the disclosed bead processor with fixed and vertically positioned platforms for assay plates. The upper platform(s) have an opening at center that allows the magnetic pole and sleeve to go through it to access the plate on lower platform. More specifically, FIGS. 13A and 13B show respectively show a plate on the top and lower floor may be accessed.

FIGS. 14A-F shows an exemplary design of the concept of the fixed plate platforms illustrated in FIGS. 13A-B.

FIGS. 15A-C depicts the “ear-like” alignment feature on a sleeve comb (FIG. 15A) and mechanism on the disclosed bead processor used to align the sleeve comb to the magnetic head (FIGS. 15B, 15C). More specifically, FIG. 15A shows a sleeve comb with “ear-like” structures on base plate, as indicated by circles. FIG. 15B shows a “finger” engages the “ear” to align the comb to its carrier. FIG. 15B shows the “ear” 15002 on comb base plate, comb carrier 15004, rotary part 15006, sleeve 15008, and “finger” 15010 on the rotary part. FIG. 15C shows the “flapper” 15012 engages the underside of the comb to hold it to its carrier.

FIG. 16 illustrates the basic conceptual structure of the disclosed plate washer, which comprises one or more stripe dispensing head 16004 and a matrix aspiration head 16002.

FIG. 17 illustrates a matrix aspiration head with a partitioned manifold atop the needle matrix with each manifold connecting a portion of the needles in the entire matrix.

FIG. 18 depicts an exemplary system architecture of the disclosed washer, comprising two dispensing stripes where the dispensing strip #1 is shared by Buffers 1 & 2 (B1 & B2) selected through a 3-way valve and strip #2 is dedicated to Buffer 3. The aspiration manifold is partitioned into two separate portions, each connected to an aspiration pump.

FIGS. 19A-B illustrate an invented approach to eliminate fluid residuals trapped at dead ends of aspiration manifold without increasing the power of the aspiration pump. More specifically, FIG. 19A shows that aspiration from one end or center of the manifold results in residuals 19002 at “dead end” portion of the manifold. FIG. 19B shows “residual free” aspiration from both ends of the manifold.

FIGS. 20A-B shows a particular embodiment for the system-level piping and instrumentation diagram (PID) of the disclosed plate washer. More specifically, FIGS. 20A and 20B show the dispensing side and the aspiration side respectively.

FIGS. 21A and B shows the details of the design embodiment of the disclosed plate washer. More specifically, FIGS. 21A and 21B show a perspective and side view of the of the plate washer.

FIGS. 22A-22B illustrates the conceptual structure of the disclosed incubator & sealer module, which comprises a temperature-controlled top and bottom block and a thermal isolation enclosure.

FIGS. 23A-23B illustrates the operating process for the use of the disclosed incubation & sealing module for incubation.

FIGS. 24A-E illustrates the operating process for the use of the disclosed incubation & sealing module for sealing of assay or sample plates.

FIGS. 25A-D show an exemplary design of the disclosed incubator & sealer module.

FIGS. 26A-G show a particular embodiment of the bulk station comprising a bulk station door, bulk nests and a tube manifold. More specifically, FIG. 26A shows the bulk station with door closed, and the tube manifold carries tubes to dip into bulk fluid bottles (door panel removed for clarity). FIG. 26B shows the tube manifold rises to open the bulk door (panel removed). Tubes are lifted out of bulk fluid bottles to allow bottles to pull out of or insert into their respective nests. FIG. 26C shows a general view of the Safety Switch Mechanism. FIG. 26D shows the Safety Switch Mechanism in the extended “SAFE” position, which lifts and lowers the door position (switches “open”). FIG. 26E shows the Safety Switch Mechanism in the compressed “STOP” position, which lowers the door with obstruction position (switches “closed”). FIGS. 26F and 26G show the two possible positions of the bulk bottle caps.

FIG. 27 illustrates a specific embodiment of the frame design in the disclosed Framed Sealing Film (FSF). [better to have the film mounted on frame]

FIG. 28 shows a specific embodiment design of the perforation pattern on the disclosed FSF.

FIGS. 29A-C show a specific design embodiment of the disclosed universal reagent cartridge, which comprises a base, a lid and multiple reagent containers of different types. More specifically, FIG. 29A shows the parts of the cartridge, including containers 29002, TRIB 29004, MONO 29006, base 29008, and lid 29010. FIG. 29B depicts the base loaded with containers. FIG. 29C depicts an assembled cartridge.

FIGS. 30A-C shows one specific embodiment of the disclosed consumable carrier. More specifically, FIG. 30A shows a carrier with a lid and 7 consumable items. FIG. 30B shows a locked lid with spring fingers to hold a consumable stack for transportation. FIG. 30C shows the consumable carrier ready to be loaded into the instrument after the lid is removed.

FIG. 31 illustrates bulk fluid bottles with mechanical features preventing it being inserted into incorrect positions in the instrument and a device to reduce fluid splashing.

FIG. 32 illustrates an embodiment of the Alamar Biosciences ARGO HT Instrument.

FIG. 33 illustrates an embodiment of the Alamar Biosciences ARGO HT Instrument.

FIG. 34 illustrates a single-plex workflow in the ARGO HT Instrument.

FIG. 35 illustrates an embodiment of a multi-plex workflow in the ARGO HT Instrument.

FIG. 36 illustrates ARGO HT Software Buttons, Icons and Symbols.

FIG. 37 illustrates Alamar NULISA Analysis (ANA) Software Buttons, Icons and Symbols.

FIG. 38 illustrates a front view of the Alamar Biosciences ARGO HT Instrument.

FIG. 39 illustrates bay components of the ARGO HT Instrument.

FIG. 40 illustrates bulk liquid components of the ARGO HT Instrument.

FIG. 41 illustrates a rear view of the ARGO HT Instrument.

FIG. 42 illustrates the ARGO HT home screen after start-up.

FIG. 43 illustrates a single-plex workflow in the ARGO HT Instrument ARGO HT Instrument.

FIG. 44 illustrates a Home Screen of the Alamar NULISA Analysis (ANA) software.

FIG. 45 illustrates an Experiments Screen of the Alamar NULISA Analysis (ANA) software.

FIG. 46 illustrates an Experiment Screen of the Alamar NULISA Analysis (ANA) software with a selected experiment.

FIG. 47 illustrates an Experiments Screen of the Alamar NULISA (ANA) Analysis software showing well assignments.

FIG. 48 illustrates an Experiments Screen of the Alamar NULISA Analysis (ANA) software showing a Create a New Project feature.

FIG. 49 illustrates an Experiments Screen of the Alamar NULISA Analysis (ANA) software showing a Create New Experiment feature.

FIG. 50 illustrates an Experiments Screen of the Alamar NULISA Analysis (ANA) software showing an Edit Experiment feature.

FIG. 51 illustrates an Experiments Screen of the Alamar NULISA Analysis (ANA) software showing an Experiment Scheduling feature.

FIG. 52 illustrates an Experiments Screen of the Alamar NULISA Analysis (ANA) software showing a scheduled experiment.

FIG. 53 illustrates an Experiments Screen of the Alamar NULISA Analysis (ANA) software showing a prompt to load the experiment into the first bay.

FIG. 54 illustrates an Experiments Screen of the Alamar NULISA Analysis (ANA) software showing a prompt that experiment is loaded in the first bay and is ready to run.

FIG. 55 illustrates an Experiments Progress Bar of the Alamar NULISA Analysis (ANA) software.

FIG. 56 illustrates an Experiments Screen of the Alamar NULISA Analysis (ANA) software showing that the experiment is ready to unload.

FIG. 57 illustrates a reagent loading overview of a bay of the ARGO HT Instrument.

FIG. 58 illustrates a bay of the ARGO HT Instrument which shows loading of the tip boxes.

FIG. 59 illustrates a bay of the ARGO HT Instrument which shows loading of consumables cartridge.

FIG. 60 illustrates a bay of the ARGO HT Instrument which shows loading of the sample and reagent kit cartridges.

FIG. 61 illustrates the Experiments Screen of the Alamar NULISA Analysis (ANA) software showing an option for viewing of experiment results.

FIG. 62 illustrates the Experimental Results screen of the Alamar NULISA Analysis (ANA) software showing a prompt to load the results.

FIG. 63 illustrates the Experimental Results Loaded screen of the Alamar NULISA Analysis (ANA) software.

FIG. 64 illustrates the Alamar NULISA Analysis (ANA) Screen with Results Selected.

FIG. 65 illustrates the Alamar NULISA Analysis (ANA) Screen with QC Results.

FIG. 66 illustrates the Alamar NULISA Analysis (ANA) Screen with Heatmap Results.

FIG. 67 illustrates the Alamar NULISA Analysis (ANA) Screen with Results Table.

FIG. 68 illustrates the Alamar NULISA Analysis (ANA) Instruments Screen with an Instrument selected.

FIG. 69 illustrates the Alamar NULISA Analysis (ANA) Instruments Screen with an Instrument selected.

FIG. 70 illustrates the Alamar NULISA Analysis (ANA) Instruments Screen with an Experiment selected.

FIG. 71 illustrates the Alamar NULISA Analysis (ANA) Settings Screen with user groups tab selection.

FIG. 72 illustrates the Alamar NULISA Analysis (ANA) Settings Screen with adding and deleting group members.

FIG. 73 illustrates the Alamar NULISA Analysis (ANA) Settings Screen with instrument tabs selection.

FIG. 74 illustrates the Account Settings drop-down menu.

FIG. 75 illustrates the Manage your account screen.

FIG. 76 illustrates the User Management Screen.

FIG. 77 illustrates the Create New User Screen.

FIG. 78 illustrates the User Details Screen.

FIG. 79 illustrates the Upload user file Dialog Box.

FIG. 80 illustrates the Template File in Microsoft Excel.

FIG. 81 illustrates bulk liquid components.

FIG. 82 illustrates removing waste water bottle.

FIG. 83 illustrates removing consumable bottles.

FIG. 84A illustrates a Quick Reference Guide for running an experiment.

FIG. 84B illustrates a Quick Reference Guide for analyzing data, powering on/off the instrument, and maintenance.

FIG. 85 illustrates a flowchart of steps performed by a compact fully-automated instrument designed to complete a NULISA multi-plex analysis.

FIG. 86 illustrates a flowchart of steps performed by a compact fully-automated instrument designed to complete a NULISA multi-plex analysis.

FIG. 87 illustrates a flowchart of steps performed by a compact fully-automated instrument designed to complete a NULISA multi-plex analysis.

FIG. 88 illustrates a flowchart of steps performed by a compact fully-automated instrument designed to complete a NULISA multi-plex analysis.

FIG. 89 illustrates a flowchart of steps performed by a compact fully-automated instrument designed to execute a NULISA dual-capture and release multi-plex mechanism.

FIG. 90 illustrates a flowchart of steps performed by a compact fully-automated instrument designed to complete a NULISA multi-plex analysis.

FIG. 91A illustrates a standard curve of IFNL1 generated on the ARGO HT Instrument.

FIG. 91B illustrates a standard curve of CSF2 generated on the ARGO HT Instrument.

### 4. DETAILED DESCRIPTION

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, molecular biology, immunology, genetics and protein and nucleic acid chemistry and hybridization described herein are those well-known and commonly used in the art.

As used herein, the term “detect” or its grammatical equivalents are used broadly to include any means of determining the presence of the analyte (i.e. if it is present or not) or any form of measurement of the analyte. Thus “detecting” can include determining, measuring, or assessing the presence or absence or amount or location of analyte. Quantitative, semi-quantitative and qualitative determinations, measurements or assessments are included. Such determinations, measurements or assessments can be relative, for example, when two or more different analytes in a sample are being detected, or absolute. As such, the term “quantifying” when used in the context of quantifying a target analyte(s) in a sample can refer to absolute or to relative quantification. Absolute quantification can be accomplished by inclusion of known concentration(s) of one or more control analytes and/or referencing the detected level of the target analyte with known control analytes (e.g., through generation of a standard curve). Alternatively, relative quantification can be accomplished by comparison of detected levels or amounts between two or more different target analytes to provide a relative quantification of each of the two or more different analytes, i.e., relative to each other.

As used herein, the term “analyte” can be any substance (e.g. molecule) or entity to be detected by the assay methods provided herein. The analyte is the target of the assay method provided herein. Accordingly, the analyte can be any biomolecule or chemical compound that need to be detected, for example a peptide or protein, a nucleic acid molecule or a small molecule, including organic and inorganic molecules. The analyte can be a cell or a microorganism, including a virus, or a fragment or product thereof. The analyte can be any substance or entity for which a specific binder can be developed, and which is capable of simultaneously binding at least two “binders.” In some embodiments, the analytes are proteins or polypeptides. As such, analytes of interest include proteinaceous molecules such as polypeptides, proteins or prions or any molecule which contains a protein or polypeptide component, or fragments thereof. In some embodiments, the analyte is a wholly or partially proteinaceous molecule. The analyte can also be a single molecule or a complex that contains two or more molecular subunits, which may or may not be covalently bound to one another, and which may be the same or different. Thus, the analyte that can be detected by assay methods described herein can be a complex analyte, which can be a protein complex. Such a complex can thus be a homo- or hetero-multimer. Aggregates of molecules (e.g. proteins) can also be target analytes. The aggregate analytes can be aggregates of the same protein or different proteins. The analyte can also 3Abe a complex composed of proteins or peptides, or nucleic acid molecules such as DNA or RNA. In some embodiments, the analyte is a complex composed of both proteins and nucleic acids, e.g. regulatory factors, such as transcription factors.

As used herein, the term “sample” can be any biological and clinical samples, included, e.g. any cell or tissue sample of an organism, or any body fluid or preparation derived therefrom, as well as samples such as cell cultures, cell preparations, cell lysates, etc. Environmental samples, e.g. soil and water samples or food samples are also included. The samples can be freshly prepared or prior-treated in any convenient way (e.g. for storage).

Representative samples thus include any material that contains a biomolecule, or any other desired or target analyte, including, for example, foods and allied products, clinical and environmental samples. The sample can be a biological sample, including viral or cellular materials, including prokaryotic or eukaryotic cells, viruses, bacteriophages, mycoplasmas, protoplasts and organelles. Such biological material comprise all types of mammalian and non-mammalian animal cells, plant cells, algae including blue-green algae, fungi, bacteria, protozoa etc. Representative samples also include whole blood and blood-derived products such as plasma, serum and buffy coat, blood cells, urine, faeces, cerebrospinal fluid or any other body fluids (e.g. respiratory secretions, saliva, milk, etc.), tissues, biopsies, cell cultures, cell suspensions, conditioned media or other samples of cell culture constituents, etc. The sample can be pre-treated in any convenient or desired way to prepare for use in the method disclosed herein. For example, the sample can be treated by cell lysis or purification, isolation of the analyte, etc.

As used herein, the term “bind” or its grammatical equivalents refer to an interaction between molecules (e.g. a binder and an analyte, or a presenting group and a receiving group) to form a complex. Interactions can be, for example, non-covalent interactions including hydrogen bonds, ionic bonds, hydrophobic interactions, and/or van der Waals interactions. A “binder,” as used herein in connection with an analyte, is any molecule or entity capable of binding to the analyte. In some embodiments, a binder binds specifically to its target analyte, namely, the binder binds to the target analyte with greater affinity than to other components in the sample. In some embodiments, the binder's binding to the target analyte can be distinguished from that to non-target analytes in that the binder either does not bind to non-target analytes or does so negligibly or non-detectably, or any such non-specific binding, if it occurs, is at a relatively low level that can be distinguished. The binding between the target analyte and its binder is typically non-covalent. The binder used in methods provided herein can be covalently conjugated to a presenting group (e.g. a nucleic acid tag) without substantially abolishing the binding affinity of the binder to its target analyte.

The binder can be selected to have a high binding affinity for a target analyte. In some embodiments, the binder can have a binding affinity to the target analyte of at least about 10⁻⁴ M, at least about 10⁻⁶ M, or at least 10⁻⁹ M or higher. The binder can be a variety of different types of molecules, so long as it exhibits the requisite binding affinity for the target analyte. In other embodiments, the binder can have a medium or even low affinity for its target analyte, e.g., less than about 10⁻⁴ M.

The binder can be a large molecule. In some embodiments, the binders are antibodies, or binding fragments, derivatives or mimetics thereof. Where antibodies are the binder, they can be derived from polyclonal compositions, such that a heterogeneous population of antibodies differing by specificity are each conjugated with the same presenting group, or monoclonal compositions, in which a homogeneous population of identical antibodies that have the same specificity for the target analyte are each conjugated with the same presenting group. As such, the binder can be either a monoclonal or polyclonal antibody.

In some embodiments, the binder is an antibody fragment, derivative or mimetic thereof, where these fragments, derivatives and mimetics have the requisite binding affinity for the target analyte. Such antibody fragments or derivatives generally include at least the V_{H} and V_{L} domains of the subject antibodies, so as to retain the binding characteristics of the subject antibodies. In some embodiments, the binder is an antibody fragment that binds the analyte. An antibody fragment as used herein refers to a molecule other than an intact antibody that comprises a portion of an antibody and generally an antigen-binding site. Examples of antibody fragments include, but are not limited to, Fab, Fab′, F(ab′)2, Fv, single chain antibody molecules (e.g., scFv), disulfide-linked scFv (dsscFv), diabodies, tribodies, tetrabodies, minibodies, dual variable domain antibodies (DVD), single variable domain antibodies (e.g., camelid antibodies, alpaca antibodies), single variable domain of heavy chain antibodies (VHH), and multispecific antibodies formed from antibody fragments. In some embodiments, the binder is an Fab. In some embodiments, the binder is a scFv. In some embodiments, the binder is a single variable domain antibody.

In some embodiments, the binder is an antibody mimetic. An antibody mimetic can be molecules that, like antibodies, can specifically bind antigens, but that are not structurally related to antibodies. The antibody mimetics are usually artificial peptides within a molar mass of about 2 to 20 kDa. Nucleic acids and small molecules are sometimes considered antibody mimetics as well. Antibody mimetics known in the art include affibodies, affilins, affimers, affitins, alphabodies, anticalins, aptamers, avimers, DARPins, Fynomers, Kunitz domain peptides, monobodies, and nanoCLAMPs.

In some embodiments, suitable for use as binders are polynucleic acid aptamers. Polynucleic acid aptamers can be RNA oligonucleotides which can act to selectively bind proteins, much in the same manner as a receptor or antibody (Conrad et al., Methods Enzymol. (1996), 267 (Combinatorial Chemistry), 336-367). The above described antibodies, fragments, derivatives and mimetics thereof can be obtained from commercial sources and/or prepared using any convenient technology, where methods of producing polyclonal antibodies, monoclonal antibodies, fragments, derivatives and mimetics thereof, including recombinant derivatives thereof, are known to those of the skill in the art (e.g. U.S. Pat. Nos. 5,851,829 and 5,965,371).

In addition to antibody-based peptide/polypeptide or protein-based binding domains, the binder can also be a lectin, a soluble cell-surface receptor or derivative thereof, an affibody or any combinatorically derived protein or peptide from phage display or ribosome display or any type of combinatorial peptide or protein library.

The binder can also be a ligand. The ligand binder can have different sizes. In some embodiments, the ligand binder has a size from about 50 to about 10,000 daltons, from about 50 to about 5,000 daltons, or from about 100 to about 1000 daltons. In some embodiments, the ligand binder has a size of about 10,000 daltons or greater in molecular weight.

In some embodiments, the binder is a small molecule that is capable of binding with the requisite affinity to the target analyte. The small molecule can be a small organic molecule. The small molecule can include one or more functional groups necessary for structural interaction with the target analyte, e.g. groups necessary for hydrophobic, hydrophilic, electrostatic or even covalent interactions. Where the target analyte is a protein, the small molecule binder can include functional groups necessary for structural interaction with proteins, such as hydrogen bonding, hydrophobic-hydrophobic interactions, electrostatic interactions, etc., and typically include at least an amine, amide, sulfhydryl, carbonyl, hydroxyl or carboxyl group. In some embodiments, at least two of the functional groups are included. The small molecule binder can also comprise a region that can be modified and/or participate in covalent linkage to a presenting group (e.g. a nucleic acid tag), without substantially adversely affecting the small molecules ability to bind to its target analyte.

Small molecule binders can also comprise cyclical carbon or heterocyclic structures and/or aromatic or polyaromatic structures substituted with one or more of the above functional groups. Small molecule binders can also contain structures found among biomolecules, including peptides, saccharides, fatty acids, steroids, purines, pyrimidines, derivatives, structural analogs or combinations thereof. Such compounds can be screened to identify those of interest. A variety of different screening protocols are known in the art.

The small molecule binder can also be derived from a naturally occurring or synthetic compound that can be obtained from a wide variety of sources, including libraries of synthetic or natural compounds. For example, numerous means are available for random and directed synthesis of a wide variety of organic compounds and biomolecules. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known small molecules can be subjected to directed or random chemical modifications, such as acylation, alkylation, esterification, amidification, etc., to produce structural analogs. As such, the small molecule binders can be obtained from a library of naturally occurring or synthetic molecules, including a library of compounds produced through combinatorial means, i.e. a compound diversity combinatorial library. When obtained from such libraries, the small molecule binders are selected for demonstrating some desirable affinity for the protein target in a convenient binding affinity assay.

The assay methods provided herein use a first binder and a second binder that bind non-interfering “epitopes” of an analyte. An epitope of an analyte, as understood in the art, refers to a site on the surface of an analyte to which a binder binds. An epitope can be a localized region on the surface of an analyte. An epitope can consist of chemically active surface groupings of molecules such as amino acids or sugar side chains. An epitope can have specific three dimensional structural characteristics and specific charge characteristics. An epitope can be a continuous fragment of the analyte molecule. An epitope can also be a molecule having more than one non-continuous fragments of the antigen linked together. If the analyte is a polypeptide or a protein, its epitope can include continuous or non-continuous sequence along the primary sequence of the polypeptide chain. In some embodiments, the first and the second binders used in the assay methods disclosed herein are of the same type of molecule. For example, the first and second binders can both be monoclonal antibodies that bind non-interfering epitopes of the analyte. In some embodiments, the first and the second binders can be different. For example, the first binder can be an antibody, and the second binder can be a small molecule.

The term “binding moiety,” when used in reference to an analyte, refers to a moiety including a molecule or a collection of more than one molecules, such that the moiety as a whole is capable of binding specifically to an analyte. The binding moiety can include one or more binder, one or more target label, one or more sample label, and/or one or more presenting group. The binding moiety can also include a binder, a target label, a sample label, and/or a presenting group. Alternatively, the binding moiety can include a binder, a target label, and/or a presenting group. The molecules in the binding moiety can be held together as a moiety by covalent, non-covalent, or a combination of covalent or non-covalent intermolecular interactions. Alternatively, the molecules in the binding moiety can be held together via interactions with a molecule that is not part of the binding moiety, for example the analyte or one or more receiving groups. Additionally, the molecules in the binding moiety can be held together as a moiety via (i) intermolecular interactions among the molecules within the binding moiety and (ii) via interactions with a molecule that is not part of the binding moiety, for example the analyte or one or more receiving groups. In one embodiment, the binding moiety comprises or consists of a binder. In some embodiments, the binding moiety comprises or consists of a target label. In certain embodiments, the binding moiety comprises or consists of a presenting group. In other embodiments, the binding moiety comprises or consists of a sample label. In one embodiment, the binding moiety comprises or consists of a binder and a target label. In some embodiments, the binding moiety comprises or consists of a binder and a presenting group. In certain embodiments, the binding moiety comprises or consists of a binder and a sample label. In further embodiments, the binding moiety comprises or consists of a target label and a presenting group. In one embodiment, the binding moiety comprises or consists of a target label and a sample label. In other embodiments, the binding moiety comprises or consists of a presenting group and a sample label. In yet other embodiments, the binding moiety comprises or consists of a binder, a target label, and a presenting group. In some embodiments, the binding moiety comprises or consists of a binder, a target label and a sample label. In certain embodiments, the binding moiety comprises or consists of a binder, a presenting group and a sample label. In some embodiments, the binding moiety comprises or consists of a target label, a presenting group and a sample label. In other embodiments, the binding moiety comprises or consists of a binder, a target label, a presenting group, and a sample label. In some embodiments, the binding moiety comprises or consists of any one of a binder, a target label, a presenting group, and a sample label. In some embodiments, the binding moiety comprises or consists of any two of a binder, a target label, a presenting group, and a sample label, in any combination or permutation. In some embodiments, the binding moiety comprises or consists of any three of a binder, a target label, a presenting group, and a sample label, in any combination or permutation. In some embodiments, the binding moiety comprises or consists of all four of a binder, a target label, a presenting group, and a sample label.

The terms “presenting group” and “receiving group” are used herein in reference to each other, which refer to a binding pair that can form, a complex under appropriate conditions. As used in the assay methods disclosed herein, presenting groups can be conjugated to binders for the target analyte, and receiving groups can be coupled to a solid surface. Therefore, the binding between presenting group and receiving group allows the analyte to be captured on the solid surface. In some embodiments, the bond formed between the presenting group and receiving group is “releasable,” allowing the captured binder to be released from the solid surface. In some embodiments, the bond formed between the presenting group and receiving group is “renewable,” allowing the binder to be recaptured to another solid surface coupled with the same receiving group. Same as the binding pair of a “binder” and its “analyte” discussed above, the binding pair of “presenting group” and “receiving group” can take a variety of forms. Examples of binding pairs of “presenting groups” and “receiving groups” include, but are not limited to, an antigen and an antibody against the antigen (including its fragments, derivatives or mimetics), a ligand and its receptor, complementary strands of nucleic acids, biotin and avidin (or streptavidin or neutravidin), lectin and carbohydrates, and vice versa. Additional binding pairs of “presenting groups” and “receiving groups” include fluorescein and anti-fluorescein, digioxigenin/anti-digioxigenin, and DNP (dinitrophenol)/anti-DNP, and vice versa. In some embodiments, binding pairs of “presenting groups” and “receiving groups” are complementary strands of nucleic acids, and are referred to as “tags” and “probes.” In some embodiments, binding pairs of “presenting groups” and “receiving groups” are antigens and antibodies, or antigens and antibody fragments.

The term “target label” refers to a moiety that facilitates detection and identification of a target molecule. The term “sample label” refers to a moiety that facilitates detection and identification of sample origin of the target. Suitable labels for target label and sample label include labels that can provide identifiers that can be correlated with the particular target or sample. A common label that can be used for target label and/or sample label in the context of the present disclosure is sequences of nucleotides which can be correlated with the target or sample via sequencing. In some embodiments, a target label comprises a target ID. In certain embodiments, a target label consists of a target ID. In other embodiments, the target label is the target ID. In some embodiments, a sample label comprises a sample ID. In certain embodiments, a sample label consists of a sample ID. In other embodiments, the sample label is the sample ID. Additional labels suitable for target label and sample label of the present disclosure include other identifiable or correlative information containing molecules, such as fluorescent molecules or the combination or sequences of fluorescent molecules, and/or colorimetric moieties or the combination or sequences of colorimetric moieties. Other labels contemplated for the present disclosure include luminescent, light-scattering, radionuclides, substrates, cofactors, inhibitors, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Many labels are commercially available and can be used in the context of the invention.

The term “identity barcode” or “ID,” when used in reference with target or sample, refers to a molecule or a series of molecules that can be used to identify, directly or indirectly through the identification information contained in the molecule or the series of the molecules, the target or the sample. Such an ID can be a nucleic acid molecule with a given sequence, a unique fluorescent label, a unique colorimetric label, a sequence of the fluorescent labels, a sequence of the colorimetric label, or any other molecules or combination of molecules, so long as molecules or the combination of molecules used as IDs can identify or otherwise distinguish a particular target or sample from other targets or samples and be correlated with the intended target or sample. Nucleic acid molecules used as such IDs are also known as barcode sequences. Such an ID can also be a further derivative molecule that contains the information derived from but is non-identical to the original ID, so long as such derived molecules or the derived information can identify or otherwise distinguish a particular target or sample from other targets or samples and be correlated with the intended target or sample. For example, a nucleic acid ID can include both the original nucleic acid barcode sequence and/or the reverse complement of the original nucleic acid barcode sequence, as both can distinguish and be correlated with the intended target or sample. The barcode sequence can be any sequences, natural or non-natural, that are not present without being introduced as barcode sequences in the intended sample, the intended target, or any part of the intended sample or target, so that the barcode sequence can identify and be correlated with the sample or target. A barcode sequence can be unique to a single nucleic acid species in a population or a barcode sequence can be shared by several different nucleic acid species in a population. Each nucleic acid probe in a population can include different barcode sequences from all other nucleic acid probes in the population. Alternatively, each nucleic acid probe in a population can include different barcode sequences from some or most other nucleic acid probes in a population. For a specific example, all the reporters generated from immunocomplexes from one sample can have the same sample barcode sequence (sample ID). For another example, all the reporters generated from immunocomplexes from the same sample can have different target barcode sequences (target IDs). Furthermore, all the reporters generated from immunocomplexes from the same sample, for the same target, and with the same binders can have the same target barcode sequences (target IDs).

The term “bench-top” or “compact”, when used in reference to system or instrument or housing or enclosure, refers to a system or instrument or housing or enclosure that has a base depth (from front to back) dimension of no more than 36 inches, no more than 32 inches, no more than 30 inches, no more than 28 inches, no more than 26 inches, no more than 24 inches, no more than 22 inches, or no more than 20 inches. For example, in some embodiments, the bench-top system or instrument provided herein may have a base depth (from front to back) dimension no more than 36 inches, no more than 32 inches, no more than 30 inches, no more than 28 inches, no more than 26 inches, no more than 24 inches, no more than 22 inches, or no more than 20 inches and a height (from top to base) dimension of no more than 50 inches, no more than 48 inches, no more than 46 inches, no more than 44 inches, no more than 42 inches, no more than 40 inches, no more than 38 inches, no more than 36 inches, no more than 34 inches, no more than 32 inches, or no more than 30 inches.

In some embodiments, the bench-top system or instrument provided herein may have a base depth (from front to back) dimension no more than 36 inches, no more than 32 inches, no more than 30 inches, no more than 28 inches, no more than 26 inches, no more than 24 inches, no more than 22 inches, or no more than 20 inches; a height (from top to base) dimension of no more than 50 inches, no more than 48 inches, no more than 46 inches, no more than 44 inches, no more than 42 inches, no more than 40 inches, no more than 38 inches, no more than 36 inches, no more than 34 inches, no more than 32 inches, or no more than 30 inches and a width (from the left side to the right side) dimension of no more than more than 50 inches, no more than 48 inches, no more than 46 inches, no more than 44 inches, no more than 42 inches, no more than 40 inches, no more than 38 inches, no more than 36 inches, no more than 34 inches, no more than 32 inches, or no more than 30 inches.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Provided herein are assay methods that address the limitations of existing immunoassays and enable single molecule detection of immunocomplex through nucleic acid-based signal amplification. The immunocomplex comprises the specific analyte bound between a first antibody or first antibody fragment of a first moiety and a second antibody or second antibody fragment of a second moiety wherein the second moiety may be pre-selected to be paired with or complimentary to the first moiety. Each of the first antibody and the second antibody to non-overlapping target epitopes on the target analyte. As a result the analyte is “sandwiched” between the two antibodies. The methods provided herein reduce background signal through a capture-and-release mechanism. Accordingly, provided herein are assay methods for detecting an analyte in a sample comprising the capture-and-release mechanism. In some embodiments, the capture-and-release mechanism is based on the hybridization and dissociation of nucleic acid pairs.

Assay methods provided herein use a capture-and-release mechanism to reduce non-specific background signals. The processes of capturing the immunocomplex onto solid surface and releasing it back to solution can be applied to different assay formats as disclosed herein.

In some embodiments, assay methods provided herein assay methods provided herein use a capture-and-release mechanism that involves two binders, which can be captured by two receiving groups on two solid surfaces, respectively. In some embodiments, provided herein are assay methods for detecting an analyte in a sample comprising the following steps:
(1) mixing a first binder, a second binder, and the sample in a solution, wherein the first and second binders bind non-interfering epitopes on the analyte and form an immunocomplex, and wherein the immunocomplex is captured on a first solid surface in contact with the solution via binding between a first presenting group conjugated to the first binder and a first receiving group coupled to the first surface;
(2) washing the first solid surface to remove unbound molecules;
(3) releasing the immunocomplex from the first solid surface by disrupting the binding between the first presenting group and the first receiving group;
(4) introducing a second solid surface and recapturing the immunocomplex via binding between a second presenting group conjugated to the second binder and the second receiving group coupled to the second solid surface;
(5) washing the second solid surface to remove unbound molecules; and
(6) detecting the immunocomplex.

The capture/release of the first binder (“Binder 1”) to/from the first solid surface (“Surface 1”) and the capture/release of the second binder (“Binder 2”) to/from the second solid surface (“Surface 2”) are achieved through two bonds between the presenting groups (“PG”) and the receiving groups (“RG”) that are bio-orthogonal (i.e. each independent and specific). The bond between the first Presenting Group (“PG1”) and the first Receiving Group (“RG1”), namely, the first bond (“Bond 1”), is releasable. In some embodiments, the bond between the second Presenting Group (“PG2”) and the second Receiving Group (“RG2”), namely, the second bond (“Bond 2”), is also releasable, and the immunocomplex can be detected either on Surface 2, or after being released from Surface 2. In some embodiments, Bond 2 is not releasable, and the immunocomplex can be detected on Surface 2.

As a person of ordinary skill in the art would understand, additional round(s) of capture/release would further reduce nonspecific background signal. In some embodiments, Bond 1 is renewable, and at least one additional round of capture/release can be performed via Binder 1. Specifically, the immunocomplex released from Surface 2 can be recaptured by a new Surface 1 by forming another bond between PG1 on Binder 1 and RG1 on the new Surface 1. In some embodiments, Bond 2 is renewable, and at least one additional round of capture/release can be performed via Binder 2. Specifically, the immunocomplex released from either Surface 1 or Surface 2 can be recaptured by a new Surface 2 by forming another bond between PG2 on Binder 2 and RG2 on the new Surface 2. In some embodiments, both Bond 1 and Bond 2 are renewable, and more than one cycle of recapture can be performed Bond 1, Bond 2, or both. In some embodiments, neither Bond 1 nor Bond 2 is renewable, and only one cycle of capture/release is performed.

In some embodiments, the releasable and renewable bond is formed via nucleic acid hybridization, wherein the presenting group and the receiving group include nucleic acids that are complementary to each other. In some embodiments, the presenting group is a nucleic acid, which can bind a receiving group that is a DNA/RNA specific protein or aptamer binding partner (e.g. U.S. Pat. No. 5,312,730). In some embodiments, the receiving group is a nucleic acid, which can bind a presenting group that is a DNA/RNA specific protein or aptamer binding partner.

After formation of the immunocomplex, first capture.

In some embodiments, the bench-top system or instrument provided herein may perform, after the formation of a plurality of immunocomplexes in at least one or a plurality of samples, the first capture of at least one of the immunocomplexes of the plurality of immunocomplexes by introducing a plurality of first paramagnetic beads into at least one of the plurality of samples, into at least two of the plurality of samples, into at least three of the plurality of samples, in at least four of the plurality of samples, in at least five of the plurality of samples, in at least ten of the plurality of samples, in at least twenty of the plurality of samples, in at least fifty of the plurality of samples and incubating the at least one of the plurality of samples, into at least two of the plurality of samples, into at least three of the plurality of samples, in at least four of the plurality of samples, in at least five of the plurality of samples, in at least ten of the plurality of samples, in at least twenty of the plurality of samples, in at least fifty of the plurality of samples with the plurality of first paramagnetic beads, for less than 90 minutes, for less than 80 minutes, for less than 70 minutes, for less than 65 minutes, for less than 60 minutes, for less than 55 minutes, for less than 50 minutes, for less than 45 minutes or in less than 40 minutes.
1^{st} capture & release:

In some embodiments, the bench-top system or instrument provided herein may perform, after the formation of a plurality of immunocomplexes in at least one or a plurality of samples, the extraction of the immunocomplex from at least one of the plurality of samples, at least two of the plurality of samples, at least three of the plurality of samples, at least four of the plurality of samples, at least five of the plurality of samples, at least ten of the plurality of samples, at least twenty of the plurality of samples, at least fifty of the plurality of samples by capturing at least one of the immunocomplexes of the plurality of immunocomplexes on at least one first paramagnetic beads of the plurality of first paramagnetic beads and removing, washing and re-introducing the captured immunocomplex or immunocomplexes from at least one of the plurality of samples, at least two of the plurality of samples, at least three of the plurality of samples, at least four of the plurality of samples, at least five of the plurality of samples, at least ten of the plurality of samples, at least twenty of the plurality of samples, at least fifty of the plurality of samples; into at least one of a plurality of second solutions, at least two of a plurality of second samples, at least three of a plurality of second samples, at least four of a plurality of second samples, at least five of a plurality of second samples, at least ten of a plurality of second samples, at least twenty of a plurality of second samples, at least fifty of a plurality of second samples, wherein the at least one of the immunocomplexes, or at least a portion of the immunocomplexes, of the plurality of immunocomplexes is released from the at least one first paramagnetic beads of the plurality of first paramagnetic beads and at least one first paramagnetic beads of the plurality of first paramagnetic beads is removed from the at least one of a plurality of second solutions, at least two of a plurality of second solutions, at least three of a plurality of second solutions, at least four of a plurality of second solutions, at least five of a plurality of second solutions, at least ten of a plurality of second solutions, at least twenty of a plurality of second solutions, at least fifty of a plurality of second solutions; in less than 130 minutes, in less than 120 minutes, in less than 110 minutes, in less than 100 minutes, in less than 90 minutes, in less than 85 minutes, in less than 80 minutes, in less than 75 minutes, in less than 70 minutes, in less than 65 minutes, in less than 60 minutes, in less than 55 minutes, in less than 50 minutes, or in less than 45 minutes.

### Second Capture:

In some embodiments, the bench-top system or instrument provided herein may perform, after the formation of at least one second solution of the plurality of second solutions comprising at least one immunocomplex of the plurality of immunocomplexes with at least one of the first paramagnetic beads of the plurality of first paramagnetic beads removed from second solutions; the second capture of at least one of the immunocomplexes of the plurality of immunocomplexes by introducing a plurality of second paramagnetic beads into at least one of the plurality of second solutions, into at least two of the plurality of second solutions, into at least three of the plurality of second solutions, in at least four of the plurality of second solutions, in at least five of the plurality of second solutions, in at least ten of the plurality of second solutions, in at least twenty of the plurality of second solutions, in at least fifty of the plurality of second solutions and incubating the at least one of the plurality of second solutions, the at least two of the plurality of second solutions, at least three of the plurality of second solutions, at least four of the plurality of second solutions, at least five of the plurality of second solutions, at least ten of the plurality of second solutions, at least twenty of the plurality of second solutions, or at least fifty of the plurality of second solutions with the plurality of second paramagnetic beads, for less than 60 minutes, for less than 50 minutes, for less than 40 minutes, for less than 30 minutes, for less than 20 minutes, for less than 15 minutes, for less than 12 minutes for less than 10 minutes for less than 8 minutes, or for less than 5 minutes.

The assay methods disclosed herein include step (1): formation of an immunocomplex by mixing a first binder, a second binder, and a sample in a solution, wherein the first and second binders bind non-interfering epitopes on the analyte, and wherein the immunocomplex is captured on a first solid surface in contact with the solution via the binding between a first presenting group conjugated to the first binder and a first receiving group coupled to the first surface. In some embodiments, the first presenting group is a nucleic acid tag, and the first receiving group is a nucleic acid capture probe, wherein the probe or a fragment thereof is complementary to the tag or a fragment thereof.

As disclosed herein, the binders used in the assay methods can be any molecule or a portion of a molecule which binds a specific target analyte. As such, a binder can comprise any protein, peptide, nucleic acid, carbohydrate, lipid, or small molecule. In some embodiments, a binder comprises an antibody. In some embodiments, a binder comprises an antibody fragment. In some embodiments, a binder comprises an antibody mimetic. In some embodiments, a binder comprises a small molecule.

The binders used in assay methods disclosed herein can be conjugated to presenting groups (e.g. nucleic acid tags). The binder and presenting group can be joined together either directly through a bond or indirectly through a linking group. Where linking groups are employed, such groups can be chosen to provide for covalent attachment of the presenting groups and binders, as well as to maintain the desired binding affinity of the binder for its target analyte. Linking groups can vary depending on the binder. The linking group, when present, is typically biologically inert. A variety of linking groups are known to those of skill in the art and can be used in the assay methods disclosed herein. In some embodiments, a linking group comprises a spacer group terminated at either end with a reactive functionality capable of covalently bonding to the presenting group or the binder.

The binder/presenting group conjugates employed in the assay methods disclosed herein can be prepared using any methods known in the art. In some embodiments, the presenting groups (e.g. nucleic acid tags) can be conjugated to the binder, either directly or through a linking group. The components can be covalently bound to one another through functional groups, as is known in the art, where such functional groups can be present on the components or introduced onto the components using one or more steps, e.g. oxidation reactions, reduction reactions, cleavage reactions and the like. Functional groups that can be used in covalently bonding the components together include: hydroxy, sulfhydryl, amino, and the like. The particular portion of the different components that are modified to provide for covalent linkage can be chosen so as not to substantially adversely interfere with that component's desired binding affinity for the target analyte. Where necessary and/or desired, certain moieties on the components can be protected using blocking groups, as is known in the art, see e.g. Green & Wuts, Protective Groups in Organic Synthesis (John Wley & Sons) (1991); U.S. Pat. No. 5,733,523.

The presenting groups and receiving groups can be any binding pairs disclosed herein or otherwise known in the art, which include, but are not limited to, an antigen and an antibody against the antigen (including its fragments, derivatives or mimetics), a ligand and its receptor, complementary strands of nucleic acids, biotin and avidin (or streptavidin or neutravidin), lectin and carbohydrates, and vice versa. Additional binding pairs of “presenting groups” and “receiving groups” include fluorescein and anti-fluorescein, digioxigenin/anti-digioxigenin, and DNP (dinitrophenol)/anti-DNP, and vice versa. In some embodiments, binding pairs of “presenting groups” and “receiving groups” are complementary strands of nucleic acids, and are referred to as “tags” and “probes.” In some embodiments, binding pairs of “presenting groups” and “receiving groups” are antigens and antibodies, or antigens and antibody fragments.

As described above, the sample that can be assayed in the assay methods disclosed herein can be a material or mixture of materials containing one or more components of interest. In some embodiments, the sample is derived from a biological source. For example, the sample can be obtained from a subject, which can be a biological tissue or fluid, obtained, reached, or collected in vivo or in situ. Exemplary samples include a biological fluid, such as a blood sample, a urine sample, a plasma sample, a saliva sample, a cerebrospinal fluid sample, a semen sample, a sputum sample, a mucus sample, a dialysis fluid sample, an intestinal fluid sample, a synovial fluid sample, a serous fluid sample. In some embodiments, the sample is a blood sample. In some embodiments, the sample is a urine sample. In some embodiments, the sample is a saliva sample. In some embodiments, the sample is a plasma sample. In some embodiments, the sample is a cerebrospinal fluid sample. Exemplary samples include a tissue sample. The tissue sample can be a liquid tissue sample. The tissue sample can be a homogenized tissue sample. The tissue sample can be obtained from a diseased tissue. In some embodiments, the sample is a cancer sample.

The solid surface can also include any support known in the art on which can be used for immobilization of molecules. In some embodiments, the solid surface can be any surfaces suitable of attaching nucleic acid and facilitates the assay step. Examples of solid surfaces include beads (e.g., magnetic beads, xMAP® beads), particles, colloids, single surfaces, tubes, chips, multiwell plates, microtiter plates, slides, membranes, cuvettes, gels, and resins. Exemplary solid surfaces can include surfaces of magnetic particles, and wells of microtiter plates. When the solid phase is a particulate material (e.g., beads), it can be distributed in the wells of multi-well plates to allow for parallel processing. In some embodiments, the solid surface is the surface of a magnetic bead. The magnetic beads can be coupled with a presenting group. In some embodiments, the magnet beads can be carboxylate-modified magnetic beads, amine-blocked magnetic beads, Oligo (dT)-coated magnetic beads, streptavidin-coated magnetic beads, Protein A/G coated magnetic beads, or silica-coated magnetic beads. In some embodiments, the solid surface is a well of a microtiter plate. In some embodiments, the first and second solid surfaces are the same. In some embodiments, the first and the second solid surfaces are different. In some embodiments, both the first and second solid surfaces used in the assay methods disclosed herein are surfaces of magnetic particles. In some embodiments, both the first and second surfaces used in the assay methods disclosed herein are surfaces of microtiter plates.

As described above, the analyte measured in assay methods disclosed herein can be any biological molecule. In some embodiments, the analyte is a protein analyte. In some embodiments, the analyte is a peptide analyte. In some embodiments, the analyte is a complex that includes at least two molecules. In some embodiments, the analyte is a protein complex that includes at least two proteins. In some embodiments, the analyte is a binding pair of two proteins. In some embodiments, the analyte is a macromolecular complex that includes at least a protein and at least a nucleic acid. In some embodiments, the analyte is a nucleic acid analyte.

In some embodiments, the first presenting group is a first nucleic acid tag (the “first tag”) and the first receiving group is a first nucleic acid capture probe (the “first probe”), wherein the probe or a fragment thereof is complementary to the tag or a fragment thereof. In some embodiments, the second receiving group is a second nucleic acid capture probe (the “second probe”), wherein the probe or a fragment thereof is complementary to the tag or a fragment thereof.

The analyte can be a nucleic acid molecule (e.g. DNA & RNA). In some embodiments, the analyte is a DNA molecule. In some embodiments, the analyte is a RNA molecule. Assay methods provided herein can detect nucleic acid molecules directly in samples such as plasma and urine without the need for nucleic acid isolation. Nucleic acid analyte can be hybridized and captured onto the first surface, released into solution, and recaptured on the second surface while the target-probe complex remains intact throughout the assay procedure. Accordingly, in some embodiments, assay methods provided herein can be used for detecting a nucleic acid analyte. For example, provided herein are assay methods for detecting a nucleic acid analyte in a sample comprising the following steps:
(1) mixing a first binder, a second binder, and the sample in a solution, wherein the first and second binders bind non-interfering epitopes on the nucleic acid analyte and form an immunocomplex, and wherein the immunocomplex is captured on a first solid surface in contact with the solution via binding between a first presenting group conjugated to the first binder and a first receiving group coupled to the first surface; wherein the first and second binders for the nucleic acid analyte are nucleic acids that are complementary to different fragments of the nucleic acid analyte;
(2) washing the first solid surface to remove unbound molecules;
(3) releasing the immunocomplex from the first solid surface by disrupting the binding between the first presenting group and the first receiving group;
(4) introducing a second solid surface and recapturing the immunocomplex via binding between a second presenting group conjugated to the second binder and the second receiving group coupled to the second solid surface;
(5) washing the second solid surface to remove unbound molecules; and
(6) detecting the immunocomplex.

In some embodiments, the bench-top system or instrument provided herein may perform, after the plurality of samples are dispensed and set-up in the assay plate, the immunocomplex formation of introducing a plurality of the first binding moieties and a plurality of the second binding moieties into at least one of the plurality of samples, into at least two of the plurality of samples, into at least three of the plurality of samples, in at least four of the plurality of samples, in at least five of the plurality of samples, in at least ten of the plurality of samples, in at least twenty of the plurality of samples, in at least fifty of the plurality of samples and incubating the at least one of the plurality of samples, into at least two of the plurality of samples, into at least three of the plurality of samples, in at least four of the plurality of samples, in at least five of the plurality of samples, in at least ten of the plurality of samples, in at least twenty of the plurality of samples, in at least fifty of the plurality of samples with the plurality of the first binding moieties and the plurality of the second binding moieties, in less than 100 minutes, in less than 95 minutes, in less than 90 minutes, in less than 85 minutes, in less than 80 minutes, in less than 75 minutes, in less than 70 minutes, in less than 65 minutes, in less than 60 minutes, or in less than 45 minutes.

In some embodiments, the detectable marker is a nucleic acid, which can be amplified and detected by Polymerase Chain Reaction (PCR). Immuno-PCR technology can be used, which harasses the power of nucleic acid technology for protein detection by converting protein analyte detection into a nucleic acid reporter (e.g. U.S. Pat. No. 5,665,539). A segment of nucleic acid pre-conjugated to the detection binder can be used as the reporter of the immunocomplex and PCR is used to amplify the reporter and generate detectable signal. In some embodiments, the nucleic acid tags used for capture/release can be detected, and no additional detectable marker is needed. In some embodiments, the first tag is used for detection by PCR. In some embodiments, the second tag is used for detection by PCR.

The nucleic acid reporters can take various forms. For example, the first tag and the second tag can be ligated to generate the nucleic acid reporter. As a person of ordinary skill in the art would understand, any of the reporter generation methods disclosed herein or otherwise known in the art can be deployed in this step, including such as ligation, polymerization extension or collaborative hybridization. Reporters comprise a first nucleic acid target label of a first moiety ligated with a second nucleic acid target label of a second moiety wherein the second moiety may be pre-selected to be paired with or complimentary to the first moiety. These nucleic acid target labels may generate reporters either by direct or indirect ligation, by ligating the first and second nucleic acid target labels directly together, in-part together and/or by using a connector sequence. The connector sequence may also have a sample-specific target label, that may, for example, be used for next generation sequencing.

In some embodiments, the second capture does not have to be releasable, and the nucleic acid reporter can be generated with the immunocomplex captured on the second surface. Alternatively, the immunocomplex can first be released back to solution before the nucleic acid reporters are generated.

Proximity Ligation Assay (PLA) and Proximity Extension Assay (PEA) are known in the art (e.g. U.S. Pat. Nos. 6,511,809, 6,878,515, 7,306,904, 9,777,315, 10,174,366, WO9700446, Greenwood C, Biomol. Det. & Quan. 4 (2015) 10⁻¹⁶). Proximity-based detection differ from immuno-PCR in that they depend on the simultaneous recognition of target analyte by two nucleic acid-conjugated binders in order to trigger the formation of amplifiable products. Therefore, individual nucleic acid-conjugated binders that are not part of the immunocomplex will not generate reports, thus avoiding background from single nonspecifically bound binder. In some embodiments, proximity ligation is used to generate the nucleic acid reporter, wherein, upon the formation of the immunocomplex, the first tag and the second tag are brought into sufficient proximity to be ligated, and a fragment of the ligation product, which composes a fragment of the first tag and a fragment of the second tag, is used as an amplicon to generate the signal for detection. In some embodiments, proximity extension is used to generate the nucleic acid reporter, wherein, upon the formation of the immunocomplex, the first tag and the second tag are brought into sufficient proximity to interact with each other and form a duplex, such that the 3′ end of at least one nucleic acid tag of the duplex can be extended to generate an extension product, which can be used as an amplicon to generate the signal for detection. In some embodiments, collaborative hybridization is used to generate the nucleic acid reporter, wherein, upon the formation of the immunocomplex, the first tag and the second tag are brought into sufficient proximity to interact each other and form a hybridization product, which can be used as an amplicon to generate the signal for detection.

Although some assay methods use proximity ligation, as described above, proximity extension, collaborative hybridization, or other methods known in the art can also be used for generating the nucleic acid reporter for detection.

The reporters generated in the last step can be detected using any existing nucleic acid detection technologies, which include, but are not limited to, PCR, quantitative PCR (qPCR), digital PCR (dPCR) or next-generation sequencing (NGS). In some embodiments, the detection is qualitative detection. In some embodiments, the detection is quantitative detection. In some embodiments, the nucleic acid reporter is detected by qPCR. In some embodiments, the nucleic acid reporter is detected by dPCR. In some embodiments, the nucleic acid reporter is detected by NGS.

Each analyte is assigned with a unique ID. As such, provided herein are assay methods comprising simultaneously detecting at least two analytes in the sample by simultaneously detecting the unique target IDs associated with each analyte. In some embodiments, the assay methods provided herein simultaneously detect at least three, at least four, at least five, at least six, at least seven, at least eight, at least nine, at least ten, at least twelve, at least fifteen, at least twenty, at least thirty, at least forty, at least fifty, at least sixty, at least seventy, at least eighty, at least ninety, or at least one hundred analytes in the sample by simultaneously detecting the unique target IDs associated with each analyte.

In some embodiments, the analytes are proteins. In some embodiments, the analytes include at least one protein and at least one nucleic acid. The nucleic acid can be DNA or RNA. The fact that the assay methods provided herein can be used for analysis of protein, DNA and RNA makes it an ideal platform for multi-omic analysis.

Incorporation of IDs in the nucleic acid reporters in the assay methods provided herein can also help improve the specificity of the assay. Each of the first binder and the second binder is associated with a unique ID, and only the signals generated from reporter containing the IDs of both binders are counted as true signal. This scheme can be used to reduce or eliminate false positive signal generated by cross-reactivity or non-specific binding among different binding pairs. Accordingly, provided herein are assay methods for detecting analyte in a sample by the co-detection of the first and the second target IDs each associated with the first and second binders, respectively.

Incorporation of IDs in the nucleic acid reporters in the assay methods provided herein can also be used to detect interactions between molecules, for example, protein-protein interactions. The co-detection and quantification of reporters containing IDs of two binders designed for different molecules would indicate the interaction and affinity of these two associated molecules under the assay condition. In some embodiments, the assay methods provided herein detect protein-protein interactions. Accordingly, provided herein are assay methods for detecting analyte in a sample, wherein the analyte is a binding pair of two different proteins; wherein the first binder binds one protein, and the second binder binds the other protein of the binding pair; and wherein the binding pair is detected by the co-detection of the first and the second target IDs.

In addition to analyte-specific “target IDs,” the nucleic acid reporters generated in the assay methods provided herein can also include sample-specific “sample IDs.” When the assay method provided herein is performed on a particular sample, a sample ID can be introduced during the assay at or before reporter generation step to identify the sample. With such sample ID incorporated into the reporter, reporters from many samples can be pooled together and read by NGS in parallel. In some embodiments, the sample ID can be carried on a nucleic acid independent of the binder tags and incorporated into the reporter at the ligation step. Accordingly, in some embodiments, the nucleic acid reporter formed in each sample contains an ID that is a sample ID, wherein the sample ID is inserted between the first tag or surrogate, and the second tag or surrogate.

In some embodiments, the nucleic acid reporters generated in the assay methods provided herein can comprise both a target ID and a sample ID. In some embodiments, the nucleic acid reporter comprises (a) a target ID in the first tag or the first surrogate, or in the second tag or the second surrogate, and (b) a sample ID that is (1) inserted between the first tag or surrogate thereof, and the second tag or surrogate thereof, (2) included in the first surrogate or the second surrogate or (3) ligated to the first tag or surrogate thereof, or the second tag or surrogate thereof.

In some embodiments, the assay methods provided herein simultaneously detect at least three analytes in at least three, at least four, at least five, at least six, at least seven, at least eight, at least nine, at least ten, at least 15, at least 20, at least 30, at least 40, at least 50, at least 60, at least 70, at least 80, at least 90, at least 100, at least 150, at least 200, at least 250, at least 300, at least 350, at least 400, at least 450, or at least 500 samples by simultaneously detecting unique sample IDs and unique target IDs in the nucleic acid reporters with each sample.

In some embodiments, the assay methods provided herein simultaneously detect at least five analytes in at least three, at least four, at least five, at least six, at least seven, at least eight, at least nine, at least ten, at least 15, at least 20, at least 30, at least 40, at least 50, at least 60, at least 70, at least 80, at least 90, at least 100, at least 150, at least 200, at least 250, at least 300, at least 350, at least 400, at least 450, or at least 500 samples by simultaneously detecting unique sample IDs and unique target IDs in the nucleic acid reporters with each sample.

In some embodiments, the assay methods provided herein simultaneously detect at least ten analytes in at least three, at least four, at least five, at least six, at least seven, at least eight, at least nine, at least ten, at least 15, at least 20, at least 30, at least 40, at least 50, at least 60, at least 70, at least 80, at least 90, at least 100, at least 150, at least 200, at least 250, at least 300, at least 350, at least 400, at least 450, or at least 500 samples by simultaneously detecting unique sample IDs and unique target IDs in the nucleic acid reporters with each sample.

In some embodiments, the assay methods provided herein simultaneously detect at least twenty analytes in at least three, at least four, at least five, at least six, at least seven, at least eight, at least nine, at least ten, at least 15, at least 20, at least 30, at least 40, at least 50, at least 60, at least 70, at least 80, at least 90, at least 100, at least 150, at least 200, at least 250, at least 300, at least 350, at least 400, at least 450, or at least 500 samples by simultaneously detecting unique sample IDs and unique target IDs in the nucleic acid reporters with each sample.

In some embodiments, the assay methods provided herein simultaneously detect at least fifty analytes in at least three, at least four, at least five, at least six, at least seven, at least eight, at least nine, at least ten, at least 15, at least 20, at least 30, at least 40, at least 50, at least 60, at least 70, at least 80, at least 90, at least 100, at least 150, at least 200, at least 250, at least 300, at least 350, at least 400, at least 450, or at least 500 samples by simultaneously detecting unique sample IDs and unique target IDs in the nucleic acid reporters with each sample.

In some embodiments, the assay methods provided herein simultaneously detect at least eighty analytes in at least three, at least four, at least five, at least six, at least seven, at least eight, at least nine, at least ten, at least 15, at least 20, at least 30, at least 40, at least 50, at least 60, at least 70, at least 80, at least 90, at least 100, at least 150, at least 200, at least 250, at least 300, at least 350, at least 400, at least 450, or at least 500 samples by simultaneously detecting unique sample IDs and unique target IDs in the nucleic acid reporters with each sample.

In some embodiments, the assay methods provided herein simultaneously detect at least one hundred analytes in at least three, at least four, at least five, at least six, at least seven, at least eight, at least nine, at least ten, at least 15, at least 20, at least 30, at least 40, at least 50, at least 60, at least 70, at least 80, at least 90, at least 100, at least 150, at least 200, at least 250, at least 300, at least 350, at least 400, at least 450, or at least 500 samples by simultaneously detecting unique sample IDs and unique target IDs in the nucleic acid reporters with each sample.

Since NGS is a single molecule detection and counting method, the sequencing instrument imposes an upper limit on the total number of molecules that can be sequenced. The MiSeq system from Illumina, for example, is capable of 25 million reads per run, which limits the total number of molecules to be sequenced in a run to 25 million. This limit is not restrictive in most applications where the target is present at low concentration or even near limit-of-detection (“LOD”). In multiplexed tests, however, some targets are known to express at a level that is many magnitudes higher than others, consuming the sequencing bandwidth without providing useful clinical/biological information. Therefore, there is a need to purposefully reducing the signal generated from these abundant analytes, while maintaining sensitivity for the rest of the analytes in a multiplexed assay format.

This assay methods provided herein additionally address this need and provide related advantages. In some embodiments, provided herein are assay methods wherein the number of reporter molecules generated from a high-concentration target analyte is reduced in a precise and known proportion so that the limited bandwidth of detection can be efficiently assigned to different target analytes. The assay methods disclosed herein capture the immunocomplexes on a solid surface using a receiving group (e.g. a nucleic acid capture probe) before the reporter is generated, which provides a unique opportunity to reduce the signal of highly abundant analytes by selectively capturing only a portion of the immunocomplexes generated therefrom to the surface.

For example, in some embodiments, the binders can be conjugated with and without their presenting groups (e.g. nucleic acid tags) in a known proportion. If, for example, binders conjugated with a presenting group are mixed with the same binder without the presenting group at 1% concentration, then only 1% immunocomplex can be captured onto the surface and the reporter to target analyte ratio will be 1%. Nonfunctional presenting groups, namely, presenting groups that do not bind receiving groups, can also be used. For example, if only 0.1% of first binder is conjugated to a first presenting group (e.g. the first tag) that is functional, and the rest 99.9% of the first binders are conjugated to a first presenting group (e.g. the first tag) that that cannot be captured by the first receiving group (e.g. the first probe), the reporter to immunocomplex ratio will be 1:1000, effectively reducing the signals generated by this analyte by 1000 fold.

An alternative approach for such partial capture can be used, which introduces a known portion of nonfunctional receiving groups (e.g. nucleic acid capture probes). In the indirect capture methods, for example, a certain proportion of the first capture probe can be included that does not have the segment complementary to the universal capture probe or is not biotinylated. As a result, the same proportion of immunocomplexes cannot be captured on the surface and thus cannot generate nucleic acid reporters for detection. For example, if the receiving group contains only 0.1% functional molecule that can be coupled to the solid surface (the rest 99.9% are non-functional dummy molecules), the reporter to immunocomplex ratio will also be 1:1000, reducing the signals generated by this analyte by 1000 fold.

Accordingly, provided herein are also assay methods that include proportionally reducing the amount of an analyte detected by the assay, by adding a non-functional binder to the solution in step (1), wherein the non-functional binder competes with the first binder for binding to the analyte but is either unconjugated, or conjugated to a presenting group that does not bind the first receiving group. In some embodiments, the non-functional binder is unconjugated. In some embodiments, the non-functional binder is conjugated to a presenting group that does not bind the first receiving group. In some embodiments, the non-functional binder is conjugated to a nucleic acid tag that cannot hybridize with the first probe coupled to the first solid surface.

Accordingly, provided herein are also assay methods that include proportionally reducing the amount of an analyte detected by the assay, by adding a non-functional receiver to the solution in step (1), wherein the non-functional receiver competes with the first receiving for binding to the first presenting group but cannot be coupled with the first solid surface.

In some embodiments, provided herein are also assay methods that include proportionally reducing the amount of an analyte detected by the assay, by adding a non-functional binder to the solution in step (1), wherein the non-functional binder competes with either the first binder or the second binder for binding to the analyte but forms a immunocomplex that cannot be detected. In some embodiments, the assay methods provided herein detect the immunocomplex by detecting a detectable marker conjugated to either the first binder or the second binder, and the non-functional binder is either unconjugated to the detectable marker, or is conjugated to a defective detectable marker that cannot produce the signal for detection. In some embodiments, the immunocomplex is detected via the nucleic acid reporter, and the non-functional binder can be conjugated to a nucleic acid tag that lacks the proper segment for generating the nucleic acid reporter. A person of ordinary skill in the art would understand that different approaches that are variants of the methods disclosed herein can be taken to proportionally reduce the signal generated by highly-abundant analytes in the sample, therefore allowing the concurrent detection of multiple analytes that may be present at concentrations that differ by even orders of magnitude.

IDs include both the original ID molecules and derivative ID molecules that contain the information derived from but is non-identical to the original ID, so long as such derived molecules or the derived information can identify or otherwise distinguish a particular target or sample from other targets or samples and be correlated with the intended target or sample.

The disclosure further provides that the step (4) of the methods provided in this Section 4.2.5 further comprises PCR amplification of the nucleic acid reporter. As is clear from the disclosure, such PCR can be any PCR as provided in this and other sections, such as Sections 4.2.3 (including 4.2.3.1 and 4.2.3.2), 4.2.3 and 4.2.5, as well as any suitable PCR known and practiced in the field.

The binders can bind to an analyte indirectly through an intermediary, e.g., a primary antibody against the analyte. As such, in some embodiments of the method provided herein.

The first and second binders can bind epitopes on the analyte that permit simultaneous binding, thereby increasing the specificity of the detection. In some embodiments, the first and second binders bind to non-interfering epitopes on the analyte. In other embodiments, the first and second binders bind to non-overlapping epitopes on the analyte. In other embodiments, the first and second binders bind to different epitopes on the analyte. In yet other embodiments, the first and second binders bind to separate epitopes on the analyte. In still yet other embodiments, the first and second binders bind to two epitopes on the analyte to which the two binders can simultaneously and separately bind without having any steric hindrance.

Additionally, such first tag, first probe, second tag, and second probe can be combined as provided in the preceding paragraph in various ways. Accordingly, in one embodiment, the first probe is a protein that specifically binds to the first tag and the second probe is a protein that specifically binds to the second tag. In another embodiment, the first probe is a protein that specifically binds to the first tag and the second probe is a protein and nucleic acid complex that specifically binds to the second tag. In yet another embodiment, the first probe is a protein that specifically binds to the first tag and the second probe is a nucleic acid molecule, wherein the second probe or a fragment thereof is complementary to the second tag or a fragment thereof. In a further embodiment, the first probe is a protein that specifically binds to the first tag and the second probe is a nucleic acid molecule, wherein the second probe or a fragment thereof hybridizes with the second tag or a fragment thereof.

Additionally, in one embodiment, the first probe is a protein and nucleic acid complex that specifically binds to the first tag and the second probe is a protein that specifically binds to the second tag. In another embodiment, the first probe is a protein and nucleic acid complex that specifically binds to the first tag and the second probe is a protein and nucleic acid complex that specifically binds to the second tag. In yet another embodiment, the first probe is a protein and nucleic acid complex that specifically binds to the first tag and the second probe is a nucleic acid molecule, wherein the second probe or a fragment thereof is complementary to the second tag or a fragment thereof. In a further embodiment, the first probe is a protein and nucleic acid complex that specifically binds to the first tag and the second probe is a nucleic acid molecule, wherein the second probe or a fragment thereof hybridizes with the second tag or a fragment thereof.

In one specific aspect, provided herein is an assay method for detecting an analyte in a sample, comprising:
(1) mixing a first binding moiety comprising a first binder and a first presenting group, a second binding moiety comprising a second binder and a second presenting group, and the sample in a solution, wherein:
   (i) the first and second binders bind to the analyte and form an immunocomplex,
   (ii) the immunocomplex is captured on a first solid surface in contact with the solution via binding between the first presenting group and a first receiving group coupled to the first solid surface, and
   (iii) the first binding moiety further comprises a first target label comprising a first identity barcode (“ID”) that is analyte-specific (“target ID”) and the second binding moiety further comprises a second target label comprising a second target ID;
(2) washing the first solid surface to remove unbound molecules;
(3) releasing the immunocomplex from the first solid surface by disrupting the binding between the first presenting group and the first receiving group;
(4) introducing a second solid surface and recapturing the immunocomplex on the second solid surface via binding between the second presenting group and the second receiving group coupled to the second solid surface;
(5) washing the second solid surface to remove unbound molecules;
(6) binding a sample label comprising an ID that is sample-specific (“sample ID”) (i) to the first target label, (ii) to the second target label, or (iii) to both the first target label and the second target label;
(7) generating a nucleic acid reporter from the immunocomplex based on proximity between the first target label and the second target label, wherein the nucleic acid reporter comprises the first target ID, the second target ID, and the sample ID;
(8) releasing the immunocomplex from the second solid surface by disrupting the binding between the second presenting group and the second receiving group; and
(9) detecting the nucleic acid reporter by qPCR, thereby detecting the analyte.

In another specific aspect, provided herein is an assay method for detecting an analyte in at least two samples, comprising:
(1) mixing a first binding moiety comprising a first binder and a first presenting group, a second binding moiety comprising a second binder and a second presenting group, and the samples in a solution, wherein:
   (i) the first and second binders bind to the analyte and form an immunocomplex,
   (ii) the immunocomplex is captured on a first solid surface in contact with the solution via binding between the first presenting group and a first receiving group coupled to the first solid surface, and
   (iii) the first binding moiety further comprises a first target label comprising a first identity barcode (“ID”) that is analyte-specific (“target ID”) and the second binding moiety further comprises a second target label comprising a second target ID;
(2) washing the first solid surface to remove unbound molecules;
(3) releasing the immunocomplex from the first solid surface by disrupting the binding between the first presenting group and the first receiving group;
(4) introducing a second solid surface and recapturing the immunocomplex on the second solid surface via binding between the second presenting group and the second receiving group coupled to the second solid surface;
(5) washing the second solid surface to remove unbound molecules;
(6) binding a sample label comprising an ID that is sample-specific (“sample ID”) (i) to the first target label, (ii) to the second target label, or (iii) to both the first target label and the second target label;
(7) generating a nucleic acid reporter from the immunocomplex based on proximity between the first target label and the second target label, wherein the nucleic acid reporter comprises the first target ID, the second target ID, and the sample ID;
(8) pooling the nucleic acid reporters from the at least two samples;
(9) releasing the immunocomplex from the second solid surface by disrupting the binding between the second presenting group and the second receiving group;
(9) amplifying the nucleic acid reporters;
(10) purifying the nucleic acid reporters; and
(11) detecting the nucleic acid reporters by next generation sequencing (NGS), thereby detecting the analyte.

Provided herein are also systems for carrying out the assay methods disclosed herein. The systems disclosed herein can be used for detecting an analyte in a sample. The systems disclosed herein can also be used for detecting multiple analytes in a sample, an analyte in multiple samples, or multiple analytes in multiple samples. In some embodiments, the systems provided herein are for carrying out the assay methods for qualitatively detecting an analyte in a sample. In some embodiments, the systems provided herein are for carrying out the assay methods for quantitatively detecting an analyte in a sample. In some embodiments, the system provided herein is contained in a kit.

Any detection marker known in the art can be included in a system of this disclosure. In some embodiments, the detection marker is a colorimetric detection reagent, a fluorescent detection reagent, or a chemiluminescent detection reagent. In some embodiments, the colorimetric detection reagent includes PNPP (p-nitrophenyl phosphate), ABTS (2,2′-azino-bis (3-ethylbenzothiazoline-6-sulphonic acid)) or OPD (o-phenylenediamine). In some embodiments, the fluorescent detection reagent includes QuantaBlu™ or QuantaRed™ (Thermo Scientific, Waltham, MA). In some embodiments, the luminescent detection reagent includes luminol or luciferin. In some embodiments, the detection reagent includes a trigger (e.g., H2O2) and a tracer (e.g., isoluminol-conjugate).

For any of the embodiments of the systems provided herein that involves nucleic acid binding or hybridization, the complementary fragments of two nucleic acid can be a pair of complementary A and/or T rich sequences or a pair of complementary G and/or C rich sequences for the purpose of binding the presenting group (e.g. the first tag and/or the second tag) and the receiving group (e.g. the first probe and/or the second probe) or binding the receiving group (e.g. the first probe and/or the second probe) and the solid surface (e.g. the first solid surface and/or the second solid surface).

Additionally, the disclosure provides sample label comprising sample ID, wherein the sample label binds to the immunocomplex for various embodiments of the methods provided herein. The sample label can bind to any component of the binding moiety, the immunocomplex formed by the two binding moieties (e.g. the first binding moiety and the second binding moiety), the receiving group, or a component coupled to the solid surface. In one embodiment, the sample label binds to a target label. In another embodiment, the sample label binds to the presenting group (e.g. presenting group that is a nucleic acid molecule). In a further embodiment, the sample label binds to a binder. In yet another embodiment, the sample label binds to a receiving group. In one embodiment, the sample label binds to the first target label. In another embodiment, the sample label binds to the first presenting group (e.g. presenting group that is a nucleic acid molecule). In still another embodiment, the sample label binds to the first tag. In a further embodiment, the sample label binds to the first binder. In yet another embodiment, the sample label binds to the first receiving group. In one embodiment, the sample label binds to the second target label. In another embodiment, the sample label binds to the second presenting group (e.g. presenting group that is a nucleic acid molecule). In still another embodiment, the sample label binds to the second tag. In a further embodiment, the sample label binds to the second binder. In yet another embodiment, the sample label binds to the second receiving group.

When the sample label is a double-stranded nucleic acid molecules and has one or two overhangs, the sample label can hybridize via its overhangs with any nucleic acid component of the binding moiety, the immunocomplex formed by the two binding moieties, the receiving group, or a nucleic acid component coupled to the solid surface, in various embodiments of the methods provided herein. When the sample label is a single-stranded nucleic acid molecules, the sample label can hybridize with any nucleic acid component of the binding moiety, the immunocomplex formed by the two binding moieties, the receiving group, or a nucleic acid component coupled to the solid surface, in various embodiments of the methods provided herein.

A person of ordinary skill in the art would understand that when the a double-stranded sample label hybridizes with two items, in some embodiments, the double-stranded sample label hybridizes with the first item via its 5′ overhang and the second item via its 3′ overhang. In other embodiments, the double-stranded sample label hybridizes with the first item via its 3′ overhang and the second item via its 5′ overhang. In other embodiments, the double-stranded sample label hybridizes with the first item via its 5′ overhang and the second item via its 5′ overhang. In still further embodiments, the double-stranded sample label hybridizes with the first item via its 3′ overhang and the second item via its 3′ overhang. When the a double-stranded sample label hybridizes with one item, in some embodiments, the double-stranded sample label hybridizes with the item via its 5′ overhang. In certain embodiments, the double-stranded sample label hybridizes with the item via its 3′ overhang. When the sample label is a single-stranded sample label, such sample label can hybridizes with any one item or two item, via any parts or its nucleic acid sequence, including either end, both ends (5′ end and 3′ end), and any internal sequences.

As the systems provided herein can correlate each analyte with the one or more target IDs provided herein, the systems can simultaneously detect at least two analytes in the sample by simultaneously detecting the target IDs associated with each analyte and correlating target IDs with analytes. Accordingly, in some embodiments, the systems provided herein simultaneously detect at least three, at least four, at least five, at least six, at least seven, at least eight, at least nine, at least ten, at least twelve, at least fifteen, at least twenty, at least thirty, at least forty, at least fifty, at least sixty, at least seventy, at least eighty, at least ninety, or at least one hundred analytes in the sample by simultaneously detecting the unique target IDs associated with each analyte. In further embodiments, the systems provided herein simultaneously detect about three, about four, about five, about six, about seven, about eight, about nine, about ten, about twelve, about fifteen, about twenty, about thirty, about forty, about fifty, about sixty, about seventy, about eighty, about ninety, or about one hundred analytes in the sample by simultaneously detecting the unique target IDs associated with each analyte. In certain embodiments, the systems provided herein simultaneously detect at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1100, at least 1200, at least 1300, at least 1400, at least 1500, at least 1600, at least 1700, at least 1800, at least 1900, or at least 2000 analytes in the sample by simultaneously detecting the unique target IDs associated with each analyte. In further embodiments, the systems provided herein simultaneously detect about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, or about 2000 analytes in the sample by simultaneously detecting the unique target IDs associated with each analyte.

In one aspect, provided herein is a system for detecting an analyte in a sample comprising (1) a first binding moiety comprising a first binder, a first presenting group, and a first target label comprising a first identity barcode (“ID”) that is analyte-specific (“target ID”); (2) a second binding moiety comprising a second binder, a second presenting group, and a second target label comprising a second target ID; (3) a first receiving group, a first solid surface, a second receiving group, and a second solid surface; (4) reagents for ligation and a sample label comprising an ID that is sample-specific (“sample ID”); and (5) reagents for quantitative PCR; wherein (i) the first and second binders bind to the analyte and form an immunocomplex; (ii) the first receiving group is coupled to the first solid surface and is configured to capture the first presenting group; (iii) the second receiving group is coupled to the second solid surface and is configured to capture the second presenting group; (iv) the first target label is directly or indirectly bound to the first binder and the second target label is directly or indirectly bound to the second binder; (v) the first presenting group is directly or indirectly bound to the first binder and the second presenting group is directly or indirectly bound to the second binder; and (vi) the sample label binds to both the first target label and the second target label.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

Described herein are systems, apparatus, and methods for performing fluid and sample manipulation. In certain embodiments, the systems, apparatus, and methods are configured for use in assays relating to the detection and/or the quantification of analyte molecules or particles in a sample fluid. In some cases, the systems, methods, and apparatus are automated. The subject matter of the present invention involves, in some cases, interrelated products, alternative solutions to a particular problem, and/or a plurality of different uses of one or more systems and/or articles.

The systems, apparatus, and methods may include at least a portion thereof configured to be used to analyze a sample fluid comprising a plurality of analyte molecules and particles. The systems, apparatus and methods, in some embodiments, are directed towards determining the concentration of analyte molecules or particles in the sample fluid. Various aspect or portions of the apparatus and systems may include one or more of at least a robotic gantry (“Gantry”), a reagent/consumable hotel (“Hotel”), a plate staging station (“Stand”), a magnetic bead processor and mixer (“Mixer”), a plate washer (“Washer”), bulk fluid station (“Bulk”), and/or a computer control system. In addition, the apparatus and systems may additionally comprise and readout unit (“Reader”), a incubator and sealer (“IncuSealer”), and/or other components, examples of which are described herein. In some embodiments, automated apparatus and systems may allow for fast and/or precise input of samples and/or may reduce errors or variations due to human error and/or manipulation of an assay sample, as compared to non-automated systems.

In some embodiments, an assay method performed by an apparatus or system described herein may comprise at least the following steps. First a sample fluid is provided comprising a plurality of analyte molecules or particles (i.e. molecules and/or particles whose quantity and/or presence is desired to be determined). The sample fluid is exposed to a plurality of beads, wherein at least a portion of the analyte molecules (or particles) in the sample fluid associate with a bead. In some cases, the ratio of beads to analyte molecules is such that statistically, zero or one analyte molecules associate with a bead, as described herein. In some cases, the ratio of beads to analyte molecules is such that statistically, multiple analyte molecules associate with a bead, as described herein. The beads are then processed. In some cases, the beads are magnetic or can be induced to be magnetic (e.g., paramagnetic). The beads may be interrogated or analyzed (e.g., using a quantitative PCR module) to determine the concentration of at least one analyte molecule or particle. The qPCR module, and in certain embodiments other components of the system as well, may be associated with a computer control system that is capable of analyzing the data obtained by the qPCR module. A measure of the concentration of analyte molecules may be determined based at least in part on the output of the qPCR module.

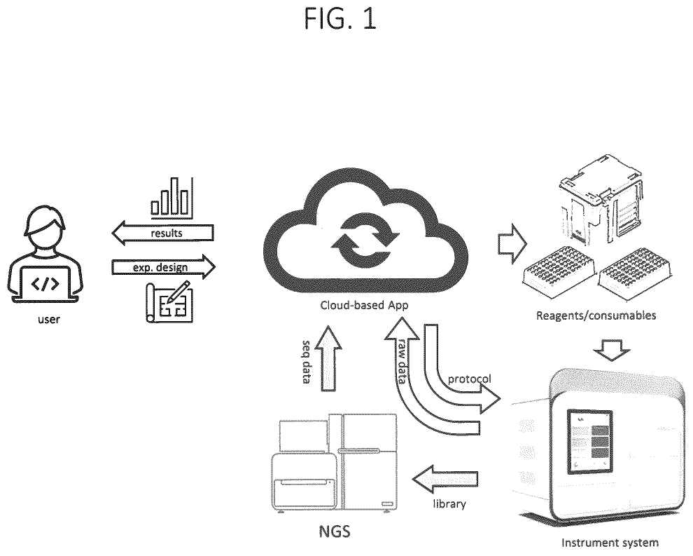

The disclosed instrument is an integrated part of an assay eco-system illustrated in FIG. 1, comprising a Cloud-based app (“App”), a fully automated assay instrument and reagents/consumables. A user would first use the App on any internet or intranet connected computing device to design an experiment, setup an assay protocol and book a time slot on an instrument for the assay run. The App would also assist the user to identify or optionally purchase the needed reagents and consumables. The user would manually load the reagents and consumables into the instrument together with the samples. The instrument scans the barcodes of the reagents and consumables to check the correct items are loaded before executing the assay protocol to generate test data. The test data are automatically sent to the App via internet or intranet on which the user can view and analyze the results. In addition, the instrument status, assay run logs, error messages, are uploaded to the App to enable remote diagnostics, trouble shooting and other customer support services.

In certain embodiments, for example, the user of the instrument uses the App to download an assay “recipe” from a remote server. In certain embodiments, for example, the downloaded recipe comprises information regarding the reagents and consumables required, the analytical protocol, and/or the algorithm for analyzing experimental data. In certain embodiments, for example, the downloaded recipe is both computer-readable and human-readable. In certain embodiments, for example, the instrument verifies the presence of any required reagents and consumables according to the information in the download recipe before executing the assay protocol. In certain embodiments, for example, the instrument does not execute the assay protocol in the download recipe unless all required reagents and consumables are present. In certain embodiments, for example, the App analyzes experimental data according to the algorithm contained in the downloaded recipe. In certain embodiments, for example, the App uploads experimental data to a remote server for analysis, and downloads the results of such analysis for the user. In certain embodiments, for example, data transmissions from and to the App are encrypted for security.

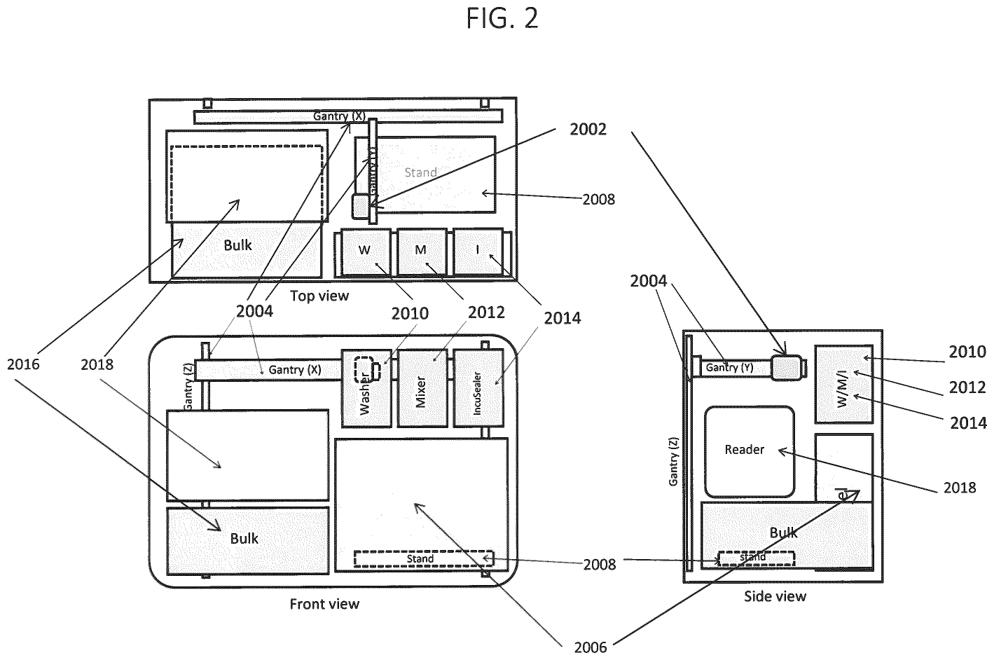

FIG. 2 illustrates an exemplary conceptual hardware architecture of the disclosed instrument, comprising multiple modules, each performing a critical procedure or function commonly used in biochemical assays. These may include robotic gantry (“Gantry”) 2004, reagent/consumable hotel (“Hotel”) 2006, plate staging station (“Stand”) 2008, plate washer (“Washer”) 2010, magnetic bead processor and mixer (“Mixer”) 2012, incubator and sealer (“IncuSealer”) 2014, bulk fluid station (“Bulk”) 2016 and readout unit (“Reader”) 2018. The gantry 2004 is the core of the instrument, carrying multiple tools on its end-effector 2002, which optionally include pipettors, a plate gripper, a barcode scanner and a position sensor. It integrates the functional modules into an automated assay system by transferring fluids between plates and transporting assay plates to different modules on a timely basis according to the assay protocol.

For example, in certain embodiments, the disclosed instrument has a bench-top housing 3000 with a touchscreen display 3302, a user-accessible compartment for bulk reagents 3306, and a compartment for user-accessible set of bays 3308. The bench-top housing may include a controller 3018, a robotic gantry 2004 equipped with an end-effector 2002 that is capable of moving the end-effector in three degrees of freedom in X, Y and Z axis, comprising pipettors 6002, a plate gripper 6004, a laser position sensor 6006, and a barcode scanner 6008. The bench-top housing may include a hotel 2006 comprising a set of plate bays for receiving and holding at least one universal reagent cartridge 29016, and a consumables carrier 30012. The bench-top housing may include a stand 2008 that provides for up to 6-plate positions, wherein the stand is movable along the Y-axis (from front-to-back within the instrument housing), a washer 2010 for washing plates, a mixer 2012 that operates as a magnetic bead processor and mixer comprising multiple assay plate platforms 11006 vertically positioned on top of each other, an IncuSealer 2014 for incubating and sealing at least one plate, a bulk fluid station 2016 for receiving and holding bulk materials comprising a first buffer container 18018, a second buffer container 18020, a third buffer container 18022 and waste container(s) 18016, and a reader 2018 that comprises a qPCR unit for either qPCR readout or pool library ready for next-generation sequencing (NGS).

In some embodiments, the instrument and/or process comprises a target kit comprising a plurality of paired-binding moieties that are pre-selected to bind specific analytes. The paired-binding moieties comprise a first moiety comprising a first antibody or a first antibody fragment that is pre-selected to bind a specific analyte, a first nucleic acid target label comprising a first identity that is analyte-specific to the specific analyte, and a first nucleic tag; and a second moiety comprising a second antibody or a second antibody fragment that is pre-selected to bind the same specific analyte as the first antibody or antibody fragment of the first moiety of the paired-binding moieties, a second nucleic acid target label comprising a second identity that is analyte-specific to the specific analyte, and a second nucleic acid tag.

The instrument and/or process also comprises a detection kit comprising a plurality of wells, wherein a first well of at least one of the plurality of wells comprises a first substrate solution comprising a plurality of first substrates, wherein a second well of at least one of the plurality of wells comprises a second substrate solution comprising a plurality of second substrates; and wherein a third well of at least one of the plurality of wells comprises a ligation reagent and optionally a nucleic acid sample specific label.

In certain embodiments, the gantry 2004 introduces in parallel in a portion of the plurality of first wells in the first plate 8012 a portion of the plurality of multiplexed paired-binding moieties from the target kit 29012, 29014 with a portion of the plurality of biological samples to form a plurality of immunocomplex forming solutions in the portion of the plurality of first wells in the first plate 8012. The gantry 2004 and the substrate extractor/mixer 2012 incubate in parallel the plurality of immunocomplex forming solutions in the portion of the plurality of first wells in the first plate 8012 to form a plurality of multiplexed immunocomplexes, wherein the immunocomplexes comprises a first antibody or first antibody fragment of a first moiety of a paired-binding moieties bound to a specific analyte and a second antibody or second antibody fragment of a second moiety of the paired-binding moieties bound to the specific analyte, this may also be referred to as an immunocomplex sandwich.

The gantry 2004 combines a portion of the first substrate solution comprising the plurality of the first substrates from the detection kit with the plurality of immunocomplex forming solutions in the portion of the plurality of first wells in the first plate 8012. The gantry 2004 and the substrate extractor/mixer 2012 enable the first nucleic acid tag of the first moiety of the multiplexed paired-binding moieties to bind to a portion of the first substrates in the portion of the plurality of first wells in the first plate 8012. The substrate extractor/mixer 2012 extracts in parallel, via the portion of the plurality of first substrates, the plurality of multiplexed immunocomplexes from the plurality of immunocomplex forming solutions to form in at least a portion of the plurality of second wells in the second plate 8014 a plurality of first immunocomplex purification solutions, wherein the portion of the plurality of first substrates are eluted from the plurality of multiplexed immunocomplexes in the plurality of first immunocomplex purification solutions.

The substrate extractor/mixer 2012 to extract in parallel the portion of the plurality of first substrates from the plurality of first immunocomplex purification solutions. The gantry 2004 combines a portion of the second substrate solution comprising the plurality of second substrates from the detection kit with the plurality of first immunocomplex purification solutions in the portion of the plurality of second wells in the second plate 8014. The gantry 2004 and the substrate extractor/mixer 2012 enable a portion of the second nucleic acid tag of the second moiety of the paired-binding moieties to bind to a portion of the second substrates in the portion of the plurality of first immunocomplex purification solutions in the portion of the plurality of second wells in the second plate 8014.

The substrate extractor/mixer 2012 extracts in parallel, via the portion of the plurality of second substrates, the portion of the second substrates in the portion of the plurality of first immunocomplex purification solutions in the portion of the plurality of second wells in the second plate 8014 to form in a portion of the plurality of third wells in the third plate 8016 a plurality of multiplexed analyte-specific reporters by ligating in parallel a plurality of the first nucleic acid target label from the first moiety of the paired binding moieties (directly or indirectly) with a plurality of the second nucleic acid target label from the second moiety of the paired binding moieties. The plate washer 2010 washes the recently used second plate to prepare it for reuse in subsequent steps.

The substrate extractor/mixer 2012 extracts in parallel, via the portion of the plurality of second substrates, the plurality of multiplexed analyte-specific reporters from the portion of the plurality of third wells in the third plate 8016 back to the portion of the plurality of second wells in the second plate 8014 to elute in parallel the plurality of second substrates from the plurality of multiplexed analyte-specific reporters in the portion of the plurality of second wells in the second plate 8014.

The gantry 2004 and the IncuSealer 2014 prepare a fourth plate 8018 with the plurality of multiplexed analyte-specific reporters and seal the plate. The thermocycler/qPCR (Reader) 2018 replicates the plurality of multiplexed analyte-specific reporters. The qPCR (Reader) 2018 detects the replicated plurality of multiplexed analyte-specific reporters to identify (and quantify) the specific analytes in the plurality of biological samples.

In another embodiment, the gantry 2004 introduces in parallel in a portion of the plurality of first wells in the first plate 8012 a portion of the plurality of multiplexed paired-binding moieties from the target kit 29012, 29014 with a portion of the plurality of biological samples to form a plurality of immunocomplex forming solutions in the portion of the plurality of first wells in the first plate 8012. The gantry 2004 and the substrate extractor/mixer 2012 incubate in parallel the plurality of immunocomplex forming solutions in the portion of the plurality of first wells in the first plate 8012 in the IncuSealer 2014 to form a plurality of multiplexed immunocomplexes.

The gantry 2004 combines a portion of the first substrate solution comprising the plurality of the first substrates from the detection kit with the plurality of immunocomplex forming solutions in the portion of the plurality of first wells in the first plate 8012. The gantry 2004 and the substrate extractor/mixer 2012 enable the first nucleic acid tag of the first moiety of the multiplexed paired-binding moieties to bind to a portion of the first substrates in the portion of the plurality of first wells in the first plate 8012 after incubating in the IncuSealer 2014. The substrate extractor/mixer 2012 extracts in parallel, via the portion of the plurality of first substrates, the plurality of multiplexed immunocomplexes from the plurality of immunocomplex forming solutions to form in at least a portion of the plurality of second wells in the second plate 8014 a plurality of first immunocomplex purification solutions, wherein the portion of the plurality of first substrates are eluted from the plurality of multiplexed immunocomplexes in the plurality of first immunocomplex purification solutions.

The substrate extractor/mixer 2012 to extract in parallel the portion of the plurality of first substrates from the plurality of first immunocomplex purification solutions. The gantry 2004 combines a portion of the second substrate solution comprising the plurality of second substrates from the detection kit with the plurality of first immunocomplex purification solutions in the portion of the plurality of second wells in the second plate 8014. The gantry 2004 and the substrate extractor/mixer 2012 enable a portion of the second nucleic acid tag of the second moiety of the paired-binding moieties to bind to a portion of the second substrates in the portion of the plurality of first immunocomplex purification solutions in the portion of the plurality of second wells in the second plate 8014.

The substrate extractor/mixer 2012 extracts in parallel, via the portion of the plurality of second substrates, the portion of the second substrates in the portion of the plurality of first immunocomplex purification solutions in the portion of the plurality of second wells in the second plate 8014 to form in a portion of the plurality of third wells in the third plate 8016 a plurality of multiplexed analyte-specific reporters by ligating in parallel a plurality of the first nucleic acid target label from the first moiety of the paired binding moieties (directly or indirectly), a plurality of the second nucleic acid target label from the second moiety of the paired binding moieties, with a plurality of sample-specific target labels. The plate washer 2010 washes the recently used second plate to prepare it for reuse in subsequent steps.

The substrate extractor/mixer 2012 extracts in parallel, via the portion of the plurality of second substrates, the plurality of multiplexed analyte-specific reporters from the portion of the plurality of third wells in the third plate 8014 back to the portion of the plurality of second wells in the second plate 8014 to elute in parallel the plurality of second substrates from the plurality of multiplexed analyte-specific reporters in the portion of the plurality of second wells in the second plate 8014.

The gantry 2004 and the IncuSealer 2014 prepare a fourth plate 8018 with the plurality of multiplexed analyte-specific reporters and seal the plate. The thermocycler/qPCR (Reader) 2018 replicates the plurality of multiplexed analyte-specific reporters. The gantry 2004 pools the solutions and prepares them for next generation sequencing (NGS).

It should be noted that in certain embodiments, one or more of the modules or their functions may be integrated into a single module. In certain embodiments, one module with multiple functions may be separated into multiple modules. For example, in certain cases, two or more of the functions of IncuSealer may be combined in a single module of the system, or separated into two modules. Therefore, reference herein to any one of the modules does not preclude such module from performing other functions of the system unless specifically so indicated. Similarly, reference to an instrument system comprising a series of separately recited components does not require the components to be physically distinct structural elements unless specifically so illustrated or described as such (e.g., multiple components may share the same structural elements or have structural elements in common but be configured to function as multiple components of the overall instrument system). Furthermore, there may be multiple copies of some components of the instrument system.

Before an assay run, the user loads samples and all the reagents and dry consumables needed for the run into the Hotel. He/she also loads bulk fluids, such as wash buffer, rinse buffer, etc., into the Bulk. The user may also remove, empty and reload the bottle(s) holding waste fluid where necessary. The instrument performs a self-diagnosis to ensure that all functional modules are operating normally. In addition, the gantry uses its barcode scanner to scan barcodes printed on the labels of the reagents and consumables to make sure that the correct items are used for the assay. After completion of the self-check, the instrument would start to execute the assay step-by-step in accordance with the assay protocol. These assay steps may comprise adding reagents to the sample, mixing, capturing analytes to the magnetic beads, incubation at set temperature, washing to remove unbound molecules, releasing analytes from the beads, etc. The assay plate may need to be sealed before reading or storage. All these steps can be performed using the functional modules in the system. The gantry uses the gripper to transport assay plates from one functional module to another according to the needs in a particular assay step. At the end of the assay, the assay plate is moved to the readout module. The readout results, together with the run log, can be stored, displayed on screen, or sent to user via internet or intranet. Further, any physical products of the assay, such as nucleic acid reports or NGS (Next Generation Sequencing) library or spent reagents/consumables, can be retrieved from the instrument.

It should be understood, that for the instrument system described herein, each of the modules (e.g., the gantry, washer, and the IncuSealer) may operate simultaneously, or substantially simultaneously, thereby carrying out functions on different spatially separated locations at about the same time. In some embodiments, for example, the gantry may be applying a reagent fluid to a microtiter plate on the stand, while the plate washer is rinsing a different plate. In some embodiments, for example, the readout module may be analyzing the contents of a microtiter plate via qPCR while the IncuSealer is incubating a microtiter plate of the next assay run.

The disclosed instrument optionally comprises devices to prevent the accumulation of biological contamination, which include a UV light and a HEPA air filtration sub-system. In addition, the instrument optionally comprises a deactivation buffer as one of the bulk fluids. It utilizes the plate washer and/or pipettor to wash all used consumables after each assay run.

One important benefit of this modular architecture is that one or more modules can be modified or replaced so that the instrument can be adapted to run an assay that requires a different functional module not available above. Further, if any module malfunctions in the field, it can be quickly replaced to achieve maximum up time for the end user.

Another important feature of the invented instrument is that the container for the samples (“Sample Plate”) is separate from the assay plates. In many prior art instruments, the user must set up the initial assay plate before loading it to the instrument, which may comprise setting up controls, including dilution curves and pre-dilution of samples. This step can be very laborious and error prone. In the invented instrument, the samples are contained in the sample plate in their original status. The instrument can set up the assay plate automatically before the execution of assay protocol.

Many prior art instruments dump used consumables into a waste bin, which is a major source of pollution or run-to-run cross contamination. In the disclosed instrument, used consumables are cleaned using the plate washer and staged back to the hotel, which are disposed by the user before the next assay run.

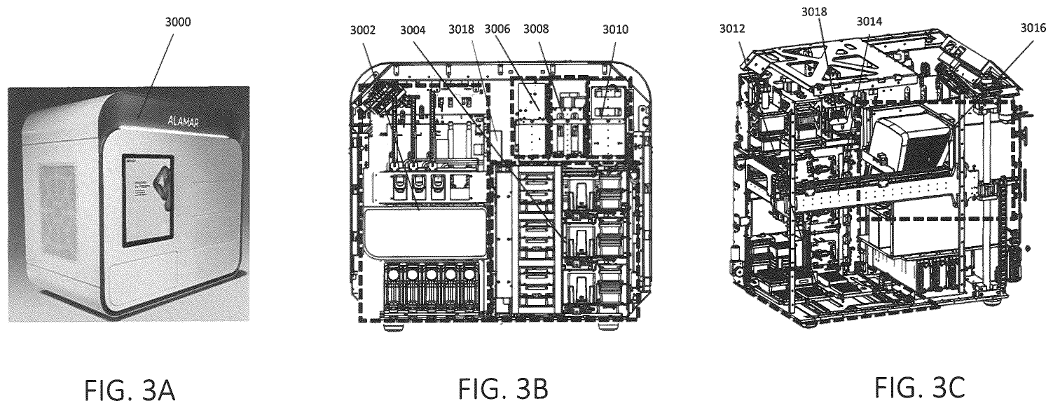

FIGS. 3A-C shows an exemplary design embodiment of the disclosed system. FIG. 3A shows a photograph of a near-launch prototype of the instrument. FIG. 3B shows a front view of the instrument, in which the instrument's housing 3000 is removed to reveal bulk reagents 3002, Hotel 3004, plate washer 3006, mixer 3008, and IncuSealer 2010. FIG. 3C shows a perspective view from the back of the instrument, in which the instrument's housing is removed to reveal stand 3012, gantry 3014, reader 3016 and controller 3018. More detailed description of key functional modules in the system are as follows.

### Hotel

The Hotel in the disclosed instrument is a user/instrument interface module that provides user easy access to load samples, reagents, and other dry consumables into the instrument. It has mechanisms to ensure user safety during such loading. At the same time, the Hotel also provide robotic devices in the instrument with the access to a) examine labels on the loaded items to confirm that they have been loaded correctly; b) to move the reagents and/or consumables to appropriate function modules to carry out assay steps in accordance with the protocol. The Hotel may optionally comprise a refrigerated section that cools the inside temperature to a lower temperature below ambient to preserve temperature sensitive samples and/or reagents. The set lower temperature can be 4° C., 0° C. or even −20° C.

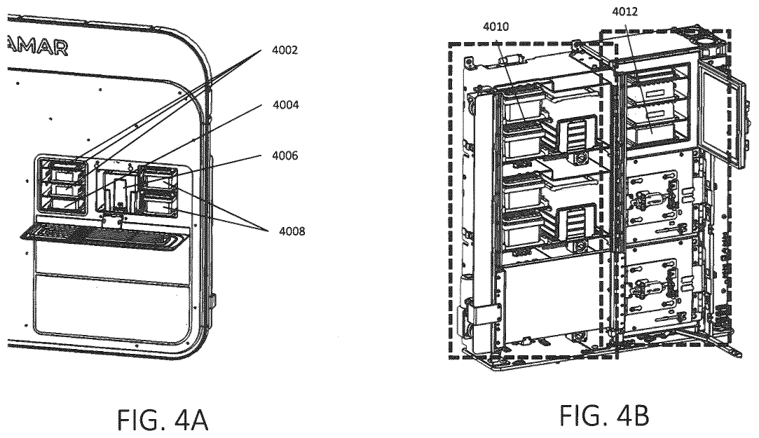

FIGS. 4A and 4B show a particular design embodiment of the Hotel. FIG. 4A shows the Hotel behind the instrument's front panel with the front door at top bay open to reveal loaded reagents and consumables. FIG. 4B shows a rear perspective view of the Hotel with the back door at refrigerated section 4012 of the top bay open. FIG. 4B also shows the back door at the ambient section 4010 at lower bay. The Hotel comprises three almost identically constructed “bays”, each providing shelfing spaces to stage a set of reagents and consumables to execute an assay run. Guiding structures are constructed on the shelves to help user to correctly position the reagents and consumables. In addition, each bay comprises two sections: a chiller section used to stage temperature sensitive samples and reagents that are required to be cooled until use and an ambient section storing room temperature compatible items including dry plates and other consumables. Further, each bay has a front door facing the user on the front panel of the instrument and a rear door facing the gantry inside the instrument chassis. The front door opens to allow user loading reagents and consumables during which the rear door is closed to ensure user safety. The back door opens as needed by the assay protocol to provide the gantry full access to the reagents and consumables. The front door of a particular bay is locked to ensure user safety when the robotic gantry accesses the particular hotel bay. In an exemplary bay shown in FIG. 4A, a sample plate 4004, a detection kit and a target kit 4002 have been loaded to the Chiller portion of the bay. A consumable carrier 4006 (to be disclosed in detail later) and two tip boxes 4008 have been loaded to the RT portion of the bay.

In certain embodiments, for example, the Hotel (supply storage shelves) in the instrument system comprises a single bay of shelfing space. In certain embodiments, for example, the Hotel in the instrument system comprises multiple bays of identically constructed shelfing spaces. In certain embodiments, for example, the Hotel in the instrument system comprises 2, 3, 4, 5, 6, 7, 8, 9, or 10 of such bays.

In FIGS. 4A and 4B, the bays are stacked on top of each other as separate floors. Alternatively, bays can also be positioned side-by-side. Each bay is in a separate drawer, which can be pulled out to allow the user to load from the top. Once the drawer is closed, the gantry can also access the reagents and consumables from the top.

### Gantry

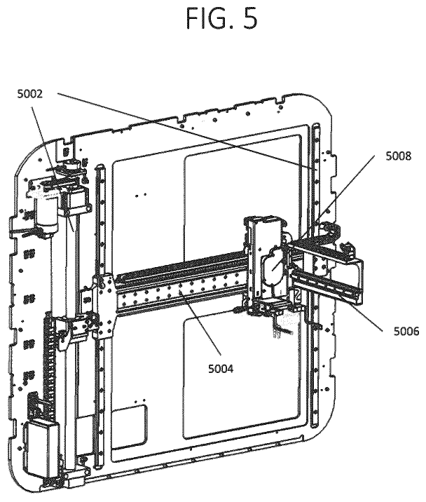

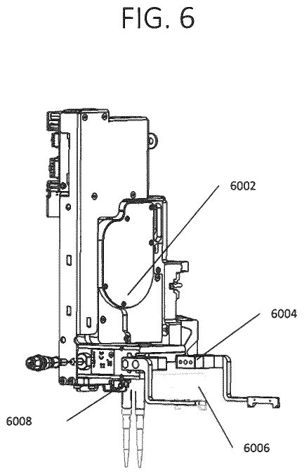

The gantry is a robotic arm with an end-effector carrying one or more tools needed to perform an assay. FIG. 5 shows a particular embodiment of the gantry design, which moves the end-effector 5008 in three degrees of freedom in X, Y and Z axis. The Z axis 5002 is driven by a ball screw mechanism while piezo motors are used to drive the X axis 5004 and the Y axis 5006. FIG. 6 shows a particular embodiment of the end-effector, on which 2 air displacement pipettors (ADP) 6002, a plate gripper 6004, a laser distance sensor 6006, and a barcode scanner 6008 are installed. The gantry is the core of the instrument, responsible for not only transferring fluids but also moving assay plates from one module to another to execute assay steps. In an alternative embodiment, the gantry may comprise two or more mini-gantries with various combinations of tools carried on separate mini-gantries. It is also possible to have a gantry with only 2 degrees of freedom while the 3rd degree of freedom is provided by additional mechanisms in other functional modules.

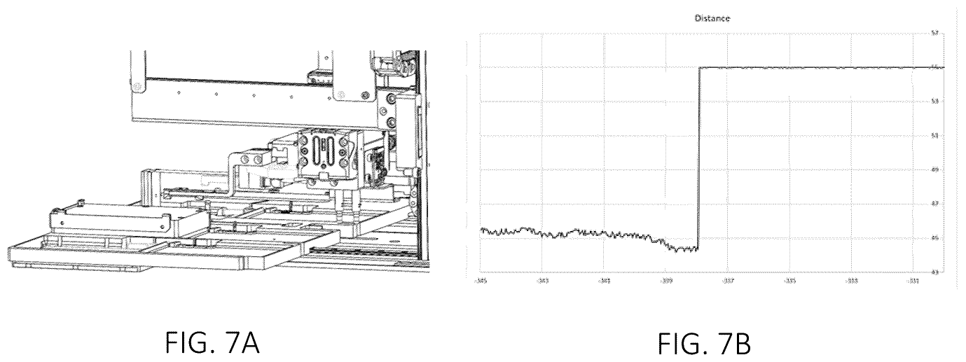

Another innovative feature of the instrument is the incorporation of a position sensor on the end-effector of the gantry. To achieve robust and reliable operation, the pipettor and gripper must achieve sub-millimeter level positional accuracy relative to functional modules in multiple spatial dimensions. Many prior art instruments, such as fluid transfer robots marketed by Hamilton or Tecan, achieve such performance through positional calibration, or so called “teaching”, after the instrument is installed. Such teaching activity is usually very labor intensive and must be repeated each time the instrument is moved and may be required periodically, even if the instrument has not been moved. The disclosed instrument solves this problem by setting up a position target at the module where calibration is required and incorporating a position sensor on the end-effector of the gantry. The position calibration between the end-effector and the module can be achieved by sensing the relative positions of the target and the sensor as needed or periodically in an automated fashion. Any sensor capable of measuring the relative positions between the sensor and the target in 3 dimensions can be used for this application. One particular embodiment of such a position sensor is Keyence IA-030 (https://www.keyence.com/products/sensor/positioning/ia/models/ia-030/). This low-cost sensor is originally designed to measure the distance between the sensor and a surface at high precision thus can only determine the relative position of the target and the sensor in one dimension. FIGS. 7A-B depict a specific implementation where the relative position between such a sensor and a specifically designed target can be determined in all 3 dimensions. The gantry first moves the sensor approaching the front facet of the target block (FIG. 7A). By measuring the distance between the sensor and the front facet, the relative position in Y axis is determined. Then the gantry scans the sensor passing across the top edge of the target block, the precise position of the top edge is detected by monitoring the sudden change of the measured distance (FIG. 7B). The relative position in Z axis is determined. The relative position in X axis can be determined in a similar way by scanning the sensor across the vertical edge of the target block.

For any automated assay instrument, it is important to control all reagents and consumables to be used in the instrument to ensure robust and reliable operation. Many prior art instruments require a user to scan barcodes labeled on reagents and consumables manually during loading. In the disclosed instrument, the reagents and consumables are staged in the Hotel after loading. A barcode scanner is mounted on the end-effector of the gantry. Before the start of the assay run, the instrument moves the barcode scanner to the rear opening of the Hotel and checks every barcode in the set of reagents and consumables to ensure that the correct items have loaded and, where appropriate, in the correct locations. Optionally, the instrument can use the position sensor in conjunction with the barcode scanner to detect potential mis-positioning of the reagents and consumables.

In some embodiments, for example, a suitable computer readable identifier tag may be used instead of barcode. Non-limiting examples of suitable identifier tags may bar codes or radio frequency identification (RFID) chips. The identifier tag may be used for a variety of purposes, such as to authentication and/or verification of identity, type, lot number, expiration date, etc. of the assay consumable and/or its contents. Verification can be achieved, for example, via an optical scanner or RFID proximity reader (depending on the type of identifier tag(s) employed). In certain embodiments, the information from an identifier can be stored for future reference and record-keeping purposes. Any suitable identifier such as a radio frequency identification (RFID) tag, a serial number, a color tag, a fluorescent or optical tag (e.g., using quantum dots), chemical compounds, a radio tag, or a magnetic tag can be used. Detection of identifiers can be accomplished by a variety of methods known to those of ordinary skill in the art. The detection method depends in part on the particular identifier and can include, for example, imaging, fluorescence detection, spectroscopy, microscopy, etc. In one embodiment, a RFID tag is used as an identifier. The RFID tag can include an integrated circuit (e.g., for storing and processing information, modulating and demodulating a radio frequency (RF) signal) and an antenna for receiving and transmitting the signal. The RFID tag may be passive, semi-passive (e.g., battery-assisted), or active. It should be understood that RFID tags are known in the art and that any suitable RFID tag can be incorporated into components of an assay consumable described herein.

### Stand

The plate staging station comprises multiple stands where plates and reagent kits are placed when fluids are transferred from one to another. It also offers temporary staging spaces for plates waiting to be processed in the next assay step. The pipette tip box is also staged on the Stand to enable pipettors on the gantry to pick up tips.

In certain embodiments, for example, the instrument system can accommodate more than 3, 4, 5, 6, 7, 8, 9, 10, 12, 15, or 20 plates internally. In certain embodiments, for example, the instrument system can accommodate between 3 and 10, between 4 and 9, or between 5 and 8 plates internally. In certain embodiments, for example, the instrument system can accommodate between 8 and 10, between 7 and 9, between 6 and 8, between 5 and 7, between 4 and 6, or between 3 and 5 plates internally. In certain embodiments, for example, the instrument system comprises a capacity to store 3, 4, 5, 6, 7, 8, 9, 10, 12, 15, or 20 plates internally.

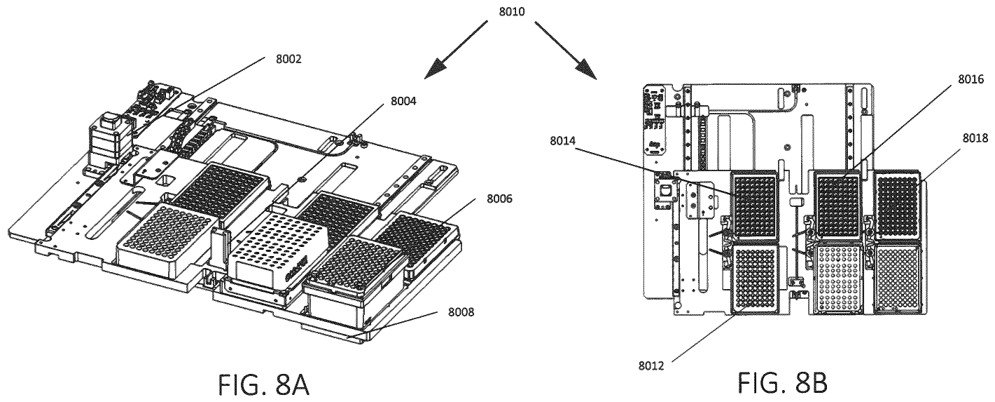

FIGS. 8A-B show a particular embodiment of the stand 8010, which provides six plate positions. FIG. 8A shows a perspective view of the stand 8010, including a motor 8002 that drives the stand 8010 moving along the Y-axis, target block 8004, tip box 8006, and load-cell unit 8008. The entire Stand is movable along one axis (Y). This feature is useful in reducing the footprint of the instrument in the Y dimension. Clearly, the pipettor must be able to access all wells on every plate position on the Stand. Extra clear spaces are needed around the Stand to avoid other tools, such as the gripper, from physically hitting other items. Moving the Stand can resolve this interference without increasing the instrument footprint. Optionally, load cells are installed under one or more plate positions, which can be used to monitor the force applied to ADP during tip pickup. This ensures the operational robustness of tip picking-up and ejection.

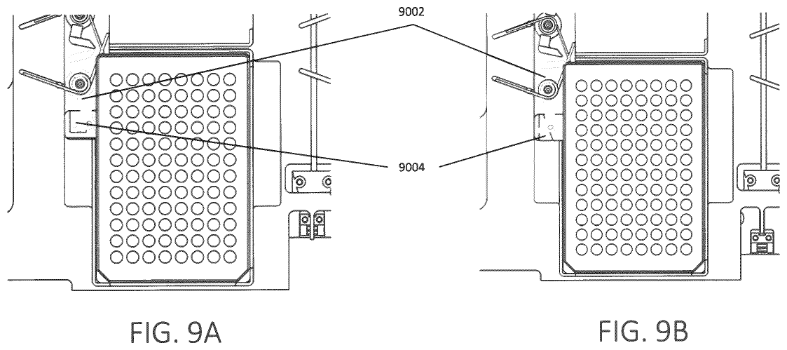

Another important function of the Stand is to increase the positional accuracy of consumables, which in turn improves the operational reliability in the automated instrument. As described before, reagents and consumables are loaded into the Hotel manually by the user. The positional accuracy and consistency of these items are not high due to the manual operating process. The instrument would first transport the reagent kits or consumables from the Hotel to the Stand. Each plate position on the Stand provides a pocket to receive the plate, which is sufficiently large to accommodate the positional variation in the Hotel. Once the plate is in its pocket, a “corner pusher” mechanism is activated to push the plate towards one corner of the pocket, thus forcing the plate to align with the pocket in a highly repeatable manner. FIGS. 9A-B shows a particular implementation of the corner pusher mechanism where each pusher 9002 is spring loaded to the “close” position. A cam mechanism 9004 opens the pusher 9002 when the Stand is moved to a specific position where the plate is placed into or taken out of the pocket (FIG. 9A). After the Stand moves away from the plate loading position, the pusher 9002 is closed by the spring, pushing the loaded plate towards one corner of the pocket, thus aligning the plate precisely with the Stand (FIG. 9B).

An alternative approach to ensure that a plate is placed on the Stand with high accuracy is to ensure that when the gantry approaches a plate to move it out of the Hotel, the relative position between the gripper and the plate to be grabbed is accurate. This can be achieved by using the position sensor on the end-effector to measure the distance between the gripper and the plate (Y axis). In addition, the gripping action naturally centers the plate to the gripper, ensuring positional accuracy in X axis.

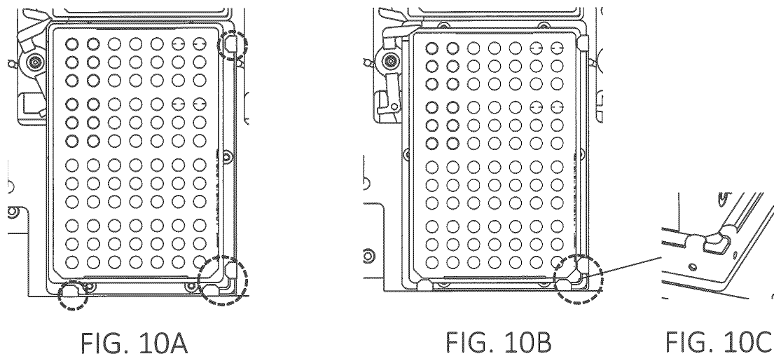

The stand may optionally comprise a mechanism that prevents the plate staged on a plate position from moving up. This is important because some plates or kits have containers sealed by a sealing film. When pierced by a pipette tip, the film tends to grab on the tip and forces the plate to move up when the pipettor withdraw. FIGS. 10A-C show a particular implementation of the mechanism, where parts are installed on the edges of a plate staging pocket, which produces an “overhang” feature over the flange feature of the microtiter plate, preventing it from moving up. When taking a plate out of such a pocket, the gantry uses the gripper to grab the plate, slide sideways to clear the overhang before moving up out of the pocket.

### Mixer

Magnetic beads are widely used in biochemistry assays as solid surfaces that selectively bind to one specific type of molecules from a mixture of different molecules in a sample to achieve separation, purification or signal enhancement. There are basically two approaches to process magnetic beads in assay: 1) fluid transfer, in which, after completing an assay step, beads are held within the reaction vessel while the used fluid is removed from the vessel and then, if needed, a new reagent is added; 2) bead transfer, in which beads are captured from a solution in a vessel and released into a different vessel containing a new reagent for the next step of the assay. The disclosed instrument may optionally include a functional module dedicated to processing magnetic beads, which can take either of these two approaches.

In most bead processing units that deploy the bead transfer approach, the bead capture step is achieved by dipping a sleeve-covered magnetic pole into the first reaction vessel thus attracting the beads in the solution to adhere to the sleeve. Then the sleeve/pole is moved to dip into the next vessel. The beads are released into the new reagent by withdrawing the magnetic pole out of the sleeve. To process multiple samples contained in multiple vessels in a plate, a magnetic head comprises multiple poles are used. Correspondingly, multiple sleeves are integrated into a single piece, referred to as a comb. The KingFisher™ family of instruments made by ThermoFisher Scientific represents typical prior art devices that employ bead transfer approach. Their operating mechanisms are covered in issued U.S. Pat. Nos. 6,040,192, 6,207,463, 6,447,729, 6,448,092, 6,596,162.

One advantage of the bead transfer over fluid transfer approach for bead processing is that the bead processing module can also serve the function of stirring and mixing, which is critical in almost all biochemical assays. One important requirement for the module taking the bead transfer approach is that the length of time between the beads leaving the first reagent to re-submerging into the next reagent must be minimized to reduce the risk of bead drying. In many instruments, this is achieved by placing a set of assay plates pre-filled with reagents on a rotary or translational platform. As soon as the comb is raised out of the first reagent, the platform moves the next plate to the position under the comb so that the comb can be lowered immediately to submerge the beads back to solution again. One problem with this configuration is that the instrument requires rather large footprint to stage the pre-filled assay plates, especially for complex assays comprising many assay plates.

Disclosed herein is a more compact configuration for the magnetic bead processor based on the bead transfer approach.

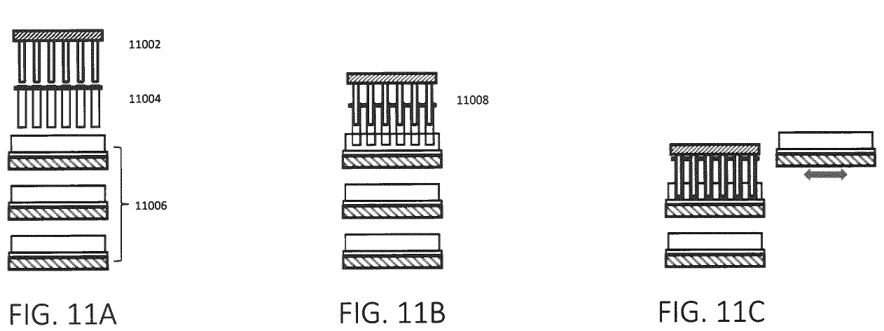

As shown in FIGS. 11A-11C, the disclosed mixer stages the prefilled assay plates on multiple platforms vertically positioned on top of each other. The sleeve comb first accesses the assay plate on the upper platform (FIG. 11B). After completing an assay step, the platform slides sideways to allow the magnetic head to access to the assay plate on the next platform (FIG. 11C). If needed, the used assay plate can be removed manually or by a robotic gripper and replaced by a new plate with fresh reagents. After finishing the assay step associated with the plate on the lower platform, the magnetic head/comb moves to the original position above the upper platform, the upper platform slides back to its original position above the lower platform, then the magnetic head/comb is lowered again to access the fresh plate. By repeating the above steps, the device can perform bead processing functions through an infinite number of assay steps using a minimum of two vertically positioned platforms. The design depicted in FIGS. 11A-C will occupy a footprint of at least two microtiter plates. More specifically, FIG. 11A depicts a front view of the structure, showing magnetic head with poles 11002, plastic comb with sleeves 11004, and assay plates staged on vertically positioned platforms 11006. FIG. 11B illustrates the bead processing device when accessing a plate on the upper platform, where magnetic poles are inserted in sleeves 11008. FIG. 11C shows the bead processing device when accessing a plate on a lower floor.

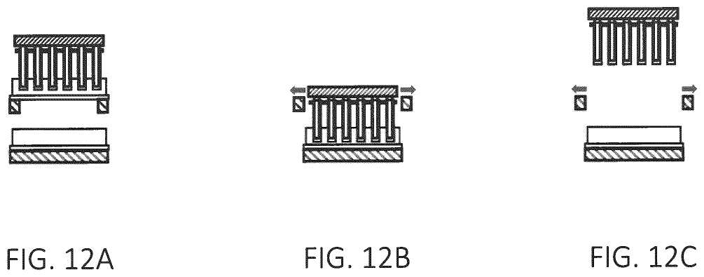

FIGS. 12A-C show a design that can further reduce the footprint where the upper assay plate is placed on a “splitting” platform. After the completion of an assay step, the used assay plate is first removed from the upper platform using a robotic gripper or plate stacker. The upper platform then splits to enable the magnetic head and/or the sleeve comb to access the next plate staged on the lower platform. Similar to the design concept depicted in FIGS. 11A-C, there can be many such vertically positioned platforms as long as that all upper platforms, except the lowest one, are splitable. And like the description of the concept in FIGS. 11A-C, two platforms can be used in turn to perform an infinite number of assay steps.

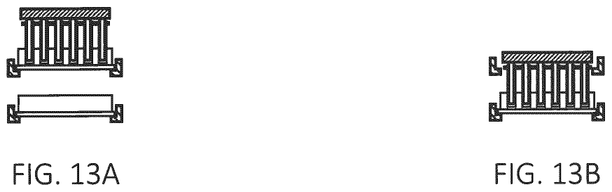

FIGS. 13A and 13B show another concept with two or more fixed and vertically positioned platforms for assay plates. The upper platform(s) has a large opening at center covering the well area of the assay plate. When the assay plate is placed on the top platform, the magnetic poles/sleeves would access it directly as described above. When the assay plate is placed on the lower platform(s), the magnetic pole and sleeve would go through the opening(s) to access the plate on lower platform. The device is greatly simplified as there is no need to move or split the upper platforms. Although the total number of platforms is limited by the length of the magnetic poles/sleeves in this design, it can also, as described before, utilize two platforms in rotation, to perform an infinite number of bead processing steps in an assay.

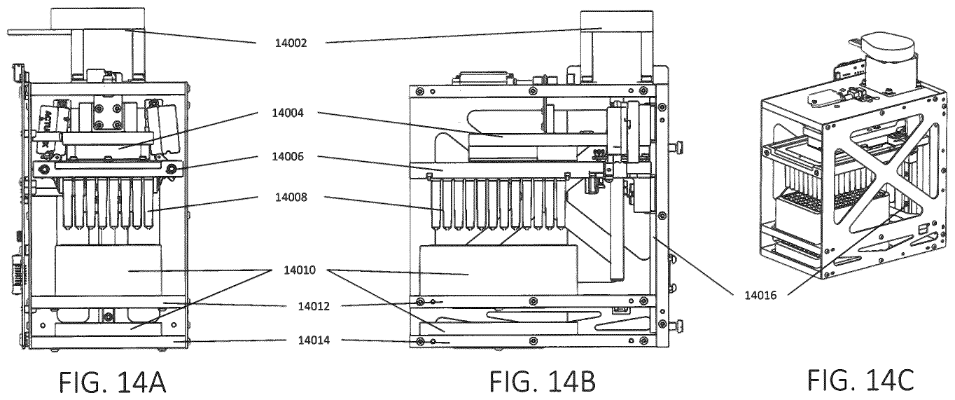

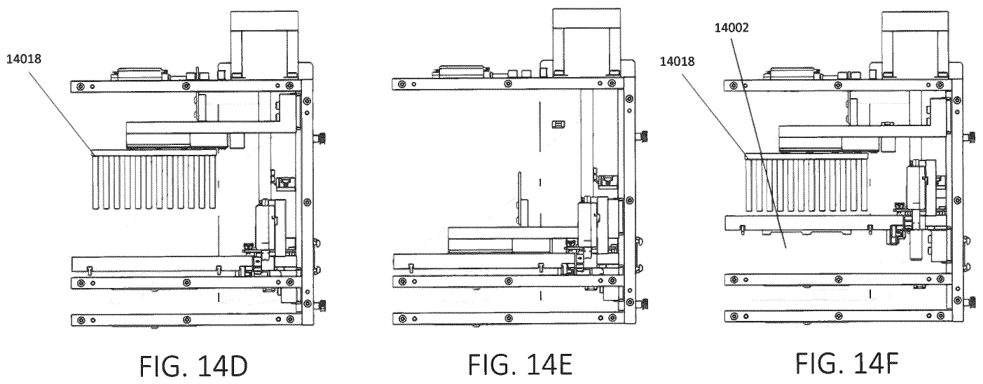

FIGS. 14A-F shows an exemplary detailed design of the concept with two fixed plate platforms. FIGS. 14A and B show respectively the front and side view of the design, including motor 14002, magnetic head carrier 14004, sleeve comb carrier 14006, magnetic head inserted in sleeve comb 14008, microtiter plates 14010, upper platform 14012, and lower platform 14014. FIG. 14C is a prospect view that shows rail 14016. FIG. 14D shows that the magnetic head 14018 is held at the high position, and the comb dips into a plate on lower rack for mixing or bead release. FIG. 14E shows the magnetic head inserts into comb and the head carrier piggy-backs on the comb carrier, and the head/comb dips into a plate on lower rack for bead collection. FIG. 14F shows the magnetic head 14018 is held at the high position, and the comb dips into a deep well plate 14002 on upper rack for bead release or mixing.

As mentioned before, the core operations of any bead process are 1) to collect beads onto sleeve from a solution; 2) to release the beads from a sleeve to a different solution. The bead collection is achieved by inserting a magnetic pole into a sleeve and move the pole/sleeve combo up and down in the vessel containing the solution with beads. The up/down motion is normally slow, giving time for beads to be collected to the magnetic pole. The bead release is achieved by withdrawing the magnetic pole from the sleeve with beads adhered to it and then moves the empty sleeve up/down in a vessel containing a different solution. The frequency of the up/down movement must be high in order to efficiently dislodge the beads from the sleeve surface.

To implement these operations, the magnetic head and the sleeve comb must be mounted on two separate, movable carriers. In prior art devices, the head carrier is mounted on the comb carrier as a sub-assembly so that the head can insert into or withdraw from the comb. When the comb carrier moves, the head carrier, together with the head, moves with it so that the head and the comb combo can be moved up/down together in the vessel to complete the bead collection. The problem with such designs is that, during bead release, the comb carrier has to move with the heavy magnetic head at a high frequency, generating significant vibration. The prior art bead processors employ a large and heavy base to mitigate the vibration. In addition, the head and comb carriers are each driven by an independent motor, resulting in higher cost and complexity.

In the mixer design disclosed herein, as shown by example in FIG. 14, the head and comb carriers are mounted to two separate carriages sliding on the same rail 14016 with the head carrier 14004 positioned above the comb carrier 14006. The comb carrier 14006 is powered to move up and down along the rail 14016 driven by the motor 14002 while the head carrier 14004 is not powered. During bead collection, the magnetic head carrier sits on the comb carrier by its own weight with magnetic poles inserted into their respective sleeves. The comb carrier moves the head/comb combo up/down slowly in the vessels to attract beads to the sleeves. During bead release, the comb carrier first moves to a high up position with the head carrier piggy backed on it. A mechanism is activated to hold the head carrier at the high-up position. Then the comb carrier moves down with the comb only to insert the sleeves into vessels and cycle up/down at high frequency to dislodge the beads from the sleeve surface to fresh reagents in the vessels. Since the comb is very light, such high frequency oscillation will not cause severe vibration. In addition, the device is greatly simplified in the invented design since the head carrier is passive and does not require a power chain.

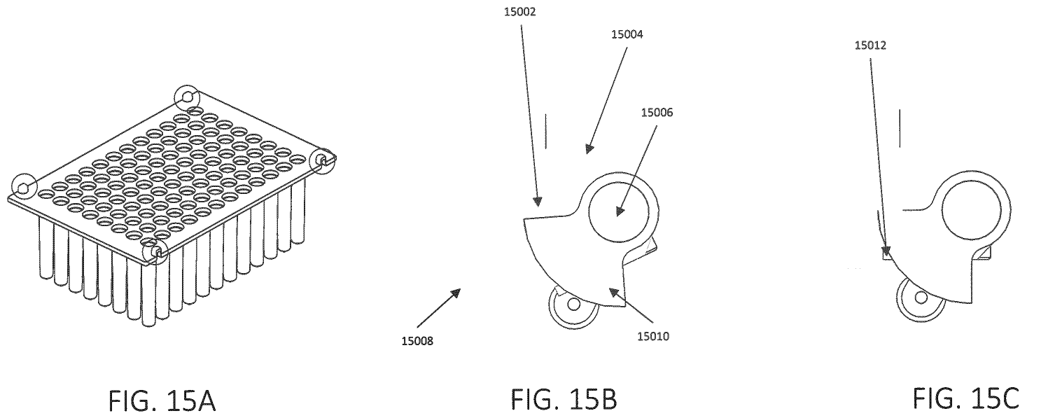

Another critically important task in a bead processor is to align the magnetic poles with the sleeves. The magnetic poles are made of rare earth magnet material and very fragile. Unlike the magnetic head, which is permanently assembled on the head carrier, the sleeve comb is a plastic consumable and is temporarily attached to and held by the comb carrier. The most popular combs have “ear-like” structures built into four corners of their top plate (FIG. 15A). In prior-art bead processors such as KingFisher™, two rotary parts are built on two sides of the comb carrier, each has two “fingers” engaging into the four “ears” on the comb thus aligns and holds the comb to its carrier. The problem with this prior-art design is that the comb carrier cannot hold the comb properly when any one of the comb “ears” is broken during transport, causing a crash between the magnetic head and the comb. The detailed design of the rotary part in the invented processor is shown in FIGS. 15B & 15C (some parts are omitted or made transparent for clarity).

The two rotary parts 15006 on two sides of the comb carrier 15004 also have two fingers 15010 each to engage the ear-like structures 15002 on the comb to align the comb to its carrier 15004 (FIG. 15B). With further rotation of the rotary part 15006 (FIG. 15C), a flapper 15012 behind the “finger” 15010 engages the underside of the comb base plate to hold the comb to its carrier 15004. The desired secure attachment of the comb to its carrier 15004 can be achieved as long as any two out of the four ear-like structures 15002 are not broken. The robustness of the operation is greatly improved.

### Washer

Washing a microtiter plate is one of the most common assay steps in which a reagent, such as a wash buffer is dispensed to wells of the plate and, after optional soaking or shaking, is aspired from them.

There are two types of plate washers on the market: a stripe washer that dispenses a fresh reagent to and aspires the used reagent from wells of the plate one stripe (column or row) at a time; a matrix washer that dispenses and aspires from all wells of the plate in parallel. Typically, the stripe washer is simpler and cheaper, but it takes longer time to complete a wash cycle to a plate. More importantly, since aspiration needles have to be inserted into the fluid in the wells, stripe washer may introduce well-well cross contamination, which limits its applicability. On the other hand, matrix washers are faster with minimum risk of cross well contamination on the plate. However, it is more complex and expensive. Another issue with the matrix washer is that it has a large dead volume and wastes a large amount of reagent during stages of priming or switching to a different reagent.

Disclosed herein is a plate washer that avoids the shortcomings of stripe and matrix washers.

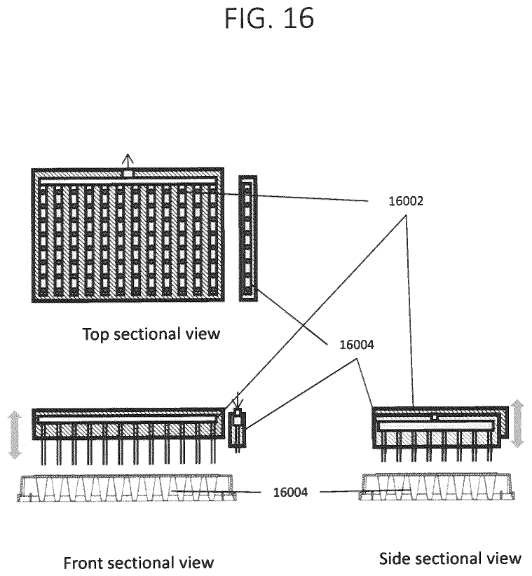

FIG. 16 illustrates the basic conceptual structure of the invented plate washer, which comprises one or more stripe dispensing head 16004 and a matrix aspiration head 16002. The dispensing head 16004 comprises one or more stripes of hollow dispensing needles, in alignment to the wells in one column or row. During dispensing cycle, the reagent is pumped to the manifold connected to the dispensing needles to fill a column or row of wells, then the microtiter plate 16006 moves under the dispense head 16004 to enable the reagent to be dispensed to wells of the next column or row until all wells are filled in the plate 16006. The aspiration head 16002 comprises a 2D matrix of aspiration needles in alignment to each well in the microtiter plate 16006. During aspiration cycle, a relative vacuum is applied to the manifold connected to the aspiration needles and the aspiration head 16002 is lowered to insert the aspiration needle into fluid in wells and aspires the fluid. Since the dispensing needles do not have to be lowered to contact the liquid in wells, there is minimal risk of well-to-well contamination. FIG. 16 depict one exemplary configuration designed for 96 well plates with the dispensing head 16004 having a stripe of 8 needles and the aspiration head 16002 having 96 (8×12) needles. The same concept can easily be adopted for other plate formats, with 24 (4×6), 384 (16×24) or even 1536 (32×48) wells. Using the disclosed plate washer, microtiter plates used in the disclosed instrument system can be washed and re-used in multiple steps of the same assay without worrying about the well-to-well cross contamination, which makes the instrument system more versatile for a wider range of assay protocols.

Many applications require the washer to dispense multiple different reagents. In prior art plate washers, this was achieved by connecting the dispense manifold to different reagents through a multi-way selection valve. Multiple reagents are dispensed one at a time through the same set of needles. The problem with this type of design is that there is always residual volume in the fluid path and some reagents are not compatible to each other and cannot mix. Another important advantage of the disclosed “stripe dispense+matrix aspiration” washer architecture is that the dispensing head can optionally comprise multiple stripes of dispensing needles. Each stripe is fluidically isolated with its own manifold connecting to a dedicated reagent source. Optionally, multiple reagents can be dispensed in parallel.

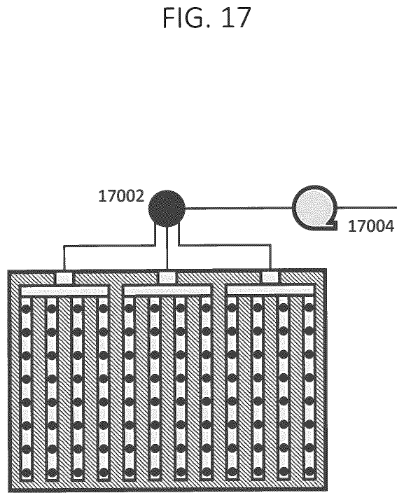

A very powerful pump is required to aspire in parallel all wells on a plate, which is inevitably bulky and noisy. As shown in FIG. 17, the matrix aspiration head provided herein further optionally comprises a partitioned manifold atop the needle matrix with each manifold connecting a portion of the needles in the entire matrix. Using a 3-to-1 selection valve 17002, a portion of the wells in the microtiter plate to be aspirated at a time using a smaller, quieter pump 17004.

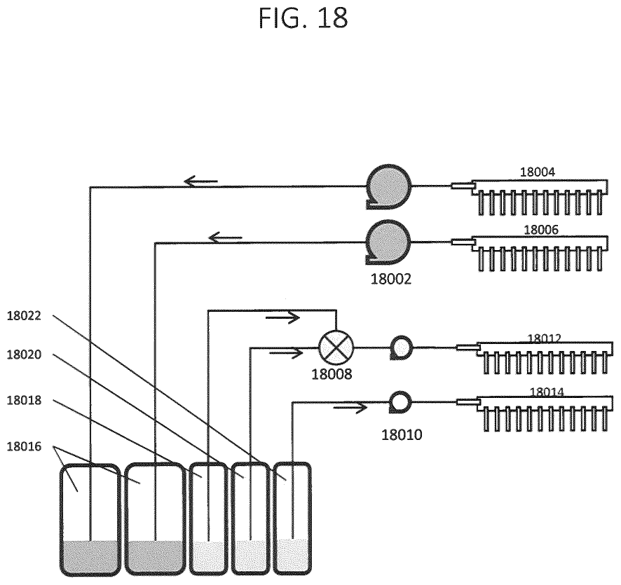

FIG. 18 depicts an exemplary system architecture of the disclosed washer, comprising two dispensing stripes where the dispensing strip #1 18012 is shared by Buffers 1 & 2 (18018 & 18020) selected through a 3-way valve 18008 and strip #2 18014 is dedicated to Buffer 3 (18022). Each dispensing strip is connected to a dispensing pump 18010. The aspiration manifold is partitioned into two separate portions 18004 and 18006, each connected to a waste container 18016 via an aspiration pump 18002.

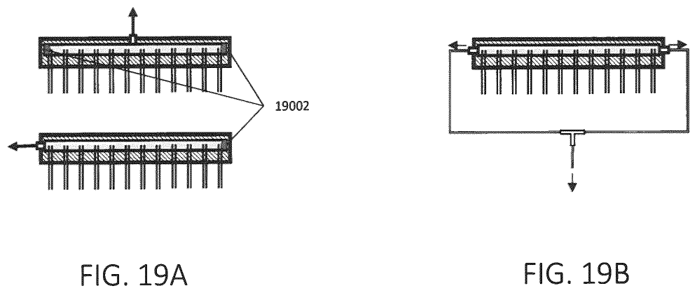

In prior art plate washer designs, as shown in FIG. 19A, the aspiration pump is connected to one side of the aspiration manifold. Sometimes, a small amount of fluid is trapped at the far end of the manifold during aspiration, which may drip down the nearest needle after the aspiration pump stops. Such dripping may introduce well-to-well cross contamination. Again, the power of the pump has to be increased significantly in order to clear the trapped volume completely. More specifically, FIG. 19A shows that aspiration from one end or center of the manifold results in residuals 19002 at “dead end” portion of the manifold. FIG. 19B shows “residual free” aspiration from both ends of the manifold.

The disclosed plate washer solves this problem by connecting both ends of the aspiration manifold to the same pump via a “T” joint (FIG. 19B), which eliminates the trapped fluid at the dead end of the manifold without the need to increase the power of the pump.

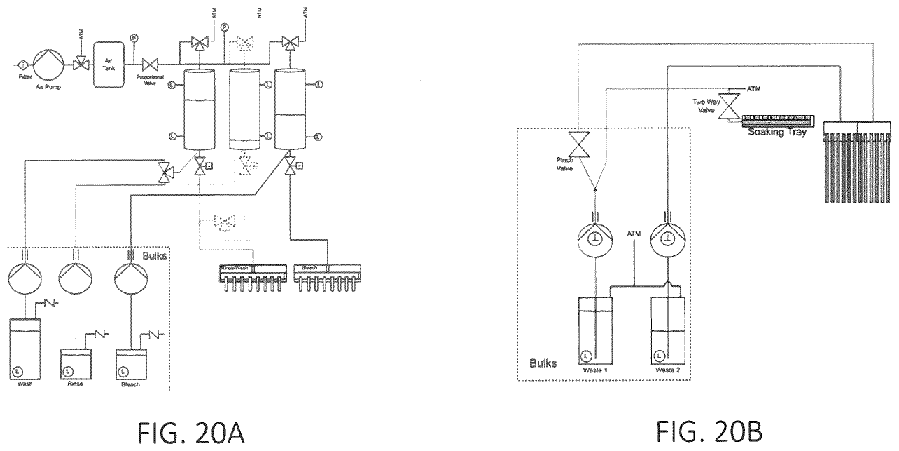

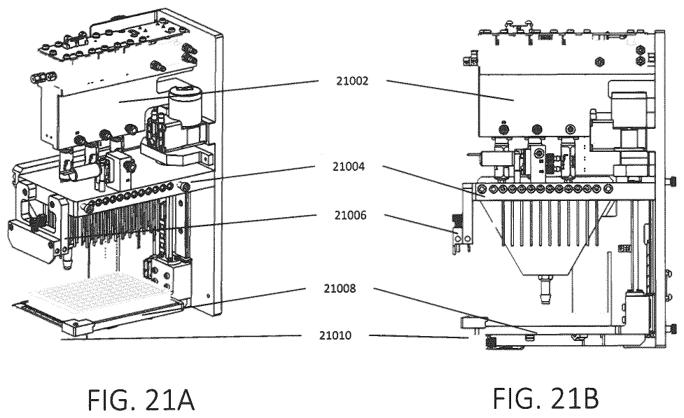

FIGS. 20A-B show a particular embodiment for the system-level piping and instrumentation diagram (PID) of the disclosed plate washer. FIGS. 21A-B shows the details of the design embodiment, where an optional soak tray 21010 is provided so that the needles of a dispense head 21006 can be soaked between dispenses to prevent clogging. Furthermore, FIGS. 21A and B depict reservoirs 21002 for dispensing fluids, matrix aspiration head 21004, two stripe dispensing heads 21006, vertically movable plate platform 21008, and soak tray for dispense needles 21010.

In certain embodiments, for example, the instrument system consumes only a small amount of a cleaning buffer when washing plates during the execution of a NULISA assay cycle. In certain embodiments, for example, the instrument system dispenses no more than 100, 110, 120, 130, 140, 150, 160, 170, 180, 190, or 200 microliter of the cleaning buffer to each well of the plate when washing the plate. In certain embodiments, for example, the instrument system dispenses between 100 and 200, between 110 and 190, between 120 and 180, between 130 and 170, or between 140 or 160 microliter of the cleaning buffer to each well of the plate when washing the plate. In certain embodiments, for example, the instrument system dispenses between 100 and 120, between 110 and 130, between 120 and 140, between 130 and 150, between 140 or 160, between 150 or 170, between 160 or 180, between 170 or 190, or between 180 or 200 microliter of the cleaning buffer to each well of the plate when washing the plate. In certain embodiments, for example, the instrument system dispenses 100, 110, 120, 130, 140, 150, 160, 170, 180, 190, or 200 microliter of the cleaning buffer to each well of the plate when washing the plate.

In certain embodiments, for example, the instrument system flushes its internal fluid lines with a decontamination solution at the end of each NULISA assay cycle. In certain embodiments, for example, the instrument system washes each of the multiple plates that has been used with the decontamination solution at the end of each NULISA assay cycle, which neutralizes infectious agents or hazardous biologic materials that present a risk or potential risk to the health of humans, animals, or the environment, such that the used plates may be disposed of in regular non-hazardous waste recycling stream. In certain embodiments, for example, the decontamination solution is solution of bleach. In certain embodiments, for example, the decontamination solution is an aqueous solution of sodium hypochlorite. In certain embodiments, for example, the decontamination solution is an aqueous solution of sodium hypochlorite at a concentration of 0.1%, 0.2%, 0.5%, 1%, 2%, or 3%.

### IncuSealer

Incubation is one of the most common assay steps in which reaction solutions are kept under a certain temperature for a defined period. Since it is often the case that the incubation temperature is well above ambient and sample volume is small, an incubator must have means to prevent evaporation and condensation of evaporated liquid.

Sealing an assay plate with a multi-layer thin film is often a required step at the end of the assay just before the assay plate is sent for readout or storage. These sealing films come in two categories based on sealing mechanism: 1) Pressure sensitive adhesive (PSA) films are glued to the plate by pressure; 2) Heat sealing films are welded onto the microtiter plate using a heated block.

The plate incubation and sealing functions are normally performed by two separate devices. Disclosed herein is a compact incubator/sealer that performs both incubation and sealing functions to a microtiter plate in a fully automated process.

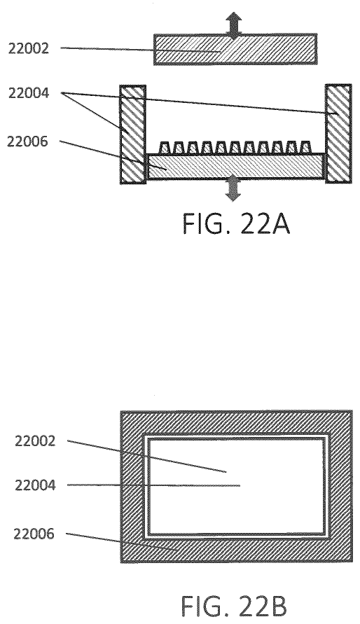

FIG. 22 illustrates the basic conceptual structure of the disclosed module, which comprises three sub-assemblies: 1) a top block 22002 of which the temperature could be controlled to a set point appropriate for incubation. The set temperature can also be a temperature appropriate for welding a sealing film on to the sample plate, which is typically in the range of 150 C to 200 C; 2) a bottom block 22006 that can also be controlled to a set temperature and optionally comprises a surface profile that conforms to the contour of the wells at the bottom of the assay plate to maximize the heat transfer efficiency; 3) a thermal isolating enclosure 22004 surrounding the top and bottom blocks. Optionally, the enclosure 22004 can also be controllably heated to a temperature appropriate for incubation. The top and bottom blocks 22002 and 22006 can move up and down, clamping an assay plate between them. A pressure sensor is placed either on the top block 22002 or bottom block 22006 to measure the clamping force. By controlling the movement of the top block 22002 and bottom block 22006 in sync, the entire sandwich (top block 22002, assay plate and bottom block 22006) can move relative to the enclosure while maintaining the clamping force.

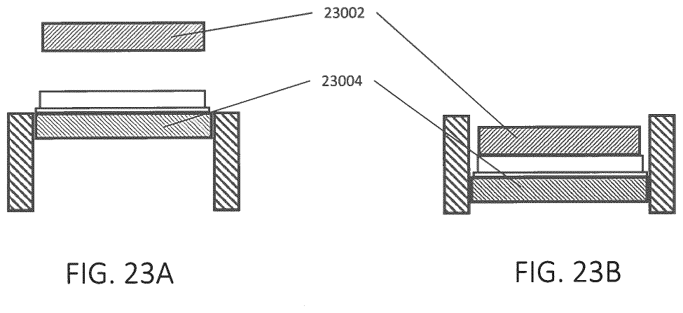

FIGS. 23A and 23B depict the use of the disclosed module for incubation. The bottom block 23004 is controllably heated to and held at a set temperature appropriate for incubation and the top block 23002 is controllably heated to and held at a temperature slightly higher than the set incubation temperature. The reason for the higher temperature at the top block 23002 is to prevent fluid condensation on the surface of the top block 23002. An assay plate is placed on the bottom block 23004 (FIG. 23A), where the bottom block 23004 may optionally move out of the enclosure to facilitate the placement. The top block 23002 is lowered to clamp on the assay plate with sufficient pressure to seal the plate, preventing evaporation during the incubation. The sandwich formed by the top block 23002, assay plate and bottom block 23004 is moved together into the enclosure and held there for the duration of the incubation (FIG. 23B). Optionally, the enclosure is also heated to improve the temperature uniformity across the assay plate during the incubation period. After incubation, the sandwich is moved out of the enclosure and the top and bottom blocks 23002 and 23004 separate to release the assay plate.

In certain embodiments, for example, IncuSealer module in the instrument system can keep reaction solutions at a set temperature range of between 3° and 40° C., between 35 and 40° C., between 35 and 39° C., between 36 and 38° C., or between 36.5 and 37.5° C. In certain embodiments, for example, IncuSealer module in the instrument system can keep reaction solutions at a set temperature point of 25, 30, 33, 35, 37, 39, or 40° C. In certain embodiments, for example, IncuSealer module in the instrument system can programmable keep reaction solutions at any temperature between room temperature and 100° C. In certain embodiments, for example, IncuSealer module in the instrument system can programmable keep reaction solutions at a low temperature point and a high temperature point, wherein the low temperature point is 25, 30, 33, 35, 37, 39, or 40° C., and the high temperature point is 80, 83, 85, 87, 89, 90, 93, 95, 97, or 100° C. In certain embodiments, for example, IncuSealer module in the instrument system can programmable keep reaction solutions at a low temperature point and a high temperature point, wherein the low temperature point is 37° C., and the high temperature point is 95° C.

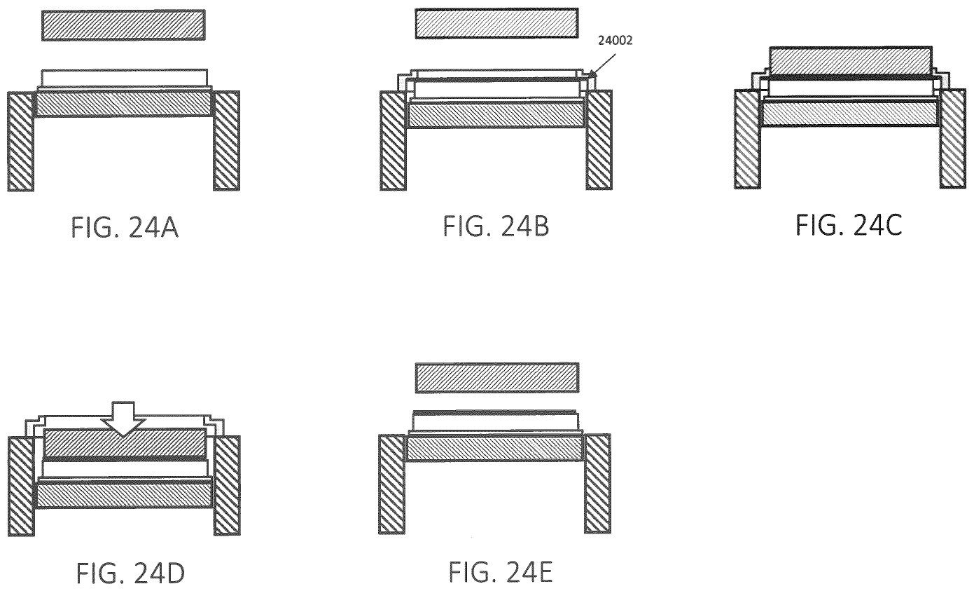

FIGS. 24A-E illustrate the use of the disclosed module to seal an assay plate with a sealing film pre-assembled to a rigid frame. Details of this framed sealing film (“FSF”) are disclosed in the “Consumables” section. After the assay plate is placed on the bottom block (FIG. 24A), the FSF is placed on a separate platform with the film suspended just above the assay plate (FIG. 24B). The top block moves down to press the sealing film onto the top of the assay plate at a set pressure (FIG. 24C). If the FSF has heat based sealing film, the top block additionally has to be pre-heated to a set sealing temperature (usually 150-200 C). If the FSF has PSA based sealing film, the set pressure has to be sufficient to ensure sealing. After the attachment of the film to the assay plate by pressure or heat, the top and bottom blocks move down through the opening portion of the FSF frame in a synchronized speed to maintain the clamping force on the assay plate. Since the frame is held in a fixed position, the sealing film is torn off from the frame (FIG. 24D). The used frame (without the film) and the sealed assay plate can be removed from the module after sealing (FIG. 24E).

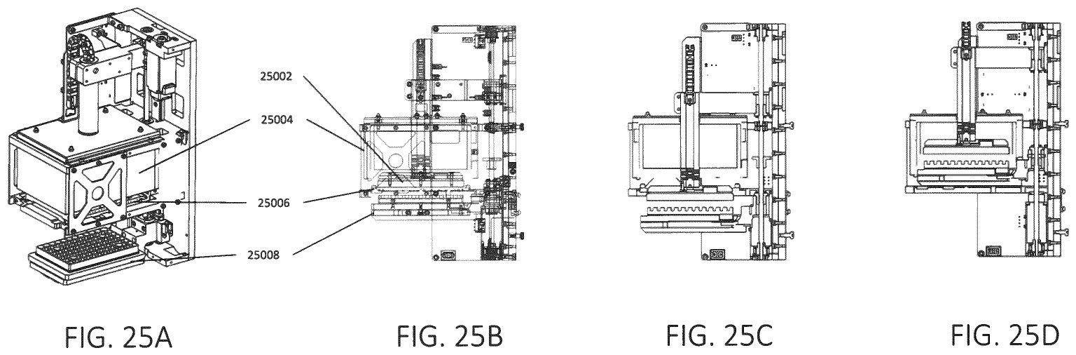

FIG. 25 shows an exemplary design of the disclosed module. More specifically, FIG. 25A shows a perspective view of the module. FIG. 25B shows a side view of the module for sealing a plate using a framed sealing film (FSF). FIG. 25C shows a sectional side view of the module for de-framing the FSF. FIG. 25D shows a sectional side view of the module for incubating a plate. FIGS. 25A-B depict top block 25002 and bottom block 25008, thermal enclosure 25004, and framed sealing film 25006.

### Bulk

The bulk fluids typically refer to certain common reagents that are consumed in an assay in large quantities, which may include wash buffers, rinse buffers and sanitization fluids. The waste fluid generated from the assay procedure can also be regarded as one of the bulk fluids. The disclosed instrument optionally includes a bulk fluid station to house and manage these bulk fluids.

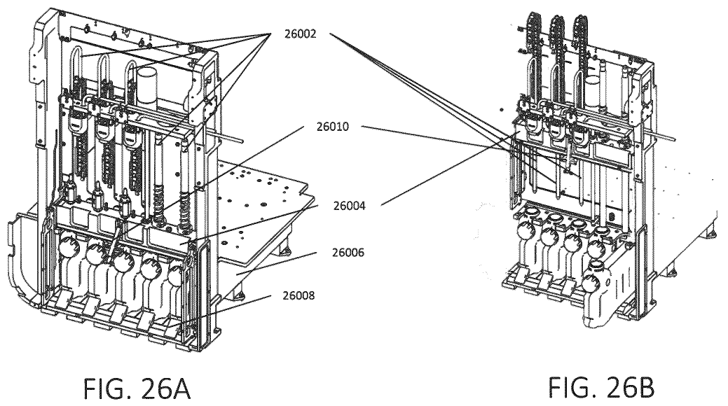

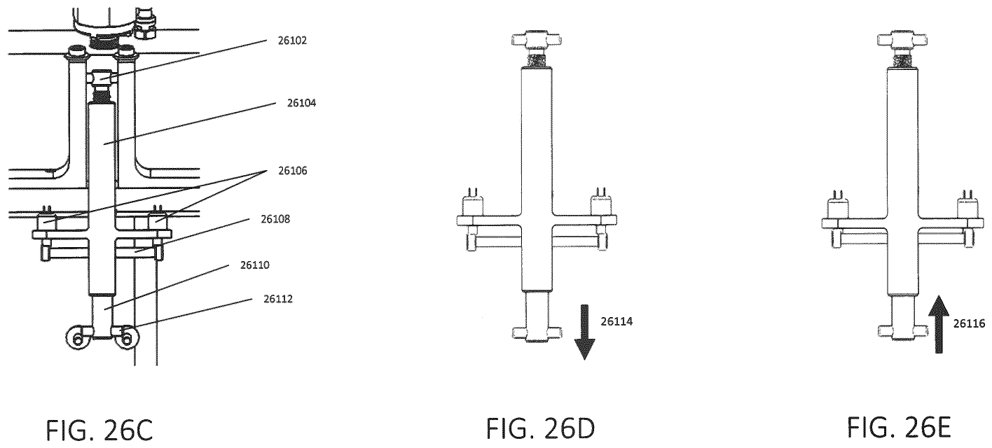

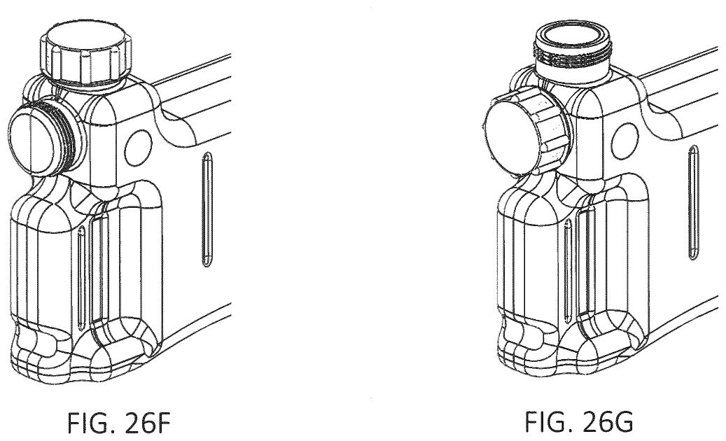

FIGS. 26A and 26B show a particular embodiment of the bulk station comprising a bulk station door, bulk nests 26006 and a tube manifold 26004. The bulk station further comprises sip tubes 26002 and a link 26010 connecting manifold to a door of the bulk station. The bulk station door rises to reveal multiple bulk nests 26006 to allow the user to load a bulk bottle 26008 into its dedicated nest 26006. The nest 26006 has a locking mechanism to hold the bottle 26008 in place and a weight sensor underneath to monitor the quantity of the fluid in the bottle 26008. After the completion of the manual loading, the bulk door closes, and the tube manifold 26004 is lowed to insert a set of sip tubes 26002 into the bulk bottle 26008. The tubes 26002 are connected to the plate washer module or other fluidic management devices in the instrument.

FIGS. 26C-E illustrate the bulk door safety switch mechanism. As depicted in FIG. 26 C, the mechanism comprise upper support 26102 that is fixed to moving lift, housing 26104, two pushbutton switches 26106, and two switch actuators 26108, slider rod 26010 that moves with the door, and lower support 26112 that is connected to the door. The tube manifold supports the top end of a collapsible linkage comprising the safety switch mechanism. The bulk door hangs by gravity on the bottom end of the safety switch mechanism. The safety switch mechanism incorporates two pushbutton switches that are actuated when an obstruction impairs the downward closing movement of the bulk door. The actuation of the switches causes the bulk door closing to stop, but is ignored in the final closing distance when there is no possible danger from finger pinching or other injury to operators. As shown in FIG. 26D, gravity pulls door and slider rod down (26114). As shown in FIG. 26E, obstruction pushes door and slider rod up (26116).

FIGS. 26F and 26G show the two possible positions of the bulk bottle caps. The bottles have two threaded features that can accommodate a screw-on cap—the open top and closed front. After a cap is removed from the open top position of the bottle, it can be stored in the closed front position, and visa-versa. There is a bottle cap detector in the bulk station to prevent the bulk bottles from being installed with the cap in the open top position and damaging the sip tubes and/or mechanism during operation.

In certain embodiments, for example, the instrument system contains sufficient amounts of reagents and other consumables for running several cycles of assays without reloading of reagents and other consumables by users. In certain embodiments, for example, the instrument system contains sufficient amounts of reagents and other consumables for running more than 5, 6, 7, 8, 9, 10, 11, or 12 cycles of assays. In certain embodiments, for example, the instrument system contains sufficient amounts of reagents and other consumables for continuous operation of an extended period of time without requiring reloading of reagents and other consumables. In certain embodiments, for example, the instrument system contains sufficient amounts of reagents and other consumables for continuous operation of 12 hours, 24 hours, 36 hours, 48 hours, 3 days, 4 day, 5 days, 6 days, 7 days, 8 day, 9 days, 10 days, 11 days, 12 day, 13 days, 14 days, 15 days, 20 days, or 30 days.

### Reader

The readout module in the disclosed instrument can be any assay readout devices based on various detection principles, which may include qPCR unit, ddPCR unit, flow cytometer, fluorescent or luminescent plate reader, mass spectrometer etc.

In certain embodiments, for example, a quantitative polymerase chain (qPCR) reaction is used at the end of each NULISA assay cycle to quantify the results of the NULISA assay. In certain embodiments, for example, the microplate containing the products of the NULISA assay is removed from the bench-top instrument by the user and is placed into a separate thermocycler for execution of the qPCR reaction. In certain embodiments, for example, the microplate containing the products of the NULISA assay is automatically transferred, such as by a robotic arm, from the bench-top instrument to a separate thermocycler for execution of the qPCR reaction. In certain embodiments, for example, the bench-top instrument runs the qPCR reaction in an internally integrated qPCR module. In certain embodiments, for example, the internally integrated qPCR module has a form factor of approximately 13 inches W×22 inches D×14 inches H. In certain embodiments, for example, the internally integrated qPCR module is a commercially available off-the-shelf qPCR system, such as Bio-Rad Opus 96.

### Consumables

The disclosed instrument executes assays on multiple samples in parallel using a multi-vessel carrier plate, which includes microtiter plates in standard 24, 96, 384 etc. formats. It optionally uses plastic sleeve combs to transfer magnetic beads in its Mixer module.

In certain embodiments, for example, the dimensions of the microtiter plates used in the instrument system can conform to standards specified by the Society for Laboratory Automation and Screening (SLAS) and the American National Standards Institute (ANSI), published January 2004 (ANSI SLAS 1-2004 to 4-2004). In certain embodiments, for example, the footprint dimensions the microtiter plates are about 127.76 mm (5.0299 inches) in length and about 85.48 mm (3.3654 inches) in width.

In certain embodiments, for example, the multi-vessel carrier plates used in the instrument system are conventional multi-vessel carrier plates that comprise 96 wells. In certain embodiments, for example, the multi-vessel carrier plates used in the instrument system are comprise an increased quantity of the plurality of wells beyond 96 in order to increase the throughput of the NULISA assay of the instrument system. In certain embodiments, for example, the multi-vessel carrier plates used in the instrument system can have, but is not limited to, any of the array configurations of wells described in Table 1.

TABLE 1

| Number of Wells | Arrangement | Volume (mL) |
|---|---|---|
| 6 | 2 × 3 | 2-5 |
| 12 | 3 × 4 | 2-4 |
| 24 | 4 × 6 | 0.5-3 |
| 48 | 6 × 8 | 0.5-1.5 |
| 96 | 8 × 12 | 0.1-0.3 |
| 384 | 16 × 24 | 0.03-0.1 |
| 1536 | 32 × 48 | 0.005-0.015 |
| 3456 | 48 × 72 | 0.001-0.005 |

In addition to the common consumables, the disclosed instrument also deploys special consumables disclosed below to facilitate fully automated assay run and/or to improve user experience.

### Framed Sealing Film

Sealing of an assay or sample plate using a biochemically compatible sealing film is a widely used process either in the middle of an assay before incubation or target amplification by PCR or at the end of the assay in preparation for long-term storage. The sealing film typically comprises multiple layers of materials with the layer facing the assay plate facilitates adhesion and other layers providing structure or protection. Some films have a metallic layer to prevent evaporation and/or to protect against light. For the purpose of this disclosure, all these film or foil are referred to as “sealing films”.

Incorporating plate sealing into a fully automated assay flow is challenging because the sealing film is highly flexible and stretchable thus difficult to handle in an automated instrument. The common prior art method is to organize the film into a large roll format and deploy dedicated mechanical and pneumatic mechanisms to pull the film out of the roll, lay it flat on plate, seal and then cut out the size. Such a device is bulky, complex and costly, only suitable for applications where a large number of plates need to be sealed.

U.S. application publication No. US20120058516 has disclosed a different approach where the sealing film is pre-mounted on a rigid frame so that the framed sealing film (FSF) assembly can be handled by a robotic gripper commonly available in automated assay instrument systems. The frame often has to be detached (“de-framed”) from the film after sealing so that the sealed plate can be used in the follow-on assay procedure. US20120058516 describes a number of approaches to construct the FSF and to de-frame, all of which seem feasible in theory but difficult to achieve the robustness and reliability required for automated operation. The applicant of US20120058516 announced the launch of a commercial product, “FrameSeal”, based on the patent application in 2017 but later withdrew the product from the market and the above-mentioned application was later abandoned.

We herein disclose a different FSF, which has been proven to operate robustly in a fully automated environment. A functional module designed to seal and de-frame using the disclosed FSF is described in Section 4.6 (IncuSealer) of this application.

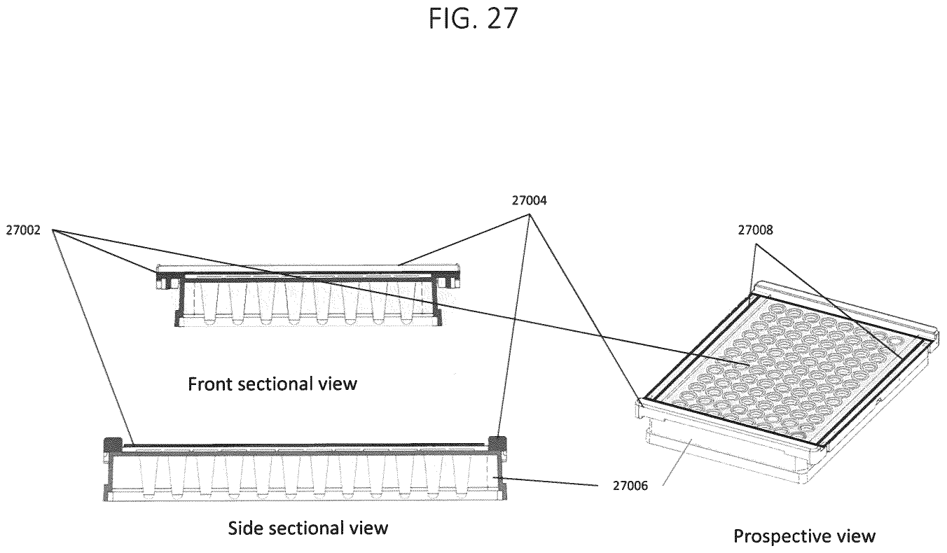

Similar to the prior art referenced above, the frame can be made of any material providing sufficient rigidity and the film can be mounted on the frame using a variety of methods including mechanical clamping, heat welding, laser welding, epoxy, pressure sensitive adhesion etc. FIG. 27 illustrates a specific embodiment of the frame design, which comprises mechanical features to stack on a standard microtiter plate 27006 so that the pre-mounted film 27002 is laid flat above the top surface of the assay plate to be sealed. It may optionally include features for self-stacking. In one embodiment, the frame 27004 is molded using glass fiber enforced polypropylene. The material provides sufficient mechanical rigidity for the frame 27004 and at the same time, enables most sealing films to be heat welded onto the frame 27004 along the weld lines 27008.

Unlike the prior art, certain perforation patterns are pre-cut on the sealing film using die cutting or other cutting techniques. The perforation comprises a series of openings and connections. The location and length of each perforation and the widths of opening and connection in the perforation pattern are all carefully designed so that the film is attached to the frame at sufficient strength that the FSF can be handled safely during fabrication, transportation and operation in the instrument. On the other hand, once the central portion of the film is sealed on the assay plate, the film can be easily torn from the frame by the sealing block pushing down at center portion of the sealing film, as described in Section 4.5.

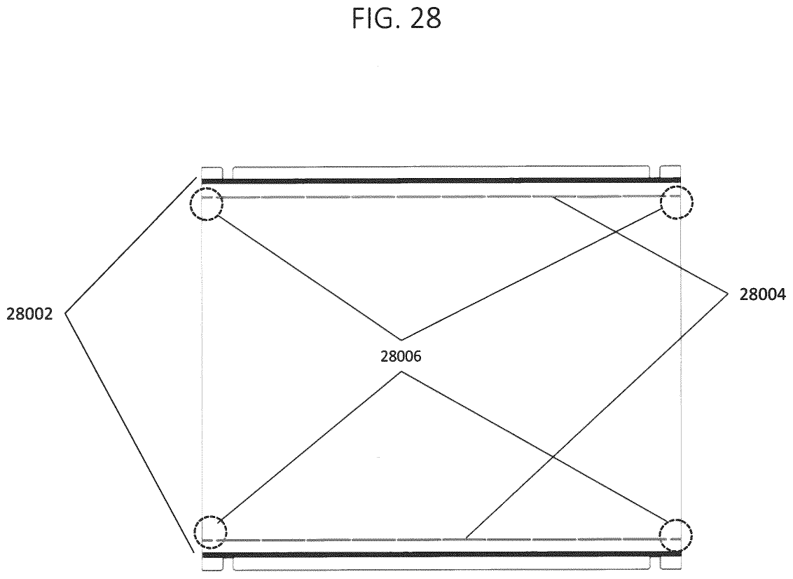

FIG. 28 shows a specific embodiment of the perforation design, in which the film is welded on the frame on two sides and the perforation lines 28004 are positioned right next to the weld lines 28002 closer to the center of the film. In addition, the opening section of the perforations extends all the way to the edges of the film at all four corners. This design ensures that during de-framing, the tear lines will reliably initiate at these corners and develop an opening 28006 along the perforation lines 28004 until the film is completely separated from the frame.

The FSF disclosed in the prior art uses only the sealing film that seals the plates by heat welding and most de-framing methods are facilitated by film softened by heat. The FSF disclosed herein, however, can use both heat sealing and PSA based films. In many applications involving heat sensitive reagents, PSA based sealing film has an advantage over the heat-based sealing.

### Universal Reagent Cartridge

A biochemical assay always consumes a set of reagents, which are often assembled into one or multiple “reagent kit(s)”. It is highly beneficial in an automated assay instrument to handle these kits in a consistent manner. However, since the number reagents in the kit and their types and volumes are all assay specific, it becomes challenging to design an instrument capable of running a range of different assays.

Disclosed herein is a “universal” reagent cartridge designed with the flexibility to carry multiple different reagents as required by the assay, and at the same time, has the footprint and format suitable to be handled in a fully automated assay instrument.

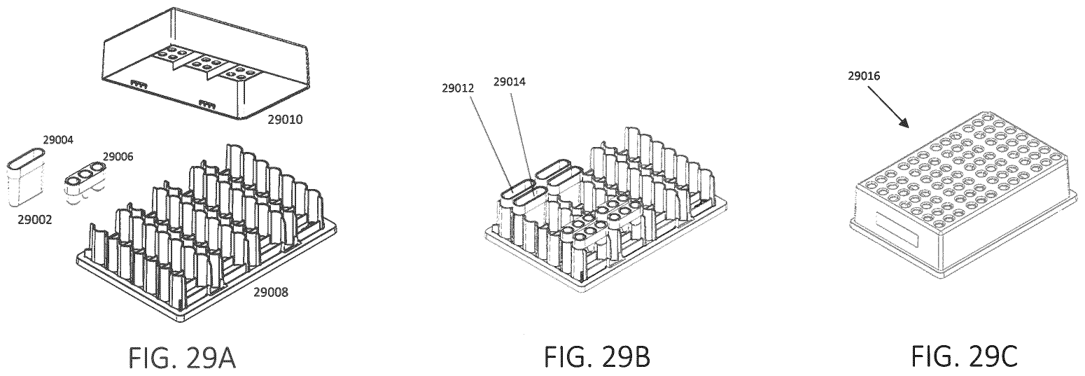

FIGS. 29A-C show a specific design embodiment of the cartridge 29016, which comprises a base 29008, a lid 29010 and multiple reagent containers 29002 of different types (e.g. MONO 29006 and TRIB 29004). The base 29008 comprises multiple positions arranged in a two-dimensional spatial array. Each position is constructed to have a “slot” that holds a container 29002 in place. The different types of containers 29002 have the same outer profile to match with that of the slot so that they can be held by the base 29008 at a specific location. On the other hand, different container types have different volume capacity, and they may comprise one or multiple vessels or wells. The embodiment shown in FIG. 29A comprises two types of containers, one of which has a single vessel with a capacity of approximately 2.7 ml (29006). The other (29004) comprises three vessels. The two side vessels have volume capacity of approximately 300 ul each and the central vessel has volume capacity of approximately 200 ul. Each reagent in a kit is filled into one or multiple suitable containers 29002, sealed with film or foil and assembled on to the base 29008 of the cartridge. The lid 29010 is then applied to the base 29008 which clamps the containers 29002 securely on the cartridge. The lid 29010 has an array of holes on its top surface that are aligned to the container positions underneath and allows pipettor tips to penetrate the sealing films to access the reagents in the containers 29002. By using different combinations of containers 29002 at different positions, the disclosed cartridge can assemble many different reagents into a wide range of different assay kits in accordance with specific assays. The uniform footprint and pipette window spacing enables the assembled kit to be handled and processed in the instrument in a consistent and fully automated manner.

### Consumable Carrier

In addition to reagents, a biochemical assay also deploys a set of dry items, such as microtiter plates or other empty containers, sleeve combs etc., as “single use” consumables. The FSF disclosed in Section 4.9.1 is also one such consumable.

Loading many consumable items into an automated instrument is a tedious and error prone process. Even if the items are loaded to the right location, their positions may not be sufficiently accurate for the automated handling by the instrument. So delicate items, such as FSF described above, may get damaged during manual handling.

Disclosed herein is a consumable carrier that holds multiple consumable items, enabling the user to load them into the instrument in a single action. Some of these items may be compliant to the microtiter plate standard but some may not. In any case, the carrier comprises internal features to hold each item within a specified positional accuracy as required for the automated handling. It also provides features to protect the delicate consumable items during transportation and user handling.

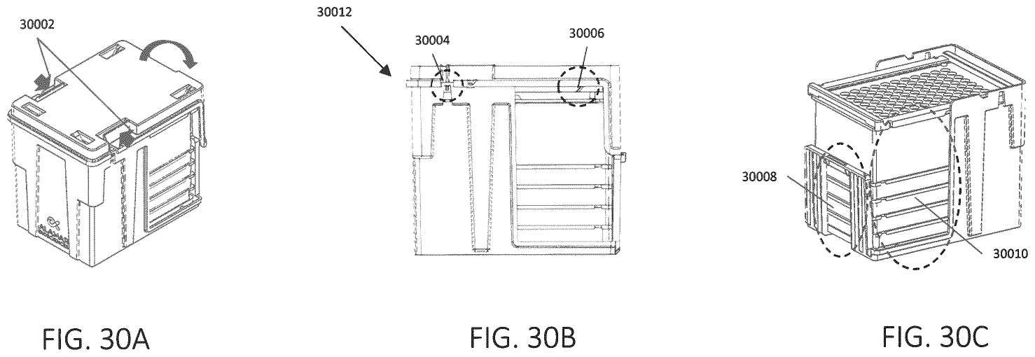

FIGS. 30A-C shows one specific embodiment of the consumable carrier 30012, which comprises a base and a lid and holds eight consumable items including 4 standard microtiter plates, one deep well plate, one KingFisher™ sleeve comb and the FSF. After the consumable items are assembled into the base in the factory, a lid is locked on the base through two spring arms (30004 and 30006) inserting into two small holes on the side walls of the base. The user would open and discard the lid but squeezing the spring-loaded locking mechanism 30002 before loading the base together with the consumable items into the hotel of the instrument. The base also provides large openings 30010 on both side walls that allow the gripper to grab the consumable items and move them one-by-one out of the carrier into the instrument to be used to run the assay. The base also provides front openings 30008 for reading barcodes on the consumable items.

The carrier holds consumable items in place within the required positional accuracy in XY plane using the carefully designed ridges on the internal walls of the base. In Z axis, however, since there are a total of 8 pieces of consumables in the stack and each item has dimensional tolerance of +/−0.125 mm, the total height of the stack may change in the range of +/−1 mm. The plate stack can disintegrate if a plate is separated from the next by more than 0.5 mm. To solve this problem, multiple springy fingers are constructed on the top roof of the lid that push down on the stack to keep the stack stable during transportation.

Bulk Fluid Bottle with Splash Guard

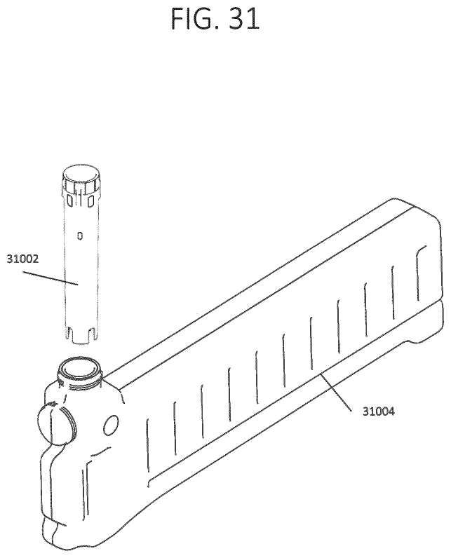

In a specific embodiment shown in FIG. 31, the disclosed instrument comprises one or more bottles each holding different bulk fluids, such as wash buffer, rinse buffer and deactivation buffer. Each bottle optionally comprises mechanical features that, coupled with the corresponding keying features on the Bulk Station, would prevent a bottle from inserting into a wrong nest. More specifically, a slot molded on the body of the bottle that engages with a tab installed on the corresponding bulk nest (31004). Bottles of different bulk fluids have this slot at different height or different sides of the bottle to prevent a bottle from inserting into a wrong nest.

The bulk bottles are in an elongated shape to maximize the space utilization in the instrument. When the bottle is full and is being moved around, the elongated space causes the fluid to surge back and forth along the length of the bottle and splash out of the opening on the top. A splash guard 31002 is designed. The guard has a cylindrical shape and can be inserted into the bottle from the opening of the bottle. It blocks most surging flow inside the bottle to reduce splash. At the same time, the strategically positioned small windows on the cylinder wall allow fluids to flow in or out of the bottle slowly.

### Form Factors and Capabilities

In certain embodiments, for example, the instrument system may be a bench-top (compact) instrument that can be accommodated on a typical 30-inch deep laboratory bench. In certain embodiments, for example, the bench-top instrument is less than 30 inches deep, less than 29 inches deep, less than 28 inches deep, less than 27 inches deep, less than 26 inches deep, less than 25 inches deep, less than 24 inches deep, less than 23 inches deep, less than 22 inches deep, less than 21 inches deep, less than 20 inches deep. In certain embodiments, for example, the depth of the bench-top instrument is between 30 and 20 inches, between 29 and 21 inches, between 28 and 22 inches, between 27 and 23 inches, or between 26 and 24 inches. In certain embodiments, for example, the depth of the bench-top instrument is between 30 and 28 inches, between 29 and 27 inches, between 28 and 26 inches, between 27 and 25 inches, between 26 and 24 inches, between 25 and 23 inches, between 24 and 22 inches, between 23 and 21 inches, or between 22 and 20 inches. In certain embodiments, for example, the depth of the bench-top instrument is 30, 29, 28, 27, 26, 25, 24, 23, 22, 21, or 20 inches.

In certain embodiments, for example, the instrument system may have a suitable form factor for a bench-top instrument such that the users operate the instrument efficiently and safely. In certain embodiments, for example, the instrument is designed for use on top of a laboratory bench that is 30 inches above the floor. In certain embodiments, for example, the bench-top instrument (not including any stand) is less than 30 inches tall, less than 29 inches tall, less than 28 inches tall, less than 27 inches tall, less than 26 inches tall, less than 25 inches tall, less than 24 inches tall, less than 23 inches tall, less than 22 inches tall, less than 21 inches tall, less than 20 inches tall. In certain embodiments, for example, the height of the bench-top instrument is between 30 and 20 inches, between 29 and 21 inches, between 28 and 22 inches, between 27 and 23 inches, or between 26 and 24 inches. In certain embodiments, for example, the height of the bench-top instrument is between 30 and 28 inches, between 29 and 27 inches, between 28 and 26 inches, between 27 and 25 inches, between 26 and 24 inches, between 25 and 23 inches, between 24 and 22 inches, between 23 and 21 inches, or between 22 and 20 inches. In certain embodiments, for example, the height of the bench-top instrument is 30, 29, 28, 27, 26, 25, 24, 23, 22, 21, or 20 inches.

In certain embodiments, for example, the bench-top instrument's containers for reagents, consumables, and wastes do not exceed 10 lbs. in weight such that the users can swap out the containers efficiently and safely. In certain embodiments, the maximum weight of a reagent container, consumable container, or waste container is less than 10 lbs., less than 9 lbs., less than 8 lbs., less than 7 lbs., less than 6 lbs., or less than 5 lbs. In certain embodiments, for example, the maximum weight of a reagent container, consumable container, or waste container is between 5 and 10 lbs., between 6 and 9 lbs., or between 7 and 8 lbs. In certain embodiments, for example, the maximum weight of a reagent container, consumable container, or waste container is between 10 and 9 lbs., between 9 and 8 lbs., between 8 and 7 lbs., between 7 and 6 lbs., or between 6 and 5 lbs. In certain embodiments, for example, the maximum weight of a reagent container, consumable container, or waste container is 10, 9, 8, 7, 6, or 5 lbs.

In certain embodiments, for example, the bench-top instrument is a fully automated instrument that is capable of assaying samples without human intervention, other than loading the samples into the instrument, and occasional replacement of reagents and other consumables and disposal of wastes. In certain embodiments, for example, the fully automated bench-top instrument can execute a cycle of NULISA and generate results without human intervention in less than 10, 9 8, 7, 6, 5, 4, or 3 hours. In certain embodiments, for example, the fully automated bench-top instrument can execute a cycle of NULISA assay in between 10 and 3, 9 and 4, or 8 and 5 hours. In certain embodiments, for example, the fully automated bench-top instrument can execute a cycle of NULISA assay in between 10 and 8, 9 and 7, 8 and 6, or 7 and 5 hours. In certain embodiments, for example, the fully automated bench-top instrument can execute a cycle of NULISA assay in 10, 9, 8, 7, 6, 5, 4, or 3 hours.

In certain embodiments, for example, the bench-top instrument is capable of a range of multiplex levels when executing the NULISA assay. In certain embodiments, for example, the bench-top instrument is capable of both single-plex and multiplex workflow when executing the NULISA assay. In certain embodiments, for example, the bench-top instrument is capable of executing the NULISA assay in 10-plex or more. In certain embodiments, for example, the bench-top instrument is capable of executing the NULISA assay in up to 200-plex.

In certain embodiments, for example, the bench-top instrument is capable of detecting analytes of extremely low concentrations when executing the NULISA assay. In certain embodiments, for example, the bench-top instrument is capable of detecting protein analytes at a concentration of less than 1 μg/mL using the NULISA assay. In certain embodiments, for example, the bench-top instrument has a limit of detection at an attomolar-level when using the NULISA assay. In certain embodiments, for example, the bench-top instrument has a limit of detection of 1, 2, 5, 10, 20, 50, or 100 attomolar.

### Beads

As described above, certain of the systems provided by the invention are particularly suited for assays using beads for analyte capture. Beads which may be used for analyte capture may be of any suitable size or shape. Non-limiting examples of suitable shapes include spheres (i.e. essentially spherical), cubes (i.e. essentially cubic), ellipsoids (i.e. essentially ellipsoidal), tubes, sheets, irregular shapes, etc. In certain embodiments, the average diameter (if substantially spherical) or average maximum cross-sectional dimension (for other shapes) of a bead may be greater than about 0.1 μm (micrometer), greater than about 1 μm, greater than about 10 μm, greater than about 100 μm, greater than about 1 mm, or the like. In other embodiments, the average diameter of a bead or the maximum dimension of a bead in one dimension may be between about 0.1 μm and about 100 μm, between about 1 μm and about 100 μm, between about 10 μm and about 100 μm, between about 0.1 μm and about 1 mm, between about 1 μm and about 10 mm, between about 0.1 μm and about 10 μm, or the like. The “average diameter” or “average maximum cross-sectional dimension” of a plurality of beads, as used herein, is the arithmetic average of the diameters/maximum cross-sectional dimensions of the beads. Those of ordinary skill in the art will be able to determine the average diameter/maximum cross-sectional dimension of a population of bead, for example, using laser light scattering, microscopy, sieve analysis, or other known techniques. For example, in some cases, a Coulter counter may be used to determine the average diameter of a plurality of beads.

The beads used for analyte capture may be fabricated from one or more suitable materials, for example, plastics or synthetic polymers (e.g., polyethylene, polypropylene, polystyrene, polyamide, polyurethane, phenolic polymers, or nitrocellulose etc.), naturally derived polymers (latex rubber, polysaccharides, polypeptides, etc), composite materials, ceramics, silica or silica-based materials, carbon, metals or metal compounds (e.g., comprising gold, silver, steel, aluminum, copper, etc.), inorganic glasses, silica, and a variety of other suitable materials.

In some embodiments, more than one type of bead for analyte capture may be employed. In some cases, each type of bead may include a surface with differing binding specificity. In addition, each type of bead may have a unique optical (or other detectable) signal, such that each type of bead is distinguishable for each of the other types of beads, for example to facilitate multiplexed assays. In these embodiments, more than one type of analyte molecule may be quantified and/or detected in a single, multiplexed assay method. Of course, as discussed previously, in certain embodiments, the beads are magnetic beads.

### Computer Implemented Control Systems

As described above, certain embodiments of the inventive systems include one or more controllers/computer implemented control systems for operating various components/subsystems of the system, performing data/image analysis, etc. In general, any calculation methods, steps, simulations, algorithms, systems, and system elements described herein may be implemented and/or controlled using one or more computer implemented control system(s), such as the various embodiments of computer implemented systems described below. The methods, steps, control systems, and control system elements described herein are not limited in their implementation to any specific computer system described herein, as many other different machines may be used.

The computer implemented control system(s) can be part of or coupled in operative association with the readout module and/or other automated system components, and, in some embodiments, is configured and/or programmed to control and adjust operational parameters, as well as analyze and calculate values, for example analyte molecule or particle concentrations as described above. In some embodiments, the computer implemented control system(s) can send and receive reference signals to set and/or control operating parameters of system apparatus. In other embodiments, the computer implemented system(s) can be separate from and/or remotely located with respect to the other system components and may be configured to receive data from one or more remote assay systems of the invention via indirect and/or portable means, such as via portable electronic data storage devices, such as magnetic disks, or via communication over a computer network, such as the Internet or a local intranet.

The computer implemented control system(s) may include several known components and circuitry, including a processing unit (i.e., processor), a memory system, input and output devices and interfaces (e.g., an interconnection mechanism), as well as other components, such as transport circuitry (e.g., one or more busses), a video and audio data input/output (I/O) subsystem, special-purpose hardware, as well as other components and circuitry, as described below in more detail. Further, the computer system(s) may be a multi-processor computer system or may include multiple computers connected over a computer network.

The computer implemented control system(s) may include a processor, for example, a commercially available processor such as one of the series ×86, ×86-64, Celeron, Pentium, and Core processors, available from Intel, similar devices from AMD and VIA Technologies, and the Cortex series of processors available from ARM. Many other processors are available, and the computer system is not limited to a particular processor.

A processor typically executes a program called an operating system, of which Windows NT, Windows 11 or 10, UNIX, Linux, and MacOS are examples, which controls the execution of other computer programs and provides scheduling, debugging, input/output control, accounting, compilation, storage assignment, data management and memory management, communication control and related services. The processor and operating system together define a computer platform for which application programs in high-level programming languages are written. The computer implemented control system is not limited to a particular computer platform.

The computer implemented control system(s) may include a memory system, which typically includes a computer readable and writeable non-volatile recording medium, of which a magnetic disk, optical disk, a flash memory and tape are examples. Such a recording medium may be removable, for example, a USB flash drive, or may be permanent, for example, a hard drive.

Such a recording medium stores signals, typically in binary form (i.e., a form interpreted as a sequence of one and zeros). A disk (e.g., magnetic or optical) has a number of tracks, on which such signals may be stored, typically in binary form, i.e., a form interpreted as a sequence of ones and zeros. Such signals may define a software program, e.g., an application program, to be executed by the microprocessor, or information to be processed by the application program.

The memory system of the computer implemented control system(s) also may include an integrated circuit memory element, which typically is a volatile, random access memory such as a dynamic random access memory (DRAM) or static memory (SRAM). Typically, in operation, the processor causes programs and data to be read from the non-volatile recording medium into the integrated circuit memory element, which typically allows for faster access to the program instructions and data by the processor than does the non-volatile recording medium.

The processor generally manipulates the data within the integrated circuit memory element in accordance with the program instructions and then copies the manipulated data to the non-volatile recording medium after processing is completed. A variety of mechanisms are known for managing data movement between the non-volatile recording medium and the integrated circuit memory element, and the computer implemented control system(s) that implements the methods, steps, systems control and system elements control described above is not limited thereto. The computer implemented control system(s) is not limited to a particular memory system.

At least part of such a memory system described above may be used to store one or more data structures (e.g., look-up tables) or equations such as calibration curve equations. For example, at least part of the non-volatile recording medium may store at least part of a database that includes one or more of such data structures. Such a database may be any of a variety of types of databases, for example, a file system including one or more flat-file data structures where data is organized into data units separated by delimiters, a relational database where data is organized into data units stored in tables, an object-oriented database where data is organized into data units stored as objects, another type of database, or any combination thereof.

The computer implemented control system(s) may include a video and audio data I/O subsystem. An audio portion of the subsystem may include an analog-to-digital (A/D) converter, which receives analog audio information and converts it to digital information. The digital information may be compressed using known compression systems for storage on the hard disk to use at another time. A typical video portion of the I/O subsystem may include a video image compressor/decompressor of which many are known in the art. Such compressor/decompressors convert analog video information into compressed digital information, and vice-versa. The compressed digital information may be stored on hard disk for use at a later time.

The computer implemented control system(s) may include one or more output devices. Example output devices include a liquid crystal displays (LCD) and other video output devices, printers, communication devices such as a network interface, storage devices such as hard disk or solid-state drive, and audio output devices such as a speaker.

The computer implemented control system(s) also may include one or more input devices. Example input devices include a keyboard, keypad, track ball, mouse, pen and tablet, and touchscreen, communication devices such as described above, and data input devices such as audio and video capture devices and sensors. The computer implemented control system(s) is not limited to the particular input or output devices described herein.

It should be appreciated that one or more of any type of computer implemented control system may be used to implement various embodiments described herein. Aspects of the invention may be implemented in software, hardware or firmware, or any combination thereof. The computer implemented control system(s) may include specially programmed, special purpose hardware, for example, an application-specific integrated circuit (ASIC). Such special-purpose hardware may be configured to implement one or more of the methods, steps, simulations, algorithms, systems control, and system elements control described above as part of the computer implemented control system(s) described above or as an independent component.

The computer implemented control system(s) and components thereof may be programmable using any of a variety of one or more suitable computer programming languages. Such languages may include procedural programming languages, for example, LabView, C, object-oriented languages, for example, C++ and Java, scripting languages, for example, Python, and other languages, such as a functional language or even assembly language.

The methods, steps, simulations, algorithms, systems control, and system elements control may be implemented using any of a variety of suitable programming languages, including procedural programming languages, object-oriented programming languages, other languages and combinations thereof, which may be executed by such a computer system. Such methods, steps, simulations, algorithms, systems control, and system elements control can be implemented as separate modules of a computer program, or can be implemented individually as separate computer programs. Such modules and programs can be executed on separate computers.

Such methods, steps, simulations, algorithms, systems control, and system elements control, either individually or in combination, may be implemented as a computer program product tangibly embodied as computer-readable signals on a computer-readable medium, for example, a non-volatile recording medium, an integrated circuit memory element, or a combination thereof. For each such method, step, simulation, algorithm, system control, or system element control, such a computer program product may comprise computer-readable signals tangibly embodied on the computer-readable medium that define instructions, for example, as part of one or more programs, that, as a result of being executed by a computer, instruct the computer to perform the method, step, simulation, algorithm, system control, or system element control.

### Alamar Biosciences ARGO HT Instrument: User Guide.

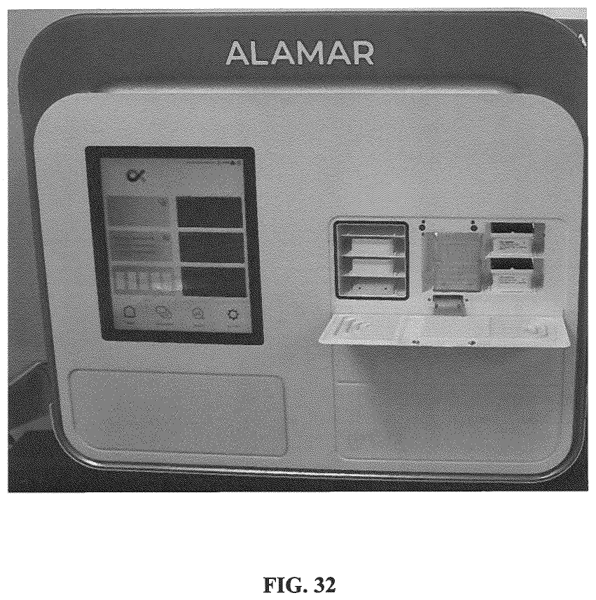

FIG. 32 illustrates an embodiment of the Alamar Biosciences ARGO HT Instrument.

Table of Contents.

| | |
|---|---|
| Preface | iii |
| About This Guide | iii |
| Safety Information | iii |
| Alamar Biosciences ARGO HT Instrument | iv |
| Table of Symbols | vi |
| Alamar Biosciences Headquarters Location | vi |
| Technical Assistance | vii |
| Manufacturer | vii |
| Revision History | vii |
| 1 Instrument Overview | 1-1 |
| Intended Purpose | 1-2 |
| Intended Use | 1-2 |
| Intended User | 1-2 |
| About this Manual | 1-2 |
| Alamar Biosciences ARGO HT Instrument at a Glance | 1-3 |
| Alamar Biosciences ARGO HT Instrument Workflows | 1-4 |
| Single-plex Workflow | 1-4 |
| Multi-plex Workflow | 1-4 |
| Network Connection | 1-4 |
| Software Buttons, Icons and Symbols | 1-5 |
| Technical Assistance | 1-6 |
| 2 Instrument Hardware | 2-1 |
| Alamar Biosciences ARGO HT Components | 2-1 |
| Front of Instrument | 2-1 |
| Rear of Instrument | 2-4 |
| Power the ARGO HT Instrument ON | 2-6 |
| Power the ARGO HT Instrument OFF | 2-6 |
| Logging In and Logging Out | 2-6 |
| Instrument Administration | 2-6 |
| 3 Reagent Kits, Supplies | 3-1 |
| Reagent Kits | 3-1 |
| Assays and Panels | 3-1 |
| Other Supplies | 3-2 |
| 4 Running an Experiment | 4-1 |
| Workflow Overview | 4-1 |
| Create the Experiment in ANA | 4-2 |
| Starting ANA | 4-2 |
| ANA EXPERIMENTS Screen | 4-4 |
| Creating a New Project | 4-6 |
| Creating a New Experiment | 4-7 |
| Running the Experiment on ARGO HT | 4-12 |
| Preparing to Run the Experiment | 4-12 |
| Running the Experiment | 4-14 |
| Unloading the Experiment | 4-16 |
| Reagent Kit Loading Overview | 4-18 |
| Loading Reagent Kits into the ARGO HT | 4-19 |
| Unloading the ARGO HT After Experiment Completion | 4-21 |
| Analyzing Experiment Data | 4-22 |
| Viewing Data on ARGO HT | 4-22 |
| Analyzing Data using ANA | 4-26 |
| Additional ANA Functions | 4-29 |
| INSTRUMENTS Screen | 4-29 |
| SETTINGS Screen | 4-32 |
| Help | 4-36 |
| 5 Analyzing Experiment Data | 5-1 |
| 6 Administrative Tasks | 6-1 |
| User Management | 6-1 |
| User Roles | 6-1 |
| Creating New Users | 6-1 |
| Editing Users | 6-3 |
| Bulk Importing of Users | 6-5 |
| 7 Service and Maintenance | 7-1 |
| Maintenance Tasks | 7-1 |
| Guidelines for Cleaning and Disinfecting | 7-1 |
| Cleaning the Work Area | 7-2 |
| Cleaning the Instrument Surfaces | 7-2 |
| Weekly Instrument Maintenance | 7-3 |
| Power Down the Instrument | 7-3 |
| Instrument Surface Cleaning | 7-3 |
| Power Up the Instrument | 7-4 |
| Annual Instrument Maintenance | 7-4 |
| In Case of Spill | 7-4 |
| Bulk Consumables Replacement | 7-5 |
| Empty the Waste Containers | 7-5 |
| Replace Bulk Consumables | 7-7 |
| 8 Safety | 8-1 |
| Safety Requirements | 8-1 |
| Safety Practices | 8-1 |
| Required Safety Equipment | 8-1 |
| Warnings and Cautions Used in this Manual | 8-2 |
| Electrical Symbols on the Alamar Biosciences ARGO | 8-2 |
| HT Instrument | |
| Electrical Safety | 8-3 |
| Biological Hazard Safety | 8-3 |
| Chemical Safety | 8-3 |
| Environmental Data | 8-4 |
| A Performance Characteristics and Specifications | A-1 |
| Instrument Classification | A-1 |
| General Specifications for the ARGO HT Instrument | A-1 |
| Electrical Specifications for the ARGO HT Instrument | A-1 |
| Operational Environmental Parameters | A-2 |
| Environmental Conditions-Storage and Transport | A-2 |
| Sound Pressure | A-2 |
| Product Energy Consumption Information | A-2 |
| B Quick Reference Guide | B-1 |

### 1. Instrument Overview.

The Alamar Biosciences ARGO™ HT instrument is a fully automated, high-throughput precision proteomics platform for ultra-high sensitivity analysis across a range of multiplex levels to support broad biomarker profiling and translation of validated biomarkers into clinical use. The instrument is powered by our NULISA technology, setting a new standard in proteomic analysis with attomolar sensitivity, allowing for unprecedented biomarker detection and quantitation. By leveraging a proprietary sequential immunocomplex capture and release mechanism and the latest advances in Next Generation Sequencing (NGS), the NULISA technology provides both ultra-high sensitivity and scalable multiplexing.

The instrument includes integrated on-board computing and data analysis, an intuitive user interface with less than 5 minutes of set up time, cartridge-based reagents capable of three consecutive runs or 288 samples, sophisticated liquid handling that will process and prepare your samples for either qPCR readout or pool library ready for next-gen sequencing in less than 6 hours. Alamar Biosciences is Powering Precision Proteomics with unrivaled sensitivity and simplicity.

The ARGO HT instrument is a research use only (RUO), tabletop instrument that integrates chemistry, hardware, and software to amplify and sequence DNA libraries.

### 1-2. Intended Purpose.

Intended Use. The Alamar Biosciences ARGO HT instrument is a research use only (RUO), bench-top instrument that integrates chemistry, hardware, and software to deliver ultra-high sensitivity analysis across a range of multiplex levels to support broad biomarker profiling.

Intended User. The Alamar Biosciences ARGO HT instrument is intended to be operated by experienced laboratory personnel in a laboratory environment.

About this Manual. The Alamar Biosystems ARGO HT User's Guide describes the user operation and maintenance of the Alamar Biosciences ARGO HT instrument. Information is provided about safely using the instrument with the ARGO HT software and performing maintenance.

Read the entire manual and become familiar with the safety information before you start to operate the instrument. Using the instrument without reading the manual can result in serious injury, damage to the instrument, invalid results, or loss of data.

This manual describes how to use, maintain, and administer the instrument. The audience for this manual is everyone who uses, administers or maintains the instrument.

To learn how to use other parts of the instrument and related products, locate the relevant publication in the following table.

TABLE A

| For . . . | See . . . |
|---|---|
| Abbreviated instructions for | Alamar Biosciences ARGO HT |
| running an experiment | Reference Guide |
| Facilities requirements and preparation | Alamar Biosciences ARGO |
| for installing the Alamar Biosciences | HT Site Preparation Guide |
| ARGO HT instrument | |
| Assay-specific instructions for | The instructions for use (IFU) |
| running an experiment | for the assay |

### 1-3. Alamar Biosciences ARGO HT Instrument at a Glance.

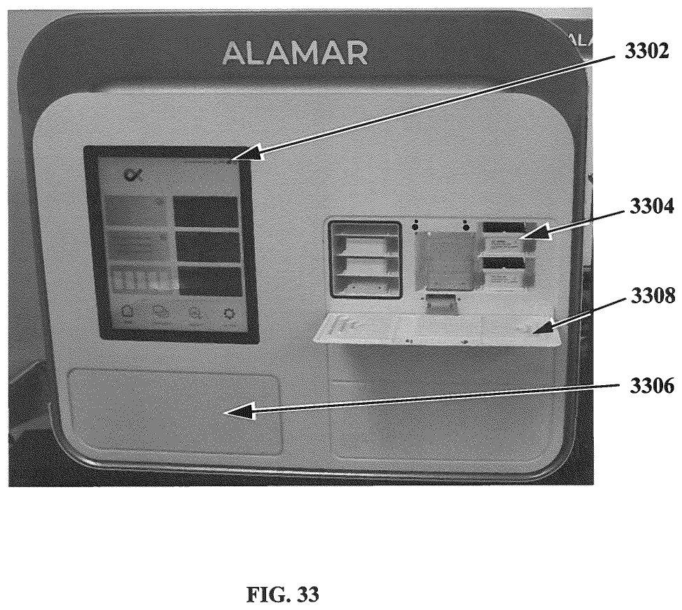

FIG. 33 illustrates an embodiment of the Alamar Biosciences ARGO HT Instrument at a glance which comprises a bench-top housing 3000 touchscreen display 3302, a set of bays 3304, and a compartment for bulk reagents 3306.

TABLE 1-1
Alamar Biosciences ARGO HT Instrument at a Glance.

| Component | Definition |
|---|---|
| Touchscreen | The touchscreen is used to make selections in the |
| Display | software. Connected to the dedicated on- board |
| | computer, the Alamar Biosciences ARGO HT instrument is |
| | fully capable of all on-board computing and data analysis. |
| Bays | The bays are for loading experiments. The ARGO HT |
| | instrument has three bays, each capable of holding one |
| | experiment to be run. They use cartridge-based reagents |
| | capable of three consecutive runs. |
| Bulk | Bulk reagents allow the ARGO HT instrument to perform |
| Reagents | up to 15 runs before requiring replacement. |

The Alamar Biosciences ARGO HT features include:

All-in-one instrument with integrated qPCR instrument takes you from sample preparation to data or to a pooled NGS library.

Fully automated workflow with <30 minutes hands-on time.

High throughput capacity processes up to 288 samples per run.

Rapid results in <6 hours for single-plex assays and <15 hours for multiplex NGS assays.

Integrated software and data analysis transforms data into biological insights and publication-ready results.

### 1-4. Alamar Biosciences ARGO HT Instrument Workflows.

The Alamar Biosciences ARGO HT instrument has a full-automated workflow for both single-plex and multiplex processing. Single-plex results are available in <6 hours and multiplex results are available in less than 15 hours with <30 minutes hands-on time. For more details on ARGO HT instrument workflows, see Chapter 4, Running an Experiment.

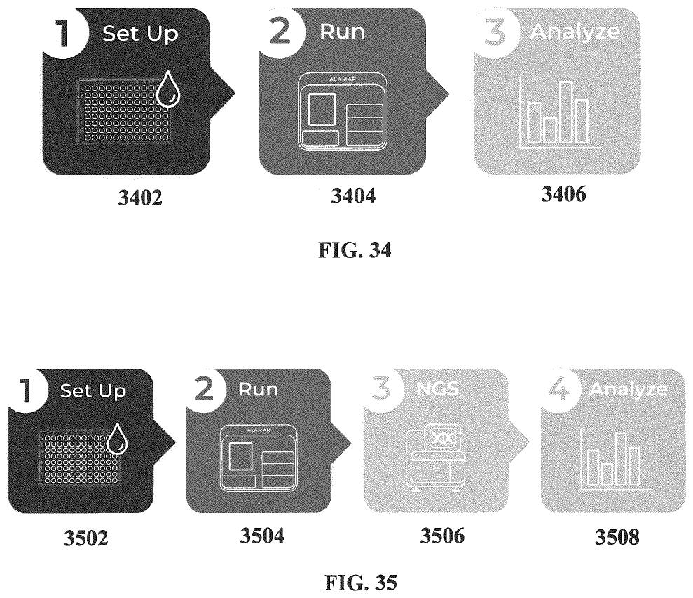

Single-plex Workflow. FIG. 34 illustrates a single-plex workflow in the ARGO HT Instrument which comprises a setup step 3402 that is simple, robust and reproducible with less than 30 minutes hands-on time, a run step 3404 that has high-throughput sample-to-results in less than 6 hours run time, and an analysis step 3406 that uses automated cloud-based analytics.

Multiplex Workflow. FIG. 35 illustrates an embodiment of a multi-plex workflow in the ARGO HT Instrument which comprises a setup step 3502 that is simple, robust and reproducible with less than 30 minutes hands-on time, a run step 3504 that has high-throughput sample-to-results in less than 6 hours run time, a NGS step 3506 which is an 8 hour sequencing run on Illumina systems, and an analysis step 3508 that uses automated cloud-based analytics.

Network Connection. The Alamar Biosciences ARGO HT instrument requires an Internet connection to communicate with the Alamar NULISA™ Analysis (ANA) software. The ANA software is used for creating the experiments, downloading the experiments to the ARGO HT instrument and for analyzing the results after the experiment has run on the ARGO HT instrument.

### 1-5. Software Buttons, Icons and Symbols.

Table 1-2 is a short description of the most common buttons icons and symbols encountered in the Alamar Biosciences ARGO HT software.

TABLE 1-2
ARGO HT Software Buttons, Icons and Symbols.

| Symbol | Definition |
|---|---|
| FIG. 3602 | HOME—Goes to the Home screen. |
| FIG. 3604 | EXPERIMENTS—Goes to the Experiments screen and |
| | displays all of the experiments in the queue to be run. |
| FIG. 3606 | RESULTS—Goes to the Results screen where results |
| | can be viewed for all completed experiments. |
| FIG. 3608 | LOAD—Loads experiments from the Experiments screen. |
| FIG. 3610 | RUN—Runs the experiment displayed in the bay. |
| FIG. 3612 | Indicates the levels of bulk liquid levels inside the |
| | ARGO HT instrument. Indicators are color-coded for each |
| | liquid with a label beneath the icon. |

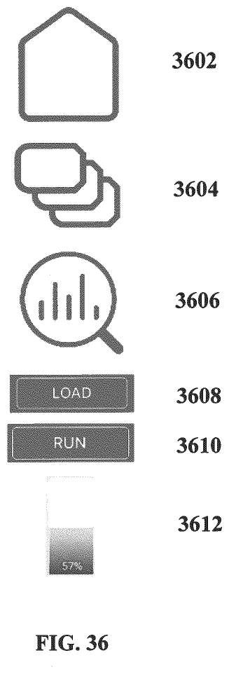

FIG. 36 illustrates ARGO HT Software Buttons, Icons and Symbols, which comprise a Home icon 3602, an Experiments icon 3604, a Results icon 3606, a Load button 3608, a Run button 3610, and bulk liquid level indicator 3612.

Table 1-3 is a short description of the most common icons and symbols encountered in the Alamar NULISA Analysis (ANA) software.

TABLE 1-3
ANA Software Buttons, Icons and Symbols.

| Symbol | Definition |
|---|---|
| FIG. 3702 | EXPERIMENTS—Goes to the Experiments screen where |
| | you can design experiments and send them to the |
| | ARGO HT instrument to be run. |
| FIG. 3704 | INSTRUMENTS—Go to the Instruments screen which shows |
| | instrument status, queue and allows the user to enable |
| | or disable connected instruments. |
| FIG. 3706 | ANALYSIS—Goes to the Analysis screen where results |
| | can be analyzed and/or exported for further analysis. |

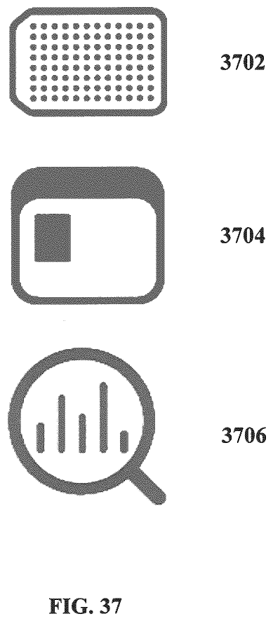

FIG. 37 illustrates Alamar NULISA Analysis (ANA) Software Buttons, Icons and Symbols which comprise an Experiments icon 3702, an Instruments icon 3704, and an Analysis icon 3706.

### 1-6. Technical Assistance.

Technical assistance is available from Alamar Biosciences if there are any questions about the Alamar Biosciences ARGO HT instrument. See the Technical Assistance section in the Preface for contact information.

### 2. Instrument Hardware.

### 2-1. Alamar Biosciences ARGO HT Components.

The Alamar Biosciences ARGO™ HT instrument is a fully automated, high-throughput precision proteomics platform for ultra-high sensitivity analysis across a range of multiplex levels to support broad biomarker profiling and translation of validated biomarkers into clinical use.

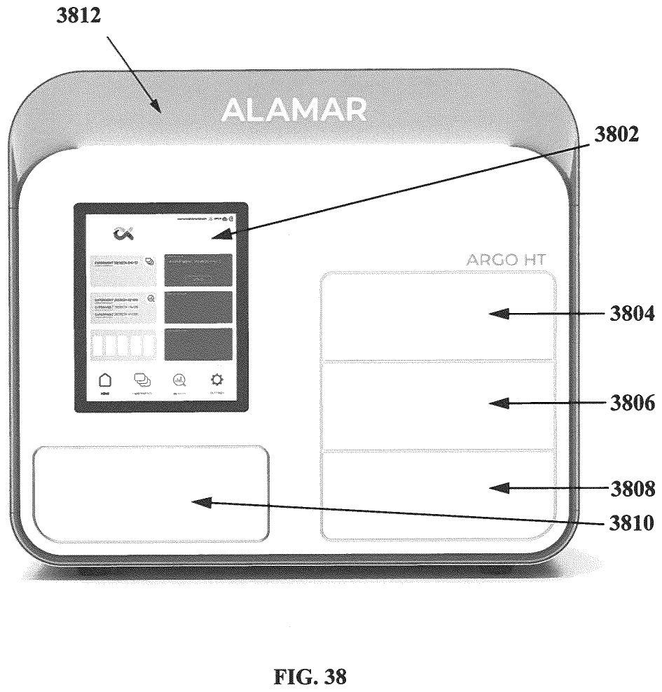

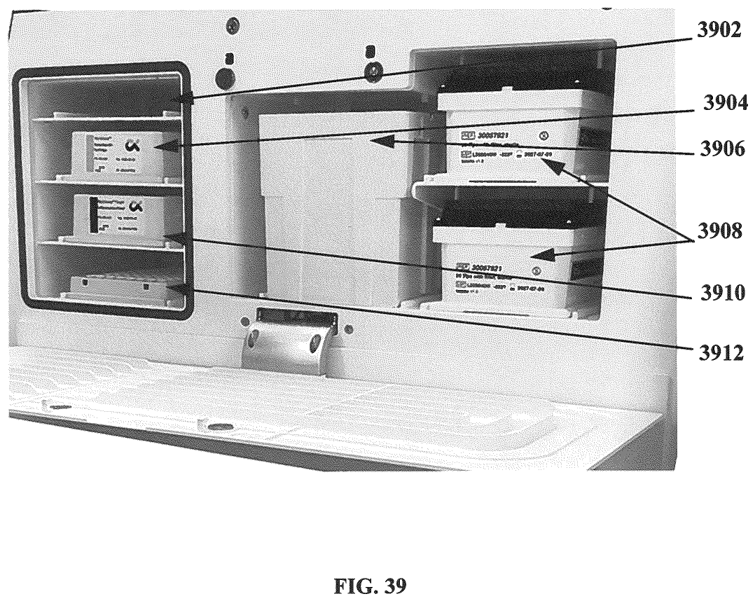

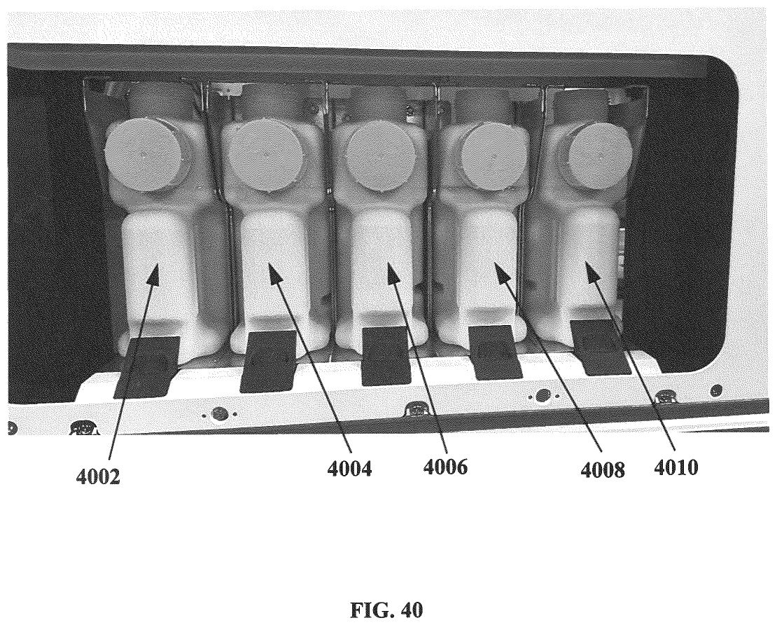

Front of Instrument. Components visible from the front of the instrument are shown in FIG. 38, FIG. 39 and FIG. 40.

FIG. 38 illustrates a front view of the Alamar Biosciences ARGO HT Instrument which comprises a touchscreen display 3802, a first bay 3804, a second bay 3806, a third bay 3808, a compartment for bulk reagents 3810, and a bench-top housing 3812.

TABLE 2-1
Alamar Biosciences ARGO HT Instrument Front View.

| Component | Definition |
|---|---|
| Touchscreen | The touchscreen is used to make selections in the |
| Display | software. Connected to the dedicated on- board |
| | computer, the Alamar Biosciences ARGO HT instrument is |
| | fully capable of all on-board computing and data analysis. |
| Bays | The bays are for loading experiments. The ARGO HT |
| | instrument has three bays, each capable of holding one |
| | experiment to be run. They use cartridge-based reagents |
| | capable of three consecutive runs. |
| Bulk | Bulk reagents allow the ARGO HT instrument to perform |
| Reagents | up to 15 runs before requiring replacement. |

FIG. 39 illustrates bay components of the ARGO HT Instrument which comprise a ligation plate for NULISAseq 3902, a NULISAseq detection kit 3904, a consumables cartridge 3906, tip boxes 3908, a NULISAseq (or PCR) target kit 3910, and a sample plate 3912.

The bay contains all of the components required to run an experiment. These components are sold in kits for various types of experiments. Contact your Alamar Biosciences representative to discuss available kits for your instrument.

There are three bays each capable of holding components for one experiment.

Experiments must be run serially but bays can be loaded and prepared while the experiment from one bay is running.

The bulk liquids area contains the bulk liquids and liquid waste bottles that are common for all experiments. It allows the ARGO HT to run up to 15 experiments before requiring service. It contains two waste liquid bottles and three bulk liquid bottles.

FIG. 40 illustrates bulk liquid components which comprise a decontamination buffer container 4002, a DI water container 4004, a wash buffer container 4006, and two waste containers 4008, 4010.

### 2-4. Rear of Instrument.

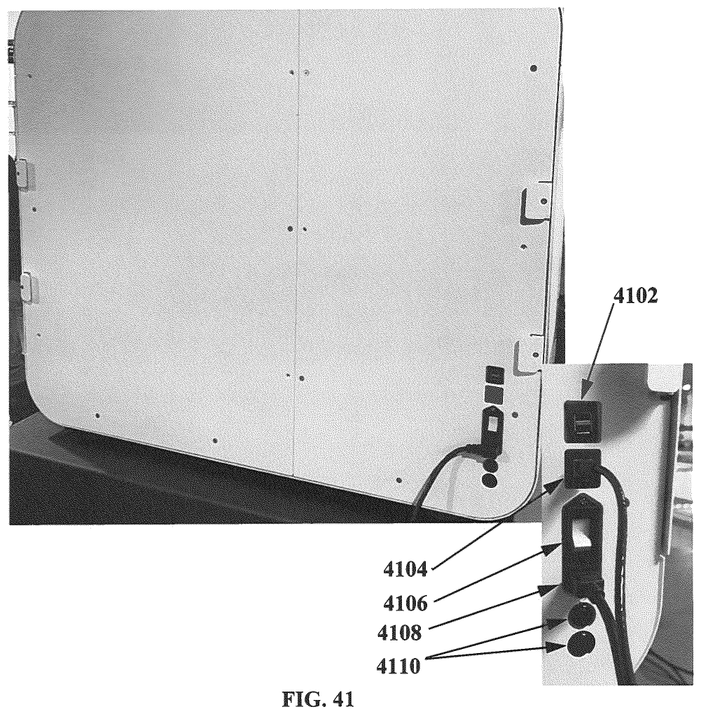

The rear of the instrument contains all of the connections. Rear components are located on the right side of the rear panel of the instrument. Rear components are shown in FIG. 41 and described in Table 2-2.

FIG. 41 illustrates a rear view of the ARGO HT Instrument which comprises USB communication port 4102, an ethernet communication port 4104, an ON/OFF switch 4106, a power connection 4108, fuses 4110.

TABLE 2-2
Alamar Biosciences ARGO HT Instrument Rear View

| Component | Definition |
|---|---|
| ON/OFF | Power switch, where O = off and \| = on |
| Switch | |
| Power | 100-240VAC connection that provides power to the |
| Connection | instrument |
| Ethernet | RJ-45 port that provides Ethernet communication |
| Port | between the instrument and a local network (Internet) |
| USB Ports | Allows connection of a USB device to the instrument |
| Fuses | Two fuses provide electrical protection to the instrument |

### 2-6. Power the ARGO HT Instrument ON.

This section describes how to power up the instrument:

Turn on the instrument. The power switch is located on the back of the instrument. Press the switch to the ON (|) position (see FIG. 41).

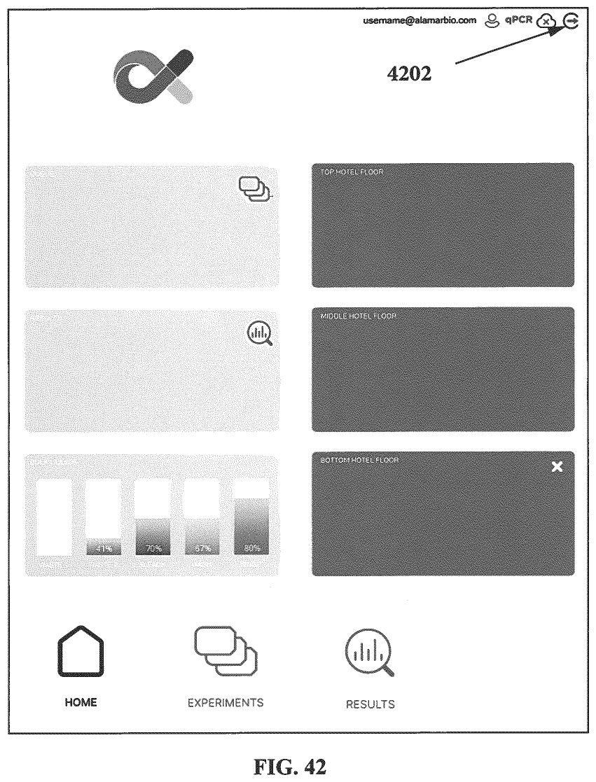

Wait until the instrument starts up. On the front of the instrument, check that the touchscreen monitor displays the Home screen. See FIG. 42. FIG. 42 illustrates the ARGO HT home screen after start-up which comprises a program exit icon 4202.

### 2-6. Power the ARGO HT Instrument OFF.

This section describes how to power off the instrument:

On the Home screen, select the Program Exit icon in the upper right corner of the touchscreen display. See FIG. 2-5. The software will close.

Turn off the instrument. The power switch is located on the back of the instrument. Press the switch to the OFF (O) position (see FIG. 41).

Important. Do NOT press the power button while an experiment is running.

Interrupting power while an experiment is running can result in run failure of the experiment and loss of samples.

### 2-6. Logging In and Logging Out.

There are no logins or logouts for the ARGO HT instrument.

### 2-6. Instrument Administration.

There are no administrative tasks for the ARGO HT instrument.

### 3. Reagent Kits, Supplies.

### 3-1. Reagent Kits.

The Alamar Biosciences ARGO HT instrument reagent kits can be ordered from Alamar Biosciences. The following reagent kits are available:

TABLE 3-1
ARGO HT Reagent Kits.

| Product Name | Part No. |
|---|---|
| NULISA Reagent Kit | 800102 |

Kit Storage Requirements. The reagent kit storage requirements are:

### 3-1. Assays and Panels.

The Alamar Biosciences ARGO HT instrument assays and panels can be ordered from Alamar Biosciences. The following assays and panels are available:

TABLE 3-2
ARGO HT Assays and Panels.

| Product Name | Product Family | Part No. |
|---|---|---|
| NULISAseq Inflammation Panel | Assay Multiplex | 800103 |
| NULISA Neurology Panel | Assay Multiplex | 800104 |
| NULISApcr NFL Assay | Assay Singleplex | 800105 |
| NULISApcr IL1B Assay | Assay Singleplex | 800106 |
| NULISApcr IL4 Assay | Assay Singleplex | 800107 |
| NULISApcr LIF Assay | Assay Singleplex | 800108 |
| NULISApcr IFNg Assay | Assay Singleplex | 800109 |
| NULISApcr IL6 Assay | Assay Singleplex | 800110 |
| NULISApcr IL8Assay | Assay Singleplex | 800111 |
| NULISApcr GFAP Assay | Assay Singleplex | 800112 |
| NULISApcr tTAU Assay | Assay Singleplex | 800113 |
| NULISApcr pTAU-181 Assay | Assay Singleplex | 800114 |
| NULISApcr pTAU-217 Assay | Assay Singleplex | 800115 |
| NULISApcr pTAU-218 Assay | Assay Singleplex | 800116 |
| NULISApcr IL2 Assay | Assay Singleplex | 800117 |
| NULISApcr TNFa Assay | Assay Singleplex | 800118 |
| NULISAper UCHL1 Assay | Assay Singleplex | 800119 |
| NULISApcr TDP43 Assay | Assay Singleplex | 800120 |
| NULISApcr AB40 Assay | Assay Singleplex | 800121 |
| NULISApcr AB42 Assay | Assay Singleplex | 800122 |
| NULISApcr NFL Assay | Assay Singleplex | 800123 |

Assay and Panel Storage Requirements. The assay and panel storage requirements are:

### 3-2. Other Supplies.

In addition to the ARGO HT, consumable storage must be provided close to the system. ARGO HT Reagents and consumables are ready to use and do not require additional preparation.

Reagent Storage. Some reagents must be stored at 2° C.-8° C. Some reagents may be stored at room temperature.

Consumable storage equipment and materials are:

Refrigerator storage, 2° C.-8° C. storage; Room temperature storage; DI water; Ethanol (70%); Disposable lint-free wipes; Centrifuge (specifications?); Household chlorine bleach; Disposable gloves; and Personal Protective Equipment (PPE).

### 4. Running an Experiment.

### 4-1. Workflow Overview.

The Alamar Biosciences ARGO HT instrument has a fully automated workflow for both single-plex and multiplex processing. Single-plex results are available in <6 hours and multiplex results are available in less than 15 hours with <30 minutes hands-on time. This chapter will focus on how to run an experiment. The single-plex workflow will be used for examples in this chapter. The primary difference between a single-plex workflow and a multiplex workflow is that there will be an additional step to perform a sequencing run on an Illumina system before analyzing the data.

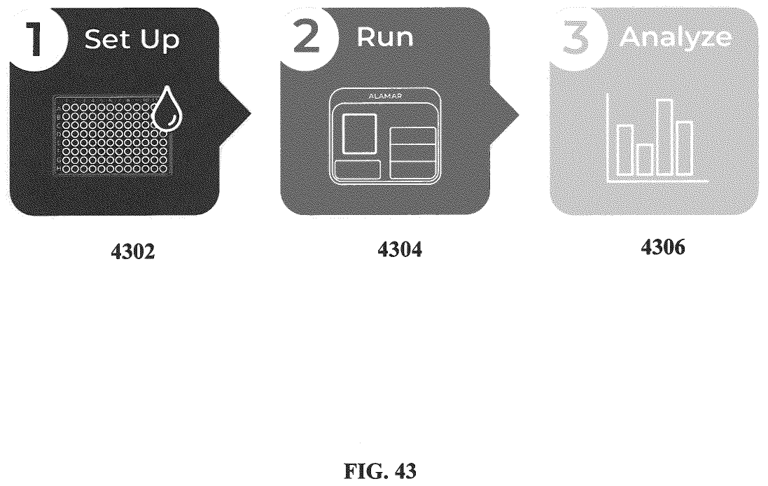

The single-plex workflow is described in FIG. 43. FIG. 43 illustrates a single-plex workflow in the ARGO HT Instrument ARGO HT Instrument which comprises a setup step 4302 that is simple, robust and reproducible with less than 30 minutes hands-on time, a run step 4304 that has high-throughput sample-to-results in less than 6 hours run time, and an analysis step 4306 that uses automated cloud-based analytics.

### 4-2. Create the Experiment in ANA.

The first step is to create the experiment using Alamar NULISA Application (ANA) software. ANA is a web application that will allow you to create an experiment and then queue the experiment to Alamar Biosciences ARGO HT instruments registered on ANA. The experiment will be run on the ARGO HT instrument and the results will be uploaded back to the ANA software.

For this example, we will create an experiment using ANA.

Note: The screens shown in the chapter have been captured with a Supervisor level login. Throughout this chapter, if there is any difference between a Supervisor login and an Operator login, it will be noted in that section.

### 4-2. Starting ANA.

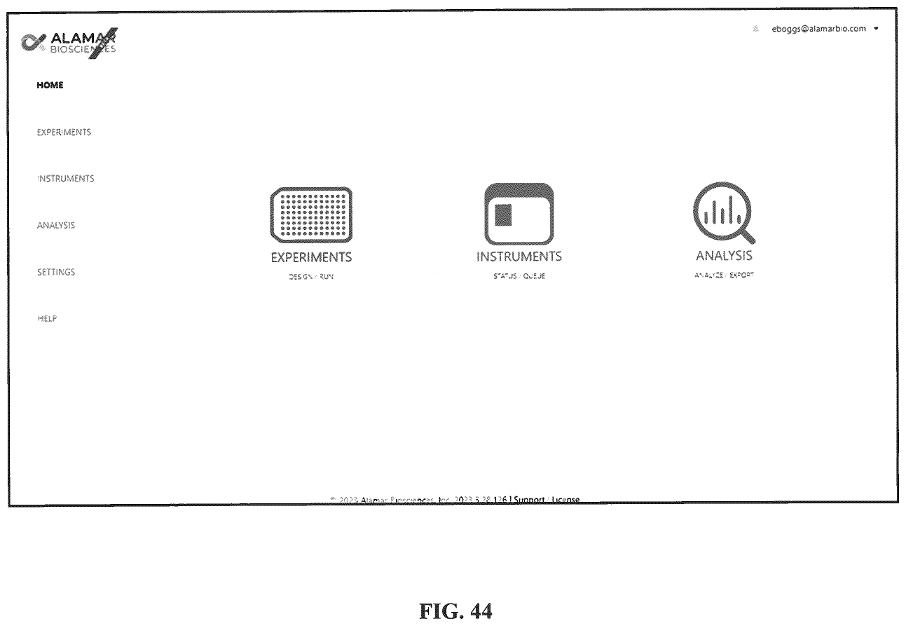

A shortcut to the ANA website should have been created onto one or more customer computers when the ARGO HT instrument was installed. Also, a login will also have been created for at least one supervisor role user. Locate the Alamar Biosciences software shortcut on the desktop and double-click the icon. The ANA software will load and display the ANA Log in screen. On the ANA Login screen, enter your email address and password and click the Log in button. If you have forgotten your password, click on Forgot your password? and follow the on-screen prompts to reset your password. After entering your password, the ANA HOME screen is displayed. See FIG. 44.

FIG. 44 illustrates a Home Screen of the Alamar NULISA Analysis (ANA) software.

The primary screens that are used in ANA are described in Table 4-1.

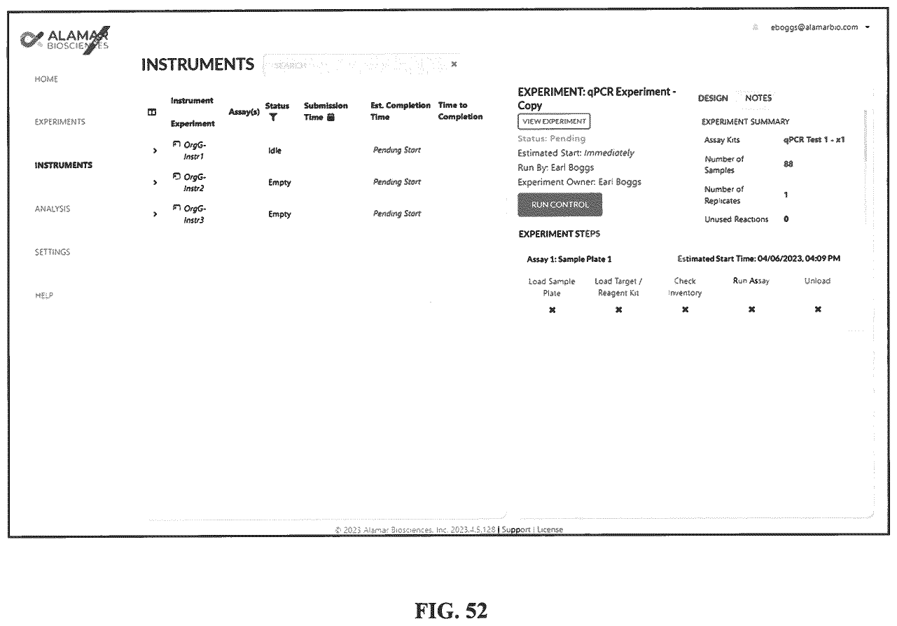

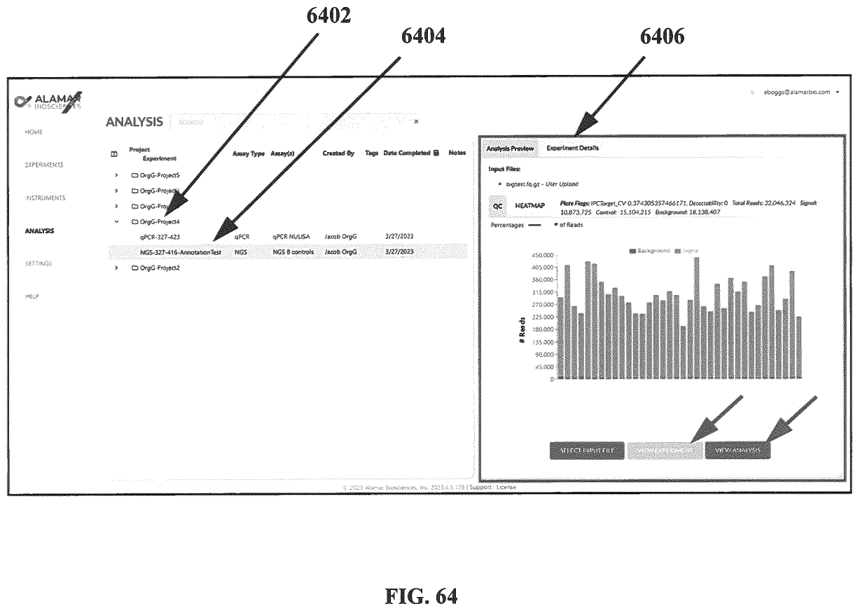

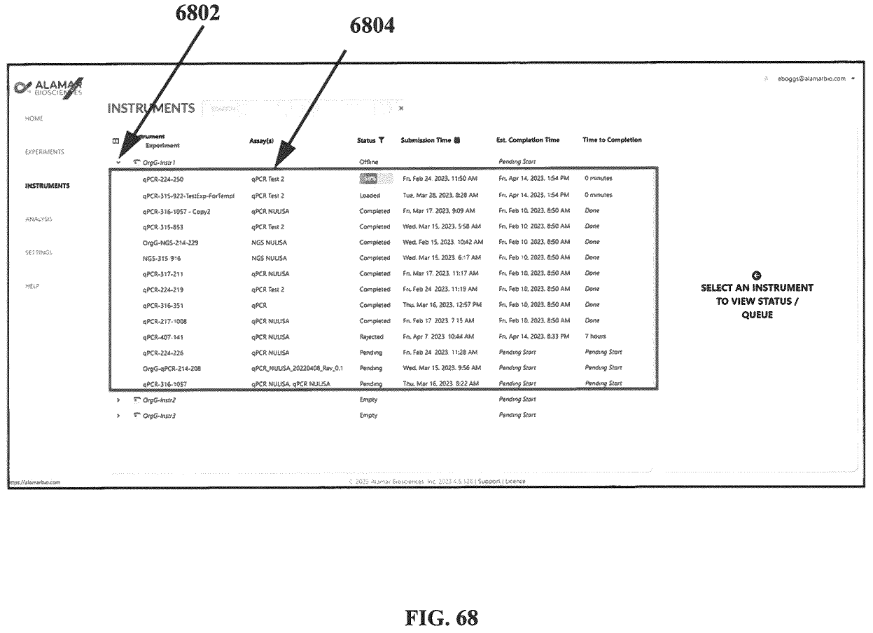

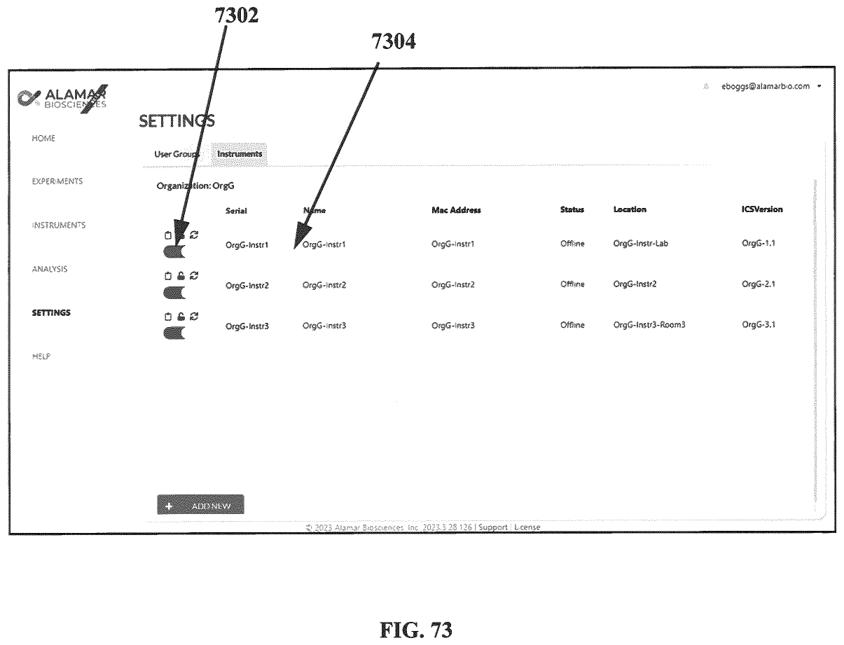

TABLE 4-1
Alamar Biosciences ANA Primary Screens

| Screen Name | Function |
|---|---|
| HOME | The HOME screen is the landing page when the ANA |
| | software is launched. It is a launch point to open |
| | other screens within ANA. See FIG. 44. |
| EXPERIMENTS | The EXPERIMENTS screen is used to create new |
| | experiments or to select an existing experiment from |
| | the library. It also displays all existing projects |
| | and experiments currently being developed. See FIG. |
| | 45 through FIG. 52. |
| INSTRUMENTS | The INSTRUMENTS screen shows the instruments |
| | currently connected to ANA and the status of |
| | each instrument. See FIG. 68. |
| ANALYSIS | The ANALYSIS screen displays the results of all |
| | experiments that have been completed and the |
| | analysis of the results. Results can also be exported |
| | to various file types for further analysis. |
| | See FIG. 64 through FIG. 67. |
| SETTINGS | Supervisor: The SETTINGS screen is used to create |
| | user groups within the ANA application and to add |
| | individuals to those groups. In addition, it allows |
| | the user to see the connected instruments and to |
| | activate or deactivate those instruments. See FIG. |
| | 71 and FIG. 73. |
| | Operator: The SETTINGS screen is used to view user |
| | groups within the ANA application but an operator |
| | cannot make changes to the groups. An operator can |
| | see the connected instruments and their status but |
| | cannot activate or deactivate the instruments. |
| HELP | The HELP screen displays help information for ANA. |
| | See FIG. 44. |

### 4-4. ANA Experiments Screen.

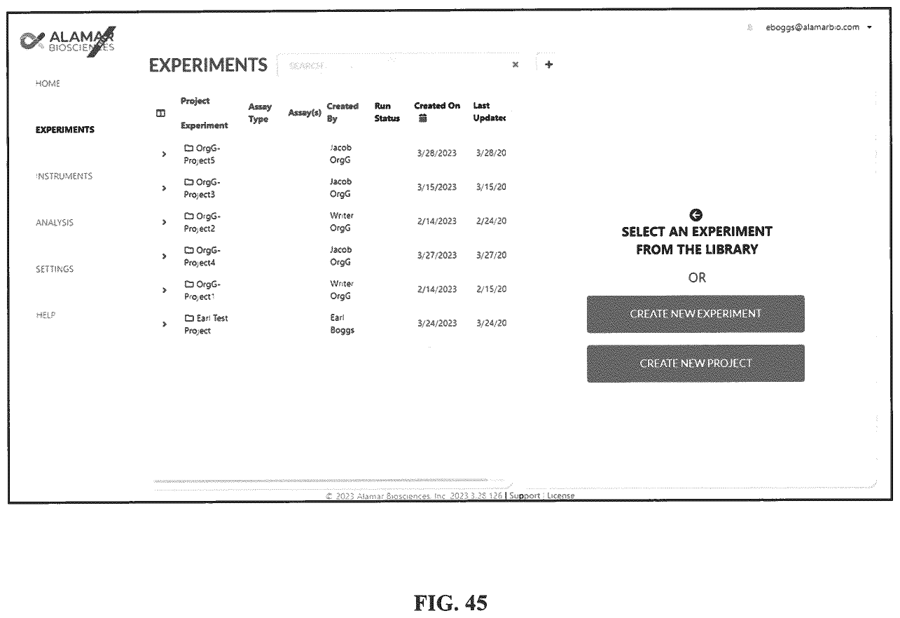

The EXPERIMENTS screen is used to create new experiments or to select an existing experiment from the library. It also displays all existing projects and experiments that the user is developing. Click the EXPERIMENTS icon from the center of the HOME screen or on the left-hand column to navigate to the EXPERIMENTS screen. See FIG. 45.

FIG. 45 illustrates an Experiments Screen of the Alamar NULISA Analysis (ANA) software.

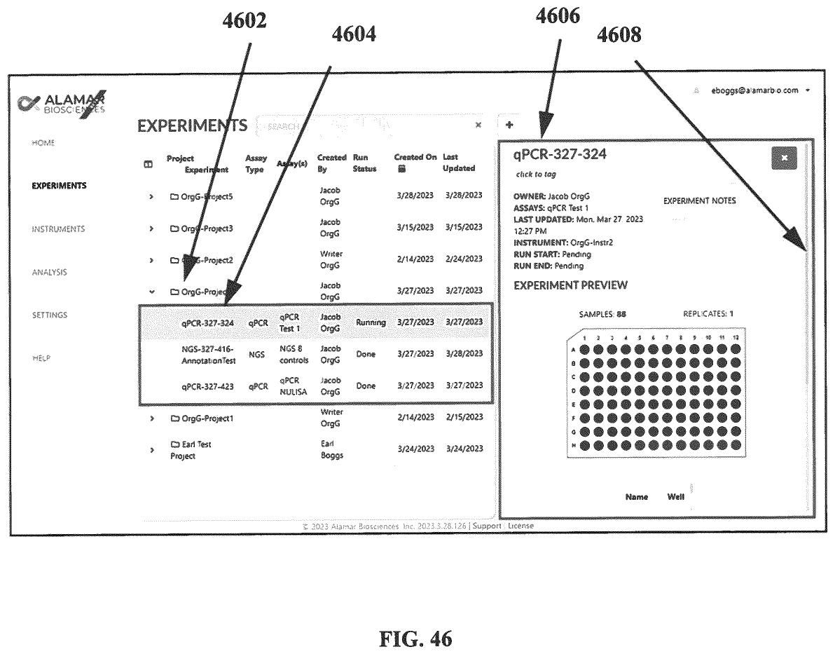

The existing projects in the library are displayed on the left-hand side of the screen. To view an existing project, click on the project and the experiments located within the project will be displayed. See FIG. 46.

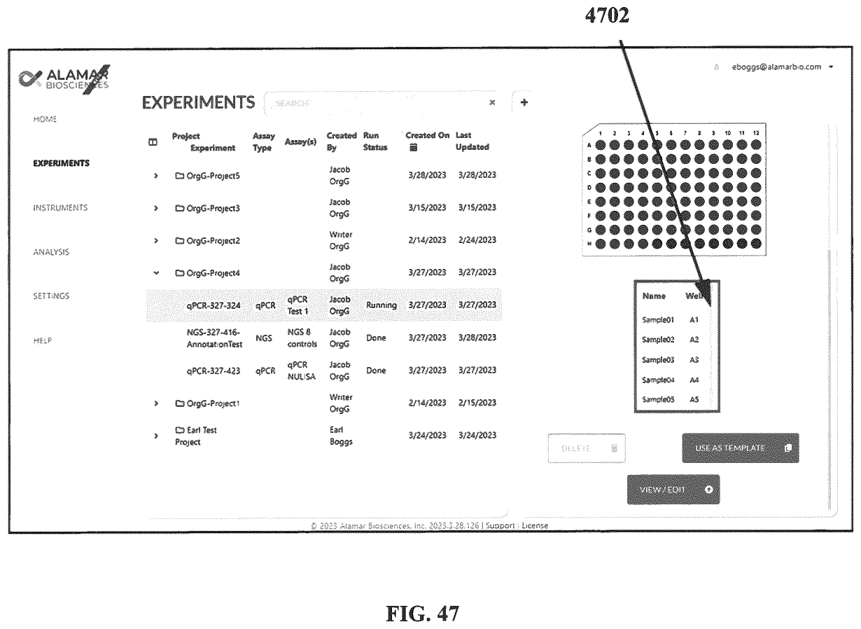

FIG. 46 illustrates an Experiment Screen of the Alamar NULISA Analysis (ANA) software with a selected experiment which comprises lists of projects 4602, lists of experiments for each project 4604, experiment details 4606, and a scroll bar to view more details 4608. Scroll down to see the experiment details. See FIG. 47.

FIG. 47 illustrates an Experiments Screen of the Alamar NULISA (ANA) Analysis software showing well assignments comprising a scroll bar to see well assignments in sample tray 4702.

Underneath the well assignments, you can select the following buttons described in Table 4-2.

TABLE 4-2
EXPERIMENTS Screen Button Functions.

| Screen Name | Function |
|---|---|
| DELETE | Select the DELETE button to delete the experiment. |
| | A dialog box will be displayed to confirm that you |
| | want to delete the experiment. Select OK to delete |
| | the experiment or Cancel to not delete the experiment. |
| | A confirmation dialog box will indicate that the |
| | experiment has been successfully deleted. |
| USE AS | Select USE AS TEMPLATE to create a new experiment |
| TEMPLATE | that can be modified using the existing experiment |
| | as a starting point. |
| VIEW/EDIT | Select VIEW/EDIT to view and/or edit the existing |
| | experiment. |

### 4-6. Creating a New Project.

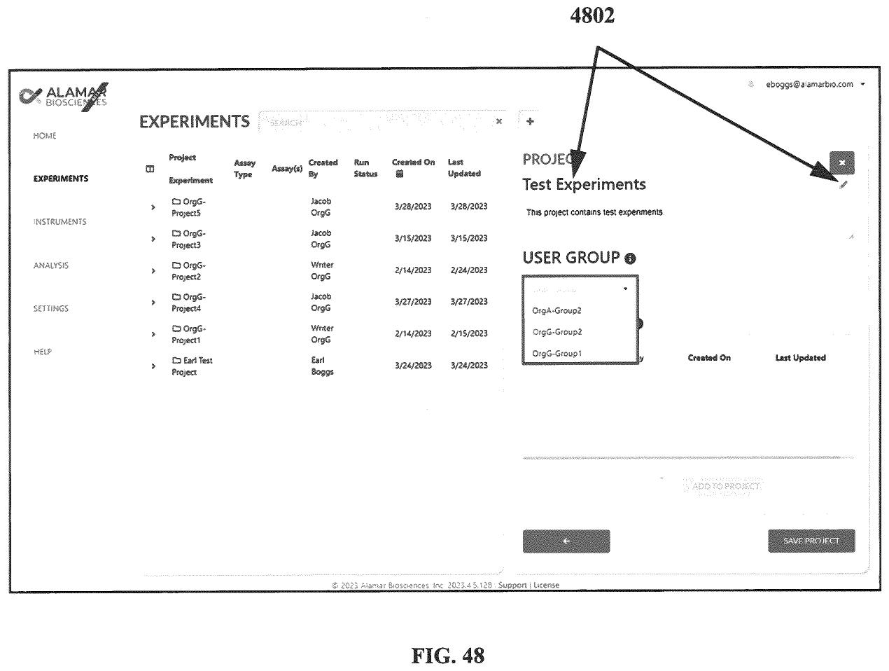

As shown in FIG. 46, projects are the top level and contain experiments. Project assignment is optional and any experiments not belonging to a project will appear under the Unassigned project header. To create a new project:
1. Select CREATE NEW PROJECT (see FIG. 45). A project workspace will be displayed to create the new project. See FIG. 48.

FIG. 48 illustrates an Experiments Screen of the Alamar NULISA Analysis (ANA) software showing a Create a New Project feature comprising a section to enter a project name 4802.
2. In the project workspace, select the pencil icon to enter a name for the project.
3. Underneath the project name, enter a description of the project.
4. From the drop-down under USER GROUP, select the group to have access to these experiments. Users in the selected group will have permission to access experiments in this project as granted by their group role. If no group is selected, experiment access will be limited to the experiment owner and organizational administrators.
5. Select SAVE PROJECT to create the new project using the project name. The new project appears in the project list.

### 4-7. Creating a New Experiment.

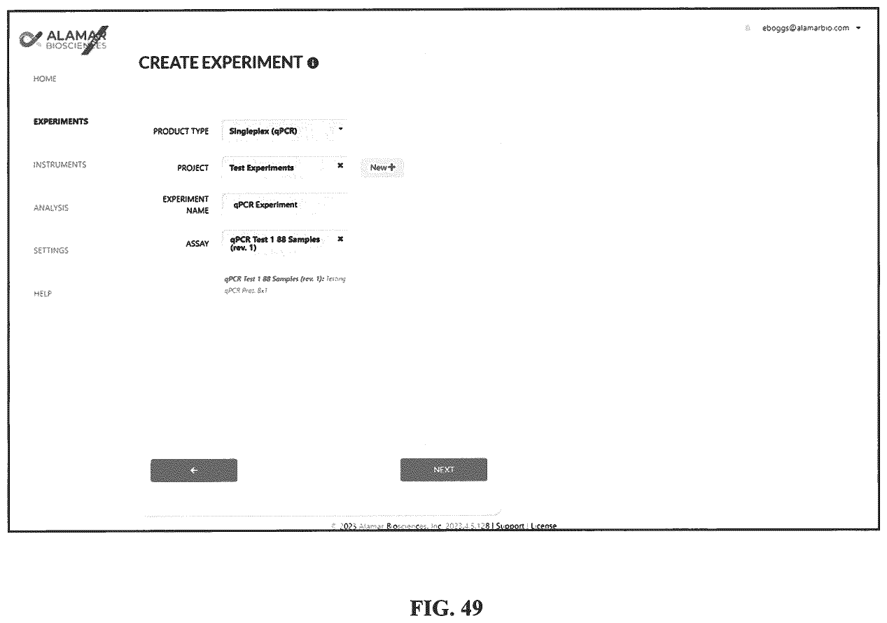

Create a new experiment by picking a product type, an experiment name and an assay:
1. Select CREATE NEW EXPERIMENT (see FIG. 45). A create experiment workspace will be displayed to create the new experiment. See FIG. 49.

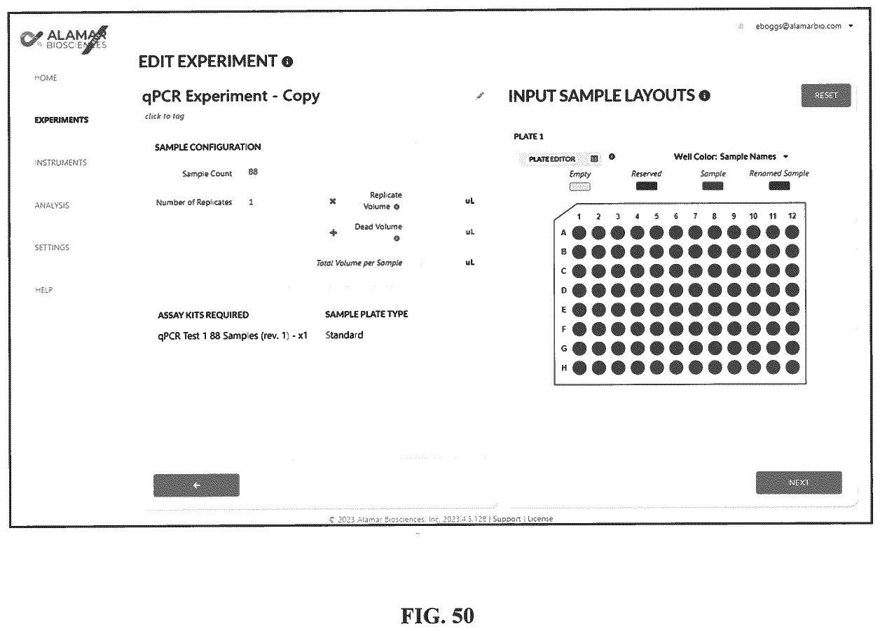

FIG. 49 illustrates an Experiments Screen of the Alamar NULISA Analysis (ANA) software showing a Create New Experiment feature.
2. On the CREATE EXPERIMENT screen, select the PRODUCT TYPE from the drop-down.
3. Optionally, select a project to assign this experiment to from the PROJECT drop-down list. Alternatively, select the New+button if you want to create a new project for this experiment.
4. Enter a name for this experiment in the text box.
5. Select an assay from the drop-down.
6. Select NEXT to display the EDIT EXPERIMENT screen. See FIG. 50.

FIG. 50 illustrates an Experiments Screen of the Alamar NULISA Analysis (ANA) software showing an Edit Experiment feature.
7. On the EDIT EXPERIMENT screen, make any necessary changes to the selected experiment. Potential changes may include:

Editing the experiment name-select the pencil icon and edit the name.

Editing the Sample Configuration data.

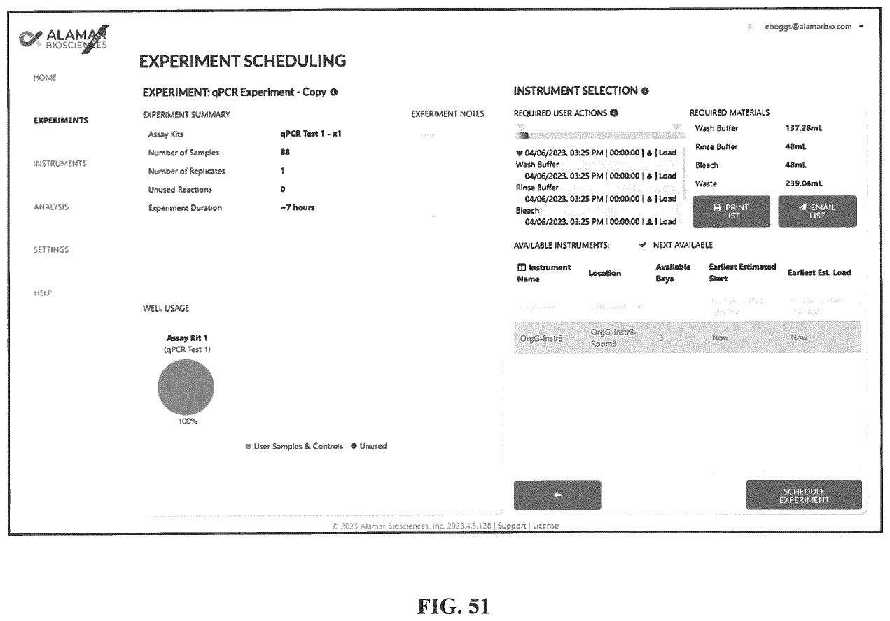

Editing the input sample layouts using the Plate Editor. The Plate Editor is used to both identify the location of the sample wells and provide names of samples within the sample plate. Annotations for each sample/well can also be assigned using the Plate Editor) e.g. sex, health status, etc.).
8. Select NEXT to display the EXPERIMENT SCHEDULING screen which includes a summary of the experiment design, note and well usage. See FIG. 51.

FIG. 51 illustrates an Experiments Screen of the Alamar NULISA Analysis (ANA) software showing an Experiment Scheduling feature.
9. On the EXPERIMENT SCHEDULING screen, review the experiment and complete instrument selection. Potential items to note on this screen are: Adding additional experiment notes; Printing the required materials list; Emailing the required materials list; Selecting the instrument to run the experiment on if there are multiple instruments available.

Select SCHEDULE EXPERIMENT to queue the experiment onto the selected instrument. When queued, the INSTRUMENTS screen will be displayed which includes a summary of the experiment queued to the equipment. See FIG. 52.

FIG. 52 illustrates an Experiments Screen of the Alamar NULISA Analysis (ANA) software showing a scheduled experiment.

The experiment is now sent to the selected instrument to be run. On this screen, you can: View the experiment summary; View/edit the experiment notes; View the experiment steps and run progress; Select VIEW EXPERIMENT which displays the experiment in the EXPERIMENTS screen; Select RUN CONTROL which allows you to pause the experiment in the queue, re-queue the experiment to another instrument or cancel the experiment.

At this point, the experiment is in the queue for the selected instrument. If the instrument is not running experiments at this time, it will be automatically ready for loading into a bay. Proceed to the ARGO HT to prepare and run the experiment.

### 4-12. Running the Experiment on ARGO HT.

After creating the experiment in ANA, it is automatically sent to the selected Alamar Biosciences ARGO HT instrument. Go to the instrument's location to run the experiment.

It is assumed that: The ARGO HT is powered on and is on-line to accept experiments. The experiment was created in ANA and was downloaded to the ARGO HT instrument.

### 4-12. Preparing to Run the Experiment.

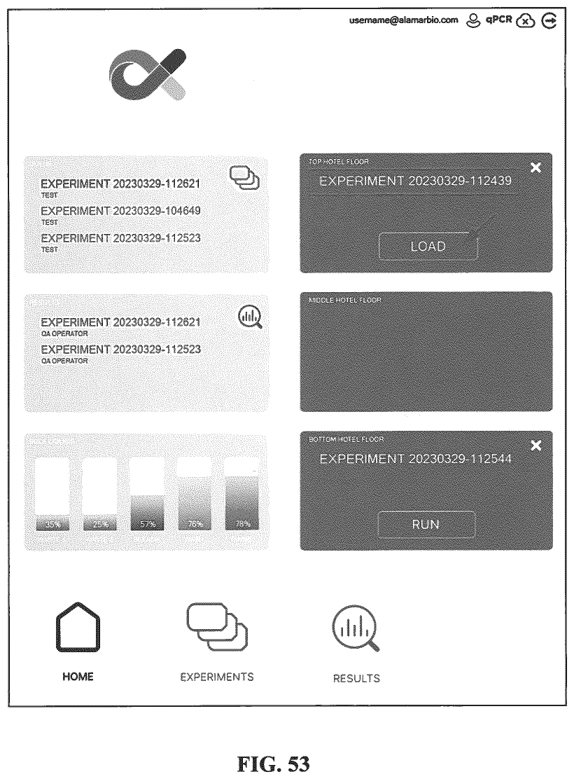

On the ARGO HT instrument, the experiment is visible in either one of the bays or in the experiments queue. Check if the experiment is in one of the bays. If the ARGO HT instrument has other experiments loaded, then determine if they need to run before your experiment is run. For this example, we will assume that your experiment downloaded from ANA and is loaded onto the top bay. See FIG. 53.

FIG. 53 illustrates an Experiments Screen of the Alamar NULISA Analysis (ANA) software showing a prompt to load the experiment into the first bay.

To load the experiment:
1. Select the LOAD button on the ARGO HT touchscreen (see FIG. 53). A confirmation dialog box will be displayed asking if you are ready to load the bay.
2. Select YES on the dialog box to proceed. The bay door will open and a dialog box will be displayed.
3. Load the consumables from the kit into the bay. See Reagent Kit Loading Overview for detailed instructions on loading the kit into the bay.

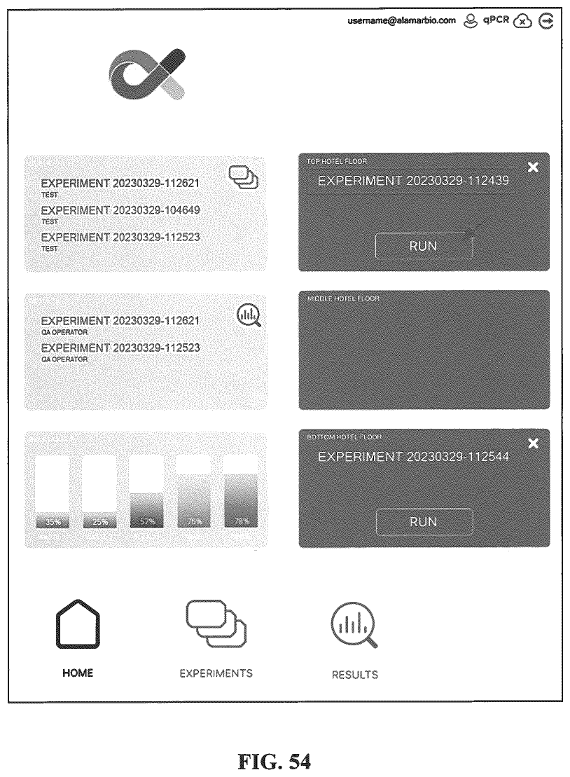

If the bay door has been open too long, a warning will be displayed indicating the door has been opened longer than expected and that the door will automatically close if it has been open for more than 10 minutes. If this message is displayed, continue loading the consumables and select the Ok button.
4. Press NEXT on the Load Consumables dialog box to proceed. A dialog box will be displayed asking whether the loading has been completed. Select YES on the dialog box to indicate that the bay has been loaded. The bay door will close and a bay loading complete dialog box will be displayed.
5. Select YES on the dialog box to proceed. The RUN button will be displayed on the bay. See FIG. 54.

FIG. 54 illustrates an Experiments Screen of the Alamar NULISA Analysis (ANA) software showing a prompt that experiment is loaded in the first bay and is ready to run.

The experiment has been loaded. Continue with Section to run the experiment.

### 4-14. Running the Experiment.

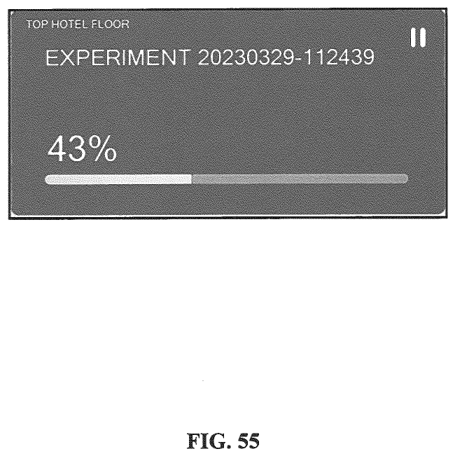

To run the experiment:
1. Press the RUN button on the ARGO HT touchscreen (see FIG. 54). A confirmation dialog box will be displayed asking if you are ready to proceed.
2. Press YES on the dialog box to proceed. A Scheduling Protocol information box will be displayed briefly followed by a check of the bulk liquid levels for the run.
3. Press NEXT on the Bulk Liquid Levels dialog box to proceed. The waste container level will be check and an information box will be displayed.
4. Press NEXT on the Waste Container dialog box to proceed. The consumables loaded dialog box will be displayed.
5. Press OK on the Consumables Loaded dialog box to proceed. The ready to start assay run dialog box will be displayed.
6. Press YES on the ready to start assay dialog box to proceed. The experiment will begin running and a progress bar will be displayed to indicate that the experiment is running. See FIG. 55.

FIG. 55 illustrates an Experiments Progress Bar of the Alamar NULISA Analysis (ANA) software.

When the experiment has completed, the UNLOAD button will be displayed.

This completes the running of the experiment. At this point, you can: Start another experiment that has been queued up in another bay to run (see Preparing to Run the Experiment). Unload the experiment that has just been completed (see Unloading the Experiment).

### 4-16. Unloading the Experiment.

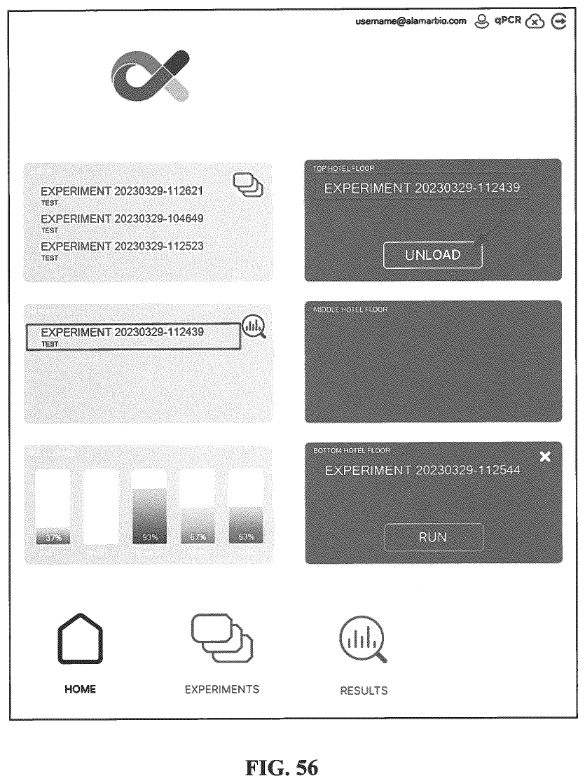

To unload the experiment:
1. Touch the UNLOAD button to begin the unload process. See FIG. 56.

FIG. 56 illustrates an Experiments Screen of the Alamar NULISA Analysis (ANA) software showing that the experiment is ready to unload.

The Unload Bay dialog box will be displayed. Note that the experiment data will be shown under the RESULTS display and the results will automatically be uploaded back to ANA for analysis.
2. On the Unload Bay dialog box, select YES. The bay door will open.
3. When the bay door is open, unload all of the consumables from the bay and touch the NEXT button. The Bay Unloading Completed dialog box is displayed.
4. When all of the consumables have been removed, touch the YES button on the dialog box. The experiment will no longer be displayed in the bay. Additional experiments may now be loaded into this bay from the experiments queue.

### 4-18. Reagent Kit Loading Overview.

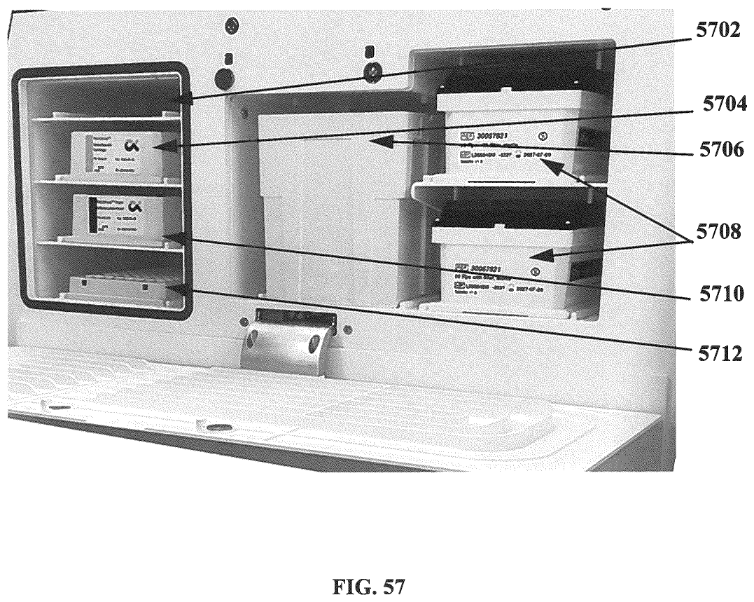

The components of the reagent kits are loaded into the bay prior to running an experiment. An overview of the loading is shown in FIG. 57.

FIG. 57 illustrates a reagent loading overview of a bay of the ARGO HT Instrument which comprises a ligation plate for NULISAseq 5702, a NULISAseq detection kit 5704, a consumables cartridge 5706, tip boxes 5708, a NULISAseq (or PCR) target kit 5710, and a sample plate 5712.

BIOLOGICAL RISKS: Wear disposable gloves, eye protection and other personal protective equipment (PPE) mandated by your institution's safety policies while performing experiments using the Alamar Biosciences ARGO HT. Wearing PPE prevents exposure to chemical and biologically hazardous materials.

4-19. Loading Reagent Kits into the ARGO HT.

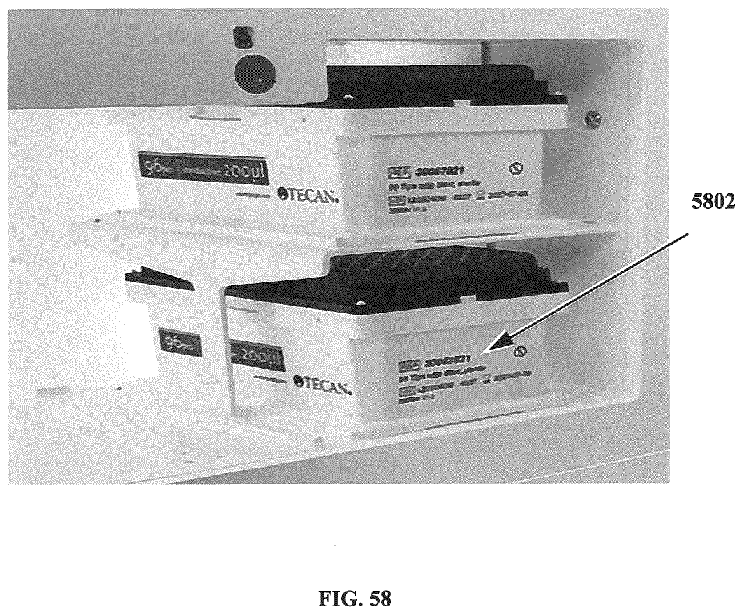

When you are ready to run an experiment, load the reagent kits into the ARGO HT instrument.
1. Remove all of the contents from the reagent kit box.
2. Load the tip boxes into the bay that will be used to run the experiment. Tip boxes should be loaded on the right side of the bay with part number label to the front which place tip A1 at the front left when the kit is inserted into the ARGO HT instrument. See FIG. 58.

FIG. 58 illustrates a bay of the ARGO HT Instrument which shows loading of the tip boxes with the label facing front 5802.

Press the box firmly into the channel until it stops.

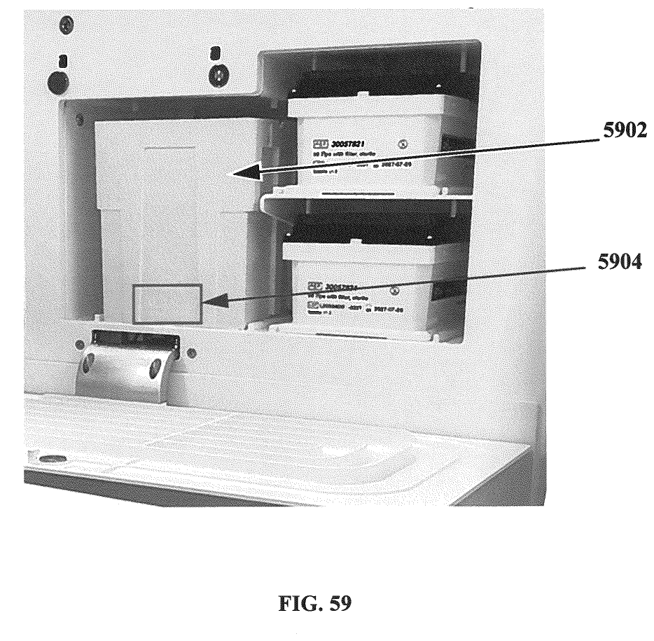

When inserting the boxes and kits into the bay, they must be inserted straight so that both sides of the box or kit are under the rails.
3. Load the second tip box into the slot above the first tip box following the instructions in Step 2.
4. Load the consumables kit into the slot beside the tip boxes. The kit is loaded so that the Alamar Biosciences logo is at the front of the box. Press the box firmly into the channel until it stops. See FIG. 59.

FIG. 59 illustrates a bay of the ARGO HT Instrument which shows loading of consumables cartridge 5902 with the Alamar Biosciences logo facing front 5904.

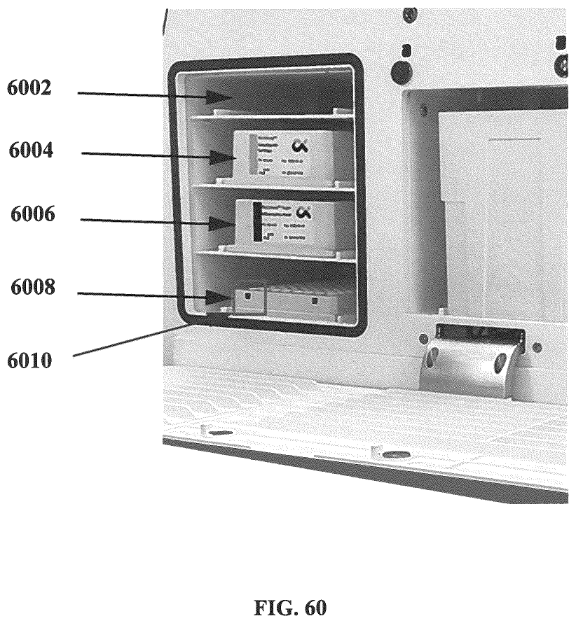

Check that the film seal frame is properly seated in the notches at the top of the carrier before loading the consumables kit into the instrument.
5. Load the sample plate into the slot bottom slot to the left of the consumables cartridge. Press the box firmly into the channel until it stops. See FIG. 60.

FIG. 60 illustrates a bay of the ARGO HT Instrument which shows loading of the sample and reagent kit cartridges comprising a ligation plate for NULISAseq 6002, a NULISAseq detection kit 6004, a NULISAseq (or PCR) target kit 6006, and a sample plate 6008 wherein position A1 of the sample plate 6010 must be at the front left when loaded.

When inserting the sample plate into the slot, it must be inserted with position A1 at the front left.
6. Load the NULISAseq (or PCR) target kit into the slot above the sample plate. Press the kit firmly into the channel until it stops. The kit is loaded so that the label is at the front. See FIG. 60.
7. Load the NULISAseq detection kit 1 into the slot above the target kit. Press the kit firmly into the channel until it stops. The kit is loaded so that the label is at the front. See FIG. 60.
8. Load the ligation plate for NULISAseq into the slot above the NULISAseq detection kit. Press the ligation plate firmly into the channel until it stops. See FIG. 60.

### 4-21. Unloading the ARGO HT After Experiment Completion.

After an experiment is completed, remove the reagent kits and consumables from the ARGO HT instrument.

BIOLOGICAL RISKS: The tip boxes and sample plate may have biological materials after running an experiment. Biological samples such as tissues, body fluids, and blood of humans and/or animals have the potential to transmit infectious diseases. Follow your local, state/provincial, and national safety regulations for handling and disposing the tip boxes.
1. Remove the tip boxes from the bay by pulling them forward from the instrument and dispose of them according to your lab's biohazard procedures.
2. Remove the consumables cartridge from the bay by pulling it forward from the instrument and dispose of it according to your lab's biohazard procedures.
3. Remove the sample plate from the bay by pulling it forward from the instrument. Dispose of it according to your lab's biohazard procedures unless it requires further processing.
4. Remove the target and detection kits from the bay by pulling them forward from the instrument and dispose of them according to your lab's biohazard procedures.

When running a pooled library, the detection kit may need further processing on another instrument. Do not dispose of the detection kit if it is needed for further processing.
5. Remove the ligation plate from the bay by pulling it forward from the instrument. Dispose of it according to your lab's biohazard procedures.

### 4-22. Analyzing Experiment Data.

After running the experiment, a snapshot of the data can be viewed on the ARGO HT instrument. The data is also uploaded back to ANA for more detailed analysis.

It is recommended that: You perform a quick view of the data on the ARGO HT instrument to ensure that the data has been extracted from the experiment. You use ANA to do a more extensive analysis of the data on your desktop computer.

### 4-22. Viewing Data on ARGO HT.

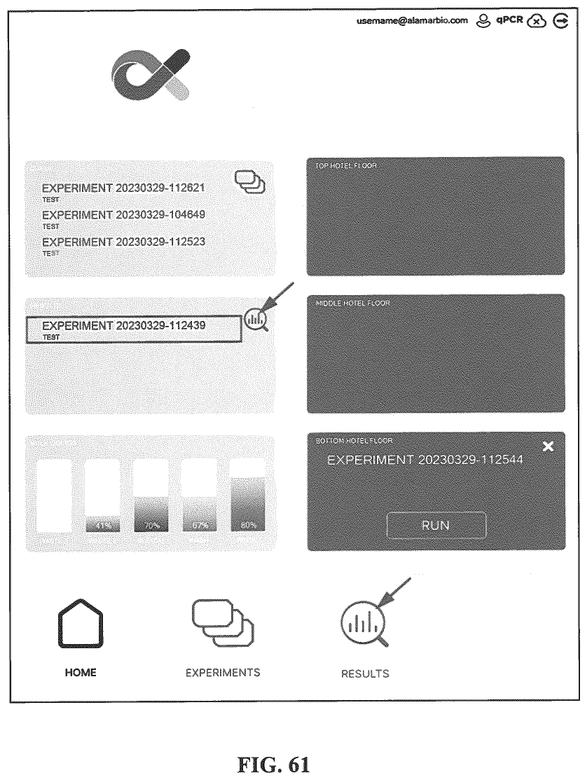

To view data from a run on the ARGO HT instrument:
1. Select the RESULTS icon at the bottom of the screen or the icon in the Results panel. See FIG. 61.

FIG. 61 illustrates the Experiments Screen of the Alamar NULISA Analysis (ANA) software showing an option for viewing of experiment results.

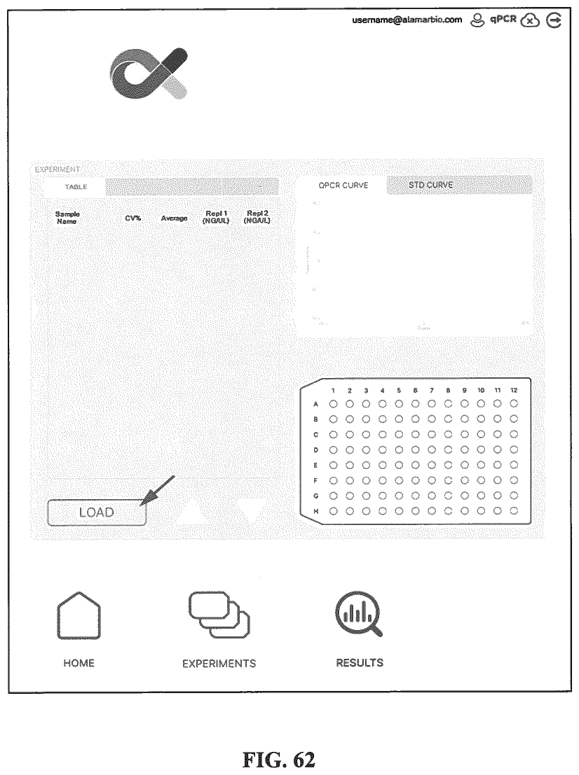

The RESULTS screen is displayed.
2. Select LOAD to display the list of available results on the ARGO HT instrument (see FIG. 62).

FIG. 62 illustrates the Experimental Results screen of the Alamar NULISA Analysis (ANA) software showing a prompt to load the results.

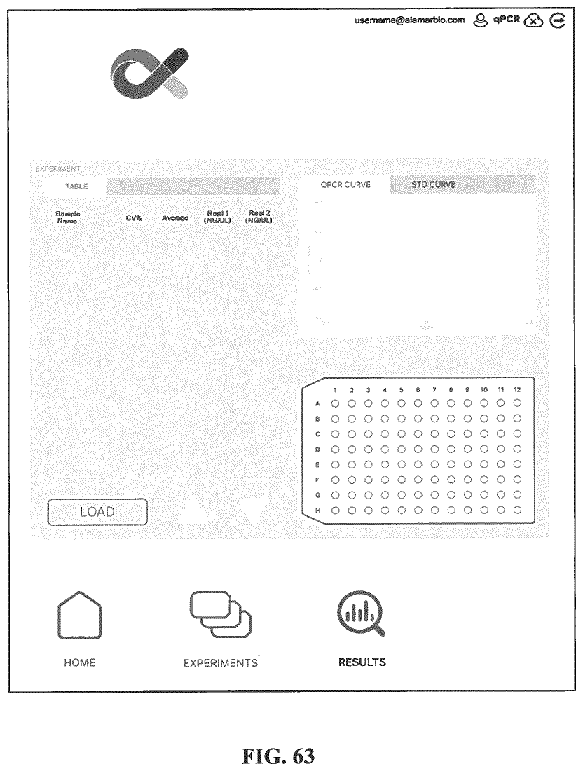

A dialog box of available results is displayed.
3. From the list of available results, select the desired results for viewing and select Ok. The results are displayed in the Results panel. See FIG. 63.

FIG. 63 illustrates the Experimental Results Loaded screen of the Alamar NULISA Analysis (ANA) software.

4-26. Analyzing Data using ANA.

To view and analyze data from a run using ANA:
1. Locate the Alamar Biosciences software shortcut on the desktop and double-click the icon. The ANA software will load and display the ANA Log in screen.
2. On the ANA Login screen, enter your email address and password, and click the Log in button. The ANA software will load and the HOME screen will be displayed. See FIG. 44.
3. On the HOME screen, select ANALYSIS to display the list of available results in the ANA software (see FIG. 44). The ANALYSIS screen is displayed showing available projects. See FIG. 64.

FIG. 64 illustrates the Alamar NULISA Analysis (ANA) Screen with Results Selected which comprises an option to click on a project to display experiments with results 6402, the results for an experiment 6404, and an analysis preview pane 6406.
4. From the list of available projects, select the desired analysis for viewing results. A preview of the results are displayed in the Analysis Preview pane. See FIG. 64.

To view the experiment parameters for the data, select the VIEW EXPERIMENT button on the Analysis Preview pane (see FIG. 64) and the experiment parameters will be displayed in the EXPERIMENTS screen (see FIG. 50).

There are several ways to view the data in the Analysis Preview Pane (see FIG. 64). To navigate to the full results view, select VIEW ANALYSIS. The format of the data will vary according to the type of experiment.

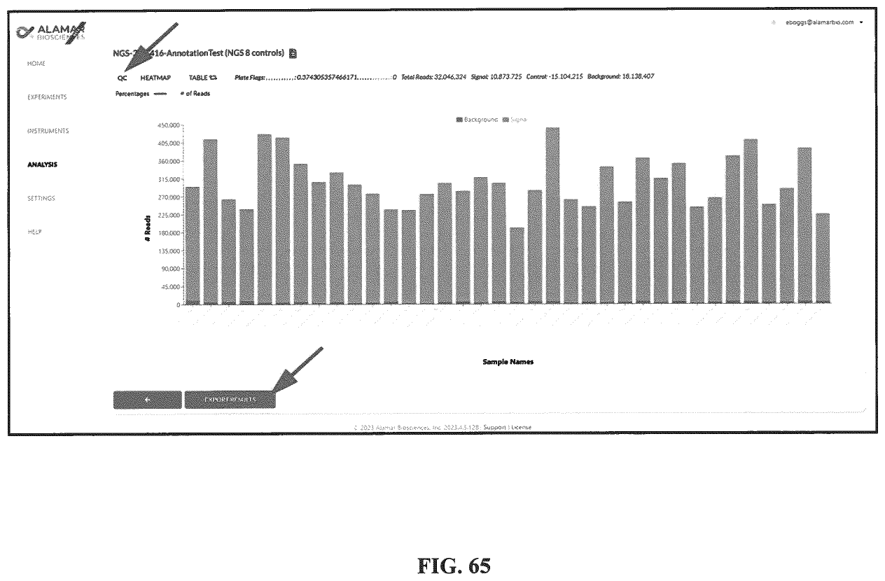

Most of the data screens allow exporting data for further analysis in various formats. For example, NGS/multiplex can export to XML. Other options may include: PDF, HTML and, for qPCR/singleplex, JSON and Microsoft Excel (.xlsx). Charts can be exported as a picture (right-click and select Export as PNG). Tables can be exported to CSV (right-click and select Export as CSV) and imported into Microsoft Excel or other spreadsheet programs. To export the data using JSON format, select the EXPORT RESULTS button on any of the data analysis screens. See FIG. 65.

FIG. 65 illustrates the Alamar NULISA Analysis (ANA) Screen with QC Results.

Select the data views using the tabs near the top of the screen. Some of data screens are shown in the following FIGS. 65-67.

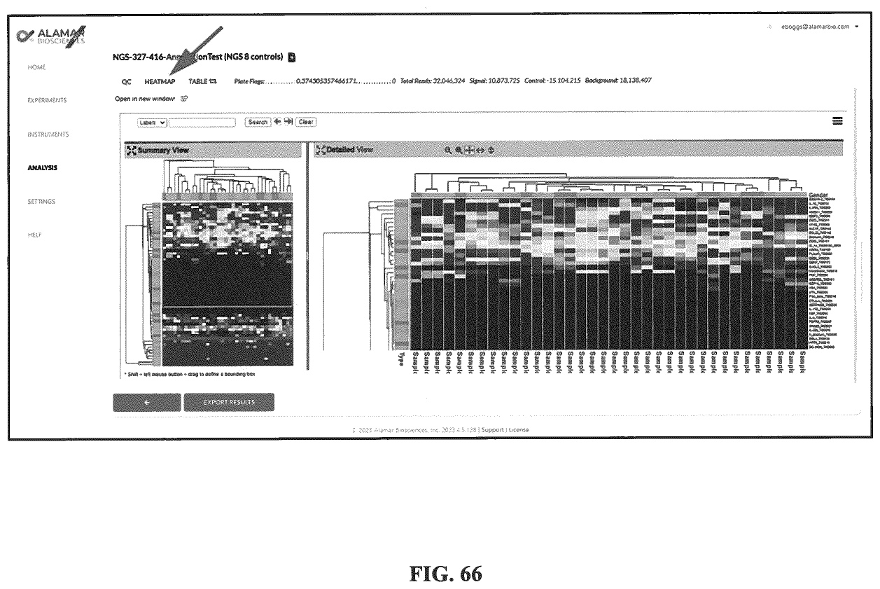

FIG. 66 illustrates the Alamar NULISA Analysis (ANA) Screen with Heatmap Results.

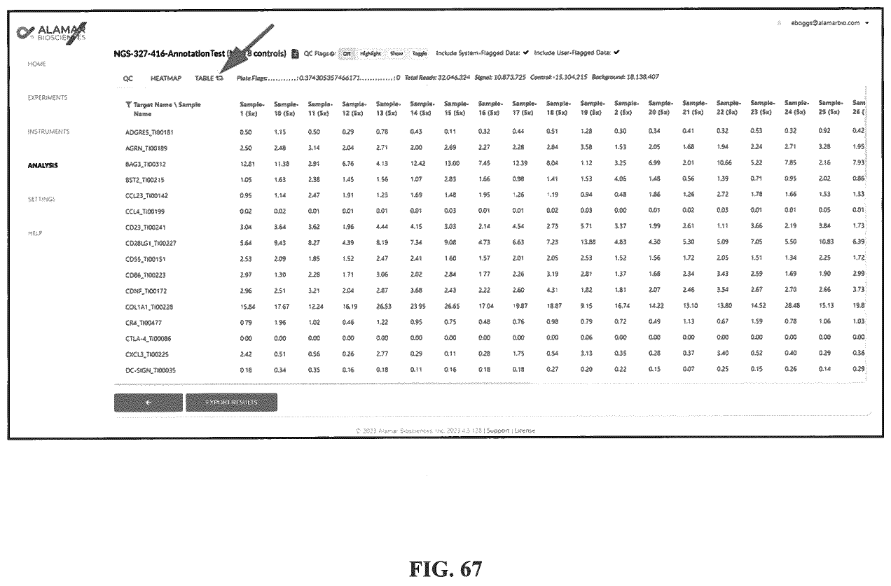

FIG. 67 illustrates the Alamar NULISA Analysis (ANA) Screen with Results Table.

### 4-29. Instruments Screen.

FIG. 68 illustrates the Alamar NULISA Analysis (ANA) Instruments Screen with an Instrument selected which comprises a tab function to display experiments 6802 and a list of experiments for that instrument 6804.

The INSTRUMENTS screen shows the status of each instrument that the ANA user has access to. It includes information about which experiments are running or have run on an instrument, assays, status of the run, when the experiment started its run, estimated completion time and time to completion if the experiment is still running. See FIG. 68.

A supervisor has access to all experiments on all instruments and can view all of the information pertaining to all experiments. An operator can only view experiments that were created by that operator or experiments belonging to a group that the operator is a member of.

TABLE 4-3
Alamar Biosciences ANA INSTRUMENTS Screen.

| Screen Name | Function |
|---|---|
| Instrument/ | The Instrument/Experiment column lists the experiments |
| Experiment | currently running and experiments that have been run |
| | on the instrument. See FIG. 4-26. |
| Assay(s) | The Assay(s) column lists the assay for each experiment. |
| | See FIG. 4-26. |
| Status | The Status column shows the current instrument status |
| | and the experiment run status of each experiment on |
| | the instrument. See FIG. 4-26. The following instrument |
| | status indications are: |
| | Offline: The instrument is currently offline. |
| | Empty: The instrument is not currently |
| | running any experiments. The following |
| | experiment status indications are: |
| | Status Bar: If an experiment is running, a status bar |
| | will show the completion percentage. |
| | Loaded: An experiment is loaded and ready to run. |
| | Status Bar: If an experiment is running, a status bar |
| | will show the completion percentage. |
| | Paused: An experiment has been paused. |
| | Pending: If an experiment has been downloaded to the |
| | instrument. |
| | Completed: An experiment has completed its run. |
| | Error: An experiment failed to complete successfully. |
| Submission | The Submission Time is the time that the experiment was |
| Time | downloaded to the instrument from ANA. See FIG. 4-26. |
| Est. Comple- | The Estimated Completion Time is the time that the |
| tion Time | experiment should complete its run. See FIG. 4-26. |
| Time to | The Time to Completion status for each experiment is |
| Completion | displayed. See FIG. 4-2. The following Time to |
| | Completion status indications are: |
| | Done: The experiment has completed and data has |
| | been uploaded to ANA. |
| | Pending Start: The experiment has not yet started |
| | but has been downloaded to the instrument from ANA. |
| | XX hours/minutes: The experiment is running and is |
| | expected to be completed in XX hours/minutes. |

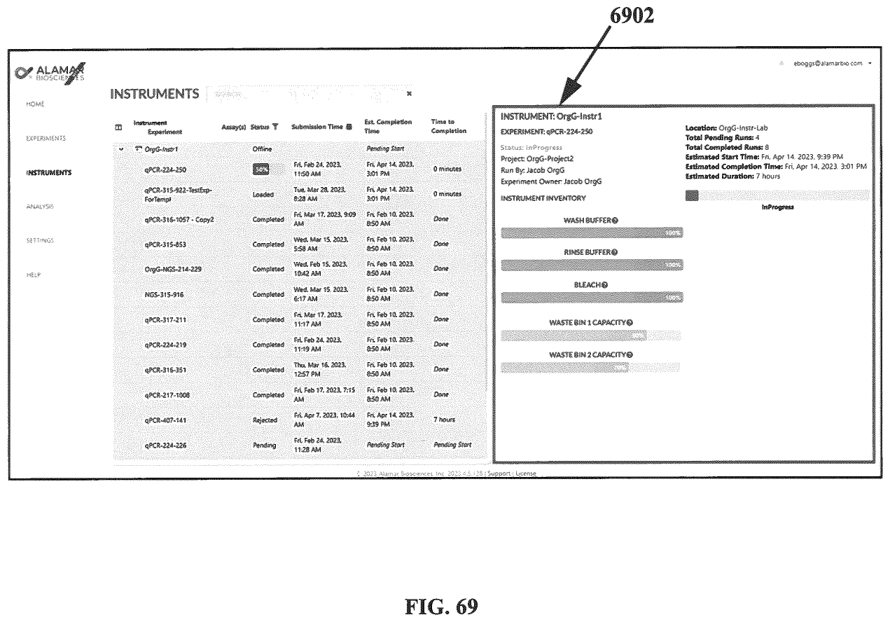

From the INSTRUMENTS screen, you can view the instrument details pane (see FIG. 69).

FIG. 69 illustrates the Alamar NULISA Analysis (ANA) Instruments Screen with an Instrument selected which comprises an instrument details pane 6902.

The instrument details pane 6902 shows instrument status including current run in progress, start time of the run, estimated completion time and status of the bulk consumables (instrument inventory). To view the Instrument Details pane, select the instrument name).

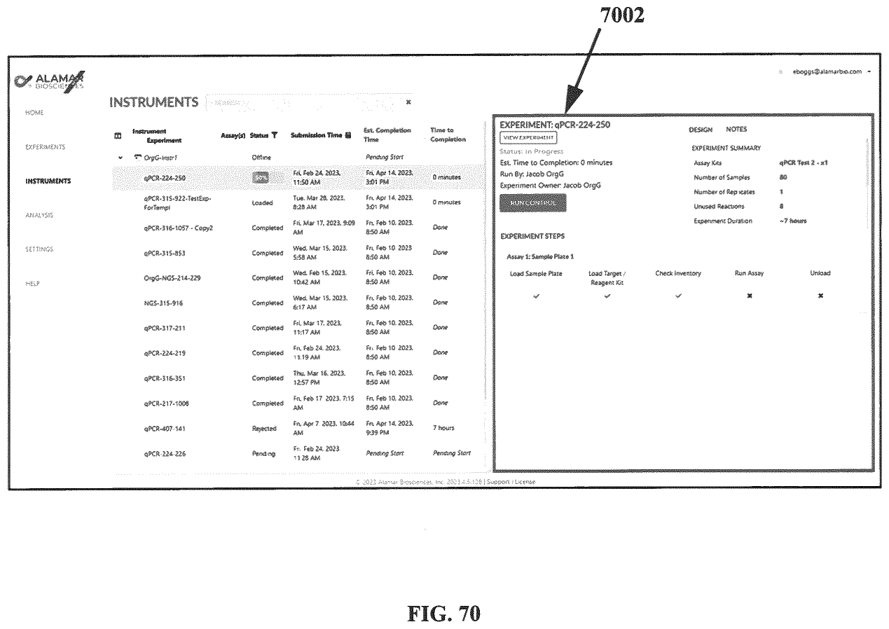

From the INSTRUMENTS screen, you can also view the experiment details pane (see FIG. 70).

FIG. 70 illustrates the Alamar NULISA Analysis (ANA) Instruments Screen with an Experiment selected which comprises an experiment details pane 7002.

The experiment details pane 7002 shows the status of the selected experiment including time to completion, experiment owner, person running the experiment, experiment summary and the status of the experiment steps (steps completed and steps remaining). Select the instrument (or expand with the caret (>)) to display experiments previously queued on that instrument. To view the Experiment Details pane, select the experiment name. You can also select RUN CONTROL which allows you to pause the experiment in the queue, re-queue the experiment to another instrument or cancel the experiment. You can select VIEW EXPERIMENT which takes you to the EXPERIMENTS screen where you can view the experiment definition details.

### 4-32. Settings Screen.

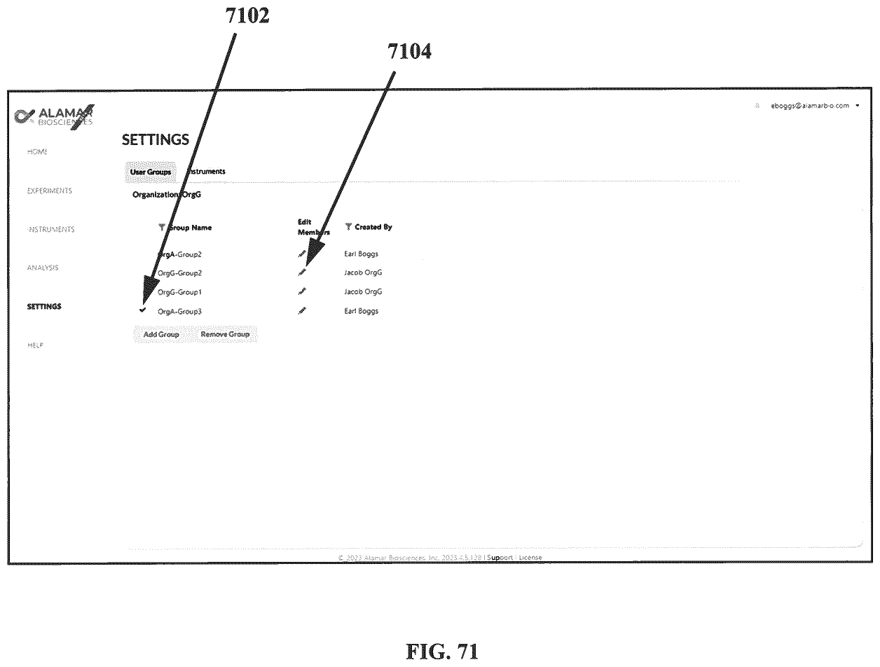

The SETTINGS screen allows the user to create and delete groups, edit group members and set instrument controls. See FIG. 71.

FIG. 71 illustrates the Alamar NULISA Analysis (ANA) Settings Screen with user groups tab selection which comprises a check box to remove a group 7102 and a pencil icon to edit group members 7104.

Select the User Groups tab to edit user groups and members or select the Instruments tab to manage instruments

### Adding and Deleting Groups.

To add a new group, select the Add Group button underneath the group list. A new line will be added to the organization table. Select the Click to edit . . . to edit the name of the new group. Select the pencil icon to add members to the group. See FIG. 71.

To delete a group, select the check box to the left of the Group Name and select the Remove Group button. A dialog box will be displayed asking if you are sure you want to delete the group. Select Yes to delete the group. The group will be deleted from the User Groups.

Multiple groups may be selected for removal by checking additional check boxes beside the group names. All groups may be selected by selecting the check box at the top of the column.

### Adding and Deleting Group Members.

Group roles apply within a project only. These roles are:

Reader: A reader can run experiments within any projects associated with the group, as well as view and perform experiment analysis for all experiments within any projects associated with the group.

Editor: An editor can, in addition to the reader permissions, create experiments within any projects associated with the group, view and queue experiments on all instruments assigned to the project's group, run experiments within the project and perform experiment analysis for all experiments within the project.

Admin: An admin can, in addition to the editor permission, manage any projects associated with the group (e.g. change project names) and can manage the group (e.g. assign users and roles within the group).

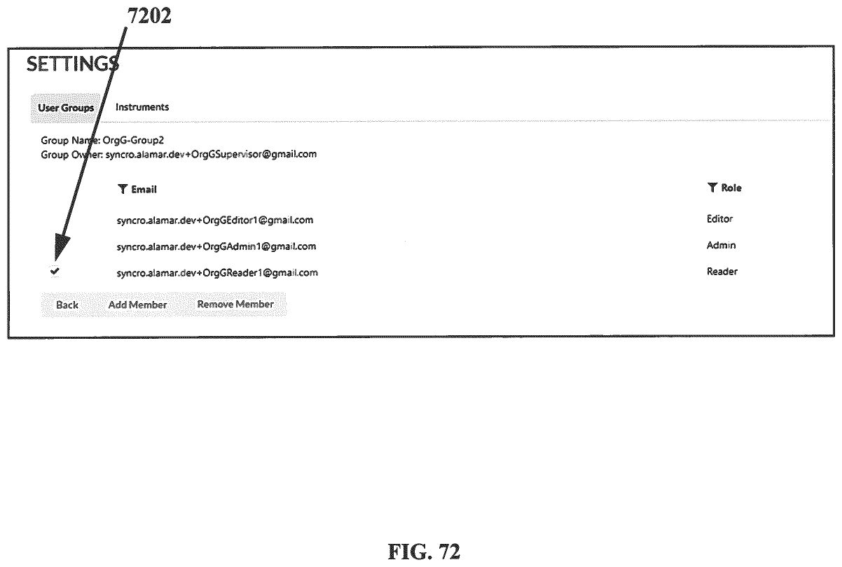

To add a new member to a group, select the Add Member button (see FIG. 72).

FIG. 72 illustrates the Alamar NULISA Analysis (ANA) Settings Screen with adding and deleting group members which comprises a check box to remove a group member 7202.

A new line will be added to the member table. Select the Choose a user . . . to display a drop down of authorized users. Select a user from the list to add them to group. Under the Role column, select the role for the added user (Admin, Editor or Reader) for that group. Repeat for adding additional users to the group.

To delete a member, select the check box to the left of the user Email and select the

Remove Member button. The user will be deleted from the group. See FIG. 72.

Multiple members may be selected for removal by checking additional check boxes beside the member email addresses. All members may be selected by selecting the check box at the top of the column

### Manage Instruments.

To manage instruments, select the Instruments tab. See FIG. 73.

FIG. 73 illustrates the Alamar NULISA Analysis (ANA) Settings Screen with instrument tabs selection which comprises a toggle to deactivate an instrument 7302 and a pencil icon to edit group members 7304.

On the Instruments tab, you can assign a name, Mac Address and location (optional) to each instrument. To make these changes, select the text box for each item and enter the information.

The Instruments tab also displays the instrument serial number, instrument status (Offline, Online, Pending Registration) and the ICSVersion (software version) for each instrument. Serial numbers are unique and are configured for each instrument upon installation.

To add additional instruments to the list, select+ADD NEW. A dialog box will ask for information about the new instrument (Name, Serial Number, Mac Address and Location). Enter the information and select ADD. A dialog box will be displayed showing the new instrument credentials. Select OK. The new instrument will be added to the list of instruments with a status of Pending Registration.

### Deactivating an Instrument

To deactivate an instrument, select the Toggle Switch. Deactivating an instrument makes it unavailable for running experiments. When deactivating an instrument, a dialog box will be displayed asking if you are sure you want to deactivate the instrument. Select OK to confirm.

You cannot deactivate an instrument if there are jobs in the queue. You must wait until all jobs have been completed or remove jobs from the queue.

### Viewing Instrument Details.

To view instrument details, select the Clipboard icon above the toggle switch. This will display a dialog box showing the status of the wash buffer, rinse buffer, bleach and waste bin capacities. For more details on each item, hover over the ? icon above each item for more details (last loaded, expiration date and remaining capacity for bulk reagents; last emptied and remaining capacity for the waste bins

Select Close when finished viewing the instrument details.

### Locking an Instrument.

To lock an instrument for a specific user group, select the Lock icon above the toggle switch. Locking an instrument makes it unavailable for running experiments to users not belonging to a specific user group. When locking an instrument, a dialog box will be displayed asking if you are sure you want to lock the instrument along with a drop down of user groups for assignment to the instrument. Select the user group to assign and select OK to confirm. The lock icon will change to a closed lock.

To unlock the instrument from a user group, select the Lock icon. A confirmation dialog box will be displayed asking if you are sure you want to unlock the instrument. Select OK to unlock the instrument

### Refresh Credentials.

To refresh the credentials to the instrument, select the circular arrow icon. When an instrument is first configured by Alamar Biosciences Field Service, initial credentials are generated and installed on the instrument.

These are “passwords” unique to each instrument and must be kept secure. Leaking these credentials can pose a major security risk.

If an instrument needs to be reconfigured, it may need a new set of credentials in case the original credentials were lost/leaked. Refreshing credentials will disable the previous credentials, thus locking out all communication from the instrument previously configured with those credentials. After selecting the icon, a warning message is displayed indicating that all communication is invalidated until the instrument is reconfigured with the new credentials. Select REFRESH to continue with refreshing the credentials. An Instrument Credentials dialog box will be displayed with Instrument Name, Identifier and Secret. Note that the Identifier and Secret will only be displayed once. They should be copied and securely used for configuring the instrument, which will re-enable instrument communication with ANA. Select OK to continue.

### 4-26. Help.

Help will link to a series of topics to enable the user to better use the ANA web application.

### 6. Administrative Tasks.

### 6-1. User Management.

Management of users is done in the Alamar NULISA Analysis (ANA) software.

When the instrument was installed, at least one person was designated with the Supervisor role by the Alamar Biosciences field service engineer. The Supervisor role manages ANA users for your organization and at least one account must be assigned this role at all times.

### User Roles.

There are two user roles for the ANA software. These roles are:

Supervisor: A supervisor can invite additional users to the software. They can also deactivate or delete users. They have full administrative rights to view all projects and experiments in the software. They can deactivate instruments or lock them for use by a specific user group and see all instruments regardless of their status.

Operator: An operator can perform all functions within the ANA software but they will not be able to access instruments that have been deactivated or locked to a user group unless the operator is a member of that group. Experiments and projects created by an operator are visible only to that operator (and any organizational supervisors) unless the experiments belong to a project and the project has been mapped to a user group. Once mapped to a user group, operator members of that group may only perform limited duties depending on their assigned role. Group roles are Reader, Editor and Admin and only apply to projects to which the group is assigned.

### Creating New Users.

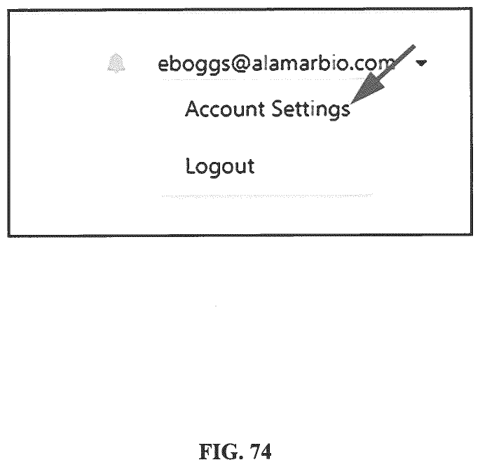

To create new users:
1. Log into the system using a supervisor role.
2. Select Account Settings from the drop-down menu under your user name (see FIG. 74).

FIG. 74 illustrates the Account Settings drop-down menu.

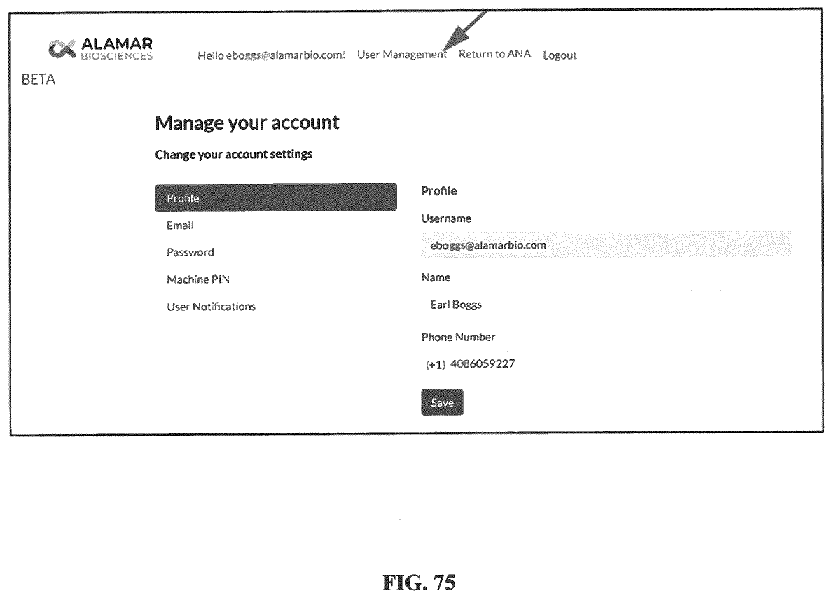

The Manage your account screen is displayed. See FIG. 75.

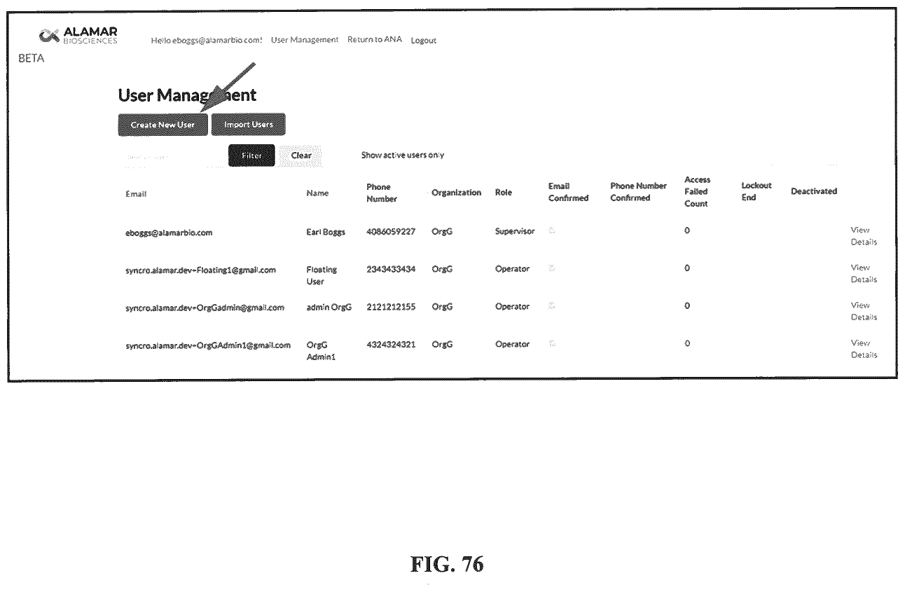

FIG. 75 illustrates the Manage your account screen.
3. Select User Management at the top of the Manage your account screen (see FIG. 6-2). The User Management screen is displayed. See FIG. 76.

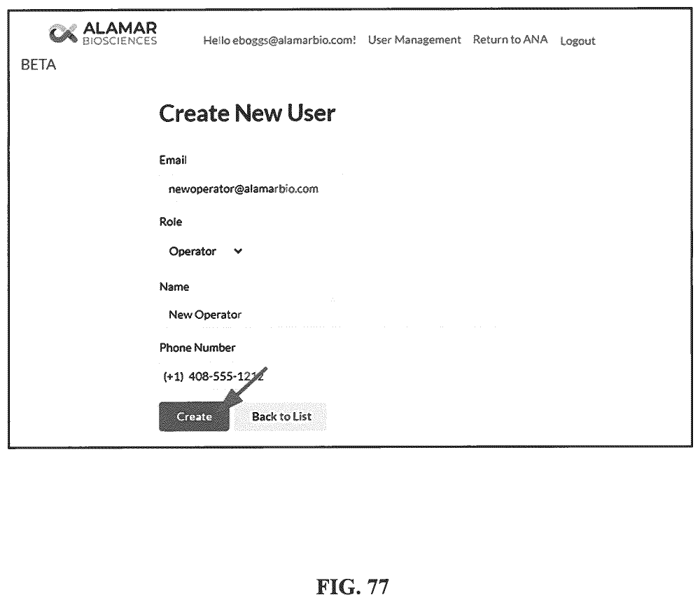

FIG. 76 illustrates the User Management Screen.
4. On the User Management screen, select Create New User. The Create New User screen is displayed. See FIG. 77.

FIG. 77 illustrates the Create New User Screen.
5. On the Create New User screen, enter the information required into the fields.
6. Select the role for the new user from the Role drop-down menu (Operator or Supervisor). For more information on roles, see Section.
7. When all of the required information has been entered, select the Create button. The new role will be created and the User Management screen will be displayed showing the new role in the user management table.
8. The new user will receive an email from ANA requesting confirmation of the email address. They must confirm their account by clicking the link in the email to complete account activation. The user will be directed to a page to set up their new account (confirm email address, create password). Once the account is activated, the user may begin to use the ANA software.

### Editing Users.

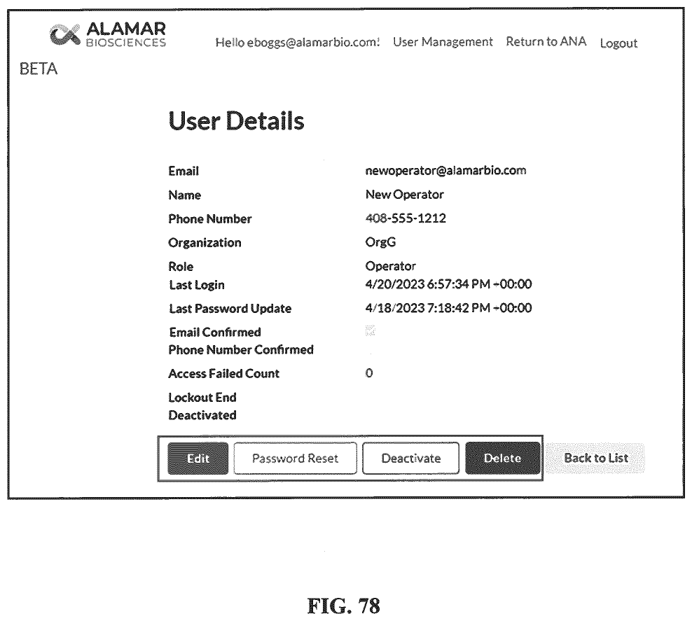

Existing users accounts may be edited by a supervisor. Edits to user accounts include resetting the user's password, deactivating the user and deleting the user from ANA. To edit users:
1. Log into the system using a supervisor role.
2. Select Account Settings from the drop-down menu under your user name (see FIG. 6-1). The Manage your account screen is displayed. See FIG. 75.
3. Select User Management at the top of the Manage your account screen (see FIG. 6-2). The User Management screen is displayed. See FIG. 76.
4. On the User Management screen, select View Details for the user account to be edited. The User Details screen is displayed. See FIG. 78.

FIG. 78 illustrates the User Details Screen.
5. On the User Details screen, select the actions to be taken for the user:

Edit: Modify user information. All fields may be edited except for the Organization name. Select Save when fields have been edited.

Access Failed Count: will display how many consecutive login failures that have occurred for the user account. After five failed attempts, the account will receive a temporary lockout for 30 minutes. The Lockout End field will show the time when the user's lockout expires. Supervisors may edit the profile and clear the Lockout End value to unlock a user immediately, if needed.

Password Reset: Allows you to reset a user's password (may be used if a user forgets their password). A dialog box will ask if you want to reset the password. Select Reset. The user will get an email to reset their password. They should follow the instructions in the email to reset their password. A user may also reset their own password by following the Forgot your password? link on the Log in screen.

Deactivate: Allows you to deactivate a user. The user account is still in ANA but they cannot log in. A dialog box will ask if you want to deactivate this user. Select Deactivate. The user account will be deactivated. The account can be reactivated by following the same process and selecting Activate.

Delete: Deletes the user account from ANA. The Delete User screen will be displayed showing all of the user information. Select Delete. The user account will be deleted and the User Management screen will be displayed.

Data Loss. Deleting a user is a permanent operation and the user cannot be restored. However, it is possible to recreate the user account using the Create User function described in Section. If a user account is created again, any previous experiments that were created or run by the deleted user will not be associated with the new account

### Bulk Importing of Users.

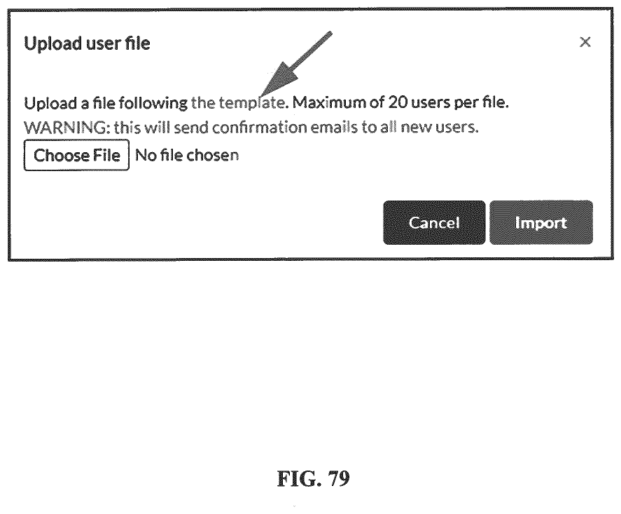

If you have a lot users to add to ANA (such as when initially setting up the system), you can create a .csv file in Microsoft Excel and import the users into ANA in batches of up to 20 users at a time. To import users:
1. Log into the system using a supervisor role.
2. Select Account Settings from the drop-down menu under your user name (see FIG. 6-1). The Manage your account screen is displayed. See FIG. 75.
3. Select User Management at the top of the Manage your account screen (see FIG. 6-2). The User Management screen is displayed. See FIG. 76.
4. On the User Management screen, select Import Users. The Upload user file dialog box is displayed. See FIG. 79.

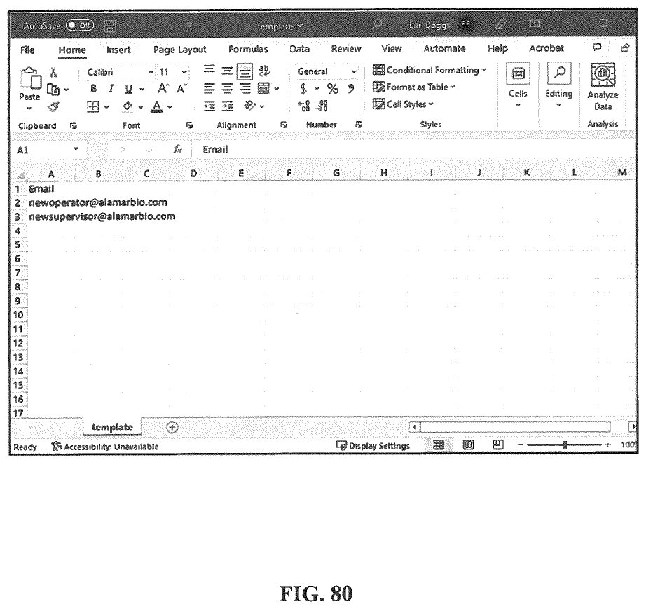

FIG. 79 illustrates the Upload user file Dialog Box.
5. If this is the first time to import users, you will need to download the template. Select the template from the dialog box. A template will be downloaded to your computer
6. Open the .csv file, insert all of the email addresses into the template and save the file on your computer. See FIG. 80.

FIG. 80 illustrates the Template File in Microsoft Excel.
7. In the Upload user file dialog box, select the Choose File button. The file will be uploaded and the filename displayed in the dialog box.
8. Select Import. The file will be imported and a Success dialog box will indicate that the file has been uploaded successfully. An email will be set to all new users after the file has been imported.
9. Close the dialog box and the new users will be displayed on the User Management screen.
10. Follow the instructions for Editing Users (see Section) to edit the new users (Role, Name, Phone Number).

### Bulk Consumables Replacement.

The bulk consumables will need to be serviced approximately every 15 experiments. The ARGO HT instrument will notify the operator when bulk consumables replacement is required. There are two components to the bulk consumables replacement: Emptying the two waste containers; Filling the decontamination buffer, DI water and wash buffer containers.

Before performing any bulk consumables procedures, be sure to wear proper personal protective equipment (PPE) because these procedures require handing hazardous materials. Proper PPE includes: Protective lab coat; Disposable gloves; and Eye protection.

Wear disposable gloves, eye protection and other personal protective equipment (PPE) mandated by your institution's safety policies while performing this procedure. Wearing PPE prevents exposure to chemical and biologically hazardous materials.

### Emptying the Waste Containers.

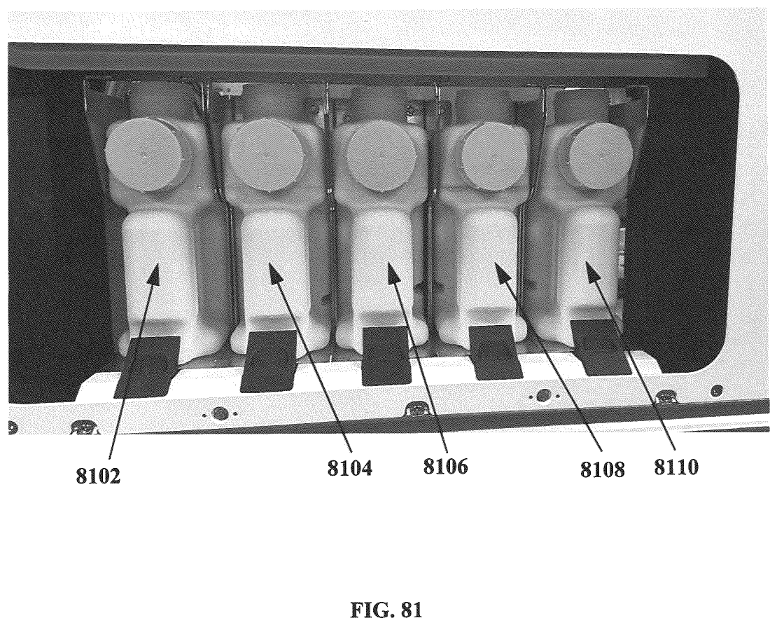

The waste containers are the two containers that are on the right-hand side of the bulk consumables area. See FIG. 81.

FIG. 81 illustrates bulk liquid components which comprise a decontamination buffer container 8102, a DI water container 8104, a wash buffer container 8106, and one or more waste containers 8108, 8110.

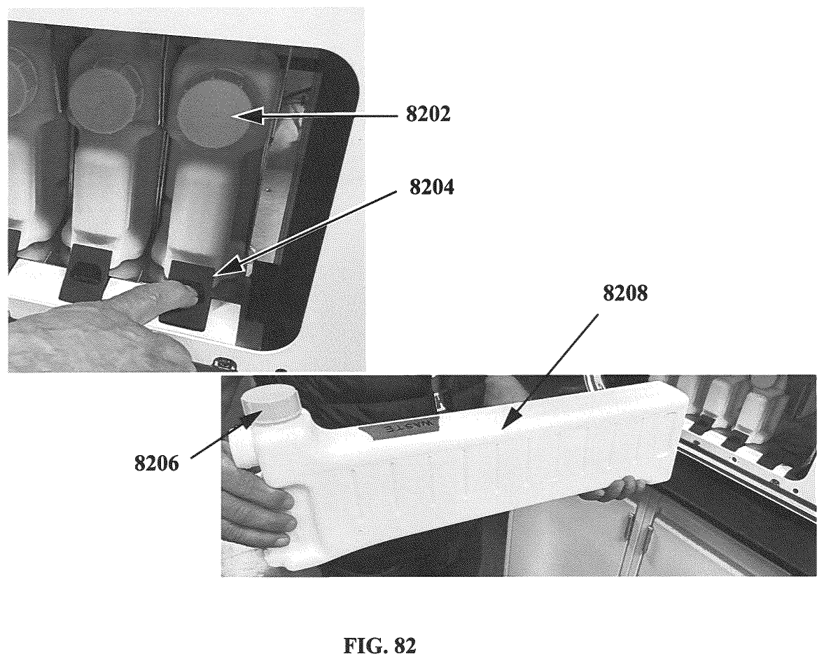

To empty the two waste container bottles:
1. Open the bulk consumables door from the ARGO HT touchscreen.
2. Press the black latch in the front of the bottle. See FIG. 82. FIG. 82 illustrates removing waste water bottle which comprises a yellow cap for bottle 8202, a press latch to release bottle 8204, a yellow cap on bottle 8206, and removing the bottle slowly 8208.
3. Slowly pull the bottle out approximately 150 mm (6 in.).
4. Unscrew the yellow cap from the front of the bottle and screw it tightly onto the opening on the top of the bottle. See FIG. 82. The waste bottles are very long. Moving a bottle too quickly will cause the liquid inside to move and possibly to splash through the top opening in the bottle. Move the bottle slowly and place the yellow cap on the bottle to prevent splashing the liquid waste. Wear disposable gloves, eye protection and other personal protective equipment (PPE) mandated by your institution's safety policies while performing this procedure. Wearing PPE prevents exposure to chemical and biologically hazardous materials.
5. Continue pulling the bottle until it is removed from the instrument. As the bottle is pulled forward, support the back of the bottle with your other hand. See FIG. 82.
6. Once the bottle has been removed, take it to the disposal location.
7. Remove the yellow cap from the bottle and empty to contents of the bottle into the disposal container.
8. Replace the yellow cap onto the waste bottle and take it back to the instrument.
9. Insert the bottle back into the slot in the instrument until it sticks out approximately 150 mm (6 in.).
10. Remove the cap from the top of the bottle and place it back onto its holder on the front of the bottle. The yellow cap must be screwed tightly to the front of bottle to prevent interference when the bulk consumables door closes.
11. Continue sliding the bottle back into the system until the latch snaps back into place.
12. Repeat Step 2 through Step 11 for the second waste bottle

### Replace Bulk Consumables.

There are three containers that contain bulk consumables on the left-hand side of the bulk consumables area. See FIG. 81. These consumables are: Decontamination Buffer; DI Water; and Wash Buffer.

Typically, the consumables are filled at the same time that the waste bottles are emptied.

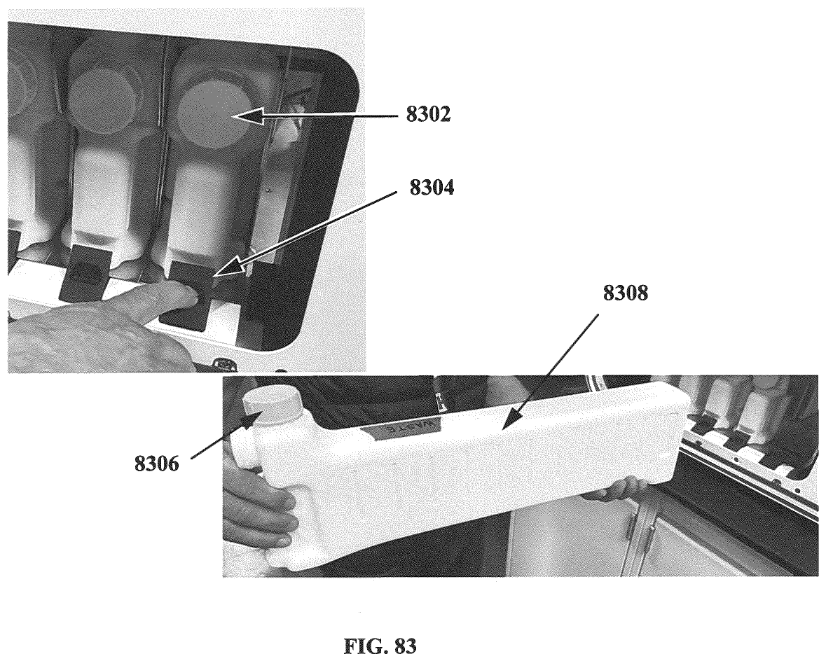

The procedure for filling the consumable bottles is the same for each bottle unless otherwise noted. To fill the three consumable bottles:
1. Open the bulk consumables door from the ARGO HT touchscreen. Skip this step if the door is already open for emptying the waste container bottles
2. Press the black latch in the front of the bottle. See FIG. 83. FIG. 83 illustrates removing consumable bottles which comprise a yellow cap for bottle 8302, a press latch to release bottle 8304, a yellow cap on bottle 8306, and removing the bottle slowly 8308.
3. Slowly pull the bottle out approximately 150 mm (6 in.).
4. Unscrew the yellow cap from the front of the bottle and screw it tightly onto the opening on the top of the bottle. See FIG. 83. The DI water bottle is very long. Moving the bottle too quickly will cause the liquid inside to move and possibly to splash through the top opening in the bottle. Move the bottle slowly and place the yellow cap on the bottle to prevent splashing the liquid waste. Wear disposable gloves, eye protection and other personal protective equipment (PPE) mandated by your institution's safety policies while performing this procedure. Wearing PPE prevents exposure to chemical and biologically hazardous materials.
5. Continue pulling the bottle until it is removed from the instrument. As the bottle is pulled forward, support the back of the bottle with your other hand. See FIG. 83.
6. Once the bottle has been removed, take it to the location of the consumables.
7. Remove the yellow cap from the bottle and fill the bottle with the consumable.
8. Replace the yellow cap onto the bottle and take it back to the instrument.
9. Insert the bottle back into the slot in the instrument until it sticks out approximately 150 mm (6 in.).
10. Remove the cap from the top of the bottle and place it back onto its holder on the front of the bottle. The yellow cap must be screwed tightly to the front of bottle to prevent interference when the bulk consumables door closes.
11. Continue sliding the bottle back into the system until the latch snaps back into place.
12. Repeat Step 2 through Step 11 for the remaining consumable bottles

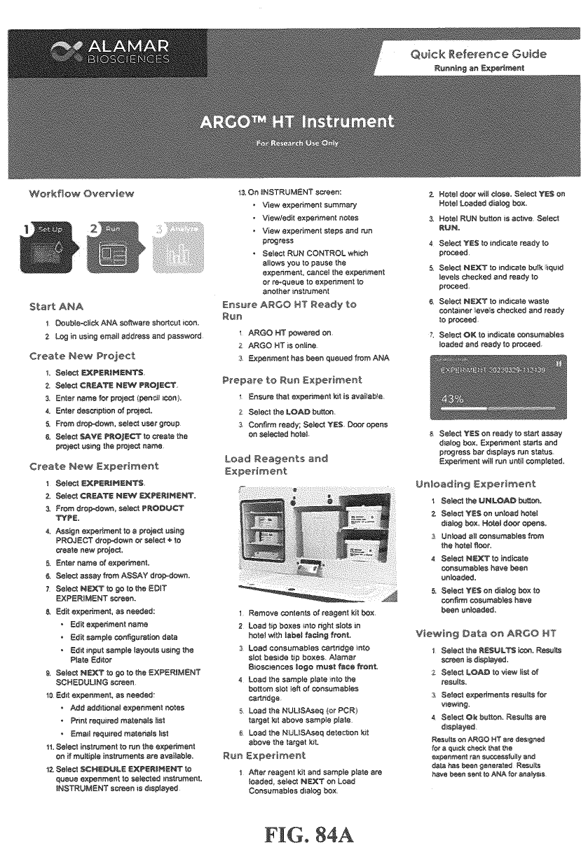

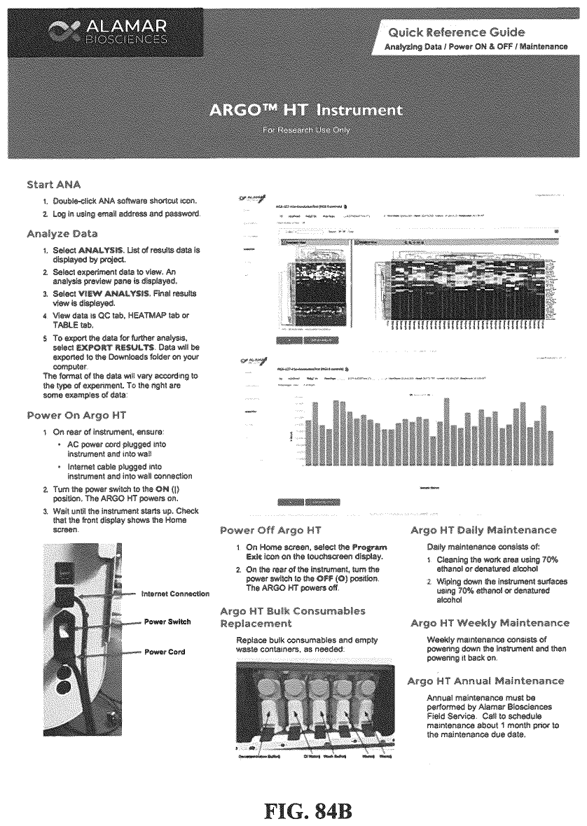

FIG. 84A illustrates a Quick Reference Guide for running an experiment.

FIG. 84B illustrates a Quick Reference Guide for analyzing data, powering on/off the instrument, and maintenance.

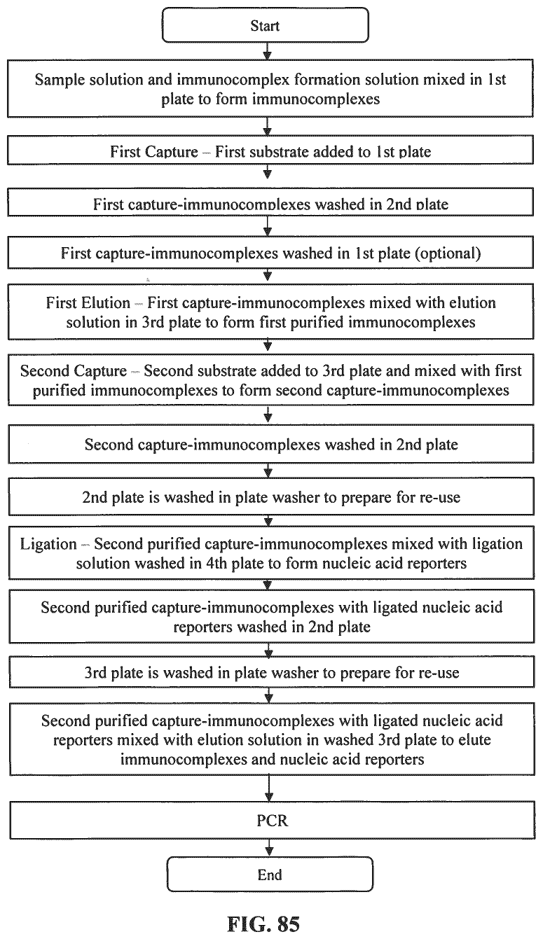

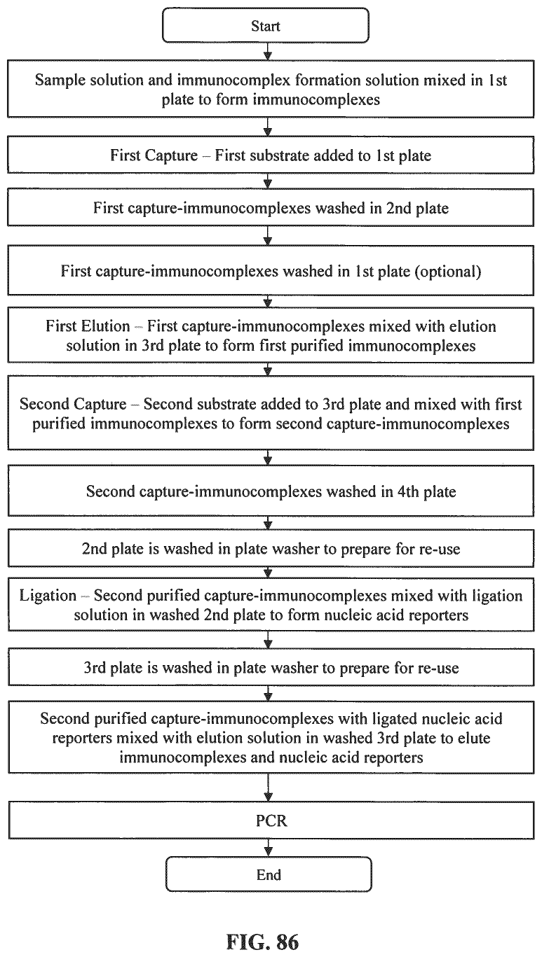

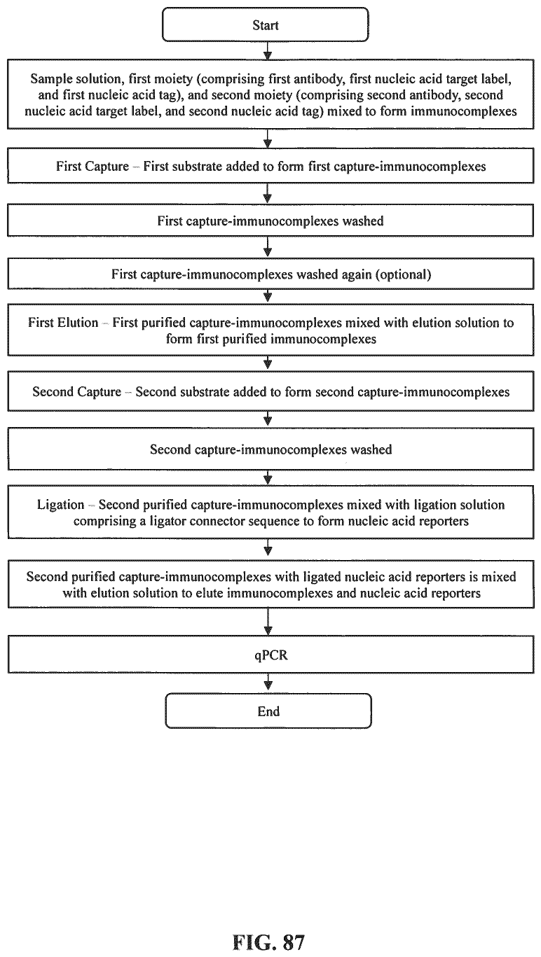

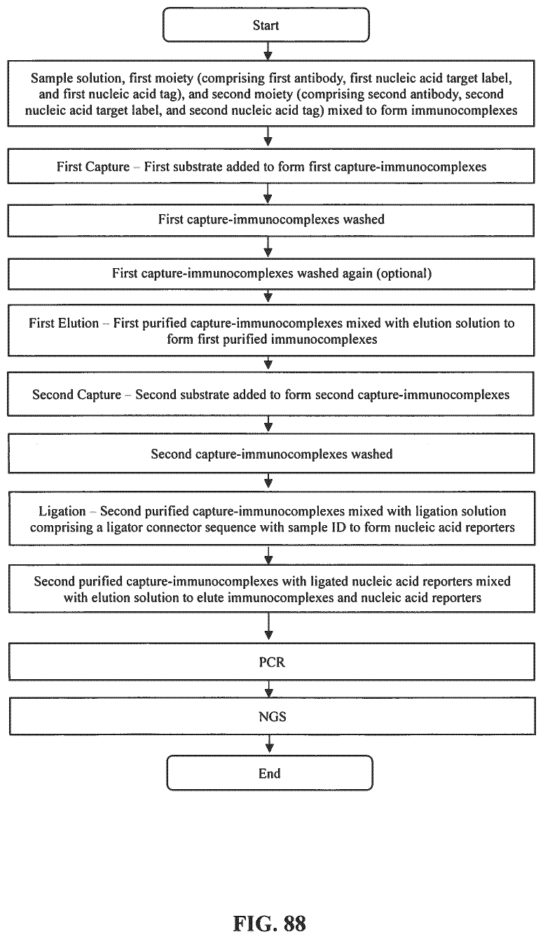

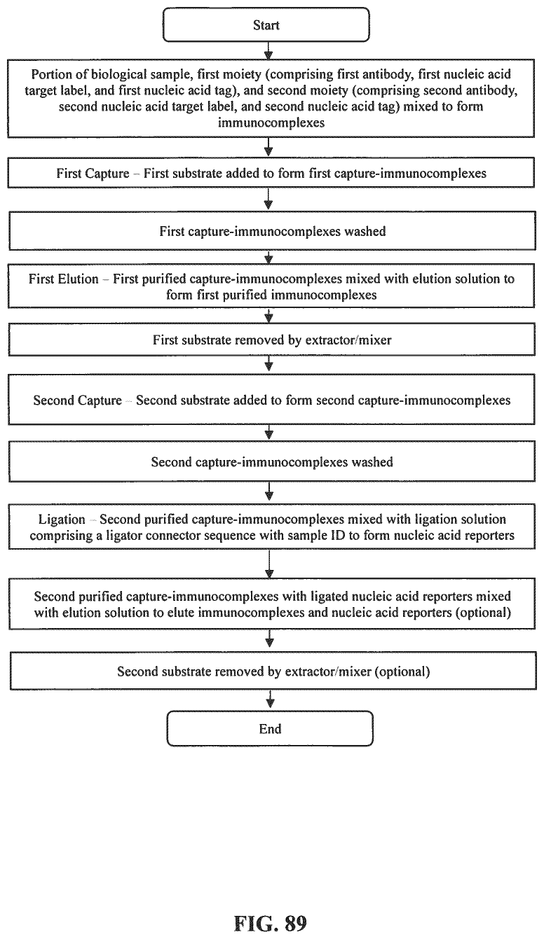

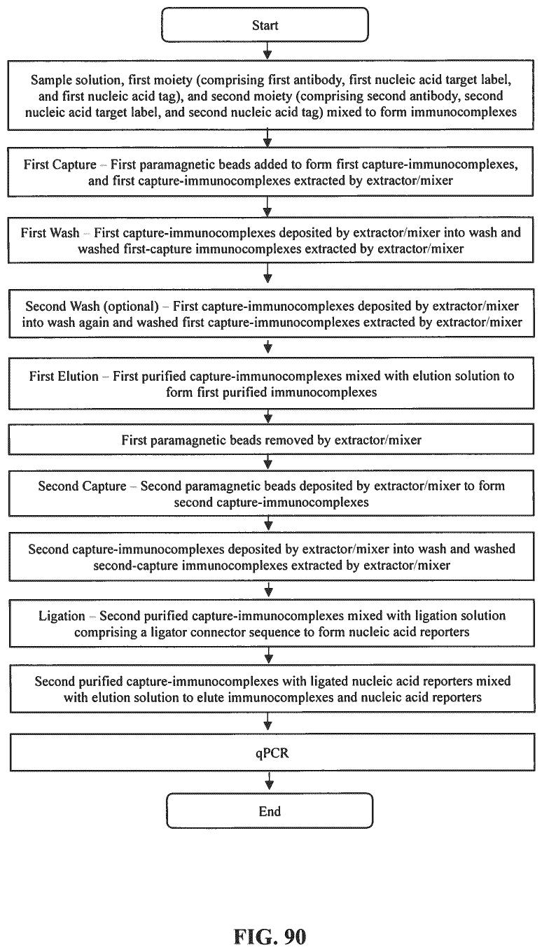

In some embodiments, the process steps presented in the flow charts in FIGS. 85-90, represent some of the executable instructions provided in the instrument controller's non-transitory machine-readable storage medium to be executed by the instrument controller's processor to provide controlled operations of the components within the instrument.

In some embodiments, for example, the NULISA assay may be executed by an instrument employing nine plates ((P1-P8 and P-qPCR)). Assay method for detecting an analyte using an bench-top automatic instrument: setting up a sample and the appropriate dilution curve on P1; transferring sample from P1 to P2; adding a first antibody conjugate, and a second antibody conjugate to P2; incubating P2 to form an immunocomplex comprising the first and the second antibody conjugate both bound to the analyte in the sample; adding a first magnetic bead to P2; incubating P2 to immobilize the immunocomplex on the surface the first magnetic bead; transferring magnetically the first magnetic bead from P2 to P3; washing the first magnetic bead in P3 to remove unbound impurities; transferring magnetically the first magnetic bead from P3 to P4; releasing the immobilized immunocomplex from the surface the first magnetic bead in P3; removing magnetically the first magnetic bead in P4; adding a second magnetic bead to P4; incubating P4 to immobilize the immunocomplex on the surface the second magnetic bead; transferring magnetically the second magnetic bead from P4 to P5; washing the second magnetic bead in P5 to remove unbound impurities; transferring magnetically the second magnetic bead from P5 to P6; generating a reporter in the immunocomplex on the surface the second magnetic bead in P6; transferring magnetically the second magnetic bead from P6 to P7; washing the second magnetic bead in P7 to remove unbound impurities; transferring magnetically the second magnetic bead from P7 to P8; releasing the immobilized immunocomplex from the surface the second magnetic bead in P8; transferring the released immunocomplex from P8 to P-qPCR; and detecting the presence of the reporter in P-qPCR.

In some embodiments, for example, an instrument system with the plate washer functionality, the number of plates necessary to execute the NULISA assay can be significantly reduced, which permits the construction of a bench-top system with the desirable form factors. As described below, the steps may be executed with as few as five plates (P1-P5 and P-qPCR). Before each plate is re-used, it is cleaned by the plate washer as appropriate. Assay method for detecting an analyte using an bench-top automatic instrument: setting up a sample and the appropriate dilution curve on P1; transferring sample from P1 to P2; adding a first antibody conjugate, and a second antibody conjugate to P2; incubating P2 to form an immunocomplex comprising the first and the second antibody conjugate both bound to the analyte in the sample; adding a first magnetic bead to P2; incubating P2 to immobilize the immunocomplex on the surface the first magnetic bead; transferring magnetically the first magnetic bead from P2 to P4; washing the first magnetic bead in P4 to remove unbound impurities; transferring magnetically the first magnetic bead from P4 to P3; releasing the immobilized immunocomplex from the surface the first magnetic bead in P3; removing magnetically the first magnetic bead in P3; adding a second magnetic bead to P3; incubating P3 to immobilize the immunocomplex on the surface the second magnetic bead; transferring magnetically the second magnetic bead from P3 to P1; washing the second magnetic bead in P1 to remove unbound impurities; transferring magnetically the second magnetic bead from P1 to P4; generating a reporter in the immunocomplex on the surface the second magnetic bead in P4; transferring magnetically the second magnetic bead from P4 to P1; washing the second magnetic bead in P1 to remove unbound impurities; transferring magnetically the second magnetic bead from P1 to P3; releasing the immobilized immunocomplex from the surface the second magnetic bead in P3; transferring the released immunocomplex from P3 to P-qPCR; and detecting the presence of the reporter in P-qPCR.

### 5. EXAMPLES

Certain embodiments are illustrated by the following non-limiting examples. The discussion below is offered to illustrate certain aspects of the present disclosure and is not intended to limit the scope of the claims. Changes can be made to the embodiments in light of the detailed description below. Although specific embodiments have been described herein for purposes of illustration, various modifications for carrying out the disclosure that are obvious to persons of skill in the art are intended to be within the scope of the claims.

### Example 1

Standard curves of IFNL1 and CSF2 of the NULISA inflammation panel were generated using standard antigen samples and 4PL curve fitting model. The standard antigen samples consisting of IFNL1 and CSF2 among a total of 237 recombinant proteins at various concentrations were prepared. The standard antigen samples were prepared by pooling each recombinant protein in the panel at desired concentration and 2-fold serial dilutions. Total 18 samples of 2-fold dilution were prepared on a sample plate.

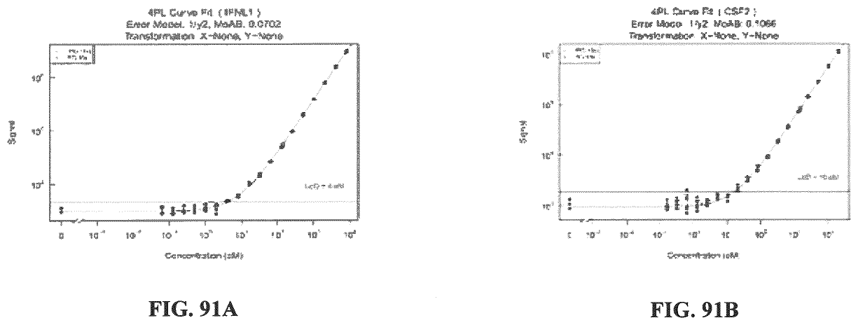

The sample plate for the 18 samples was placed into an ARGO instrument, equipped with a NULISAseq Inflammation Panel 250. A multiplexed assay library was automatically generated by the ARGO, within about 8 hours, and was subsequently analyzed by the Illumina NextSeq 1000/2000 DNA sequencing system. The read counts from the sequencing were normalized by the internal control. Normalized read counts and the protein concentrations were used to plot the standard curve for each target. Limit of detection (LOD) was calculated by mean of the blank plus 3 times of standard deviation of the blank samples. As reported in the FIGS. 91A and 91B, the LOD for the IFNL1 was 4 aM and for CSF2 was 16 aM.

What is claimed:

1. An instrument for automatically carrying out a dual-capture and release multiplexed immunoassay on a plurality of biological samples, comprising:
1) a controller;
2) a robotic gantry capable of moving in three degrees of freedom;
3) a hotel capable of holding multi-vessel carrier plates that are accessible by a user and the robotic gantry;
4) a mixer that operates as a magnetic bead processor and mixer comprising multiple multi-vessel carrier plate platforms;
5) a reader; and
6) positioned within the hotel,
a) a plurality of multi-vessel carrier plates, comprising a first plate comprising a plurality of first wells, a second plate comprising a plurality of second wells, a third plate comprising a plurality of third wells, a fourth plate comprising a plurality of fourth wells and a multi-vessel carrier assay plate comprising the plurality of biological samples; and
b) a multi-vessel carrier target kit comprising:
a plurality of multiplexed paired-binding moieties, the multiplexed paired-binding moieties comprising different paired-binding moieties that are pre-selected to bind different
specific analytes, the paired-binding moieties comprising:
i) a first moiety of the paired-binding moieties comprising a first antibody or a first antibody fragment that is pre-selected to bind a specific analyte, a first nucleic acid target label comprising a first identity that is analyte-specific to the specific analyte, and a first nucleic acid tag; and
ii) a second moiety of the paired-binding moieties comprising a second antibody or a second antibody fragment that is pre-selected to bind the same specific analyte as the first antibody or antibody fragment of the first moiety of the paired-binding moieties, a second nucleic acid target label comprising a second identity that is analyte-specific to the specific analyte, and a second nucleic acid tag;
wherein the first nucleic acid tag of the first moiety of the paired-binding moieties is preselected to bind to a first substrate that is the same for all the different paired-binding moieties in the multiplex and the second nucleic acid tag of the second moiety of the paired binding moieties is pre-selected to bind a second substrate that is the same for all the different paired binding moieties in the multiplex;
c) a multi-vessel carrier detection kit comprising:
i) a first substrate solution comprising a plurality of first substrates; and
ii) a second substrate solution comprising a plurality of second substrates.

2. The instrument of claim 1, wherein the controller comprises a processor and a non-transitory machine-readable storage medium comprising instructions executable by the processor to provide controlled operations of the components within the instrument.

3. The instrument of claim 1, wherein the controller comprises a processor and a non-transitory machine-readable storage medium comprising preprogrammed instructions executable by the processor to execute the dual-capture and release multiplexed immunoassay.

4. The instrument of claim 1, wherein the instrument further comprises an incusealer comprising framed sealing film for sealing at least one multi-vessel carrier plate.

5. The instrument of claim 4, wherein the framed sealing film has one or more perforated lines that are configured to allow easy and complete separation of the film from the frame by tearing.

6. The instrument of claim 4, wherein the framed sealing film is pierceable by a pipette tip.

7. The instrument of claim 1, wherein the robotic gantry further comprises an end-effector.

8. The instrument of claim 1, wherein the robotic gantry further comprises an end-effector and the robotic gantry is capable of moving the end-effector in three degrees of freedom in X, Y and Z axis.

9. The instrument of claim 7, wherein the end-effector, further comprises a multi-vessel carrier plates gripper.

10. The instrument of claim 7, wherein the end-effector, further comprises at least one laser position sensor.

11. The instrument of claim 10, wherein the end-effector, further comprises a barcode scanner.

12. The instrument of claim 1, wherein the reader is capable of identifying and/or quantifying nucleic acid reporters.

13. The instrument of claim 1, wherein the reader comprises a qPCR unit.

14. The instrument of claim 1, wherein the reader comprises a qPCR capable of preparing a pool library ready for next-gen sequencing (NGS).

15. The instrument of claim 1, wherein the instrument is capable of attomolar sensitivity detection of at least 5.

16. The instrument of claim 15, wherein the instrument has a broad dynamic measurement range up to 12 logs.
